(19) United States
(12) Patent Application Publication
(10) Pub. No.: US 2026/0021103 A1
(43) Pub. Date: Jan. 22, 2026

(54) IKZF2 AND CK1-ALPHA DEGRADING COMPOUNDS AND USES THEREOF

(71) Applicants: President and Fellows of Harvard College, Cambridge, MA (US); Memorial Sloan-Kettering Cancer Center, New York, NY (US); Memorial Hospital for Cancer and Allied Diseases, New York, NY (US); Sloan-Kettering Institute for Cancer Research, New York, NY (US)

(72) Inventors: Christina M. Woo, Cambridge, MA (US); David K. Miyamoto, Cambridge, MA (US); Nicole Curnutt, Cambridge, MA (US); Michael Kharas, New York, NY (US); Sun Mi Park, New York, NY (US)

(73) Assignees: President and Fellows of Harvard College, Cambridge, MA (US); Memorial Sloan-Kettering Cancer Center, New York, NY (US); Memorial Hospital for Cancer and Allied Diseases, New York, NY (US); Sloan-Kettering Institute for Cancer Research, New York, NY (US)

(21) Appl. No.: 19/111,754

(22) PCT Filed: Sep. 13, 2023
(86) PCT No.: PCT/US2023/032595

§ 371 Date: Mar. 13, 2025

Related U.S. Application Data
(60) Provisional application No. 63/406,582, filed on Sep. 14, 2022.
(60) Provisional application No. 63/439,757, filed on Jan. 18, 2023.

(51) Int. Cl. A61K 31/538; A61K 31/454; A61K 31/4709; A61P 35/02; C07D 401/04; C07D 401/14; C07D 405/14; C07D 409/14; C07D 413/14; C07D 417/14

(52) U.S. Cl. CPC A61K 31/538; A61K 31/454; A61K 31/4709; A61P 35/02; C07D 401/04; C07D 401/14; C07D 405/14; C07D 409/14; C07D 413/14; C07D 417/14

(57) ABSTRACT

Provided herein are compounds that promote targeted degradation of IKZF1, IKZF2, GSPT1, and/or CK1a, proteins whose activities are implicated in the pathology of certain cancers (e.g., acute myeloid leukemia). Also provided are pharmaceutical compositions comprising the compounds. Also provided are methods of treating cancer, and methods of promoting the degradation of IKZF1, IKZF2, GSPT1, and/or CK1a in a subject or biological sample by administering a compound or composition described herein.

### RELATED APPLICATIONS

This application claims priority under 35 U.S.C. § 119 (c) to U.S. Provisional applications, U.S. Ser. No. 63/406,582, filed Sep. 14, 2022, and U.S. Ser. No. 63/439,757, filed Jan. 18, 2023, each of which is incorporated herein by reference in its entirety.

### BACKGROUND

The IKAROS family of proteins, including IKZF1-5, are zinc finger transcription factors that are key regulators of hematopoiesis and cell-fate decisions in the development of the adaptive immune system. The IKAROS family sustains adult human stem cell character, and dysregulation is observed in a number of hematological malignancies, including lymphomas, leukemias, and myelomas. IKZF1 and IKZF3 are essential transcription factors in multiple myeloma (MM). IKZF1 has attracted attention as a target due to its role in hematopoiesis, immune function, and tumor suppression, as well as its complex role in the regulation of transcription and chromatin remodeling. IKZF2 controls lymphocyte development, promotes quiescence, and maintains the inhibitory function of regulatory T cells. IKZF2 is frequently deleted in hypodiploid B-cell acute lymphoblastic leukemias (B-ALLs) and expression of dominant negative isoforms have been reported in T-cell acute lymphoblastic leukemia (T-ALL) patients, suggesting a role for IKZF2 as a tumor suppressor in lymphoid malignancies. IKZF2 is also associated with chronic myelogenous leukemia (CML) and is overexpressed in thyroid, urothelial, and cervical cancers. However, the regulatory role of IKZF2 in adult stem cell maintenance and the difficulty in developing inhibitors for transcription factors has prevented its broader exploration as an anti-cancer target to date.

Recently, IKZF2 was established as a viable target in acute myeloid leukemia (AML) through the key role of IKZF2 in maintenance of leukemia stem cell (LSC) function. LSCs are resistant to chemotherapy and drive relapse in clinical studies, and therefore blocking self-renewal and differentiation could provide a novel therapeutic strategy. It was recently reported that IKZF2 was required for LSC function. IKZF2 depletion in human AML cell lines results in reduced proliferation, increased differentiation, increased apoptosis in vitro, and improved overall survival in mice. These properties establish IKZF2 as a bona fide anticancer target for the first time and creates a unique opportunity for the design of novel therapeutics targeting IKZF2.

Casein kinase 1 (CK1) proteins are important regulators of signal transduction, involved in diverse signaling pathways and acting in most cell types. In humans, the CK1 protein family includes seven isoforms, with CK1α being the most studied to date. All CK1 proteins are serine/threonine, monomeric protein kinases. CK1 family members are involved in various cellular processes, such as mitotic checkpoint signaling, DNA repair, apoptosis and p53 pathway, protein translation, circadian rhythm, endocytosis and autophagy, immune response and inflammation, and centrosome-associated processes, and they play a key regulatory role in several developmental pathways, such as Wnt. Hedgehog, NF-κB, and Yap/Taz signaling. Dysregulation of widespread regulatory network controlled by CK1 kinases may result in various pathological conditions, including cancer development, metastasis spreading, and neurodegenerative diseases. Recent studies have suggested that targeting CK1α is a promising approach for the treatment of cancers, such as hematological malignancies. However, investigation of such treatments have been hampered by a lack of suitable CK1α inhibitors.

GSPT1 is a small GTPase initially found essential for the G1 to S phase transition of the cell cycle and later reported to function as a polypeptide chain release factor 3 (eRF3). GSPT1 has been found to be overexpressed and oncogenic in a number of cancers, including hematopoietic malignancies. Acute leukemia cells are highly sensitive to GSPT1 degradation, strongly supporting this mechanism as a therapeutic approach to the treatment of hematological cancers.

Immunomodulatory agents (IMIDs), such as lenalidomide, have demonstrated efficacy in myelodysplastic syndromes and in multiple myeloma through targeting of IKAROS family members IKZF1 and IKZF3; however, these agents do not promote degradation of IKZF2 or CK1α, or show substantive efficacy in AML.

### SUMMARY

The present disclosure stems from the recognition that compounds that can degrade CK1α, IKZF1, IKZF2, and/or GSPT1, potential drug targets for the treatment of cancer (e.g., hematological malignancies), may be useful therapeutic agents in the treatment of disease (e.g., acute myeloid leukemia). The present disclosure describes new compounds that can induce the degradation of CK1α, IKZF1, IKZF2, and/or GSPT1 in cells. Thus, the present disclosure provides new compounds, compositions, kits, uses, and methods for the treatment of proliferative diseases (e.g., cancer, such as acute myeloid leukemia).

In one aspect, provided herein are compounds of Formula (I):

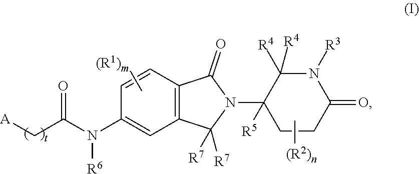

or a pharmaceutically acceptable salt, solvate, hydrate, polymorph, co-crystal, tautomer, stereoisomer, isotopically labeled derivative, or prodrug thereof, wherein:
each R¹ is independently halogen, —OR^{A}, or substituted or unsubstituted C₁-C₆ alkyl;
each R² is independently halogen or C₁-C₃ alkyl;
R³ is hydrogen or C₁-C₃ alkyl;
each R⁴ is independently hydrogen or C₁-C₃ alkyl; or two R⁴, together with the carbon atom to which they are attached, form a C═O, C₃-C₆ carbocycle, or a 4-6-membered heterocycle;
R⁵ is hydrogen, halogen, or C₁-C₃ alkyl;
R⁶ is hydrogen, substituted or unsubstituted acyl, substituted or unsubstituted alkyl, or a nitrogen protecting group;
each R⁷ is independently hydrogen or C₁-C₃ alkyl; or each R⁷, together with the carbon atom to which they are attached, form a C═O;
A is

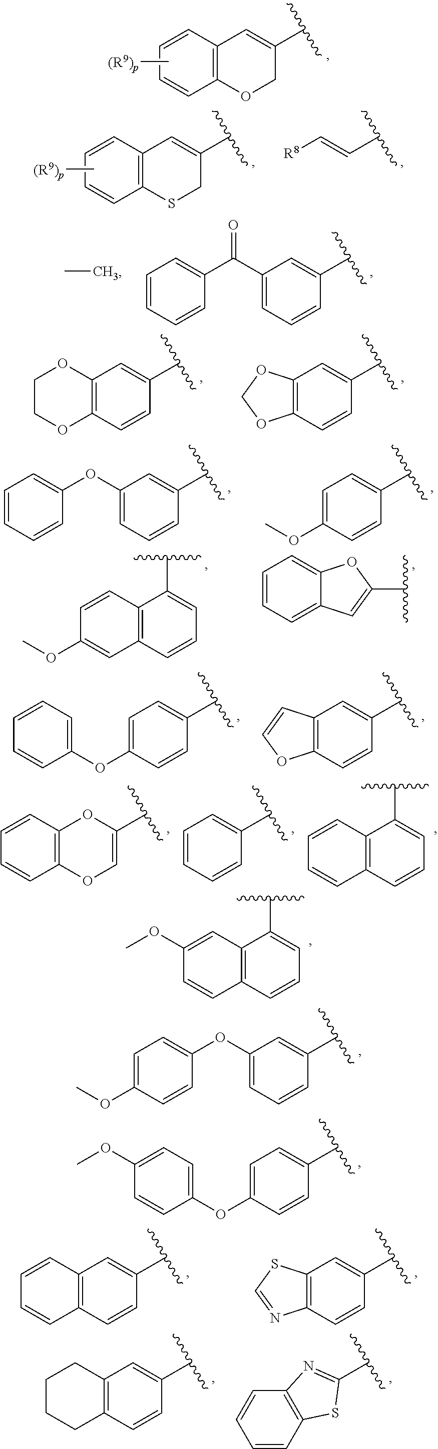

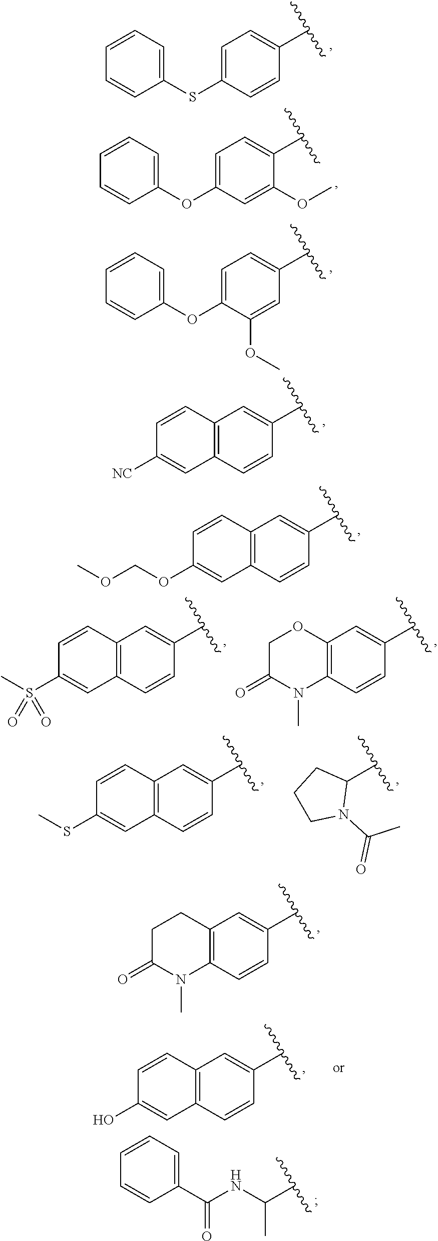

R⁸ is hydrogen, substituted or unsubstituted alkyl, substituted or unsubstituted heteroalkyl, substituted or unsubstituted carbocyclyl, substituted or unsubstituted heterocyclyl, substituted or unsubstituted aryl, or substituted or unsubstituted heteroaryl;
each R⁹ is independently halogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, substituted or unsubstituted heteroaliphatic, —OR^{A}, —N(R^{A})₂, —SR^{A}, —CN, —SCN, —C(═NR^{A})R^{A}, —C(═NR^{A})OR^{A}, —C(═NR^{A})N(R^{A})₂, C(═O)R^{A}, —C(═O)OR^{A}, —C(═O)N(R^{A})₂, —NO₂, —NR^{A}C(═O)R^{A}, —NR^{A}C(═O)OR^{A}, —NR^{A}C(═O)N(R^{A})₂, —NR^{A}C(═NR^{A})N(R^{A})₂, —OC(═O)R^{A}, —OC(═O)OR^{A}, —OC(═O)N(R^{A})₂, —NR^{A}S(O)₂R^{A}, —OS(O)₂R^{A}, or —S(O)₂R^{A}; or two R⁹ groups are joined to form a substituted or unsubstituted heterocyclyl ring, or a substituted or unsubstituted carbocyclyl ring;
each R^{A} is independently hydrogen, substituted or unsubstituted acyl, substituted or unsubstituted alkyl, substituted or unsubstituted alkenyl, substituted or unsubstituted alkynyl, substituted or unsubstituted heteroaliphatic, substituted or unsubstituted carbocyclyl, substituted or unsubstituted carbocyclylalkyl, substituted or unsubstituted heterocyclyl, substituted or unsubstituted aryl, substituted or unsubstituted aralkyl, substituted or unsubstituted heteroaryl, substituted or unsubstituted hetaralkyl, a nitrogen protecting group when attached to a nitrogen atom, an oxygen protecting group when attached to an oxygen atom, or a sulfur protecting group when attached to a sulfur atom, or two R^{A} groups are joined to form a substituted or unsubstituted heterocyclyl ring, or a substituted or unsubstituted heteroaryl ring;
m is 0, 1, 2 or 3;
n is 0, 1, or 2;
t is 0, 1, or 2; and
p is 0, 1, 2, 3, or 4;
provided that the compound is not of formula:

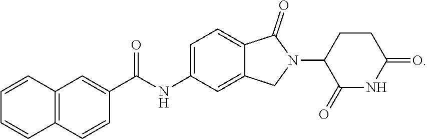 [SMILES: O=C1CCC(N2Cc3cc(NC(=O)c4ccc5ccccc5c4)ccc3C2=O)C(=O)N1]

In certain embodiments, the compound of Formula (I) is of Formula (I-a), (I-b), (I-c), (I-d), (I-e), (I-f), (I-g), (I-h), (I-i), (I-j), (I-k), (I-l), (I-m), (I-n), (I-o), (I-p), or (I-q):

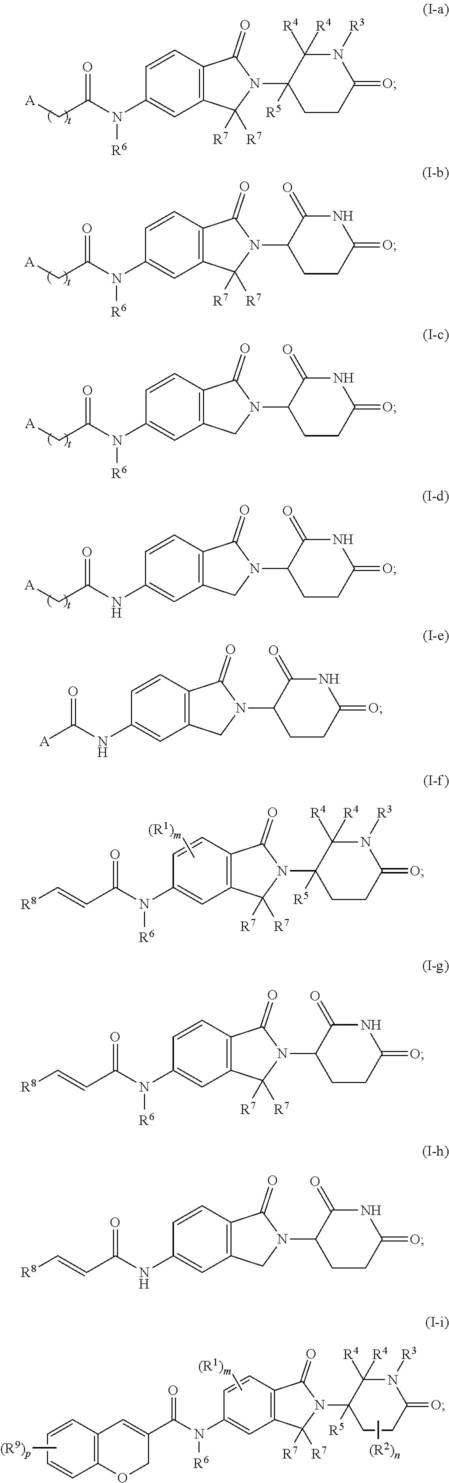

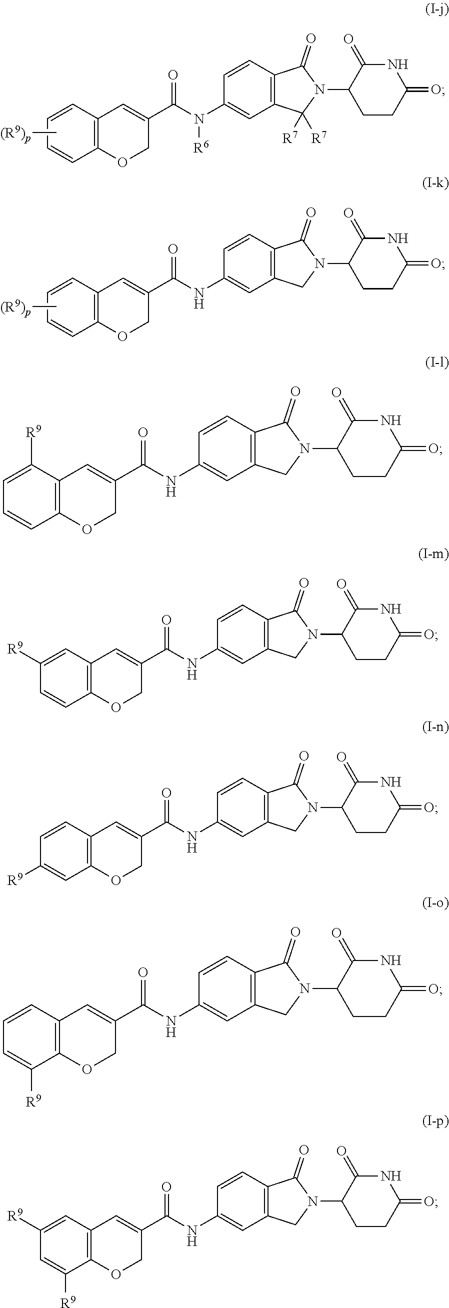

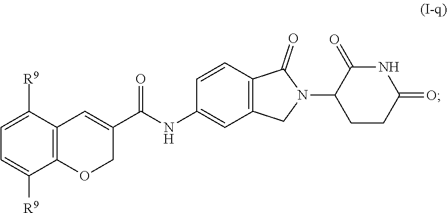

or a pharmaceutically acceptable salt, solvate, hydrate, polymorph, co-crystal, tautomer, stereoisomer, isotopically labeled derivative, or prodrug thereof.

Exemplary compounds of Formula (I) include, but are not limited to:

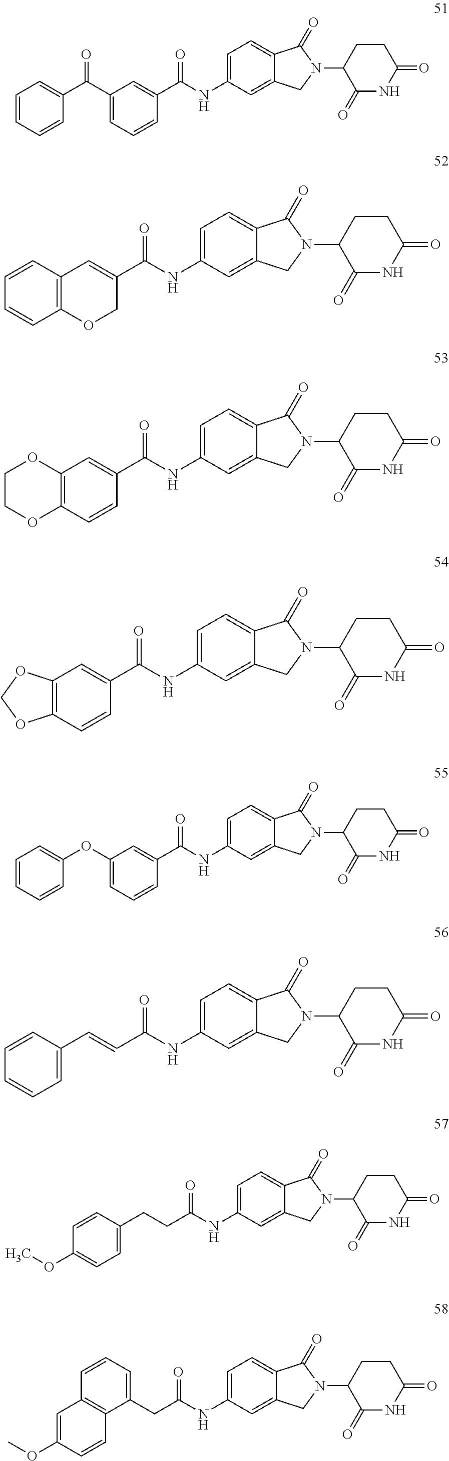

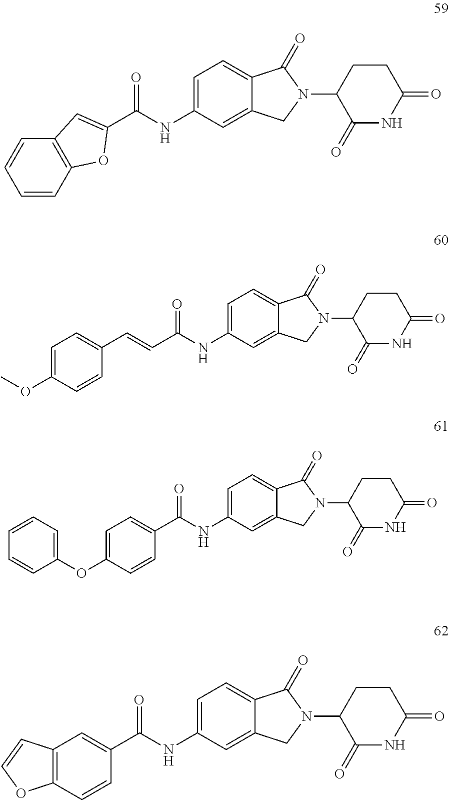 [SMILES: O=C1CCC(N2Cc3cc(NC(=O)c4cc5ccccc5o4)ccc3C2=O)C(=O)N1 ; COc1ccc(/C=C/C(=O)Nc2ccc3c(c2)CN(C2CCC(=O)NC2=O)C3=O)cc1 ; O=C1CCC(N2Cc3cc(NC(=O)c4ccc(Oc5ccccc5)cc4)ccc3C2=O)C(=O)N1 ; O=C1CCC(N2Cc3cc(NC(=O)c4ccc5occc5c4)ccc3C2=O)C(=O)N1]

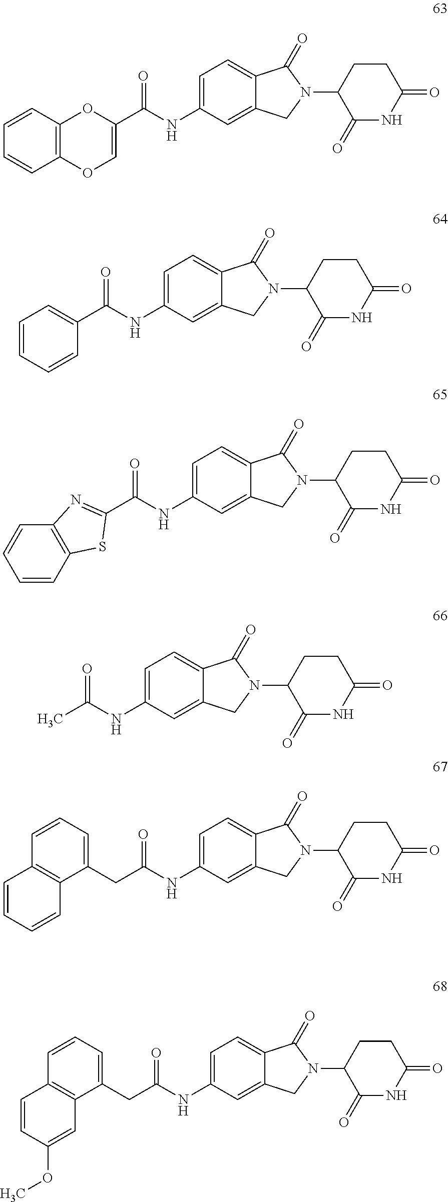 [SMILES: O=C1CCC(N2Cc3cc(NC(=O)C4=COc5ccccc5O4)ccc3C2=O)C(=O)N1 ; O=C1CCC(N2Cc3cc(NC(=O)c4ccccc4)ccc3C2=O)C(=O)N1 ; O=C1CCC(N2Cc3cc(NC(=O)c4nc5ccccc5s4)ccc3C2=O)C(=O)N1 ; CC(=O)Nc1ccc2c(c1)CN(C1CCC(=O)NC1=O)C2=O ; O=C1CCC(N2Cc3cc(NC(=O)Cc4cccc5ccccc45)ccc3C2=O)C(=O)N1 ; COc1ccc2cccc(CC(=O)Nc3ccc4c(c3)CN(C3CCC(=O)NC3=O)C4=O)c2c1]

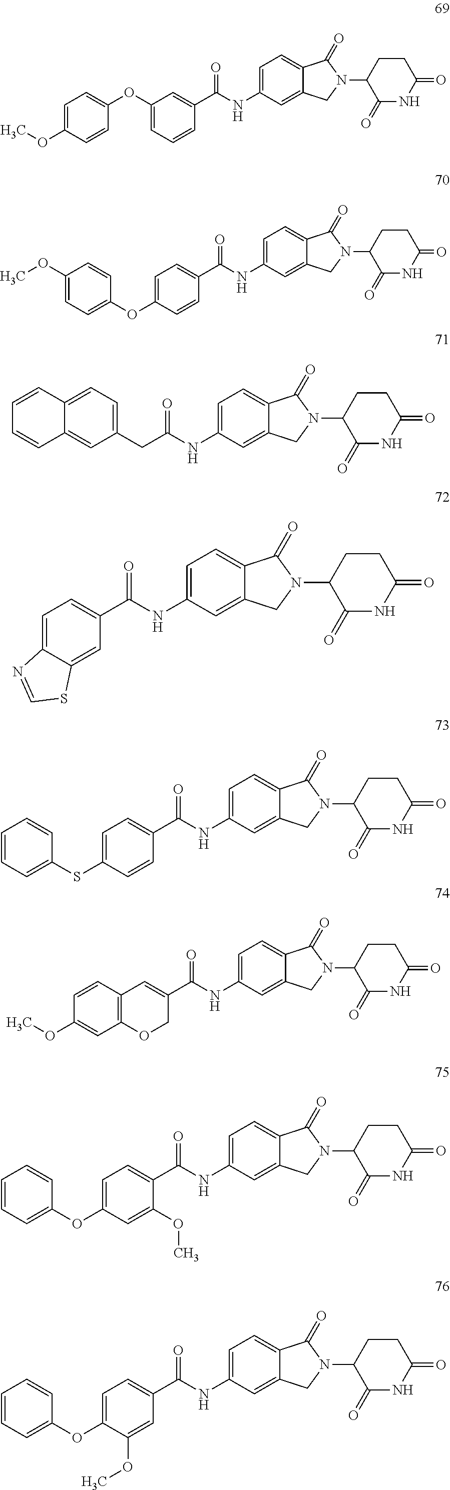

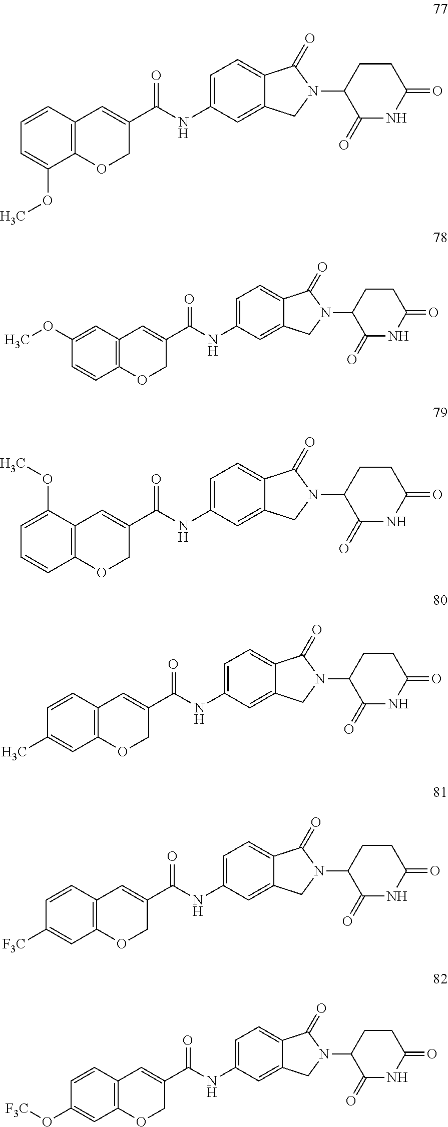 [SMILES: COc1cccc2c1OCC(C(=O)Nc1ccc3c(c1)CN(C1CCC(=O)NC1=O)C3=O)=C2 ; COc1ccc2c(c1)C=C(C(=O)Nc1ccc3c(c1)CN(C1CCC(=O)NC1=O)C3=O)CO2 ; COc1cccc2c1C=C(C(=O)Nc1ccc3c(c1)CN(C1CCC(=O)NC1=O)C3=O)CO2 ; Cc1ccc2c(c1)OCC(C(=O)Nc1ccc3c(c1)CN(C1CCC(=O)NC1=O)C3=O)=C2 ; O=C1CCC(N2Cc3cc(NC(=O)C4=Cc5ccc(C(F)(F)F)cc5OC4)ccc3C2=O)C(=O)N1 ; O=C1CCC(N2Cc3cc(NC(=O)C4=Cc5ccc(OC(F)(F)F)cc5OC4)ccc3C2=O)C(=O)N1]

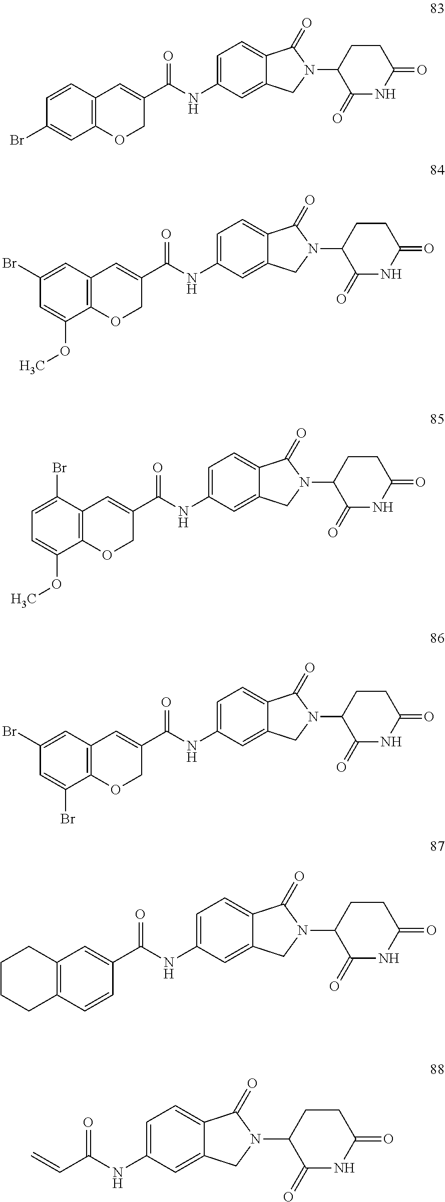 [SMILES: O=C1CCC(N2Cc3cc(NC(=O)C4=Cc5ccc(Br)cc5OC4)ccc3C2=O)C(=O)N1 ; COc1cc(Br)cc2c1OCC(C(=O)Nc1ccc3c(c1)CN(C1CCC(=O)NC1=O)C3=O)=C2 ; COc1ccc(Br)c2c1OCC(C(=O)Nc1ccc3c(c1)CN(C1CCC(=O)NC1=O)C3=O)=C2 ; O=C1CCC(N2Cc3cc(NC(=O)C4=Cc5cc(Br)cc(Br)c5OC4)ccc3C2=O)C(=O)N1 ; O=C1CCC(N2Cc3cc(NC(=O)c4ccc5c(c4)CCCC5)ccc3C2=O)C(=O)N1 ; C=CC(=O)Nc1ccc2c(c1)CN(C1CCC(=O)NC1=O)C2=O]

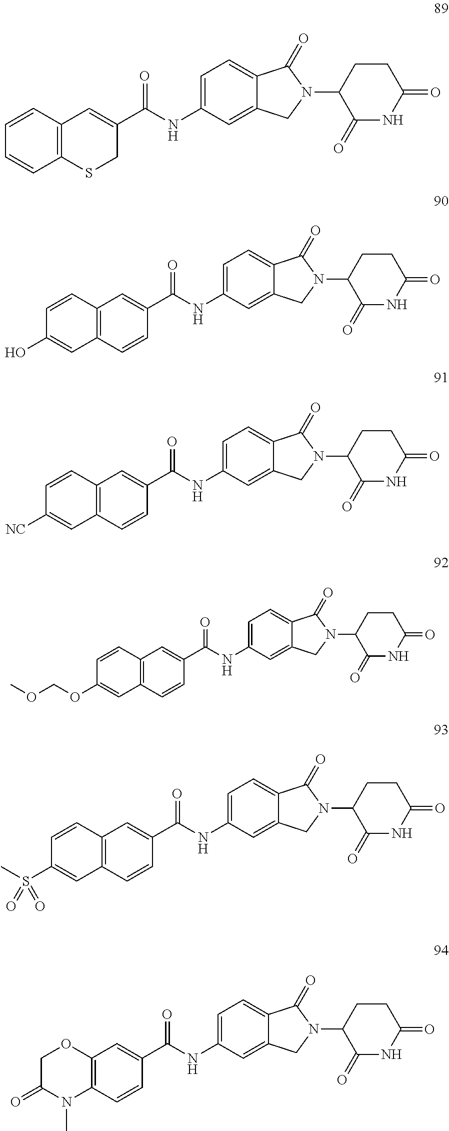 [SMILES: O=C1CCC(N2Cc3cc(NC(=O)C4=Cc5ccccc5SC4)ccc3C2=O)C(=O)N1 ; O=C1CCC(N2Cc3cc(NC(=O)c4ccc5cc(O)ccc5c4)ccc3C2=O)C(=O)N1 ; N#Cc1ccc2cc(C(=O)Nc3ccc4c(c3)CN(C3CCC(=O)NC3=O)C4=O)ccc2c1 ; COCOc1ccc2cc(C(=O)Nc3ccc4c(c3)CN(C3CCC(=O)NC3=O)C4=O)ccc2c1 ; CS(=O)(=O)c1ccc2cc(C(=O)Nc3ccc4c(c3)CN(C3CCC(=O)NC3=O)C4=O)ccc2c1 ; CN1C(=O)COc2cc(C(=O)Nc3ccc4c(c3)CN(C3CCC(=O)NC3=O)C4=O)ccc21]

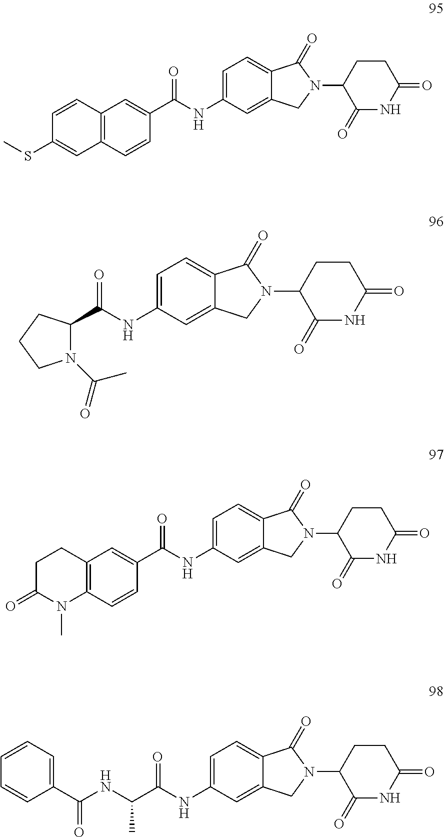 [SMILES: CSc1ccc2cc(C(=O)Nc3ccc4c(c3)CN(C3CCC(=O)NC3=O)C4=O)ccc2c1 ; CC(=O)N1CCC[C@H]1C(=O)Nc1ccc2c(c1)CN(C1CCC(=O)NC1=O)C2=O ; CN1C(=O)CCc2cc(C(=O)Nc3ccc4c(c3)CN(C3CCC(=O)NC3=O)C4=O)ccc21 ; C[C@H](NC(=O)c1ccccc1)C(=O)Nc1ccc2c(c1)CN(C1CCC(=O)NC1=O)C2=O]

and pharmaceutically acceptable salts, solvates, hydrates, polymorphs, co-crystals, tautomers, stereoisomers, isotopically labeled derivatives, and prodrugs thereof.

In another aspect, provided are pharmaceutical compositions comprising a compound of Formula (I), or a pharmaceutically acceptable salt thereof, and a pharmaceutically acceptable excipient.

In another aspect, provided are methods of treating a proliferative disease (e.g., cancer) in a subject in need thereof, the method comprising administering a compound of Formula (I), or a pharmaceutically acceptable salt thereof, or a pharmaceutical composition comprising a compound of Formula (I), to the subject.

In another aspect, provided are methods of treating cancer in a subject in need thereof, the method comprising administering a compound of Formula (I), or a pharmaceutically acceptable salt thereof, or a pharmaceutical composition comprising a compound of Formula (I), to the subject. In certain embodiments, the cancer is a hematological cancer. In certain embodiments, the cancer is a leukemia or a lymphoma. In certain embodiments, the cancer is primary effusion lymphoma (PEL), del(5q) myelodysplastic syndrome (MDS), chronic myeloid leukemia (CML), chronic lymphoid leukemia (CLL), acute myeloid leukemia (AML), hairy cell leukemia, chronic myelomonocytic leukemia (CMML), juvenile myelomonocyte leukemia (JMML), large granular lymphocytic leukemia (LGL), acute lymphoblastic leukemia (ALL), B-cell acute lymphoblastic leukemia (B-ALL), or T-cell acute lymphoblastic leukemia (T-ALL), B-cell lymphoma, diffuse large B-cell lymphoma (DLBCL), T-cell-lymphoma, Hodgkin's lymphoma, non-Hodgkin's lymphoma, hairy cell lymphoma, or Burkett's lymphoma.

In another aspect, provided are methods of promoting the degradation of a protein (e.g., a target protein), the method comprising contacting the protein with a compound of Formula (I), or a pharmaceutically acceptable salt thereof, or a pharmaceutical composition comprising a compound of Formula (I).

In another aspect, provided are methods of promoting the degradation of IKAROS family zinc finger 1 (IKZF1), the method comprising contacting IKZF1 with a compound of Formula (I), or a pharmaceutically acceptable salt thereof, or a pharmaceutical composition comprising a compound of Formula (I).

In another aspect, provided are methods of promoting the degradation of IKAROS family zinc finger 2 (IKZF2), the method comprising contacting IKZF2 with a compound of Formula (I), or a pharmaceutically acceptable salt thereof, or a pharmaceutical composition comprising a compound of Formula (I).

In another aspect, provided are methods of promoting the degradation of casein kinase 1α (CK1α), the method comprising contacting CK1α with a compound of Formula (I), or a pharmaceutically acceptable salt thereof, or a pharmaceutical composition comprising a compound of Formula (I).

In another aspect, provided are methods of promoting the degradation of G1 to S phase transition 1 protein (GSPT1), the method comprising contacting GSPT1 with a compound of Formula (I), or a pharmaceutically acceptable salt thereof, or a pharmaceutical composition comprising a compound of Formula (I).

In another aspect, provided are kits comprising a compound of Formula (I), or a pharmaceutically acceptable salt thereof, or a pharmaceutical composition comprising a compound of Formula (I). In certain embodiments, the kit further comprises instructions for administration (e.g., human administration) and/or use.

The details of certain embodiments of the invention are set forth in the Detailed Description of Certain Embodiments, as described below. Other features, objects, and advantages of the invention will be apparent from the Definitions, Examples, Figures, and Claims.

### BRIEF DESCRIPTION OF THE DRAWINGS

FIG. 1 shows western blots demonstrating the degradation of IKZF2 and CK1α by exemplary compounds.

FIG. 2 shows western blots demonstrating the degradation of IKZF2 and CK1α by exemplary compounds.

FIG. 3 shows western blots demonstrating the effect of exemplary compounds on IKZF1 and GSPT1.

FIG. 4 shows western blots demonstrating the degradation of IKZF2 and CK1α by exemplary compounds.

FIG. 5 is a plot showing cell viability of MOLM13 cells after exposure to exemplary compounds.

FIGS. 6A-K. DEG-77 has better efficacy than DEG-35 and can block leukemic stem cell activity. FIG. 6A: Western blot analysis of IKZF2 and CK1α in MOLM-13 cells treated with different concentrations of DEG-77. FIG. 6B-C: MOLM-13 cells were stained with myeloid markers (FIG. 6B) CD13/CD33 and (FIG. 6C) CD14/CD11b and then measured by flow cytometry for differentiation at day 2 posttreatment with 100 nM DEG-35 or DEG-77.

FIG. 6D: Apoptosis was measured by flow cytometry using Annexin V and DAPI as marker for apoptosis. Results shown in FIG. 6B-D were combined from at least three independent experiments, Mean±SEM. Student's t test, **p<0.01, ***p<0.001, ****p<0.0001. FIG. 6E: Cell viability assay was performed in MOLM-13 cells treated with DEG-35 and DEG-77 to obtain IC₅₀. FIG. 6F: Western blot analysis showing reduction of HOXA9 and MYC by DEG-77 treatment at 24 hr posttreatment in MOLM-13 cells. FIG. 6G: Scheme showing strategy for analyzing pharmacodynamics of targets of DEG-77 in the MLL-AF9 Crbn^{1391V} mice model. FIG. 6H: Western blot analysis of targets IKZF2 and CK1α with ACTIN as loading control of leukemic bone marrow cells from mice treated with DEG-77 at 223 mg/kg. 24 hr, 48 hr, and 72 hr post-treatment. FIG. 6I: Colony assay of leukemic cells from bone marrow and spleen of mice treated with DMSO or DEG-77 24 hrs post treatment. Student's t test, *p<0.05. FIG. 6J: Survival curve of BL6 mice transplanted with MLL-AF9 Crbn^{1391V} cells treated with DMSO or 223 mg/kg DEG-77. ****p<0.0001 log-rank test. FIG. 6K: Survival curve of BL6 mice secondarily transplanted with cells from DMSO or DEG-77 treated mice in FIG. 6I ****p<0.0001 log-rank test.

FIGS. 7A-I. DEG-77 exhibits improved efficacy in vivo over DEG-35. FIG. 7A: Western blot analysis of known neo-substrates of immunomodulatory drugs following treatment of MOLM-13 cells with DEG-77. FIG. 7B: Western blot analysis of CK1c and IKZF2 in MOLM-13 cells treated 1 h with pomalidomide followed by treatment with DEG-77. FIG. 7C: In vitro analysis of DEG-77 binding to CRBN using an HTR-FRET assay. FIG. 7D: Flow cytometry analysis of HEK293T cells expressing GFP-tagged CK1α. FIG. 7E: qRT-PCR analysis of CK1α and IKZF2 mRNA levels in MOLM-13 cells after 24 h treatment with DEG-77. FIG. 7F: Plasma concentration of DEG-77 over a 24 hr period in mice after a single intraperitoneal injection of DEG-77 at 5 mg/kg. The plasma concentrations represent the mean+/−SEM from three mice per time point. FIG. 7G: Graph showing apoptosis frequency. FIG. 7H: Graph showing Mac+Gr+ frequency of leukemic cells from peripheral blood, bone marrow and spleen from mice treated with DMSO or DEG-77 24 hrs post treatment were measured by flow cytometry. Student's t test, *p<0.05, **p<0.01. FIG. 7I: Averaged body weight of MLL-AF9 Crbn^{1391V} cells-transplanted mice treated with either DMSO or 223 mg/kg of DEG-77. Body weight was taken from first day of treatment until sacrificed.

FIGS. 8A-C. Substrate scope of new scaffolds. FIG. 8A: Substrate scope of compounds DEG-35, 51-56, 59, 61-64, 66, 67, 72, and 87 in HEK293T cells expressing the indicated protein tagged with eGFP. Cells were treated with 100 nM compound for 24 h. FIG. 8B: Substrate scope of compounds DEG-74, 77, 78, 79, 80, 81, 82, 83, 84, and 86 in HEK293T cells expressing the indicated protein tagged with eGFP. Cells were treated with 100 nM compound for 24 h. FIG. 8C: Quantitative global proteomics in MOLM-13 cells treated with 50 nM DEG-77 for 2 h.

FIGS. 9A-G. DEG-77 has efficacy in indications beyond AML. FIG. 9A: 5-day MTT assay of Me-DEG-35, DEG-35, DEG-77, and MDM2 inhibitor idasanutlin in A2780 (top) and OCI-LY3 (bottom) cells. FIG. 9B: 5-day MTT assay of A2780 and OCI-LY3 dosed with DEG-35, DEG-77, GSPT1 degrader CC-90009, and MDM2 inhibitor idasanutlin. FIG. 9C: 5-day MTT assay in small cell lung cancer cell line NCI-H1048. FIG. 9D: Western blots of CK1α and GSPT1 in A2780 and OCI-LY3 cells dosed with Me-DEG-35, DEG-35, and DEG-77 for 24 h. FIG. 9E: Western blot of IKZF2 in A2780 cells dosed with DEG-35 and DEG-77 for 24 h. FIG. 9F: RT-qPCR analysis of CK1α and p53 transcriptional targets Bax and p21 in A2780 cells dosed with 1 μM DEG-77 for a 24 h period. FIG. 9G: RT-qPCR analysis of CK1α and p53 transcriptional targets Bax and p21 in OCI-LY3 cells dosed with 1 μM DEG-77 for a 24 h period.

FIGS. 10A-C. Evaluation of NCI-H1048 cells and p53 activation in A2780 and OCI-LY3. FIG. 10A: Western blot of NCI-H1048 cells treated with Me-DEG-35, DEG-35, DEG-77 and CC-90009 for 24 h. FIG. 10B: Western blot analysis of p53 activation via p53, p21, MDM2, and Bax protein levels in A2780 and OCI-LY3 cells treated with DEG-77 for 24 h. FIG. 10C: 5-day MTT assay of Me-DEG-35, DEG-35, and DEG-77 in NCI-H1048 cells.

FIG. 11A: Western blot analysis showing degradation of CK1α and IKZF2 in MLL-AF9 Crbn^{1391V} by different concentration of DEG-77 after 24 h. FIG. 11B: plot showing apoptosis. FIG. 11C: plot showing differentiation measured by flow cytometry of cells stained with Annexin V and myeloid markers Mac1/Gr1 respectively. Flow cytometry was performed 24 h post-treatment with indicated concentrations of DEG-35 or 10 μM lenalidomide. FIG. 11D-G: mRNA levels of p53 targets measured by qPCR analysis in MLL-AF9 Crbn^{1391V} cells treated with DEG-77 and lenalidomide for 24 h. FIG. 11H: Normalized colony assay of four primary AML patient cells and five normal bone marrow/CD34+ HSPC cord blood cells treated with DEG-35 plated in H443 methylcellulose for 14 days. For statistics used in (B-C, D-G, H), results shown were combined from at least three independent experiments, Mean±SEM. Student's t test, *p<0.05, **p<0.01. ***p<0.001, ****p<0.0001.

FIG. 12A: Survival curve of NSG mice transplanted with MOLM-13 cells and treated with DMSO (n=10) or DEG-77 (n=9) treated at 223 mg/kg weekly. **p<0.01 log-rank test. FIG. 12B: Western blot analysis of MOLM-13 cells sorted from bone marrow of NSG mice 24 h after drug treatment. FIG. 12C: Survival curve of NSG mice transplanted with AML PDX #2 treated with DMSO (n=15) or DEG-77 (n=5) at dose of 223 mg/kg, weekly from 4 weeks post-transplant. **p<0.01 log-rank test. FIG. 12D: Engraftment frequency of AML PDX cells measured by flow cytometry by hCD45 using bone marrow aspirates at 4 weeks posttransplant (left panel) before and (right panel) one week after a single dose of DEG-77 treatment. Student's t test, ***p<0.001. FIG. 12E: Survival curve of non-irradiated Crbn^{1391V} mice transplanted with MLL-AF9 Crbn^{1391V} cells treated with DMSO or DEG-77 at dose of 223 mg/kg weekly. **p<0.01 log-rank test. n=10 mice for each group. FIG. 12F: Disease burden shown by frequency of GFP positive cells (MLL-AF9 Crbn^{1391V} cells) in bone marrow or spleen at time of sacrifice. Student's t test, *p<0.05.

FIG. 13A: qRT-PCR analysis of CSNK1A1 and IKZF2 mRNA levels in MOLM-13 cells after 24 h treatment with DEG-77. FIG. 13B: mRNA levels of p53 targets quantified by qPCR analysis of MOLM-13 cells treated with 1 μM DEG-77 for 24 h. Results from three independent experiments were performed. Mean±SEM. Student's t test, *p<0.05. ***p<0.001. FIG. 13C: Kinetics of p53 activation shown by Western blot analysis of p53 and its targets p21 and MDM2 in MOLM-13 cells treated with 1 μM DEG-77 for 5 days. FIG. 13D: Kinetics of cell cycle of MOLM-13 cells in FIG. 13C. FIG. 13E: Apoptosis and differentiation markers. FIG. 13F: CD13/CD11b measured by flow cytometry in MOLM-13 treated with 1 μM DEG-77. FIG. 13G: CD14/CD33b measured by flow cytometry in MOLM-13 treated with 1 μM DEG-77. For results in (A-G) three independent experiments were performed. Mean±SEM. Student's t test, *p<0.05, **p<0.01, ***p<0.001, ****p<0.0001. FIG. 13H: Wright-Giemsa Staining of MOLM-13 cells treated with DEG-77 for 5 days. Scale bar denoting 50 μM is shown in pictures.

FIG. 14A: MOLM-13 engraftment measured by hCD45% in bone marrow of mice in FIG. 12A (DMSO on left and DEG-77 on right). FIG. 14B: Averaged body weight of NSG mice transplanted with MOLM-13 cells treated with DMSO or DEG-77 in FIG. 12A. Body weight was taken from first day of treatment until sacrificed. FIG. 14C and FIG. 14D: Spleen (14C) and liver (14D) weights of AML PDX #2 mice shown in FIG. 12C. Student's t test, **p<0.01, ****p<0.0001. FIG. 14E: Flow gatings for stem and progenitor compartment in Crbn^{1391V} mice. FIG. 14F and FIG. 14G: Frequency of stem cell compartment (14F) and progenitor compartment (14G) of Crbn^{1391V} mice treated with DMSO or DEG-77 for four rounds at 223 mg/kg weekly. FIGS. 14H and 14I: Absolute numbers for LSK (14H) or HSC (14I) of Crbn^{1391V} mice treated with DMSO or DEG-77 (DMSO on left and DEG-77 on right). FIG. 14J: Flow gatings for mature populations of the crythroid, lymphoid and myeloid linkages. FIGS. 14K and 14L: Frequency of crythroid (14K), lymphoid (14L) and myeloid lineages of Crbn^{1391V} mice treated with DMSO or DEG-77 for three rounds at 223 mg/kg weekly (DMSO on left and DEG-77 on right). FIGS. 14M, 14N, and 14O: Counts for WBC (14M), platelets (14N) and RBC (14O) of Crbn^{1391V} mice treated with DMSO or DEG-77 (DMSO on left and DEG-77 on right). Results in (FIGS. 14F-O) are from 4 or more biological replicates. Mean±SEM. Student's t test, *p<0.05, **p<0.01. ***p<0.001. FIG. 14P: Body weights of Crbn^{1391V} mice treated with DMSO or DEG-77 (DMSO on left and DEG-77 on right).

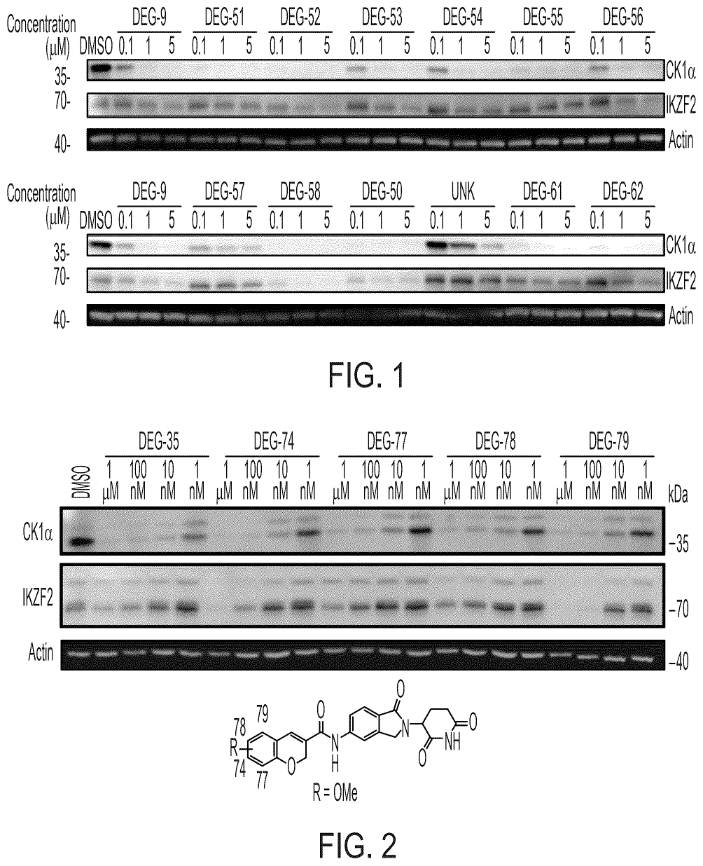

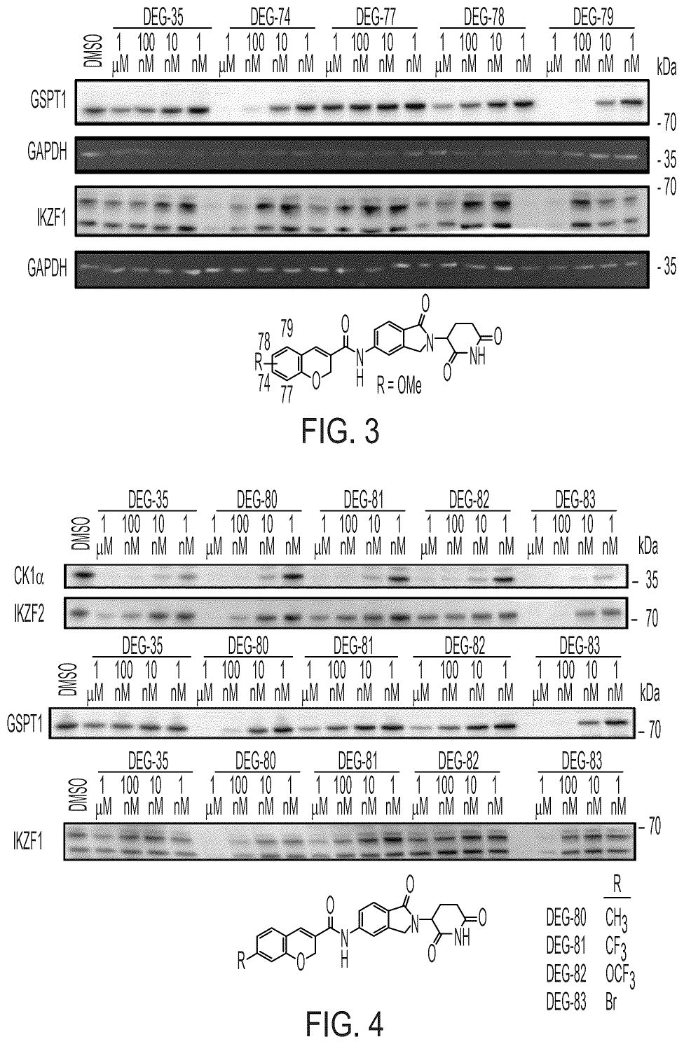

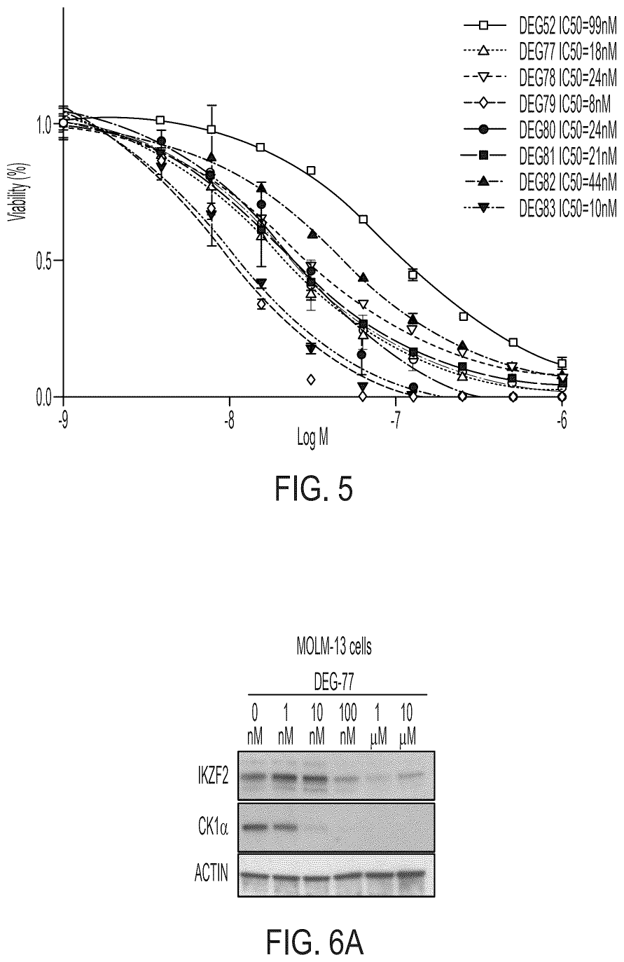

### DEFINITIONS

### Chemical Definitions

Definitions of specific functional groups and chemical terms are described in more detail below. The chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Organic Chemistry, Thomas Sorrell, University Science Books, Sausalito, 1999; Smith and March, March's Advanced Organic Chemistry, 5^{th} Edition, John Wiley & Sons, Inc., New York, 2001; Larock, Comprehensive Organic Transformations, VCH Publishers, Inc., New York, 1989; and Carruthers, Some Modern Methods of Organic Synthesis, 3^{rd} Edition, Cambridge University Press, Cambridge, 1987.

Compounds described herein can comprise one or more asymmetric centers, and thus can exist in various stereoisomeric forms, e.g., enantiomers and/or diastereomers. For example, the compounds described herein can be in the form of an individual enantiomer, diastereomer or geometric isomer, or can be in the form of a mixture of stereoisomers, including racemic mixtures and mixtures enriched in one or more stereoisomer. Isomers can be isolated from mixtures by methods known to those skilled in the art, including chiral high pressure liquid chromatography (HPLC) and the formation and crystallization of chiral salts; or preferred isomers can be prepared by asymmetric syntheses. See, for example, Jacques et al., Enantiomers, Racemates and Resolutions (Wiley Interscience, New York, 1981); Wilen et al., Tetrahedron 33:2725 (1977); Eliel, E. L. Stereochemistry of Carbon Compounds (McGraw-Hill, NY, 1962); and Wilen, S. H., Tables of Resolving Agents and Optical Resolutions p. 268 (E. L. Eliel, Ed., Univ. of Notre Dame Press, Notre Dame, IN 1972). The invention additionally encompasses compounds as individual isomers substantially free of other isomers, and alternatively, as mixtures of various isomers.

In a formula, [symbol] is a single bond where the stereochemistry of the moieties immediately attached thereto is not specified, [symbol] is absent or a single bond, and [symbol] or [symbol] is a single or double bond.

Unless otherwise stated, structures depicted herein are also meant to include compounds that differ only in the presence of one or more isotopically enriched atoms. For example, compounds having the present structures except for the replacement of hydrogen by deuterium or tritium, replacement of ¹⁹F with ¹⁸F, or the replacement of ¹²C with ¹³C or ¹⁴C are within the scope of the disclosure. Such compounds are useful, for example, as analytical tools or probes in biological assays.

When a range of values is listed, it is intended to encompass each value and sub-range within the range. For example “C₁₋₆ alkyl” is intended to encompass, C₁, C₂, C₃, C₄, C₅, C₆, C₁₋₆, C₁₋₅, C₁₋₄, C₁₋₃, C₁₋₂, C₂₋₆, C₂₋₅, C₂₋₄, C₂₋₃, C₃₋₆, C₃₋₅, C₃₋₄, C₄₋₆, C₄₋₅, and C₅₋₆ alkyl.

The term “aliphatic” refers to alkyl, alkenyl, alkynyl, and carbocyclic groups. Likewise, the term “heteroaliphatic” refers to heteroalkyl, heteroalkenyl, heteroalkynyl, and heterocyclic groups.

The term “alkyl” refers to a radical of a straight-chain or branched saturated hydrocarbon group having from 1 to 10 carbon atoms (“C₁₋₁₀ alkyl”). In some embodiments, an alkyl group has 1 to 9 carbon atoms (“C₁₋₉ alkyl”). In some embodiments, an alkyl group has 1 to 8 carbon atoms (“C₁₋₈ alkyl”). In some embodiments, an alkyl group has 1 to 7 carbon atoms (“C₁₋₇ alkyl”). In some embodiments, an alkyl group has 1 to 6 carbon atoms (“C₁₋₆ alkyl”). In some embodiments, an alkyl group has 1 to 5 carbon atoms (“C₁₋₅ alkyl”). In some embodiments, an alkyl group has 1 to 4 carbon atoms (“C₁₋₄ alkyl”). In some embodiments, an alkyl group has 1 to 3 carbon atoms (“C₁₋₃ alkyl”). In some embodiments, an alkyl group has 1 to 2 carbon atoms (“C₁₋₂ alkyl”). In some embodiments, an alkyl group has 1 carbon atom (“C₁ alkyl”). In some embodiments, an alkyl group has 2 to 6 carbon atoms (“C₂₋₆ alkyl”). Examples of C₁₋₆ alkyl groups include methyl (C₁), ethyl (C₂), propyl (C₃) (e.g., n-propyl, isopropyl), butyl (C₄) (e.g., n-butyl, tert-butyl, sec-butyl, iso-butyl), pentyl (C₅) (e.g., n-pentyl, 3-pentanyl, amyl, neopentyl, 3-methyl-2-butanyl, tertiary amyl), and hexyl (C₆) (e.g., n-hexyl). Additional examples of alkyl groups include n-heptyl (C₇), n-octyl (C₈), and the like. Unless otherwise specified, each instance of an alkyl group is independently unsubstituted (an “unsubstituted alkyl”) or substituted (a “substituted alkyl”) with one or more substituents (e.g., halogen, such as F). In certain embodiments, the alkyl group is an unsubstituted C₁₋₁₀ alkyl (such as unsubstituted C₁₋₆ alkyl, e.g., —CH₃ (Me), unsubstituted ethyl (Et), unsubstituted propyl (Pr, e.g., unsubstituted n-propyl (n-Pr), unsubstituted isopropyl (i-Pr)), unsubstituted butyl (Bu, e.g., unsubstituted n-butyl (n-Bu), unsubstituted tert-butyl (tert-Bu or t-Bu), unsubstituted sec-butyl (sec-Bu), unsubstituted isobutyl (i-Bu)). In certain embodiments, the alkyl group is a substituted C₁₋₁₀ alkyl (such as substituted C₁₋₆ alkyl, e.g., —CF₃, Bn).

The term “haloalkyl” is a substituted alkyl group, wherein one or more of the hydrogen atoms are independently replaced by a halogen, e.g., fluoro, bromo, chloro, or iodo. In some embodiments, the haloalkyl moiety has 1 to 8 carbon atoms (“C₁₋₈ haloalkyl”). In some embodiments, the haloalkyl moiety has 1 to 6 carbon atoms (“C₁₋₆ haloalkyl”). In some embodiments, the haloalkyl moiety has 1 to 4 carbon atoms (“C₁₋₄ haloalkyl”). In some embodiments, the haloalkyl moiety has 1 to 3 carbon atoms (“C₁₋₃ haloalkyl”). In some embodiments, the haloalkyl moiety has 1 to 2 carbon atoms (“C₁₋₂ haloalkyl”). Examples of haloalkyl groups include —CHF₂, —CH₂F, —CF₃, —CH₂CF₃, —CF₂CF₃, —CF₂CF₂CF₃, —CCl₃, —CFCl₂, —CF₂Cl, and the like.

The term “heteroalkyl” refers to an alkyl group, which further includes at least one heteroatom (e.g., 1, 2, 3, or 4 heteroatoms) selected from oxygen, nitrogen, or sulfur within (i.e., inserted between adjacent carbon atoms of) and/or placed at one or more terminal position(s) of the parent chain. In certain embodiments, a heteroalkyl group refers to a saturated group having from 1 to 20 carbon atoms and 1 or more heteroatoms within the parent chain (“heteroC₁₋₂₀ alkyl”). In some embodiments, a heteroalkyl group is a saturated group having 1 to 18 carbon atoms and 1 or more heteroatoms within the parent chain (“heteroC₁₋₁₈ alkyl”). In some embodiments, a heteroalkyl group is a saturated group having 1 to 16 carbon atoms and 1 or more heteroatoms within the parent chain (“heteroC₁₋₁₆ alkyl”). In some embodiments, a heteroalkyl group is a saturated group having 1 to 14 carbon atoms and 1 or more heteroatoms within the parent chain (“heteroC₁₋₁₄ alkyl”). In some embodiments, a heteroalkyl group is a saturated group having 1 to 12 carbon atoms and 1 or more heteroatoms within the parent chain (“heteroC₁₋₁₂ alkyl”). In some embodiments, a heteroalkyl group is a saturated group having 1 to 10 carbon atoms and 1 or more heteroatoms within the parent chain (“heteroC₁₋₁₀ alkyl”). In some embodiments, a heteroalkyl group is a saturated group having 1 to 8 carbon atoms and 1 or more heteroatoms within the parent chain (“heteroC₁₋₈ alkyl”). In some embodiments, a heteroalkyl group is a saturated group having 1 to 6 carbon atoms and 1 or more heteroatoms within the parent chain (“heteroC₁₋₆ alkyl”). In some embodiments, a heteroalkyl group is a saturated group having 1 to 4 carbon atoms and 1 or 2 heteroatoms within the parent chain (“heteroC₁₋₄ alkyl”). In some embodiments, a heteroalkyl group is a saturated group having 1 to 3 carbon atoms and 1 heteroatom within the parent chain (“heteroC₁₋₃ alkyl”). In some embodiments, a heteroalkyl group is a saturated group having 1 to 2 carbon atoms and 1 heteroatom within the parent chain (“heteroC₁₋₂ alkyl”). In some embodiments, a heteroalkyl group is a saturated group having 1 carbon atom and 1 heteroatom (“heteroC₁ alkyl”). In some embodiments, the heteroalkyl group defined herein is a partially unsaturated group having 1 or more heteroatoms within the parent chain and at least one unsaturated carbon, such as a carbonyl group. For example, a heteroalkyl group may comprise an amide or ester functionality in its parent chain such that one or more carbon atoms are unsaturated carbonyl groups. Unless otherwise specified, each instance of a heteroalkyl group is independently unsubstituted (an “unsubstituted heteroalkyl”) or substituted (a “substituted heteroalkyl”) with one or more substituents. In certain embodiments, the heteroalkyl group is an unsubstituted heteroC₁₋₂₀ alkyl. In certain embodiments, the heteroalkyl group is an unsubstituted heteroC₁₋₁₀ alkyl. In certain embodiments, the heteroalkyl group is a substituted heteroC₁₋₂₀ alkyl. In certain embodiments, the heteroalkyl group is an unsubstituted heteroC₁₋₁₀ alkyl.

The term “alkenyl” refers to a radical of a straight-chain or branched hydrocarbon group having from 2 to 10 carbon atoms and one or more carbon-carbon double bonds (e.g., 1, 2, 3, or 4 double bonds). In some embodiments, an alkenyl group has 2 to 9 carbon atoms (“C₂₋₉ alkenyl”). In some embodiments, an alkenyl group has 2 to 8 carbon atoms (“C₂₋₈ alkenyl”). In some embodiments, an alkenyl group has 2 to 7 carbon atoms (“C₂₋₇ alkenyl”). In some embodiments, an alkenyl group has 2 to 6 carbon atoms (“C₂₋₆ alkenyl”). In some embodiments, an alkenyl group has 2 to 5 carbon atoms (“C₂₋₅ alkenyl”). In some embodiments, an alkenyl group has 2 to 4 carbon atoms (“C₂₋₄ alkenyl”). In some embodiments, an alkenyl group has 2 to 3 carbon atoms (“C₂₋₃ alkenyl”). In some embodiments, an alkenyl group has 2 carbon atoms (“C₂ alkenyl”). The one or more carbon-carbon double bonds can be internal (such as in 2-butenyl) or terminal (such as in 1-butenyl). Examples of C₂₋₄ alkenyl groups include ethenyl (C₂), 1-propenyl (C₃), 2-propenyl (C₃), 1-butenyl (C₄), 2-butenyl (C₄), butadienyl (C₄), and the like. Examples of C₂₋₆ alkenyl groups include the aforementioned C₂₋₄ alkenyl groups as well as pentenyl (C₅), pentadienyl (C₅), hexenyl (C₆), and the like. Additional examples of alkenyl include heptenyl (C₇), octenyl (C₈), octatrienyl (C₈), and the like. Unless otherwise specified, each instance of an alkenyl group is independently unsubstituted (an “unsubstituted alkenyl”) or substituted (a “substituted alkenyl”) with one or more substituents. In certain embodiments, the alkenyl group is an unsubstituted C₂₋₁₀ alkenyl. In certain embodiments, the alkenyl group is a substituted C₂₋₁₀ alkenyl. In an alkenyl group, a C═C double bond for which the stereochemistry is not specified (e.g., —CH═CHCH₃ or

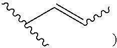

may be an (E)- or (Z)-double bond.

The term “heteroalkenyl” refers to an alkenyl group, which further includes at least one heteroatom (e.g., 1, 2, 3, or 4 heteroatoms) selected from oxygen, nitrogen, or sulfur within (i.e., inserted between adjacent carbon atoms of) and/or placed at one or more terminal position(s) of the parent chain. In certain embodiments, a heteroalkenyl group refers to a group having from 2 to 10 carbon atoms, at least one double bond, and 1 or more heteroatoms within the parent chain (“heteroC₂₋₁₀ alkenyl”). In some embodiments, a heteroalkenyl group has 2 to 9 carbon atoms at least one double bond, and 1 or more heteroatoms within the parent chain (“heteroC₂₋₉ alkenyl”). In some embodiments, a heteroalkenyl group has 2 to 8 carbon atoms, at least one double bond, and 1 or more heteroatoms within the parent chain (“heteroC₂₋₈ alkenyl”). In some embodiments, a heteroalkenyl group has 2 to 7 carbon atoms, at least one double bond, and 1 or more heteroatoms within the parent chain (“heteroC₂₋₇ alkenyl”). In some embodiments, a heteroalkenyl group has 2 to 6 carbon atoms, at least one double bond, and 1 or more heteroatoms within the parent chain (“heteroC₂₋₆ alkenyl”). In some embodiments, a heteroalkenyl group has 2 to 5 carbon atoms, at least one double bond, and 1 or 2 heteroatoms within the parent chain (“heteroC₂₋₅ alkenyl”). In some embodiments, a heteroalkenyl group has 2 to 4 carbon atoms, at least one double bond, and 1 or 2 heteroatoms within the parent chain (“heteroC₂₋₄ alkenyl”). In some embodiments, a heteroalkenyl group has 2 to 3 carbon atoms, at least one double bond, and 1 heteroatom within the parent chain (“heteroC₂₋₃ alkenyl”). In some embodiments, a heteroalkenyl group has 2 to 6 carbon atoms, at least one double bond, and 1 or 2 heteroatoms within the parent chain (“heteroC₂₋₆ alkenyl”). Unless otherwise specified, each instance of a heteroalkenyl group is independently unsubstituted (an “unsubstituted heteroalkenyl”) or substituted (a “substituted heteroalkenyl”) with one or more substituents. In certain embodiments, the heteroalkenyl group is an unsubstituted heteroC₂₋₁₀ alkenyl. In certain embodiments, the heteroalkenyl group is a substituted heteroC₂₋₁₀ alkenyl.

The term “alkynyl” refers to a radical of a straight-chain or branched hydrocarbon group having from 2 to 10 carbon atoms and one or more carbon-carbon triple bonds (e.g., 1, 2, 3, or 4 triple bonds) (“C₂₋₁₀ alkynyl”). In some embodiments, an alkynyl group has 2 to 9 carbon atoms (“C₂₋₉ alkynyl”). In some embodiments, an alkynyl group has 2 to 8 carbon atoms (“C₂₋₈ alkynyl”). In some embodiments, an alkynyl group has 2 to 7 carbon atoms (“C₂₋₇ alkynyl”). In some embodiments, an alkynyl group has 2 to 6 carbon atoms (“C₂₋₆ alkynyl”). In some embodiments, an alkynyl group has 2 to 5 carbon atoms (“C₂₋₅ alkynyl”). In some embodiments, an alkynyl group has 2 to 4 carbon atoms (“C₂₋₄ alkynyl”). In some embodiments, an alkynyl group has 2 to 3 carbon atoms (“C₂₋₃ alkynyl”). In some embodiments, an alkynyl group has 2 carbon atoms (“C₂ alkynyl”). The one or more carbon-carbon triple bonds can be internal (such as in 2-butynyl) or terminal (such as in 1-butynyl). Examples of C₂₋₄ alkynyl groups include, without limitation, ethynyl (C₂), 1-propynyl (C₃), 2-propynyl (C₃), 1-butynyl (C₄), 2-butynyl (C₄), and the like. Examples of C₂₋₆ alkenyl groups include the aforementioned C₂₋₄ alkynyl groups as well as pentynyl (C₅), hexynyl (C₆), and the like. Additional examples of alkynyl include heptynyl (C₇), octynyl (C₈), and the like. Unless otherwise specified, each instance of an alkynyl group is independently unsubstituted (an “unsubstituted alkynyl”) or substituted (a “substituted alkynyl”) with one or more substituents. In certain embodiments, the alkynyl group is an unsubstituted C₂₋₁₀ alkynyl. In certain embodiments, the alkynyl group is a substituted C₂₋₁₀ alkynyl.

The term “heteroalkynyl” refers to an alkynyl group, which further includes at least one heteroatom (e.g., 1, 2, 3, or 4 heteroatoms) selected from oxygen, nitrogen, or sulfur within (i.e., inserted between adjacent carbon atoms of) and/or placed at one or more terminal position(s) of the parent chain. In certain embodiments, a heteroalkynyl group refers to a group having from 2 to 10 carbon atoms, at least one triple bond, and 1 or more heteroatoms within the parent chain (“heteroC₂₋₁₀ alkynyl”). In some embodiments, a heteroalkynyl group has 2 to 9 carbon atoms, at least one triple bond, and 1 or more heteroatoms within the parent chain (“heteroC₂₋₉ alkynyl”). In some embodiments, a heteroalkynyl group has 2 to 8 carbon atoms, at least one triple bond, and 1 or more heteroatoms within the parent chain (“heteroC₂₋₈ alkynyl”). In some embodiments, a heteroalkynyl group has 2 to 7 carbon atoms, at least one triple bond, and 1 or more heteroatoms within the parent chain (“heteroC₂₋₇ alkynyl”). In some embodiments, a heteroalkynyl group has 2 to 6 carbon atoms, at least one triple bond, and 1 or more heteroatoms within the parent chain (“heteroC₂₋₆ alkynyl”). In some embodiments, a heteroalkynyl group has 2 to 5 carbon atoms, at least one triple bond, and 1 or 2 heteroatoms within the parent chain (“heteroC₂₋₅ alkynyl”). In some embodiments, a heteroalkynyl group has 2 to 4 carbon atoms, at least one triple bond, and 1 or 2 heteroatoms within the parent chain (“heteroC₂₋₄ alkynyl”). In some embodiments, a heteroalkynyl group has 2 to 3 carbon atoms, at least one triple bond, and 1 heteroatom within the parent chain (“heteroC₂₋₃ alkynyl”). In some embodiments, a heteroalkynyl group has 2 to 6 carbon atoms, at least one triple bond, and 1 or 2 heteroatoms within the parent chain (“heteroC₂₋₆ alkynyl”). Unless otherwise specified, each instance of a heteroalkynyl group is independently unsubstituted (an “unsubstituted heteroalkynyl”) or substituted (a “substituted heteroalkynyl”) with one or more substituents. In certain embodiments, the heteroalkynyl group is an unsubstituted heteroC₂₋₁₀ alkynyl. In certain embodiments, the heteroalkynyl group is a substituted heteroC₂₋₁₀ alkynyl.

The term “carbocyclyl” or “carbocyclic” refers to a radical of a non-aromatic cyclic hydrocarbon group having from 3 to 14 ring carbon atoms (“C₃₋₁₄ carbocyclyl”) and zero heteroatoms in the non-aromatic ring system. In some embodiments, a carbocyclyl group has 3 to 10 ring carbon atoms (“C₃₋₁₀ carbocyclyl”). In some embodiments, a carbocyclyl group has 3 to 8 ring carbon atoms (“C₃₋₈ carbocyclyl”). In some embodiments, a carbocyclyl group has 3 to 7 ring carbon atoms (“C₃₋₇ carbocyclyl”). In some embodiments, a carbocyclyl group has 3 to 6 ring carbon atoms (“C₃₋₆ carbocyclyl”). In some embodiments, a carbocyclyl group has 4 to 6 ring carbon atoms (“C₄₋₆ carbocyclyl”). In some embodiments, a carbocyclyl group has 5 to 6 ring carbon atoms (“C₅₋₆ carbocyclyl”). In some embodiments, a carbocyclyl group has 5 to 10 ring carbon atoms (“C₅₋₁₀ carbocyclyl”). Exemplary C₃₋₆ carbocyclyl groups include, without limitation, cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), and the like. Exemplary C₃₋₈ carbocyclyl groups include, without limitation, the aforementioned C₃₋₆ carbocyclyl groups as well as cycloheptyl (C₇), cycloheptenyl (C₇), cycloheptadienyl (C₇), cycloheptatrienyl (C₇), cyclooctyl (C₈), cyclooctenyl (C₈), bicyclo[2.2.1]heptanyl (C₇), bicyclo[2.2.2]octanyl (C₈), and the like. Exemplary C₃₋₁₀ carbocyclyl groups include, without limitation, the aforementioned C₃₋₈ carbocyclyl groups as well as cyclononyl (C₉), cyclononenyl (C₉), cyclodecyl (C₁₀), cyclodecenyl (C₁₀), octahydro-1H-indenyl (C₉), decahydronaphthalenyl (C₁₀), spiro[4.5]decanyl (C₁₀), and the like. As the foregoing examples illustrate, in certain embodiments, the carbocyclyl group is either monocyclic (“monocyclic carbocyclyl”) or polycyclic (e.g., containing a fused, bridged or spiro ring system such as a bicyclic system (“bicyclic carbocyclyl”) or tricyclic system (“tricyclic carbocyclyl”)) and can be saturated or can contain one or more carbon-carbon double or triple bonds. “Carbocyclyl” also includes ring systems wherein the carbocyclyl ring, as defined above, is fused with one or more aryl or heteroaryl groups wherein the point of attachment is on the carbocyclyl ring, and in such instances, the number of carbons continue to designate the number of carbons in the carbocyclic ring system. Unless otherwise specified, each instance of a carbocyclyl group is independently unsubstituted (an “unsubstituted carbocyclyl”) or substituted (a “substituted carbocyclyl”) with one or more substituents. In certain embodiments, the carbocyclyl group is an unsubstituted C₃₋₁₄ carbocyclyl. In certain embodiments, the carbocyclyl group is a substituted C₃₋₁₄ carbocyclyl.

In some embodiments, “carbocyclyl” is a monocyclic, saturated carbocyclyl group having from 3 to 14 ring carbon atoms (“C₃₋₁₄ cycloalkyl”). In some embodiments, a cycloalkyl group has 3 to 10 ring carbon atoms (“C₃₋₁₀ cycloalkyl”). In some embodiments, a cycloalkyl group has 3 to 8 ring carbon atoms (“C₃₋₈ cycloalkyl”). In some embodiments, a cycloalkyl group has 3 to 6 ring carbon atoms (“C₃₋₆ cycloalkyl”). In some embodiments, a cycloalkyl group has 4 to 6 ring carbon atoms (“C₄₋₆ cycloalkyl”). In some embodiments, a cycloalkyl group has 5 to 6 ring carbon atoms (“C₅₋₆ cycloalkyl”). In some embodiments, a cycloalkyl group has 5 to 10 ring carbon atoms (“C₅₋₁₀ cycloalkyl”). Examples of C₅₋₆ cycloalkyl groups include cyclopentyl (C₅) and cyclohexyl (C₅). Examples of C₃₋₆ cycloalkyl groups include the aforementioned C₅₋₆ cycloalkyl groups as well as cyclopropyl (C₃) and cyclobutyl (C₄). Examples of C₃₋₈ cycloalkyl groups include the aforementioned C₃₋₆ cycloalkyl groups as well as cycloheptyl (C₇) and cyclooctyl (C₈). Unless otherwise specified, each instance of a cycloalkyl group is independently unsubstituted (an “unsubstituted cycloalkyl”) or substituted (a “substituted cycloalkyl”) with one or more substituents. In certain embodiments, the cycloalkyl group is an unsubstituted C₃₋₁₄ cycloalkyl. In certain embodiments, the cycloalkyl group is a substituted C₃₋₁₄ cycloalkyl.

The term “heterocyclyl” or “heterocyclic” refers to a radical of a 3- to 14-membered non-aromatic ring system having ring carbon atoms and 1 to 4 ring heteroatoms, wherein each heteroatom is independently selected from nitrogen, oxygen, and sulfur (“3-14 membered heterocyclyl”). In heterocyclyl groups that contain one or more nitrogen atoms, the point of attachment can be a carbon or nitrogen atom, as valency permits. A heterocyclyl group can either be monocyclic (“monocyclic heterocyclyl”) or polycyclic (e.g., a fused, bridged or spiro ring system such as a bicyclic system (“bicyclic heterocyclyl”) or tricyclic system (“tricyclic heterocyclyl”)), and can be saturated or can contain one or more carbon-carbon double or triple bonds. Heterocyclyl polycyclic ring systems can include one or more heteroatoms in one or both rings. “Heterocyclyl” also includes ring systems wherein the heterocyclyl ring, as defined above, is fused with one or more carbocyclyl groups wherein the point of attachment is either on the carbocyclyl or heterocyclyl ring, or ring systems wherein the heterocyclyl ring, as defined above, is fused with one or more aryl or heteroaryl groups, wherein the point of attachment is on the heterocyclyl ring, and in such instances, the number of ring members continue to designate the number of ring members in the heterocyclyl ring system. Unless otherwise specified, each instance of heterocyclyl is independently unsubstituted (an “unsubstituted heterocyclyl”) or substituted (a “substituted heterocyclyl”) with one or more substituents. In certain embodiments, the heterocyclyl group is an unsubstituted 3-14 membered heterocyclyl. In certain embodiments, the heterocyclyl group is a substituted 3-14 membered heterocyclyl.

In some embodiments, a heterocyclyl group is a 5-10 membered non-aromatic ring system having ring carbon atoms and 1-4 ring heteroatoms, wherein each heteroatom is independently selected from nitrogen, oxygen, and sulfur (“5-10 membered heterocyclyl”). In some embodiments, a heterocyclyl group is a 5-8 membered non-aromatic ring system having ring carbon atoms and 1-4 ring heteroatoms, wherein each heteroatom is independently selected from nitrogen, oxygen, and sulfur (“5-8 membered heterocyclyl”). In some embodiments, a heterocyclyl group is a 5-6 membered non-aromatic ring system having ring carbon atoms and 1-4 ring heteroatoms, wherein each heteroatom is independently selected from nitrogen, oxygen, and sulfur (“5-6 membered heterocyclyl”). In some embodiments, the 5-6 membered heterocyclyl has 1-3 ring heteroatoms selected from nitrogen, oxygen, and sulfur. In some embodiments, the 5-6 membered heterocyclyl has 1-2 ring heteroatoms selected from nitrogen, oxygen, and sulfur. In some embodiments, the 5-6 membered heterocyclyl has 1 ring heteroatom selected from nitrogen, oxygen, and sulfur.

Exemplary 3-membered heterocyclyl groups containing 1 heteroatom include, without limitation, azirdinyl, oxiranyl, and thiiranyl. Exemplary 4-membered heterocyclyl groups containing 1 heteroatom include, without limitation, azetidinyl, oxetanyl, and thietanyl. Exemplary 5-membered heterocyclyl groups containing 1 heteroatom include, without limitation, tetrahydrofuranyl, dihydrofuranyl, tetrahydrothiophenyl, dihydrothiophenyl, pyrrolidinyl, dihydropyrrolyl, and pyrrolyl-2,5-dione. Exemplary 5-membered heterocyclyl groups containing 2 heteroatoms include, without limitation, dioxolanyl, oxathiolanyl and dithiolanyl. Exemplary 5-membered heterocyclyl groups containing 3 heteroatoms include, without limitation, triazolinyl, oxadiazolinyl, and thiadiazolinyl. Exemplary 6-membered heterocyclyl groups containing 1 heteroatom include, without limitation, piperidinyl, tetrahydropyranyl, dihydropyridinyl, and thianyl. Exemplary 6-membered heterocyclyl groups containing 2 heteroatoms include, without limitation, piperazinyl, morpholinyl, dithianyl, and dioxanyl. Exemplary 6-membered heterocyclyl groups containing 3 heteroatoms include, without limitation, triazinyl. Exemplary 7-membered heterocyclyl groups containing 1 heteroatom include, without limitation, azepanyl, oxepanyl and thiepanyl. Exemplary 8-membered heterocyclyl groups containing 1 heteroatom include, without limitation, azocanyl, oxecanyl and thiocanyl. Exemplary bicyclic heterocyclyl groups include, without limitation, indolinyl, isoindolinyl, dihydrobenzofuranyl, dihydrobenzothienyl, tetrahydrobenzothienyl, tetrahydrobenzofuranyl, tetrahydroindolyl, tetrahydroquinolinyl, tetrahydroisoquinolinyl, decahydroquinolinyl, decahydroisoquinolinyl, octahydrochromenyl, octahydroisochromenyl, decahydronaphthyridinyl, decahydro-1,8-naphthyridinyl, octahydropyrrolo[3,2-b]pyrrole, indolinyl, phthalimidyl, naphthalimidyl, chromanyl, chromenyl, 1H-benzo[e][1,4]diazepinyl, 1,4,5,7-tetrahydropyrano[3,4-b]pyrrolyl, 5,6-dihydro-4H-furo[3,2-b]pyrrolyl, 6,7-dihydro-5H-furo[3,2-b]pyranyl, 5,7-dihydro-4H-thieno[2,3-c]pyranyl, 2,3-dihydro-1H-pyrrolo[2,3-b]pyridinyl, 2,3-dihydrofuro[2,3-b]pyridinyl, 4,5,6,7-tetrahydro-1H-pyrrolo[2,3-b]pyridinyl, 4,5,6,7-tetrahydrofuro[3,2-c]pyridinyl, 4,5,6,7-tetrahydrothieno[3,2-b]pyridinyl, 1,2,3,4-tetrahydro-1,6-naphthyridinyl, and the like.

The term “aryl” refers to a radical of a monocyclic or polycyclic (e.g., bicyclic or tricyclic) 4n+2 aromatic ring system (e.g., having 6, 10, or 14 π electrons shared in a cyclic array) having 6-14 ring carbon atoms and zero heteroatoms provided in the aromatic ring system (“C₆₋₁₄ aryl”). In some embodiments, an aryl group has 6 ring carbon atoms (“C₆ aryl”; e.g., phenyl). In some embodiments, an aryl group has 10 ring carbon atoms (“C₁₀ aryl”; e.g., naphthyl such as 1-naphthyl and 2-naphthyl). In some embodiments, an aryl group has 14 ring carbon atoms (“C₁₄ aryl”; e.g., anthracyl). “Aryl” also includes ring systems wherein the aryl ring, as defined above, is fused with one or more carbocyclyl or heterocyclyl groups wherein the radical or point of attachment is on the aryl ring, and in such instances, the number of carbon atoms continue to designate the number of carbon atoms in the aryl ring system. Unless otherwise specified, each instance of an aryl group is independently unsubstituted (an “unsubstituted aryl”) or substituted (a “substituted aryl”) with one or more substituents. In certain embodiments, the aryl group is an unsubstituted C₆₋₁₄ aryl. In certain embodiments, the aryl group is a substituted C₆₋₁₄ aryl.

“Aralkyl” is a subset of “alkyl” and refers to an alkyl group substituted by an aryl group, wherein the point of attachment is on the alkyl moiety.

The term “heteroaryl” refers to a radical of a 5-14 membered monocyclic or polycyclic (e.g., bicyclic, tricyclic) 4n+2 aromatic ring system (e.g., having 6, 10, or 14 π electrons shared in a cyclic array) having ring carbon atoms and 1-4 ring heteroatoms provided in the aromatic ring system, wherein each heteroatom is independently selected from nitrogen, oxygen, and sulfur (“5-14 membered heteroaryl”). In heteroaryl groups that contain one or more nitrogen atoms, the point of attachment can be a carbon or nitrogen atom, as valency permits. Heteroaryl polycyclic ring systems can include one or more heteroatoms in one or both rings. “Heteroaryl” includes ring systems wherein the heteroaryl ring, as defined above, is fused with one or more carbocyclyl or heterocyclyl groups wherein the point of attachment is on the heteroaryl ring, and in such instances, the number of ring members continue to designate the number of ring members in the heteroaryl ring system. “Heteroaryl” also includes ring systems wherein the heteroaryl ring, as defined above, is fused with one or more aryl groups wherein the point of attachment is either on the aryl or heteroaryl ring, and in such instances, the number of ring members designates the number of ring members in the fused polycyclic (aryl/heteroaryl) ring system. Polycyclic heteroaryl groups wherein one ring does not contain a heteroatom (e.g., indolyl, quinolinyl, carbazolyl, and the like) the point of attachment can be on either ring, i.e., either the ring bearing a heteroatom (e.g., 2-indolyl) or the ring that does not contain a heteroatom (e.g., 5-indolyl).

In some embodiments, a heteroaryl group is a 5-10 membered aromatic ring system having ring carbon atoms and 1-4 ring heteroatoms provided in the aromatic ring system, wherein each heteroatom is independently selected from nitrogen, oxygen, and sulfur (“5-10 membered heteroaryl”). In some embodiments, a heteroaryl group is a 5-8 membered aromatic ring system having ring carbon atoms and 1-4 ring heteroatoms provided in the aromatic ring system, wherein each heteroatom is independently selected from nitrogen, oxygen, and sulfur (“5-8 membered heteroaryl”). In some embodiments, a heteroaryl group is a 5-6 membered aromatic ring system having ring carbon atoms and 1-4 ring heteroatoms provided in the aromatic ring system, wherein each heteroatom is independently selected from nitrogen, oxygen, and sulfur (“5-6 membered heteroaryl”). In some embodiments, the 5-6 membered heteroaryl has 1-3 ring heteroatoms selected from nitrogen, oxygen, and sulfur. In some embodiments, the 5-6 membered heteroaryl has 1-2 ring heteroatoms selected from nitrogen, oxygen, and sulfur. In some embodiments, the 5-6 membered heteroaryl has 1 ring heteroatom selected from nitrogen, oxygen, and sulfur. Unless otherwise specified, each instance of a heteroaryl group is independently unsubstituted (an “unsubstituted heteroaryl”) or substituted (a “substituted heteroaryl”) with one or more substituents. In certain embodiments, the heteroaryl group is an unsubstituted 5-14 membered heteroaryl. In certain embodiments, the heteroaryl group is a substituted 5-14 membered heteroaryl.

Exemplary 5-membered heteroaryl groups containing 1 heteroatom include, without limitation, pyrrolyl, furanyl, and thiophenyl. Exemplary 5-membered heteroaryl groups containing 2 heteroatoms include, without limitation, imidazolyl, pyrazolyl, oxazolyl, isoxazolyl, thiazolyl, and isothiazolyl. Exemplary 5-membered heteroaryl groups containing 3 heteroatoms include, without limitation, triazolyl, oxadiazolyl, and thiadiazolyl. Exemplary 5-membered heteroaryl groups containing 4 heteroatoms include, without limitation, tetrazolyl. Exemplary 6-membered heteroaryl groups containing 1 heteroatom include, without limitation, pyridinyl. Exemplary 6-membered heteroaryl groups containing 2 heteroatoms include, without limitation, pyridazinyl, pyrimidinyl, and pyrazinyl. Exemplary 6-membered heteroaryl groups containing 3 or 4 heteroatoms include, without limitation, triazinyl and tetrazinyl, respectively. Exemplary 7-membered heteroaryl groups containing 1 heteroatom include, without limitation, azepinyl, oxepinyl, and thiepinyl. Exemplary 5,6-bicyclic heteroaryl groups include, without limitation, indolyl, isoindolyl, indazolyl, benzotriazolyl, benzothiophenyl, isobenzothiophenyl, benzofuranyl, benzoisofuranyl, benzimidazolyl, benzoxazolyl, benzisoxazolyl, benzoxadiazolyl, benzthiazolyl, benzisothiazolyl, benzthiadiazolyl, indolizinyl, and purinyl. Exemplary 6,6-bicyclic heteroaryl groups include, without limitation, naphthyridinyl, pteridinyl, quinolinyl, isoquinolinyl, cinnolinyl, quinoxalinyl, phthalazinyl, and quinazolinyl. Exemplary tricyclic heteroaryl groups include, without limitation, phenanthridinyl, dibenzofuranyl, carbazolyl, acridinyl, phenothiazinyl, phenoxazinyl, and phenazinyl.

“Heteroaralkyl” is a subset of “alkyl” and refers to an alkyl group substituted by a heteroaryl group, wherein the point of attachment is on the alkyl moiety.

The term “unsaturated bond” refers to a double or triple bond.

The term “unsaturated” or “partially unsaturated” refers to a moiety that includes at least one double or triple bond.

The term “saturated” refers to a moiety that does not contain a double or triple bond, i.e., the moiety only contains single bonds.

Affixing the suffix “-ene” to a group indicates the group is a divalent moiety, e.g., alkylene is the divalent moiety of alkyl, alkenylene is the divalent moiety of alkenyl, alkynylene is the divalent moiety of alkynyl, heteroalkylene is the divalent moiety of heteroalkyl, heteroalkenylene is the divalent moiety of heteroalkenyl, heteroalkynylene is the divalent moiety of heteroalkynyl, carbocyclylene is the divalent moiety of carbocyclyl, heterocyclylene is the divalent moiety of heterocyclyl, arylene is the divalent moiety of aryl, and heteroarylene is the divalent moiety of heteroaryl.

A group is optionally substituted unless expressly provided otherwise. The term “optionally substituted” refers to being substituted or unsubstituted. In certain embodiments, alkyl, alkenyl, alkynyl, heteroalkyl, heteroalkenyl, heteroalkynyl, carbocyclyl, heterocyclyl, aryl, and heteroaryl groups are optionally substituted. “Optionally substituted” refers to a group which may be substituted or unsubstituted (e.g., “substituted” or “unsubstituted” alkyl, “substituted” or “unsubstituted” alkenyl, “substituted” or “unsubstituted” alkynyl, “substituted” or “unsubstituted” heteroalkyl, “substituted” or “unsubstituted” heteroalkenyl, “substituted” or “unsubstituted” heteroalkynyl, “substituted” or “unsubstituted” carbocyclyl, “substituted” or “unsubstituted” heterocyclyl, “substituted” or “unsubstituted” aryl or “substituted” or “unsubstituted” heteroaryl group). In general, the term “substituted” means that at least one hydrogen present on a group is replaced with a permissible substituent, e.g., a substituent which upon substitution results in a stable compound, e.g., a compound which does not spontaneously undergo transformation such as by rearrangement, cyclization, elimination, or other reaction. Unless otherwise indicated, a “substituted” group has a substituent at one or more substitutable positions of the group, and when more than one position in any given structure is substituted, the substituent is either the same or different at each position. The term “substituted” is contemplated to include substitution with all permissible substituents of organic compounds, and includes any of the substituents described herein that results in the formation of a stable compound. The present invention contemplates any and all such combinations in order to arrive at a stable compound. For purposes of this invention, heteroatoms such as nitrogen may have hydrogen substituents and/or any suitable substituent as described herein which satisfy the valencies of the heteroatoms and results in the formation of a stable moiety. The invention is not intended to be limited in any manner by the exemplary substituents described herein.

Exemplary carbon atom substituents include, but are not limited to, halogen, —CN, —NO₂, —N₃, —SO₂H, —SO₃H, —OH, —OR^{aa}, —ON(R^{bb})₂, —N(R^{bb})₂, —N(R^{bb})₃⁺X⁻, —N(OR^{cc})R^{bb}, —SH, —SR^{aa}, —SSR^{cc}, —C(═O)R^{aa}, —CO₂H, —CHO, —C(OR^{cc})₃, —CO₂R^{aa}, —OC(═O)R^{aa}, —OCO₂R^{aa}, —C(═O)N(R^{bb})₂, —OC(═O)N(R^{bb})₂, —NR^{bb}C(═O)R^{aa}, —NR^{bb}CO₂R^{aa}, —NR^{bb}C(═O)N(R^{bb})₂, —C(═NR^{bb})R^{aa}, —C(═NR^{bb})OR^{aa}, —OC(═NR^{bb})R^{aa}, —OC(═NR^{bb})OR^{aa}, —C(═NR^{bb})N(R^{bb})₂, —OC(═NR^{bb})N(R^{bb})₂, —NR^{bb}C(═NR^{bb})N(R^{bb})₂, —C(═O)NR^{bb}SO₂R^{aa}, —NR^{bb}SO₂R^{aa}, —SO₂N(R^{bb})₂, —SO₂R^{aa}, —SO₂OR^{aa}, —OSO₂R^{aa}, —S(═O)R^{aa}, —OS(═O)R^{aa}, —Si(R^{aa})₃, —OSi(R^{aa})₃—C(═S)N(R^{bb})₂, —C(═O)SR^{aa}, —C(═S)SR^{aa}, —SC(═S)SR^{aa}, —SC(═O)SR^{aa}, —OC(═O)SR^{aa}, —SC(═O)OR^{aa}, —SC(═O)R^{aa}, —P(═O)(R^{aa})₂, —P(═O)(OR^{aa})₂, —OP(═O)(R^{aa})₂, —OP(═O)(OR^{cc})₂, —P(═O)(N(R^{bb})₂)₂, —OP(═O)(N(R^{bb})₂)₂, —NR^{bb}P(═O)(R^{aa})₂, —NR^{bb}P(═O)(OR)₂, —NR^{bb}P(═O)(N(R^{bb})₂)₂, —P(R^{cc})₂, —P(OR^{cc})₂, —P(R^{cc})₃⁺X⁻, —P(OR^{cc})₃⁺X⁻, —P(R^{cc})₄, —P(OR^{cc})₄, —OP(R^{cc})₂, —OP(R^{cc})₃⁺X⁻, —OP(OR)₂, —OP(OR^{cc})₃⁺X⁻, —OP(R^{cc})₄, —OP(OR^{cc})₄, —B(R^{aa})₂, —B(OR^{cc})₂, —BR^{aa} (OR^{cc}), C₁₋₁₀ alkyl, C₁₋₁₀ perhaloalkyl, C₂₋₁₀ alkenyl, C₂₋₁₀ alkynyl, heteroC₁₋₁₀ alkyl, heteroC₂₋₁₀ alkenyl, heteroC₂₋₁₀ alkynyl, C₃₋₁₀ carbocyclyl, 3-14 membered heterocyclyl, C₆₋₁₄ aryl, and 5-14 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups; wherein X⁻ is a counterion;
or two geminal hydrogens on a carbon atom are replaced with the group ═O, ═S, ═NN(R^{bb})₂, ═NNR^{bb}C(═O)R^{aa}, ═NNR^{bb}C(═O)OR^{aa}, ═NNR^{bb}S(═O)₂R^{aa}, ═NR^{bb}, or ═NOR^{cc};
each instance of R^{aa} is, independently, selected from C₁₋₁₀ alkyl, C₁₋₁₀ perhaloalkyl, C₂₋₁₀ alkenyl, C₂₋₁₀ alkynyl, heteroC₁₋₁₀ alkyl, heteroC₂₋₁₀ alkenyl, heteroC₂₋₁₀ alkynyl, C₃₋₁₀ carbocyclyl, 3-14 membered heterocyclyl, C₆₋₁₄ aryl, and 5-14 membered heteroaryl, or two R^{aa} groups are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups;
each instance of R^{bb} is, independently, selected from hydrogen, —OH, —OR^{aa}, —N(R^{cc})₂, —CN, —C(═O)R^{aa}, —C(═O)N(R^{cc})₂, —CO₂R^{aa}, —SO₂R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{cc}, —C(═S)SR^{cc}, —P(═O)(R^{aa})₂, —P(═O)(OR^{cc})₂, —P(═O)(N(R^{cc})₂)₂, C₁₋₁₀ alkyl, C₁₋₁₀ perhaloalkyl, C₂₋₁₀ alkenyl, C₂₋₁₀ alkynyl, heteroC₁₋₁₀ alkyl, heteroC₂₋₁₀ alkenyl, heteroC₂₋₁₀alkynyl, C₃₋₁₀ carbocyclyl, 3-14 membered heterocyclyl, C₆₋₁₄ aryl, and 5-14 membered heteroaryl, or two R^{bb} groups are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups; wherein X⁻ is a counterion;
each instance of R^{cc} is, independently, selected from hydrogen, C₁₋₁₀ alkyl, C₁₋₁₀ perhaloalkyl, C₂₋₁₀ alkenyl, C₂₋₁₀ alkynyl, heteroC₁₋₁₀ alkyl, heteroC₂₋₁₀ alkenyl, heteroC₂₋₁₀ alkynyl, C₃₋₁₀ carbocyclyl, 3-14 membered heterocyclyl, C₆₋₁₄ aryl, and 5-14 membered heteroaryl, or two R^{cc} groups are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups;
each instance of R^{dd} is, independently, selected from halogen, —CN, —NO₂, —N₃, —SO₂H, —SO₃H, —OH, —OR^{ee}, —ON(R^{ff})₂, —N(R^{ff})₂, —N(R^{ff})₃⁺X⁻, —N(OR^{ee})R^{ff}, —SH, —SR^{ee}, —SSR^{ee}, —C(═O)R^{ee}, —CO₂H, —CO₂R^{ee}, —OC(═O)R^{ee}, —OCO₂R^{ee}, —C(═O)N(R^{ff})₂, —OC(═O)N(R^{ff})₂, —NR^{ff}C(═O)R^{ee}, —NR^{ff}CO₂R^{ee}, —NR^{ff}C(═O)N(R^{ff})₂, —C(═NR^{ff})OR^{ee}, —OC(═NR^{ff})R^{ee}, —OC(═NR^{ff})OR^{ee}, —C(═NR^{ff})N(R^{ff})₂, —OC(═NR^{ff})N(R^{ff})₂, —NR^{ff}C(═NR^{ff})N(R^{ff})₂, —NR^{ff}SO₂R^{ee}, —SO₂N(R^{ff})₂, —SO₂R^{ee}, —SO₂OR^{ee}, —OSO₂R^{ee}, —S(═O)R^{ee}, —Si(R^{ee})₃, —OSi(R^{ee})₃, —C(═S)N(R^{ff})₂, —C(═O)SR^{ee}, —C(═S)SR^{ee}, —SC(═S)SR^{ee}, —P(═O)(OR^{ee})₂, —P(═O)(R^{ee})₂, —OP(═O)(R^{ee})₂, —OP(═O)(OR^{ee})₂, C₁₋₆ alkyl, C₁₋₆ perhaloalkyl, C₂₋₆ alkenyl, C₂₋₆ alkynyl, heteroC₁₋₆ alkyl, heteroC₂₋₆ alkenyl, heteroC₂₋₆ alkynyl, C₃₋₁₀ carbocyclyl, 3-10 membered heterocyclyl, C₆₋₁₀ aryl, 5-10 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg} groups, or two geminal R^{dd} substituents can be joined to form ═O or ═S; wherein X⁻ is a counterion;
each instance of R^{ee} is, independently, selected from C₁₋₆ alkyl, C₁₋₆ perhaloalkyl, C₂₋₆ alkenyl, C₂₋₆ alkynyl, heteroC₁₋₆ alkyl, heteroC₂₋₆ alkenyl, heteroC₂₋₆ alkynyl, C₃₋₁₀ carbocyclyl, C₆₋₁₀ aryl, 3-10 membered heterocyclyl, and 3-10 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg} groups;
each instance of R^{ff} is, independently, selected from hydrogen, C₁₋₆ alkyl, C₁₋₆ perhaloalkyl, C₂₋₆ alkenyl, C₂₋₆ alkynyl, heteroC₁₋₆ alkyl, heteroC₂₋₆ alkenyl, heteroC₂₋₆ alkynyl, C₃₋₁₀ carbocyclyl, 3-10 membered heterocyclyl, C₆₋₁₀ aryl and 5-10 membered heteroaryl, or two R⁻ groups are joined to form a 3-10 membered heterocyclyl or 5-10 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg} groups; and
each instance of R& is, independently, halogen, —CN, —NO₂, —N₃, —SO₂H, —SO₃H, —OH, —OC₁₋₆ alkyl, —ON(C₁₋₆ alkyl)₂, —N(C₁₋₆ alkyl)₂, —N(C₁₋₆ alkyl)₃⁺X⁻, —NH(C₁₋₆ alkyl)₂⁺X⁻, —NH₂(C₁₋₆ alkyl)⁺X⁻, —NH₃⁺X⁻, —N(OC₁₋₆ alkyl)(C₁₋₆ alkyl), —N(OH)(C₁₋₆ alkyl), —NH(OH), —SH, —SC₁₋₆ alkyl, —SS(C₁₋₆ alkyl), —C(═O)(C₁₋₆ alkyl), —CO₂H, —CO₂ (C₁₋₆ alkyl), —OC(═O)(C₁₋₆ alkyl), —OCO₂(C₁₋₆ alkyl), —C(═O)NH₂, —C(═O)N(C₁₋₆ alkyl)₂, —OC(═O)NH(C₁₋₆ alkyl), —NHC(═O)(C₁₋₆ alkyl), —N(C₁₋₆ alkyl)C(═O)(C₁₋₆ alkyl), —NHCO₂(C₁₋₆ alkyl), —NHC(═O)N(C₁₋₆ alkyl)₂, —NHC(═O)NH(C₁₋₆ alkyl), —NHC(═O)NH₂, —C(═NH)O(C₁₋₆ alkyl), —OC(═NH)(C₁₋₆ alkyl), —OC(═NH)OC₁₋₆ alkyl, —C(═NH)N(C₁₋₆ alkyl)₂, —C(═NH)NH(C₁₋₆ alkyl), —C(═NH)NH₂, —OC(═NH)N(C₁₋₆ alkyl)₂, —OC(═NH)NH(C₁₋₆ alkyl), —OC(═NH)NH₂, —NHC(═NH)N(C₁₋₆ alkyl)₂, —NHC(═NH)NH₂, —NHSO₂(C₁₋₆ alkyl), —SO₂N(C₁₋₆ alkyl)₂, —SO₂NH(C₁₋₆ alkyl), —SO₂NH₂, —SO₂(C₁₋₆ alkyl), —SO₂O(C₁₋₆ alkyl), —OSO₂(C₁₋₆ alkyl), —SO(C₁₋₆ alkyl), —Si(C₁₋₆ alkyl)₃, —OSi(C₁₋₆ alkyl)₃ —C(═S)N(C₁₋₆ alkyl)₂, C(═S)NH(C₁₋₆ alkyl), C(═S)NH₂, —C(═O)S(C₁₋₆ alkyl), —C(═S)SC₁₋₆ alkyl, —SC(═S)SC₁₋₆ alkyl, —P(═O)(OC₁₋₆ alkyl)₂, —P(═O)(C₁₋₆ alkyl)₂, —OP(═O)(C₁₋₆ alkyl)₂, —OP(═O)(OC₁₋₆ alkyl)₂, C₁₋₆ alkyl, C₁₋₆ perhaloalkyl, C₂₋₆ alkenyl, C₂₋₆ alkynyl, heteroC₁₋₆ alkyl, heteroC₂₋₆ alkenyl, heteroC₂₋₆ alkynyl, C₃₋₁₀ carbocyclyl, C₆₋₁₀ aryl, 3-10 membered heterocyclyl, 5-10 membered heteroaryl; or two geminal R^{gg} substituents can be joined to form ═O or ═S; wherein X⁻ is a counterion.

The term “halo” or “halogen” refers to fluorine (fluoro, —F), chlorine (chloro, —Cl), bromine (bromo, —Br), or iodine (iodo, —I).

The term “hydroxyl” or “hydroxy” refers to the group-OH. The term “substituted hydroxyl” or “substituted hydroxyl,” by extension, refers to a hydroxyl group wherein the oxygen atom directly attached to the parent molecule is substituted with a group other than hydrogen, and includes groups selected from —OR^{aa}, —ON(R^{bb})₂, —OC(═O)SR^{aa}, —OC(═O)R^{aa}, —OCO₂R^{aa}, —OC(═O)N(R^{bb})₂, —OC(═NR^{bb}) R^{aa}, —OC(═NR^{bb})OR^{aa}, —OC(═NR^{bb})N(R^{bb})₂, —OS(═O)R^{aa}, —OSO₂R^{aa}, —OSi(R^{aa})₃, —OP(R^{cc})₂, —OP(R^{cc})₃⁺X⁻, —OP(OR^{cc})₂, —OP(OR^{cc})₃⁺X⁻, —OP(═O)(R^{aa})₂, —OP(═O)(OR^{cc})², and —OP(═O)(N(R^{bb})₂)₂, wherein X⁻, R^{aa}, R^{bb}, and R^{cc} are as defined herein.

The term “amino” refers to the group —NH₂. The term “substituted amino,” by extension, refers to a monosubstituted amino, a disubstituted amino, or a trisubstituted amino. In certain embodiments, the “substituted amino” is a monosubstituted amino or a disubstituted amino group.

The term “monosubstituted amino” refers to an amino group wherein the nitrogen atom directly attached to the parent molecule is substituted with one hydrogen and one group other than hydrogen, and includes groups selected from —NH(R^{bb}), —NHC(═O)R^{aa}, —NHCO₂R^{aa}, —NHC(═O)N(R^{bb})₂, —NHC(═NR^{bb})N(R^{bb})₂, —NHSO₂R^{aa}, —NHP(═O)(OR^{cc})₂, and —NHP(═O)(N(R^{bb})₂)₂, wherein R^{aa}, R^{bb} and R^{cc} are as defined herein, and wherein R^{bb} of the group —NH(R^{bb}) is not hydrogen.

The term “disubstituted amino” refers to an amino group wherein the nitrogen atom directly attached to the parent molecule is substituted with two groups other than hydrogen, and includes groups selected from —N(R^{bb})₂, —NR^{bb}C(═O)R^{aa}, —NR^{bb}CO₂R^{aa}, —NR^{bb}C(═O)N(R^{bb})₂, —NR^{bb}C(═NR^{bb})N(R^{bb})₂, —NR^{bb}SO₂R^{aa}, —NR^{bb}P(═O)(OR^{cc})₂, and —NR^{bb}P(═O)(N(R^{bb})₂)₂, wherein R^{aa}, R^{bb}, and R^{cc} are as defined herein, with the proviso that the nitrogen atom directly attached to the parent molecule is not substituted with hydrogen.

The term “trisubstituted amino” refers to an amino group wherein the nitrogen atom directly attached to the parent molecule is substituted with three groups, and includes groups selected from —N(R^{bb})₃ and —N(R^{bb})₃⁺X⁻, wherein R^{bb} and X⁻ are as defined herein.

The term “acyl” refers to a group having the general formula —C(═O)R^{X1}, —C(═O)OR^{X1}, —C(═O)—O—C(═O)R^{X1}, —C(═O)SR^{X1}, —C(═O)N(R^{X1})₂, —C(═S)R^{X1}, —C(═S)N(R^{X1})₂, —C(═S)O(R^{X1}), —C(═S)S(R^{X1}), —C(═NR^{X1})R^{X1}, —C(═NR^{X1})OR^{X1}, —C(═NR^{X1})SR^{X1}, and —C(═NR^{X1})N(R^{X1})₂, wherein R^{X1} is hydrogen; halogen; substituted or unsubstituted hydroxyl; substituted or unsubstituted thiol; substituted or unsubstituted amino; substituted or unsubstituted acyl, cyclic or acyclic, substituted or unsubstituted, branched or unbranched aliphatic; cyclic or acyclic, substituted or unsubstituted, branched or unbranched heteroaliphatic; cyclic or acyclic, substituted or unsubstituted, branched or unbranched alkyl; cyclic or acyclic, substituted or unsubstituted, branched or unbranched alkenyl; substituted or unsubstituted alkynyl; substituted or unsubstituted aryl, substituted or unsubstituted heteroaryl, aliphaticoxy, heteroaliphaticoxy, alkyloxy, heteroalkyloxy, aryloxy, heteroaryloxy, aliphaticthioxy, heteroaliphaticthioxy, alkylthioxy, heteroalkylthioxy, arylthioxy, heteroarylthioxy, mono- or di-aliphaticamino, mono- or di-heteroaliphaticamino, mono- or di-alkylamino, mono- or di-heteroalkylamino, mono- or di-arylamino, or mono- or di-heteroarylamino; or two R^{X1} groups taken together form a 5- to 6-membered heterocyclic ring. Exemplary acyl groups include aldehydes (—CHO), carboxylic acids (—CO₂H), ketones, acyl halides, esters, amides, imines, carbonates, carbamates, and ureas. Acyl substituents include, but are not limited to, any of the substituents described herein, that result in the formation of a stable moiety (e.g., aliphatic, alkyl, alkenyl, alkynyl, heteroaliphatic, heterocyclic, aryl, heteroaryl, acyl, oxo, imino, thiooxo, cyano, isocyano, amino, azido, nitro, hydroxyl, thiol, halo, aliphaticamino, heteroaliphaticamino, alkylamino, heteroalkylamino, arylamino, heteroarylamino, alkylaryl, arylalkyl, aliphaticoxy, heteroaliphaticoxy, alkyloxy, heteroalkyloxy, aryloxy, heteroaryloxy, aliphaticthioxy, heteroaliphaticthioxy, alkylthioxy, heteroalkylthioxy, arylthioxy, heteroarylthioxy, acyloxy, and the like, each of which may or may not be further substituted).

The term “carbonyl” refers a group wherein the carbon directly attached to the parent molecule is sp² hybridized, and is substituted with an oxygen, nitrogen or sulfur atom, e.g., a group selected from ketones (e.g., —C(═O)R^{aa}), carboxylic acids (e.g., —CO₂H), aldehydes (—CHO), esters (e.g., —CO₂R^{aa}, —C(═O)SR^{aa}, —C(═S)SR^{aa}), amides (e.g., —C(═O)N(R^{bb})₂, —C(═O)NR^{bb}SO₂R^{aa}, —C(═S)N(R^{bb})₂), and imines (e.g., —C(═NR^{bb})R^{aa}, —C(═NR^{bb})OR^{aa}), —C(═NR^{bb})N(R^{bb})₂), wherein R^{aa} and R^{bb} are as defined herein.

The term “oxo” refers to the group ═O, and the term “thiooxo” refers to the group ═S.

Nitrogen atoms can be substituted or unsubstituted as valency permits, and include primary, secondary, tertiary, and quaternary nitrogen atoms. Exemplary nitrogen atom substituents include, but are not limited to, hydrogen, —OH, —OR^{aa}, —N(R^{cc})₂, —CN, —C(═O)R^{aa}, —C(═O)N(R^{cc})₂, —CO₂R^{aa}, —SO₂R^{aa}, —C(═NR^{bb})R^{aa}, C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{cc}, —C(═S)SR^{cc}, —P(═O)(OR^{cc})₂, —P(═O)(R^{aa})₂, —P(═O)(N(R^{cc})₂)₂, C₁₋₁₀ alkyl, C₁₋₁₀ perhaloalkyl, C₂₋₁₀ alkenyl, C₂₋₁₀ alkynyl, heteroC₁₋₁₀alkyl, heteroC₂₋₁₀alkenyl, heteroC₂₋₁₀alkynyl, C₃₋₁₀ carbocyclyl, 3-14 membered heterocyclyl, C₆₋₁₄ aryl, and 5-14 membered heteroaryl, or two R^{cc} groups attached to an N atom are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups, and wherein R^{aa}, R^{bb}, R^{cc}, and R^{dd} are as defined herein.

In certain embodiments, the substituent present on the nitrogen atom is an nitrogen protecting group (also referred to herein as an “amino protecting group”). Nitrogen protecting groups include, but are not limited to, —OH, —OR^{aa}, —N(R^{cc})₂, —C(═O)R^{aa}, —C(═O)N(R^{cc})₂, —CO₂R^{aa}, —SO₂R^{aa}, —C(═NR^{cc})R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{aa})—C(═S)N(R^{cc})₂, —C(═O)SR^{cc}, —C(═S)SR^{cc}, C₁₋₁₀ alkyl (e.g., aralkyl, heteroaralkyl), C₂₋₁₀ alkenyl, C₂₋₁₀ alkynyl, heteroC₁₋₁₀ alkyl, heteroC₂₋₁₀ alkenyl, heteroC₂₋₁₀ alkynyl, C₃₋₁₀ carbocyclyl, 3-14 membered heterocyclyl, C₆₋₁₄ aryl, and 5-14 membered heteroaryl groups, wherein each alkyl, alkenyl, alkynyl, heteroalkyl, heteroalkenyl, heteroalkynyl, carbocyclyl, heterocyclyl, aralkyl, aryl, and heteroaryl is independently substituted with 0, 1, 2, 3, 4, or 5 R^{dd} groups, and wherein R^{aa}, R^{bb}, R^{cc} and R^{dd} are as defined herein. Nitrogen protecting groups are well known in the art and include those described in detail in Protecting Groups in Organic Synthesis, T. W. Greene and P. G. M. Wuts, 3^{rd} edition, John Wiley & Sons, 1999, incorporated herein by reference.

For example, nitrogen protecting groups such as amide groups (e.g., —C(═O)R^{aa}) include, but are not limited to, formamide, acetamide, chloroacetamide, trichloroacetamide, trifluoroacetamide, phenylacetamide, 3-phenylpropanamide, picolinamide, 3-pyridylcarboxamide, N-benzoylphenylalanyl derivative, benzamide, p-phenylbenzamide, o-nitophenylacetamide, o-nitrophenoxyacetamide, acetoacetamide, (N′-dithiobenzyloxyacylamino) acetamide, 3-(p-hydroxyphenyl) propanamide, 3-(o-nitrophenyl) propanamide, 2-methyl-2-(o-nitrophenoxy) propanamide, 2-methyl-2-(o-phenylazophenoxy) propanamide, 4-chlorobutanamide, 3-methyl-3-nitrobutanamide, o-nitrocinnamide, N-acetylmethionine derivative, o-nitrobenzamide, and o-(benzoyloxymethyl)benzamide.

Nitrogen protecting groups such as carbamate groups (e.g., —C(═O)OR^{aa}) include, but are not limited to, methyl carbamate, ethyl carbamate, 9-fluorenylmethyl carbamate (Fmoc), 9-(2-sulfo) fluorenylmethyl carbamate, 9-(2,7-dibromo) fluoroenylmethyl carbamate, 2,7-di-t-butyl-[9-(10,10-dioxo-10,10,10,10-tetrahydrothioxanthyl)]methyl carbamate (DBD-Tmoc), 4-methoxyphenacyl carbamate (Phenoc), 2,2,2-trichloroethyl carbamate (Troc), 2-trimethylsilylethyl carbamate (Teoc), 2-phenylethyl carbamate (hZ), 1-(1-adamantyl)-1-methylethyl carbamate (Adpoc), 1,1-dimethyl-2-haloethyl carbamate, 1,1-dimethyl-2,2-dibromoethyl carbamate (DB-t-BOC), 1,1-dimethyl-2,2,2-trichloroethyl carbamate (TCBOC), 1-methyl-1-(4-biphenylyl)ethyl carbamate (Bpoc), 1-(3,5-di-t-butylphenyl)-1-methylethyl carbamate (t-Bumcoc), 2-(2′- and 4′-pyridyl)ethyl carbamate (Pyoc), 2-(N,N-dicyclohexylcarboxamido)ethyl carbamate, t-butyl carbamate (BOC or Boc), 1-adamantyl carbamate (Adoc), vinyl carbamate (Voc), allyl carbamate (Alloc), 1-isopropylallyl carbamate (Ipaoc), cinnamyl carbamate (Coc), 4-nitrocinnamyl carbamate (Noc), 8-quinolyl carbamate, N-hydroxypiperidinyl carbamate, alkyldithio carbamate, benzyl carbamate (Cbz), p-methoxybenzyl carbamate (Moz), p-nitobenzyl carbamate, p-bromobenzyl carbamate, p-chlorobenzyl carbamate, 2,4-dichlorobenzyl carbamate, 4-methylsulfinylbenzyl carbamate (Msz), 9-anthrylmethyl carbamate, diphenylmethyl carbamate, 2-methylthioethyl carbamate, 2-methylsulfonylethyl carbamate, 2-(p-toluenesulfonyl)ethyl carbamate, [2-(1,3-dithianyl)]methyl carbamate (Dmoc), 4-methylthiophenyl carbamate (Mtpc), 2,4-dimethylthiophenyl carbamate (Bmpc), 2-phosphonioethyl carbamate (Peoc), 2-triphenylphosphonioisopropyl carbamate (Ppoc), 1,1-dimethyl-2-cyanoethyl carbamate, m-chloro-p-acyloxybenzyl carbamate, p-(dihydroxyboryl)benzyl carbamate, 5-benzisoxazolylmethyl carbamate, 2-(trifluoromethyl)-6-chromonylmethyl carbamate (Tcroc), m-nitrophenyl carbamate, 3,5-dimethoxybenzyl carbamate, o-nitrobenzyl carbamate, 3,4-dimethoxy-6-nitrobenzyl carbamate, phenyl (o-nitrophenyl)methyl carbamate, t-amyl carbamate, S-benzyl thiocarbamate, p-cyanobenzyl carbamate, cyclobutyl carbamate, cyclohexyl carbamate, cyclopentyl carbamate, cyclopropylmethyl carbamate, p-decyloxybenzyl carbamate, 2,2-dimethoxyacylvinyl carbamate, o-(N,N-dimethylcarboxamido)benzyl carbamate, 1,1-dimethyl-3-(N,N-dimethylcarboxamido) propyl carbamate, 1,1-dimethylpropynyl carbamate, di(2-pyridyl)methyl carbamate, 2-furanylmethyl carbamate, 2-iodoethyl carbamate, isoborynl carbamate, isobutyl carbamate, isonicotinyl carbamate, p-(p′-methoxyphenylazo)benzyl carbamate, 1-methylcyclobutyl carbamate, 1-methylcyclohexyl carbamate, 1-methyl-1-cyclopropylmethyl carbamate, 1-methyl-1-(3,5-dimethoxyphenyl)ethyl carbamate, 1-methyl-1-(p-phenylazophenyl)ethyl carbamate, 1-methyl-1-phenylethyl carbamate, 1-methyl-1-(4-pyridyl)ethyl carbamate, phenyl carbamate, p-(phenylazo)benzyl carbamate, 2,4,6-tri-t-butylphenyl carbamate, 4-(trimethylammonium)benzyl carbamate, and 2,4,6-trimethylbenzyl carbamate.

Nitrogen protecting groups such as sulfonamide groups (e.g., —S(═O)₂R^{aa}) include, but are not limited to, p-toluenesulfonamide (Ts), benzenesulfonamide, 2,3,6-trimethyl-4-methoxybenzenesulfonamide (Mtr), 2,4,6-trimethoxybenzenesulfonamide (Mtb), 2,6-dimethyl-4-methoxybenzenesulfonamide (Pme), 2,3,5,6-tetramethyl-4-methoxybenzenesulfonamide (Mte), 4-methoxybenzenesulfonamide (Mbs), 2,4,6-trimethylbenzenesulfonamide (Mts), 2,6-dimethoxy-4-methylbenzenesulfonamide (iMds), 2,2,5,7,8-pentamethylchroman-6-sulfonamide (Pmc), methanesulfonamide (Ms), β-trimethylsilylethanesulfonamide (SES), 9-anthracenesulfonamide, 4-(4′,8′-dimethoxynaphthylmethyl)benzenesulfonamide (DNMBS), benzylsulfonamide, trifluoromethylsulfonamide, and phenacylsulfonamide.

Other nitrogen protecting groups include, but are not limited to, phenothiazinyl-(10)-acyl derivative, N′-p-toluenesulfonylaminoacyl derivative, N′-phenylaminothioacyl derivative, N-benzoylphenylalanyl derivative, N-acetylmethionine derivative, 4,5-diphenyl-3-oxazolin-2-one, N-phthalimide, N-dithiasuccinimide (Dts), N-2,3-diphenylmaleimide, N-2,5-dimethylpyrrole, N-1,1,4,4-tetramethyldisilylazacyclopentane adduct (STABASE), 5-substituted 1,3-dimethyl-1,3,5-triazacyclohexan-2-one, 5-substituted 1,3-dibenzyl-1,3,5-triazacyclohexan-2-one, 1-substituted 3,5-dinitro-4-pyridone, N-methylamine, N-allylamine, N-[2-(trimethylsilyl) ethoxy]methylamine (SEM), N-3-acetoxypropylamine, N-(1-isopropyl-4-nitro-2-oxo-3-pyroolin-3-yl)amine, quaternary ammonium salts, N-benzylamine, N-di(4-methoxyphenyl)methylamine, N-5-dibenzosuberylamine, N-triphenylmethylamine (Tr), N-[(4-methoxyphenyl)diphenylmethyl]amine (MMTr), N-9-phenylfluorenylamine (PhF), N-2,7-dichloro-9-fluorenylmethyleneamine, N-ferrocenylmethylamino (Fcm), N-2-picolylamino N′-oxide, N-1,1-dimethylthiomethyleneamine, N-benzylideneamine, N-p-methoxybenzylideneamine, N-diphenylmethylencamine, N-[(2-pyridyl) mesityl]methyleneamine, N—(N′,N′-dimethylaminomethylene)amine, N,N′-isopropylidenediamine, N-p-nitrobenzylideneamine, N-salicylidencamine, N-5-chlorosalicylideneamine, N-(5-chloro-2-hydroxyphenyl)phenylmethyleneamine, N-cyclohexylidencamine, N-(5,5-dimethyl-3-oxo-1-cyclohexenyl)amine, N-borane derivative, N-diphenylborinic acid derivative, N-[phenyl (pentaacylchromium- or tungsten)acyl]amine, N-copper chelate, N-zinc chelate, N-nitroamine, N-nitrosoamine, amine N-oxide, diphenylphosphinamide (Dpp), dimethylthiophosphinamide (Mpt), diphenylthiophosphinamide (Ppt), dialkyl phosphoramidates, dibenzyl phosphoramidate, diphenyl phosphoramidate, benzenesulfenamide, o-nitrobenzenesulfonamide (Nps), 2,4-dinitrobenzenesulfenamide, pentachlorobenzenesulfonamide, 2-nitro-4-methoxybenzenesulfonamide, triphenylmethylsulfenamide, and 3-nitropyridinesulfenamide (Npys). In certain embodiments, a nitrogen protecting group is benzyl (Bn), tert-butyloxycarbonyl (BOC), carbobenzyloxy (Cbz), 9-flurenylmethyloxycarbonyl (Fmoc), trifluoroacetyl, triphenylmethyl, acetyl (Ac), benzoyl (Bz), p-methoxybenzyl (PMB), 3,4-dimethoxybenzyl (DMPM), p-methoxyphenyl (PMP), 2,2,2-trichloroethyloxycarbonyl (Troc), triphenylmethyl (Tr), tosyl (Ts), brosyl (Bs), nosyl (Ns), mesyl (Ms), triflyl (Tf), or dansyl (Ds).

In certain embodiments, the substituent present on an oxygen atom is an oxygen protecting group (also referred to herein as an “hydroxyl protecting group”). Oxygen protecting groups include, but are not limited to, —R^{aa}, —N(R^{bb})₂, —C(═O)SR^{aa}, —C(═O)R^{aa}, —CO₂R^{aa}, —C(═O)N(R^{bb})₂, —C(═NR^{bb})R^{aa}, —C(═NR^{bb})OR^{a}, —C(═NR^{bb})N(R^{bb})₂, —S(═O)R^{aa}, —SO₂R^{aa}, —Si(R^{aa})₃, —P(R^{cc})₂, —P(R^{cc})₃⁺X⁻, —P(OR^{cc})₂, —P(OR^{cc})₃⁺X⁻, —P(═O)(R^{aa})₂, —P(═O)(OR^{cc})₂, and —P(═O)(N(R^{bb})₂)₂, wherein X⁻, R^{aa}, R^{bb}, and R^{cc} are as defined herein. Oxygen protecting groups are well known in the art and include those described in detail in Protecting Groups in Organic Synthesis, T. W. Greene and P. G. M. Wuts, 3^{rd} edition, John Wiley & Sons, 1999, incorporated herein by reference.

Exemplary oxygen protecting groups include, but are not limited to, methyl, methoxymethyl (MOM), methylthiomethyl (MTM), t-butylthiomethyl, (phenyldimethylsilyl) methoxymethyl (SMOM), benzyloxymethyl (BOM), p-methoxybenzyloxymethyl (PMBM), (4-methoxyphenoxy)methyl (p-AOM), guaiacolmethyl (GUM), t-butoxymethyl, 4-pentenyloxymethyl (POM), siloxymethyl, 2-methoxyethoxymethyl (MEM), 2,2,2-trichloroethoxymethyl, bis(2-chloroethoxy)methyl, 2-(trimethylsilyl) ethoxymethyl (SEMOR), tetrahydropyranyl (THP), 3-bromotetrahydropyranyl, tetrahydrothiopyranyl, 1-methoxycyclohexyl, 4-methoxytetrahydropyranyl (MTHP), 4-methoxytetrahydrothiopyranyl, 4-methoxytetrahydrothiopyranyl S,S-dioxide, 1-[(2-chloro-4-methyl)phenyl]-4-methoxypiperidin-4-yl (CTMP), 1,4-dioxan-2-yl, tetrahydrofuranyl, tetrahydrothiofuranyl, 2,3,3a,4,5,6,7,7a-octahydro-7,8,8-trimethyl-4,7-methanobenzofuran-2-yl, 1-ethoxyethyl, 1-(2-chloroethoxy)ethyl, 1-methyl-1-methoxyethyl, 1-methyl-1-benzyloxyethyl, 1-methyl-1-benzyloxy-2-fluoroethyl, 2,2,2-trichloroethyl, 2-trimethylsilylethyl, 2-(phenylselenyl)ethyl, t-butyl, allyl, p-chlorophenyl, p-methoxyphenyl, 2,4-dinitrophenyl, benzyl (Bn), p-methoxybenzyl, 3,4-dimethoxybenzyl, o-nitrobenzyl, p-nitrobenzyl, p-halobenzyl, 2,6-dichlorobenzyl, p-cyanobenzyl, p-phenylbenzyl, 2-picolyl, 4-picolyl, 3-methyl-2-picolyl N-oxido, diphenylmethyl, p,p′-dinitrobenzhydryl, 5-dibenzosuberyl, triphenylmethyl, α-naphthyldiphenylmethyl, p-methoxyphenyldiphenylmethyl, di(p-methoxyphenyl)phenylmethyl, tri(p-methoxyphenyl)methyl, 4-(4′-bromophenacyloxyphenyl)diphenylmethyl, 4,4′,4″-tris(4,5-dichlorophthalimidophenyl)methyl, 4,4′,4″-tris(levulinoyloxyphenyl)methyl, 4,4′,4″-tris(benzoyloxyphenyl)methyl, 3-(imidazol-1-yl)bis(4′,4″-dimethoxyphenyl)methyl, 1,1-bis(4-methoxyphenyl)-1′-pyrenylmethyl, 9-anthryl, 9-(9-phenyl) xanthenyl, 9-(9-phenyl-10-oxo) anthryl, 1,3-benzodithiolan-2-yl, benzisothiazolyl S,S-dioxido, trimethylsilyl (TMS), triethylsilyl (TES), triisopropylsilyl (TIPS), dimethylisopropylsilyl (IPDMS), diethylisopropylsilyl (DEIPS), dimethylthexylsilyl, t-butyldimethylsilyl (TBDMS), t-butyldiphenylsilyl (TBDPS), tribenzylsilyl, tri-p-xylylsilyl, triphenylsilyl, diphenylmethylsilyl (DPMS), t-butylmethoxyphenylsilyl (TBMPS), formate, benzoylformate, acetate, chloroacetate, dichloroacetate, trichloroacetate, trifluoroacetate, methoxyacetate, triphenylmethoxyacetate, phenoxyacetate, p-chlorophenoxyacetate, 3-phenylpropionate, 4-oxopentanoate (levulinate), 4,4-(ethylenedithio) pentanoate (levulinoyldithioacetal), pivaloate, adamantoate, crotonate, 4-methoxycrotonate, benzoate, p-phenylbenzoate, 2,4,6-trimethylbenzoate (mesitoate), methyl carbonate, 9-fluorenylmethyl carbonate (Fmoc), ethyl carbonate, 2,2,2-trichloroethyl carbonate (Troc), 2-(trimethylsilyl)ethyl carbonate (TMSEC), 2-(phenylsulfonyl)ethyl carbonate (Psec), 2-(triphenylphosphonio)ethyl carbonate (Peoc), isobutyl carbonate, vinyl carbonate, allyl carbonate, t-butyl carbonate (BOC or Boc), p-nitrophenyl carbonate, benzyl carbonate, p-methoxybenzyl carbonate, 3,4-dimethoxybenzyl carbonate, o-nitrobenzyl carbonate, p-nitrobenzyl carbonate, S-benzyl thiocarbonate, 4-ethoxy-1-napththyl carbonate, methyl dithiocarbonate, 2-iodobenzoate, 4-azidobutyrate, 4-nitro-4-methylpentanoate, o-(dibromomethyl)benzoate, 2-formylbenzenesulfonate, 2-(methylthiomethoxy)ethyl, 4-(methylthiomethoxy) butyrate, 2-(methylthiomethoxymethyl)benzoate, 2,6-dichloro-4-methylphenoxyacetate, 2,6-dichloro-4-(1,1,3,3-tetramethylbutyl) phenoxyacetate, 2,4-bis(1,1-dimethylpropyl) phenoxyacetate, chlorodiphenylacetate, isobutyrate, monosuccinoate, (E)-2-methyl-2-butenoate, o-(methoxyacyl)benzoate, α-naphthoate, nitrate, alkyl N,N,N′,N′-tetramethylphosphorodiamidate, alkyl N-phenylcarbamate, borate, dimethylphosphinothioyl, alkyl 2,4-dinitrophenylsulfenate, sulfate, methanesulfonate (mesylate), benzylsulfonate, and tosylate (Ts). In certain embodiments, an oxygen protecting group is silyl. In certain embodiments, an oxygen protecting group is t-butyldiphenylsilyl (TBDPS), t-butyldimethylsilyl (TBDMS), triisoproylsilyl (TIPS), triphenylsilyl (TPS), triethylsilyl (TES), trimethylsilyl (TMS), triisopropylsiloxymethyl (TOM), acetyl (Ac), benzoyl (Bz), allyl carbonate, 2,2,2-trichloroethyl carbonate (Troc), 2-trimethylsilylethyl carbonate, methoxymethyl (MOM), 1-ethoxyethyl (EE), 2-methyoxy-2-propyl (MOP), 2,2,2-trichloroethoxymethyl, 2-methoxyethoxymethyl (MEM), 2-trimethylsilylethoxymethyl (SEM), methylthiomethyl (MTM), tetrahydropyranyl (THP), tetrahydrofuranyl (THF), p-methoxyphenyl (PMP), triphenylmethyl (Tr), methoxytrityl (MMT), dimethoxytrityl (DMT), allyl, p-methoxybenzyl (PMB), t-butyl, benzyl (Bn), allyl, or pivaloyl (Piv).

In certain embodiments, the substituent present on a sulfur atom is a sulfur protecting group (also referred to as a “thiol protecting group”). Sulfur protecting groups include, but are not limited to, —R^{aa}, —N(R^{bb})₂, —C(═O)SR^{aa}, —C(═O)R^{aa}, —CO₂R^{aa}, —C(═O)N(R^{bb})₂, —C(═NR^{bb})R^{aa}, —C(═NR^{bb})OR^{aa}, —C(═NR^{bb})N(R^{bb})₂, —S(═O)R^{aa}, —SO₂R^{aa}, —Si(R^{aa})₃, —P(R^{cc})₂, —P(R^{cc})₃⁺X⁻, —P(OR)₂, —P(OR)₃⁺X⁻, —P(═O)(R^{aa})₂, —P(═O)(OR^{c})₂, and —P(═O)(N(R^{bb})₂)₂, wherein R^{aa}, R^{bb}, and R^{cc} are as defined herein. Sulfur protecting groups are well known in the art and include those described in detail in Protecting Groups in Organic Synthesis, T. W. Greene and P. G. M. Wuts, 3^{rd} edition, John Wiley & Sons, 1999, incorporated herein by reference. In certain embodiments, a sulfur protecting group is acetamidomethyl, t-Bu, 3-nitro-2-pyridine sulfenyl, 2-pyridine-sulfenyl, or triphenylmethyl.

A “counterion” or “anionic counterion” is a negatively charged group associated with a positively charged group in order to maintain electronic neutrality. An anionic counterion may be monovalent (i.e., including one formal negative charge). An anionic counterion may also be multivalent (i.e., including more than one formal negative charge), such as divalent or trivalent. Exemplary counterions include halide ions (e.g., F⁻, Cl⁻, Br⁻, I⁻), NO₃⁻, ClO₄⁻, OH⁻, H₂PO₄⁻, HCO₃⁻, HSO₄⁻, sulfonate ions (e.g., methanesulfonate, trifluoromethanesulfonate, p-toluenesulfonate, benzenesulfonate, 10-camphor sulfonate, naphthalene-2-sulfonate, naphthalene-1-sulfonic acid-5-sulfonate, ethan-1-sulfonic acid-2-sulfonate, and the like), carboxylate ions (e.g., acetate, propanoate, benzoate, glycerate, lactate, tartrate, glycolate, gluconate, and the like), BF₄⁻, PF₄⁻, PF₆⁻, AsF₆⁻, SbF₆⁻, B[3,5-(CF₃)₂C₆H₃]₄]⁻, B(C₆F₅)₄⁻, BPh₄, Al(OC(CF₃)₃)₄⁻, and carborane anions (e.g., CB₁₁H₁₂⁻ or (HCB₁₁Me₅Br₆)⁻). Exemplary counterions which may be multivalent include CO₃²⁻, HPO₄²⁻, PO₄³⁻, B₄O₇²⁻, SO₄²⁻, S₂O₃²⁻, carboxylate anions (e.g., tartrate, citrate, fumarate, maleate, malate, malonate, gluconate, succinate, glutarate, adipate, pimelate, suberate, azelate, sebacate, salicylate, phthalates, aspartate, glutamate, and the like), and carboranes.

The term “leaving group” is given its ordinary meaning in the art of synthetic organic chemistry and refers to an atom or a group capable of being displaced by a nucleophile. See, for example, Smith, March's Advanced Organic Chemistry 6^{th} ed. (501-502). Examples of suitable leaving groups include, but are not limited to, halogen (such as F, Cl, Br, or I (iodine)), alkoxycarbonyloxy, aryloxycarbonyloxy, alkanesulfonyloxy, arenesulfonyloxy, alkyl-carbonyloxy (e.g., acetoxy), arylcarbonyloxy, aryloxy, methoxy, N,O-dimethylhydroxylamino, pixyl, and haloformates. In some cases, the leaving group is a sulfonic acid ester, such as toluenesulfonate (tosylate, —OTs), methanesulfonate (mesylate, —OMs), p-bromobenzenesulfonyloxy (brosylate, —OBs), —OS(═O)₂(CF₂)₃CF₃ (nonaflate, —ONf), or trifluoromethanesulfonate (triflate, —OTf). In some cases, the leaving group is a brosylate, such as p-bromobenzenesulfonyloxy. In some cases, the leaving group is a nosylate, such as 2-nitrobenzenesulfonyloxy. The leaving group may also be a phosphineoxide (e.g., formed during a Mitsunobu reaction) or an internal leaving group such as an epoxide or cyclic sulfate. Other non-limiting examples of leaving groups are water, ammonia, alcohols, ether moieties, thioether moieties, zinc halides, magnesium moieties, diazonium salts, and copper moieties. Further exemplary leaving groups include, but are not limited to, halo (e.g., chloro, bromo, iodo) and activated substituted hydroxyl groups (e.g., —OC(═O)SR^{aa}, —OC(═O)R^{aa}, —OCO₂R^{aa}, —OC(═O)N(R^{bb})₂, OC(═NR^{bb})R^{aa}, —OC(═NR^{bb})OR^{aa}, —OC(═NR^{bb})N(R^{bb})₂, —OS(═O)R^{aa}, —OSO₂R^{aa}, —OP(R^{cc})₂, —OP(R^{cc})₃, —OP(═O)₂R^{aa}, —OP(═O)(R^{aa})₂, —OP(═O)(OR^{cc})₂, —OP(═O)₂N(R^{bb})₂, and —OP(═O)(NR^{bb})₂, wherein R^{aa}, R^{bb}, and R^{cc} are as defined herein).

As used herein, use of the phrase “at least one instance” refers to 1, 2, 3, 4, or more instances, but also encompasses a range, e.g., for example, from 1 to 4, from 1 to 3, from 1 to 2, from 2 to 4, from 2 to 3, or from 3 to 4 instances, inclusive.

A “non-hydrogen group” refers to any group that is defined for a particular variable that is not hydrogen.

These and other exemplary substituents are described in more detail in the Detailed Description, Examples, and claims. The invention is not intended to be limited in any manner by the above exemplary listing of substituents.

### Other Definitions

The following definitions are more general terms used throughout the present application.

As used herein, the term “salt” refers to any and all salts, and encompasses pharmaceutically acceptable salts.

The term “pharmaceutically acceptable salt” refers to those salts which are, within the scope of sound medical judgment, suitable for use in contact with the tissues of humans and lower animals without undue toxicity, irritation, allergic response, and the like, and are commensurate with a reasonable benefit/risk ratio. Pharmaceutically acceptable salts are well known in the art. For example, Berge et al. describe pharmaceutically acceptable salts in detail in J. Pharmaceutical Sciences, 1977, 66, 1-19, incorporated herein by reference. Pharmaceutically acceptable salts of the compounds of this invention include those derived from suitable inorganic and organic acids and bases. Examples of pharmaceutically acceptable, nontoxic acid addition salts are salts of an amino group formed with inorganic acids, such as hydrochloric acid, hydrobromic acid, phosphoric acid, sulfuric acid, and perchloric acid or with organic acids, such as acetic acid, oxalic acid, maleic acid, tartaric acid, citric acid, succinic acid, or malonic acid or by using other methods known in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Salts derived from appropriate bases include alkali metal, alkaline earth metal, ammonium, and N⁺(C₁₋₄ alkyl)₄⁻ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions, such as halide, hydroxide, carboxylate, sulfate, phosphate, nitrate, lower alkyl sulfonate, and aryl sulfonate.

The term “solvate” refers to forms of the compound, or a salt thereof, that are associated with a solvent, usually by a solvolysis reaction. This physical association may include hydrogen bonding. Conventional solvents include water, methanol, ethanol, acetic acid, DMSO, THF, diethyl ether, and the like. The compounds described herein may be prepared, e.g., in crystalline form, and may be solvated. Suitable solvates include pharmaceutically acceptable solvates and further include both stoichiometric solvates and non-stoichiometric solvates. In certain instances, the solvate will be capable of isolation, for example, when one or more solvent molecules are incorporated in the crystal lattice of a crystalline solid. “Solvate” encompasses both solution-phase and isolatable solvates. Representative solvates include hydrates, ethanolates, and methanolates.

The term “hydrate” refers to a compound that is associated with water. Typically, the number of the water molecules contained in a hydrate of a compound is in a definite ratio to the number of the compound molecules in the hydrate. Therefore, a hydrate of a compound may be represented, for example, by the general formula R·xH₂O, wherein R is the compound, and x is a number greater than 0. A given compound may form more than one type of hydrate, including, e.g., monohydrates (x is 1), lower hydrates (x is a number greater than 0 and smaller than 1, e.g., hemihydrates (R·0.5H₂O)), and polyhydrates (x is a number greater than 1, e.g., dihydrates (R·2H₂O) and hexahydrates (R·6H₂O)).

The term “tautomers” or “tautomeric” refers to two or more interconvertible compounds resulting from at least one formal migration of a hydrogen atom and at least one change in valency (e.g., a single bond to a double bond, a triple bond to a single bond, or vice versa). The exact ratio of the tautomers depends on several factors, including temperature, solvent, and pH. Tautomerizations (i.e., the reaction providing a tautomeric pair) may catalyzed by acid or base. Exemplary tautomerizations include keto-to-enol, amide-to-imide, lactam-to-lactim, enamine-to-imine, and enamine-to-(a different enamine) tautomerizations.

It is also to be understood that compounds that have the same molecular formula but differ in the nature or sequence of bonding of their atoms or the arrangement of their atoms in space are termed “isomers”. Isomers that differ in the arrangement of their atoms in space are termed “stereoisomers”.

Stereoisomers that are not mirror images of one another are termed “diastereomers” and those that are non-superimposable mirror images of each other are termed “enantiomers”. When a compound has an asymmetric center, for example, it is bonded to four different groups, a pair of enantiomers is possible. An enantiomer can be characterized by the absolute configuration of its asymmetric center and is described by the R- and S-sequencing rules of Cahn and Prelog, or by the manner in which the molecule rotates the plane of polarized light and designated as dextrorotatory or levorotatory (i.e., as (+) or (−)-isomers respectively). A chiral compound can exist as either individual enantiomer or as a mixture thereof. A mixture containing equal proportions of the enantiomers is called a “racemic mixture”.

The term “polymorph” refers to a crystalline form of a compound (or a salt, hydrate, or solvate thereof). All polymorphs have the same elemental composition. Different crystalline forms usually have different X-ray diffraction patterns, infrared spectra, melting points, density, hardness, crystal shape, optical and electrical properties, stability, and solubility. Recrystallization solvent, rate of crystallization, storage temperature, and other factors may cause one crystal form to dominate. Various polymorphs of a compound can be prepared by crystallization under different conditions.

The term “prodrugs” refers to compounds that have cleavable groups and become by solvolysis or under physiological conditions the compounds described herein, which are pharmaceutically active in vivo. Such examples include, but are not limited to, choline ester derivatives and the like, N-alkylmorpholine esters and the like. Other derivatives of the compounds described herein have activity in both their acid and acid derivative forms, but in the acid sensitive form often offer advantages of solubility, tissue compatibility, or delayed release in the mammalian organism (see, Bundgard, H., Design of Prodrugs, pp. 7-9, 21-24, Elsevier, Amsterdam 1985). Prodrugs include acid derivatives well known to practitioners of the art, such as, for example, esters prepared by reaction of the parent acid with a suitable alcohol, or amides prepared by reaction of the parent acid compound with a substituted or unsubstituted amine, or acid anhydrides, or mixed anhydrides. Simple aliphatic or aromatic esters, amides, and anhydrides derived from acidic groups pendant on the compounds described herein are particular prodrugs. In some cases it is desirable to prepare double ester type prodrugs such as (acyloxy)alkyl esters or ((alkoxycarbonyl)oxy)alkylesters. C₁₋₈ alkyl, C₂₋₈ alkenyl, C₂₋₈ alkynyl, aryl, C₇₋₁₂ substituted aryl, and C₇₋₁₂ arylalkyl esters of the compounds described herein may be preferred.

The terms “composition” and “formulation” are used interchangeably.

A “subject” to which administration is contemplated refers to a human (i.e., male or female of any age group, e.g., pediatric subject (e.g., infant, child, or adolescent) or adult subject (e.g., young adult, middle-aged adult, or senior adult)) or non-human animal. In certain embodiments, the non-human animal is a mammal (e.g., primate (e.g., cynomolgus monkey or rhesus monkey), commercially relevant mammal (e.g., cattle, pig, horse, sheep, goat, cat, or dog), or bird (e.g., commercially relevant bird, such as chicken, duck, goose, or turkey)). In certain embodiments, the non-human animal is a fish, reptile, or amphibian. The non-human animal may be a male or female at any stage of development. The non-human animal may be a transgenic animal or genetically engineered animal. The term “patient” refers to a human subject in need of treatment of a disease. The subject may also be a plant. In certain embodiments, the plant is a land plant. In certain embodiments, the plant is a non-vascular land plant. In certain embodiments, the plant is a vascular land plant. In certain embodiments, the plant is a seed plant. In certain embodiments, the plant is a cultivated plant. In certain embodiments, the plant is a dicot. In certain embodiments, the plant is a monocot. In certain embodiments, the plant is a flowering plant. In some embodiments, the plant is a cereal plant, e.g., maize, corn, wheat, rice, oat, barley, rye, or millet. In some embodiments, the plant is a legume, e.g., a bean plant, e.g., soybean plant. In some embodiments, the plant is a tree or shrub.

The term “biological sample” refers to any sample including tissue samples (such as tissue sections and needle biopsies of a tissue); cell samples (e.g., cytological smears (such as Pap or blood smears) or samples of cells obtained by microdissection); samples of whole organisms (such as samples of yeasts or bacteria); or cell fractions, fragments or organelles (such as obtained by lysing cells and separating the components thereof by centrifugation or otherwise). Other examples of biological samples include blood, serum, urine, semen, fecal matter, cerebrospinal fluid, interstitial fluid, mucous, tears, sweat, pus, biopsied tissue (e.g., obtained by a surgical biopsy or needle biopsy), nipple aspirates, milk, vaginal fluid, saliva, swabs (such as buccal swabs), or any material containing biomolecules that is derived from a first biological sample.

The term “tissue” refers to any biological tissue of a subject (including a group of cells, a body part, or an organ) or a part thereof, including blood and/or lymph vessels, which is the object to which a compound, particle, and/or composition of the invention is delivered. A tissue may be an abnormal or unhealthy tissue, which may need to be treated. A tissue may also be a normal or healthy tissue that is under a higher than normal risk of becoming abnormal or unhealthy, which may need to be prevented. In certain embodiments, the tissue is the central nervous system. In certain embodiments, the tissue is the brain.

The term “administer.” “administering.” or “administration” refers to implanting, absorbing, ingesting, injecting, inhaling, or otherwise introducing a compound described herein, or a composition thereof, in or on a subject.

The terms “treatment,” “treat.” and “treating” refer to reversing, alleviating, delaying the onset of, or inhibiting the progress of a disease described herein. In some embodiments, treatment may be administered after one or more signs or symptoms of the disease have developed or have been observed. In other embodiments, treatment may be administered in the absence of signs or symptoms of the disease. For example, treatment may be administered to a susceptible subject prior to the onset of symptoms (e.g., in light of a history of symptoms). Treatment may also be continued after symptoms have resolved, for example, to delay or prevent recurrence.

The terms “condition,” “disease,” and “disorder” are used interchangeably.

An “effective amount” of a compound described herein refers to an amount sufficient to elicit the desired biological response. An effective amount of a compound described herein may vary depending on such factors as the desired biological endpoint, the pharmacokinetics of the compound, the condition being treated, the mode of administration, and the age and health of the subject. In certain embodiments, an effective amount is a therapeutically effective amount. In certain embodiments, an effective amount is a prophylactic treatment. In certain embodiments, an effective amount is the amount of a compound described herein in a single dose. In certain embodiments, an effective amount is the combined amounts of a compound described herein in multiple doses.

A “therapeutically effective amount” of a compound described herein is an amount sufficient to provide a therapeutic benefit in the treatment of a condition or to delay or minimize one or more symptoms associated with the condition. A therapeutically effective amount of a compound means an amount of therapeutic agent, alone or in combination with other therapies, which provides a therapeutic benefit in the treatment of the condition. The term “therapeutically effective amount” can encompass an amount that improves overall therapy, reduces, or avoids symptoms, signs, or causes of the condition, and/or enhances the therapeutic efficacy of another therapeutic agent. In certain embodiments, a therapeutically effective amount is an amount sufficient for promoting the degradation of a protein (e.g., IKZF2). In certain embodiments, a therapeutically effective amount is an amount sufficient for treating a cancer.

A “prophylactically effective amount” of a compound described herein is an amount sufficient to prevent a condition, or one or more signs or symptoms associated with the condition, or prevent its recurrence. A prophylactically effective amount of a compound means an amount of a therapeutic agent, alone or in combination with other agents, which provides a prophylactic benefit in the prevention of the condition. The term “prophylactically effective amount” can encompass an amount that improves overall prophylaxis or enhances the prophylactic efficacy of another prophylactic agent.

A “proliferative disease” refers to a disease that occurs due to abnormal growth or extension by the multiplication of cells (Walker, Cambridge Dictionary of Biology;

Cambridge University Press: Cambridge, UK, 1990). A proliferative disease may be associated with: 1) the pathological proliferation of normally quiescent cells; 2) the pathological migration of cells from their normal location (e.g., metastasis of neoplastic cells); 3) the pathological expression of proteolytic enzymes such as the matrix metalloproteinases (e.g., collagenases, gelatinases, and elastases); or 4) the pathological angiogenesis as in proliferative retinopathy and tumor metastasis. Exemplary proliferative diseases include cancers (i.e., “malignant neoplasms”), benign neoplasms, angiogenesis, inflammatory diseases, and autoimmune diseases.

The term “angiogenesis” refers to the physiological process through which new blood vessels form from pre-existing vessels. Angiogenesis is distinct from vasculogenesis, which is the de novo formation of endothelial cells from mesoderm cell precursors. The first vessels in a developing embryo form through vasculogenesis, after which angiogenesis is responsible for most blood vessel growth during normal or abnormal development. Angiogenesis is a vital process in growth and development, as well as in wound healing and in the formation of granulation tissue. However, angiogenesis is also a fundamental step in the transition of tumors from a benign state to a malignant one, leading to the use of angiogenesis inhibitors in the treatment of cancer. Angiogenesis may be chemically stimulated by angiogenic proteins, such as growth factors (e.g., VEGF). “Pathological angiogenesis” refers to abnormal (e.g., excessive or insufficient) angiogenesis that amounts to and/or is associated with a disease (e.g., cancer).

The terms “neoplasm” and “tumor” are used herein interchangeably and refer to an abnormal mass of tissue wherein the growth of the mass surpasses and is not coordinated with the growth of a normal tissue. A neoplasm or tumor may be “benign” or “malignant,” depending on the following characteristics: degree of cellular differentiation (including morphology and functionality), rate of growth, local invasion, and metastasis. A “benign neoplasm” is generally well differentiated, has characteristically slower growth than a malignant neoplasm, and remains localized to the site of origin. In addition, a benign neoplasm does not have the capacity to infiltrate, invade, or metastasize to distant sites. Exemplary benign neoplasms include, but are not limited to, lipoma, chondroma, adenomas, acrochordon, senile angiomas, seborrheic keratoses, lentigos, and sebaceous hyperplasias. In some cases, certain “benign” tumors may later give rise to malignant neoplasms, which may result from additional genetic changes in a subpopulation of the tumor's neoplastic cells, and these tumors are referred to as “pre-malignant neoplasms.” An exemplary pre-malignant neoplasm is a teratoma. In contrast, a “malignant neoplasm” is generally poorly differentiated (anaplasia) and has characteristically rapid growth accompanied by progressive infiltration, invasion, and destruction of the surrounding tissue. Furthermore, a malignant neoplasm generally has the capacity to metastasize to distant sites. The term “metastasis,” “metastatic,” or “metastasize” refers to the spread or migration of cancerous cells from a primary or original tumor to another organ or tissue and is typically identifiable by the presence of a “secondary tumor” or “secondary cell mass” of the tissue type of the primary or original tumor and not of that of the organ or tissue in which the secondary (metastatic) tumor is located. For example, a prostate cancer that has migrated to bone is said to be metastasized prostate cancer and includes cancerous prostate cancer cells growing in bone tissue.

The term “cancer” refers to a class of diseases characterized by the development of abnormal cells that proliferate uncontrollably and have the ability to infiltrate and destroy normal body tissues. Sec, e.g., Stedman's Medical Dictionary, 25th ed.; Hensyl ed.; Williams & Wilkins: Philadelphia, 1990. Exemplary cancers include, but are not limited to, hematological malignancies. Additional exemplary cancers include, but are not limited to, lung cancer (e.g., bronchogenic carcinoma, small cell lung cancer (SCLC), non-small cell lung cancer (NSCLC), adenocarcinoma of the lung); kidney cancer (e.g., nephroblastoma, a.k.a. Wilms' tumor, renal cell carcinoma); acoustic neuroma; adenocarcinoma; adrenal gland cancer; anal cancer; angiosarcoma (e.g., lymphangiosarcoma, lymphangioendotheliosarcoma, hemangiosarcoma); appendix cancer; benign monoclonal gammopathy; biliary cancer (e.g., cholangiocarcinoma); bladder cancer; breast cancer (e.g., adenocarcinoma of the breast, papillary carcinoma of the breast, mammary cancer, medullary carcinoma of the breast); brain cancer (e.g., meningioma, glioblastomas, glioma (e.g., astrocytoma, oligodendroglioma), medulloblastoma); bronchus cancer; carcinoid tumor; cervical cancer (e.g., cervical adenocarcinoma); choriocarcinoma; chordoma; craniopharyngioma; colorectal cancer (e.g., colon cancer, rectal cancer, colorectal adenocarcinoma); connective tissue cancer; epithelial carcinoma; ependymoma; endotheliosarcoma (e.g., Kaposi's sarcoma, multiple idiopathic hemorrhagic sarcoma); endometrial cancer (e.g., uterine cancer, uterine sarcoma); esophageal cancer (e.g., adenocarcinoma of the esophagus, Barrett's adenocarcinoma); Ewing's sarcoma; ocular cancer (e.g., intraocular melanoma, retinoblastoma); familiar hypercosinophilia; gall bladder cancer; gastric cancer (e.g., stomach adenocarcinoma); gastrointestinal stromal tumor (GIST); germ cell cancer; head and neck cancer (e.g., head and neck squamous cell carcinoma, oral cancer (e.g., oral squamous cell carcinoma), throat cancer (e.g., laryngeal cancer, pharyngeal cancer, nasopharyngeal cancer, oropharyngeal cancer)); heavy chain disease (e.g., alpha chain disease, gamma chain disease, mu chain disease; hemangioblastoma; hypopharynx cancer; inflammatory myofibroblastic tumors; immunocytic amyloidosis; liver cancer (e.g., hepatocellular cancer (HCC), malignant hepatoma); leiomyosarcoma (LMS); mastocytosis (e.g., systemic mastocytosis); muscle cancer; myelodysplastic syndrome (MDS); mesothelioma; myeloproliferative disorder (MPD) (e.g., polycythemia vera (PV), essential thrombocytosis (ET), agnogenic myeloid metaplasia (AMM) a.k.a. myelofibrosis (MF), chronic idiopathic myelofibrosis, chronic myelocytic leukemia (CML), chronic neutrophilic leukemia (CNL), hypereosinophilic syndrome (HES)); neuroblastoma; neurofibroma (e.g., neurofibromatosis (NF) type 1 or type 2, schwannomatosis); neuroendocrine cancer (e.g., gastroenteropancreatic neuroendocrine tumor (GEP-NET), carcinoid tumor); osteosarcoma (e.g., bone cancer); ovarian cancer (e.g., cystadenocarcinoma, ovarian embryonal carcinoma, ovarian adenocarcinoma); papillary adenocarcinoma; pancreatic cancer (e.g., pancreatic adenocarcinoma, intraductal papillary mucinous neoplasm (IPMN), Islet cell tumors); penile cancer (e.g., Paget's disease of the penis and scrotum); pinealoma; primitive neuroectodermal tumor (PNT); plasma cell neoplasia; paraneoplastic syndromes; intraepithelial neoplasms; prostate cancer (e.g., prostate adenocarcinoma); rectal cancer; rhabdomyosarcoma; salivary gland cancer; skin cancer (e.g., squamous cell carcinoma (SCC), keratoacanthoma (KA), melanoma, basal cell carcinoma (BCC)); small bowel cancer (e.g., appendix cancer); soft tissue sarcoma (e.g., malignant fibrous histiocytoma (MFH), liposarcoma, malignant peripheral nerve sheath tumor (MPNST), chondrosarcoma, fibrosarcoma, myxosarcoma); sebaceous gland carcinoma; small intestine cancer; sweat gland carcinoma; synovioma; testicular cancer (e.g., seminoma, testicular embryonal carcinoma); thyroid cancer (e.g., papillary carcinoma of the thyroid, papillary thyroid carcinoma (PTC), medullary thyroid cancer); urethral cancer; vaginal cancer; and vulvar cancer (e.g., Paget's disease of the vulva).

The term “hematological cancer” refers to cancer that begins in blood-forming tissue, such as the bone marrow, or in the cells of the immune system. Examples of hematologic cancer are leukemia, lymphoma, and multiple myeloma. Hematological cancer is also called blood cancer. Exemplary hematological cancers include, but are not limited to, leukemia, such as acute lymphocytic leukemia (ALL) (e.g., B-cell ALL, T-cell ALL), acute myelocytic leukemia (AML) (e.g., B-cell AML, T-cell AML), chronic myelocytic leukemia (CML) (e.g., B-cell CML, T-cell CML), and chronic lymphocytic leukemia (CLL) (e.g., B-cell CLL, T-cell CLL)); lymphoma, such as Hodgkin lymphoma (HL) (e.g., B-cell HL, T-cell HL) and non-Hodgkin lymphoma (NHL) (e.g., B-cell NHL, such as diffuse large cell lymphoma (DLCL) (e.g., diffuse large B-cell lymphoma (DLBCL, e.g., activated B-cell (ABC) DLBCL (ABC-DLBCL))), follicular lymphoma, chronic lymphocytic leukemia/small lymphocytic lymphoma (CLL/SLL), mantle cell lymphoma (MCL), marginal zone B-cell lymphoma (e.g., mucosa-associated lymphoid tissue (MALT) lymphoma, nodal marginal zone B-cell lymphoma, splenic marginal zone B-cell lymphoma), primary mediastinal B-cell lymphoma, Burkitt lymphoma, Waldenström's macroglobulinemia (WM, lymphoplasmacytic lymphoma), hairy cell leukemia (HCL), immunoblastic large cell lymphoma, precursor B-lymphoblastic lymphoma, central nervous system (CNS) lymphoma (e.g., primary CNS lymphoma and secondary CNS lymphoma); and T-cell NHL, such as precursor T-lymphoblastic lymphoma/leukemia, peripheral T-cell lymphoma (PTCL) (e.g., cutaneous T-cell lymphoma (CTCL) (e.g., mycosis fungoides, Sezary syndrome), angioimmunoblastic T-cell lymphoma, extranodal natural killer T-cell lymphoma, enteropathy type T-cell lymphoma, subcutaneous panniculitis-like T-cell lymphoma, and anaplastic large cell lymphoma); lymphoma of an immune privileged site (e.g., cerebral lymphoma, ocular lymphoma, lymphoma of the placenta, lymphoma of the fetus, testicular lymphoma); a mixture of one or more leukemia/lymphoma as described above; myelodysplasia; and multiple myeloma (MM).

The term “leukemia” refers to broadly progressive, malignant diseases of the blood-forming organs and is generally characterized by a distorted proliferation and development of leukocytes and their precursors in the blood and bone marrow. Leukemia diseases include, for example, acute nonlymphocytic leukemia, chronic lymphocytic leukemia, acute granulocytic leukemia, chronic granulocytic leukemia, acute promyelocytic leukemia, adult T-cell leukemia, aleukemic leukemia, a leukocythemic leukemia, basophylic leukemia, blast cell leukemia, bovine leukemia, chronic myelocytic leukemia, leukemia cutis, embryonal leukemia, eosinophilic leukemia, Gross' leukemia, hairy-cell leukemia, hemoblastic leukemia, hemocytoblastic leukemia, histiocytic leukemia, stem cell leukemia, acute monocytic leukemia, leukopenic leukemia, lymphatic leukemia, lymphoblastic leukemia, lymphocytic leukemia, lymphogenous leukemia, lymphoid leukemia, lymphosarcoma cell leukemia, mast cell leukemia, megakaryocytic leukemia, micromyeloblastic leukemia, monocytic leukemia, myeloblastic leukemia, myelocytic leukemia, myeloid granulocytic leukemia, myelomonocytic leukemia, Naegeli leukemia, plasma cell leukemia, plasmacytic leukemia, promyelocytic leukemia, Rieder cell leukemia, Schilling's leukemia, stem cell leukemia, subleukemic leukemia, and undifferentiated cell leukemia.

The term “lymphoma” refers to a group of blood cancers that develop from lymphocytes. Lymphoma disease includes diffuse large B-cell lymphoma (DLBCL), B-cell immunoblastic lymphoma, small non-cleaved cell lymphoma, human lymphotropic virus-type 1 (HTLV-1) leukemia/lymphoma, adult T-cell lymphoma, peripheral T-cell lymphoma (PTCL), cutaneous T-cell lymphoma (CTCL), mantle cell lymphoma (MCL), Hodgkin's lymphoma (HL), non-Hodgkin's lymphoma (NHL), AIDS-related lymphoma, follicular lymphoma, small lymphocytic lymphoma, T-cell/histiocyte rich large B-cell lymphoma, transformed lymphoma, primary mediastinal (thymic) large B-cell lymphoma, splenic marginal zone lymphoma, Richter's transformation, nodal marginal zone lymphoma, or ALK-positive large B-cell lymphoma.

The terms “biologic,” “biologic drug,” and “biological product” refer to a wide range of products such as vaccines, blood and blood components, allergenics, somatic cells, gene therapy, tissues, nucleic acids, and proteins. Biologics may include sugars, proteins, or nucleic acids, or complex combinations of these substances, or may be living entities, such as cells and tissues. Biologics may be isolated from a variety of natural sources (e.g., human, animal, microorganism) and may be produced by biotechnological methods and other technologies.

The term “small molecule” or “small molecule therapeutic” refers to molecules, whether naturally occurring or artificially created (e.g., via chemical synthesis) that have a relatively low molecular weight. Typically, a small molecule is an organic compound (i.e., it contains carbon). The small molecule may contain multiple carbon-carbon bonds, stereocenters, and other functional groups (e.g., amines, hydroxyl, carbonyls, and heterocyclic rings, etc.). In certain embodiments, the molecular weight of a small molecule is not more than about 1,000 g/mol, not more than about 900 g/mol, not more than about 800 g/mol, not more than about 700 g/mol, not more than about 600 g/mol, not more than about 500 g/mol, not more than about 400 g/mol, not more than about 300 g/mol, not more than about 200 g/mol, or not more than about 100 g/mol. In certain embodiments, the molecular weight of a small molecule is at least about 100 g/mol, at least about 200 g/mol, at least about 300 g/mol, at least about 400 g/mol, at least about 500 g/mol, at least about 600 g/mol, at least about 700 g/mol, at least about 800 g/mol, or at least about 900 g/mol, or at least about 1,000 g/mol. Combinations of the above ranges (e.g., at least about 200 g/mol and not more than about 500 g/mol) are also possible. In certain embodiments, the small molecule is a therapeutically active agent such as a drug (e.g., a molecule approved by the U.S. Food and Drug Administration as provided in the Code of Federal Regulations (C.F.R.)). The small molecule may also be complexed with one or more metal atoms and/or metal ions. In this instance, the small molecule is also referred to as a “small organometallic molecule.” Preferred small molecules are biologically active in that they produce a biological effect in animals, preferably mammals, more preferably humans. Small molecules include, but are not limited to, radionuclides and imaging agents. In certain embodiments, the small molecule is a drug. Preferably, though not necessarily, the drug is one that has already been deemed safe and effective for use in humans or animals by the appropriate governmental agency or regulatory body. For example, drugs approved for human use are listed by the FDA under 21 C.F.R. §§ 330.5, 331 through 361, and 440 through 460, incorporated herein by reference; drugs for veterinary use are listed by the FDA under 21 C.F.R. §§ 500 through 589, incorporated herein by reference. All listed drugs are considered acceptable for use in accordance with the present disclosure.

The term “therapeutic agent” refers to any substance having therapeutic properties that produce a desired, usually beneficial, effect. For example, therapeutic agents may treat, ameliorate, and/or prevent disease. Therapeutic agents, as disclosed herein, may be biologics or small molecule therapeutics.

### DETAILED DESCRIPTION OF CERTAIN EMBODIMENTS

Provided herein are compounds that bind to and promote the degradation of CK1α, IKZF1, IKZF2, and/or GSPT1. In one aspect, the disclosure provides compounds of Formula (I), and pharmaceutically acceptable salts, solvates, hydrates, polymorphs, co-crystals, tautomers, stereoisomers, isotopically labeled derivatives, prodrugs, and pharmaceutical compositions thereof. The compounds are useful for the treatment of diseases associated with CK1α, IKZF1, IKZF2, and/or GSPT1 (e.g., a proliferative disease) in a subject in need thereof. In certain embodiments, the compounds selectively promote the degradation of CK1α and/or IKZF2 over IKZF1 and GSPT1.

### Compounds

In one aspect, disclosed is a compound of Formula (I):

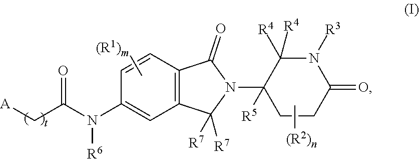

or a pharmaceutically acceptable salt, co-crystal, tautomer, stereoisomer, solvate, hydrate, polymorph, isotopically enriched derivative, or prodrug thereof, wherein:
each R¹ is independently halogen, —OR^{A}, or substituted or unsubstituted C₁-C₆ alkyl;
each R² is independently halogen or C₁-C₃ alkyl;
R³ is hydrogen or C₁-C₃ alkyl;
each R⁴ is independently hydrogen or C₁-C₃ alkyl; or two R⁴, together with the carbon atom to which they are attached, form a C═O, C₃-C₆ carbocycle, or a 4-6-membered heterocycle;
R⁵ is hydrogen, halogen, or C₁-C₃ alkyl;
R⁶ is hydrogen, substituted or unsubstituted acyl, substituted or unsubstituted alkyl, or a nitrogen protecting group;
each R⁷ is independently hydrogen or C₁-C₃ alkyl; or two R⁷, together with the carbon atom to which they are attached, form a C═O;
A is

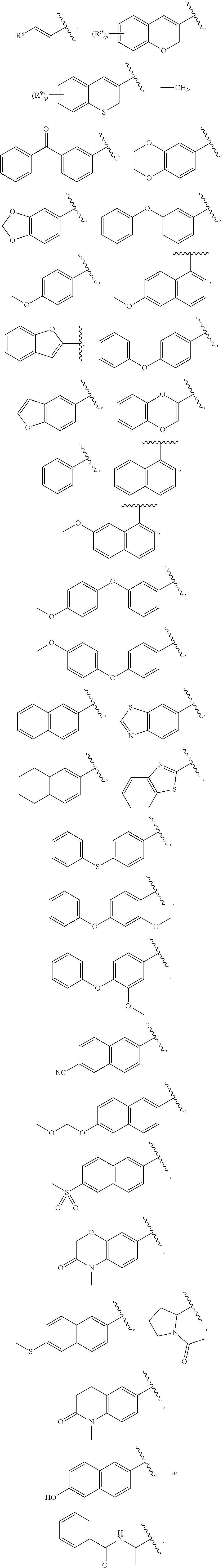

R⁸ is hydrogen, substituted or unsubstituted alkyl, substituted or unsubstituted heteroalkyl, substituted or unsubstituted carbocyclyl, substituted or unsubstituted heterocyclyl, substituted or unsubstituted aryl, or substituted or unsubstituted heteroaryl;
each R⁹ is independently halogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, substituted or unsubstituted heteroaliphatic, —OR^{A}, —N(R^{A})₂, —SR^{A}, —CN, —SCN, —C(═NR^{A})R^{A}, —C(═NR^{A})OR^{A}, —C(═NR^{A})N(R^{A})₂, —C(═O)R^{A}, —C(═O)OR^{A}, —C(═O)N(R^{A})₂, —NO₂, —NR^{A}C(═O)R^{A}, —NR^{A}C(═O)OR^{A}, —NR^{A}C(═O)N(R^{A})₂, —NR^{A}C(═NR^{A})N(R^{A})₂, —OC(═O)R^{A}, —OC(═O)OR^{A}, —OC(═O)N(R^{A})₂, —NR^{A}S(O)₂R^{A}, —OS(O)₂R^{A}, or —S(O)₂R^{A}; or two R⁹ groups are joined to form a substituted or unsubstituted heterocyclyl ring, or a substituted or unsubstituted carbocyclyl ring;
each R^{A} is independently hydrogen, substituted or unsubstituted acyl, substituted or unsubstituted alkyl, substituted or unsubstituted alkenyl, substituted or unsubstituted alkynyl, substituted or unsubstituted heteroaliphatic, substituted or unsubstituted carbocyclyl, substituted or unsubstituted carbocyclylalkyl, substituted or unsubstituted heterocyclyl, substituted or unsubstituted aryl, substituted or unsubstituted aralkyl, substituted or unsubstituted heteroaryl, substituted or unsubstituted hetaralkyl, a nitrogen protecting group when attached to a nitrogen atom, an oxygen protecting group when attached to an oxygen atom, or a sulfur protecting group when attached to a sulfur atom, or two R^{A} groups are joined to form a substituted or unsubstituted heterocyclyl ring, or a substituted or unsubstituted heteroaryl ring;
m is 0, 1, 2 or 3;
n is 0, 1, or 2;
t is 0, 1, or 2; and
p is 0, 1, 2, 3, or 4;
provided that the compound is not of formula:

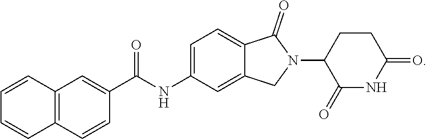 [SMILES: O=C1CCC(N2Cc3cc(NC(=O)c4ccc5ccccc5c4)ccc3C2=O)C(=O)N1]

In certain embodiments, disclosed is a compound of Formula (I):

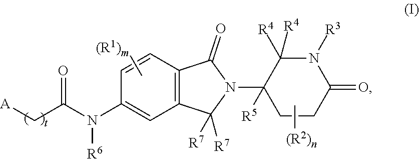

or a pharmaceutically acceptable salt, co-crystal, tautomer, stereoisomer, solvate, hydrate, polymorph, isotopically enriched derivative, or prodrug thereof, wherein:
each R¹ is independently halogen, —OR^{A}, or substituted or unsubstituted C₁-C₆ alkyl;
each R² is independently halogen or C₁-C₃ alkyl;
R³ is hydrogen or C₁-C₃ alkyl;
each R⁴ is independently hydrogen or C₁-C₃ alkyl; or two R⁴, together with the carbon atom to which they are attached, form a C═O, C₃-C₆ carbocycle, or a 4-6-membered heterocycle;
R⁵ is hydrogen, halogen, or C₁-C₃ alkyl;
R⁶ is hydrogen, substituted or unsubstituted acyl, substituted or unsubstituted alkyl, or a nitrogen protecting group;
each R⁷ is independently hydrogen or C₁-C₃ alkyl; or two R⁷, together with the carbon atom to which they are attached, form a C═O;
A is

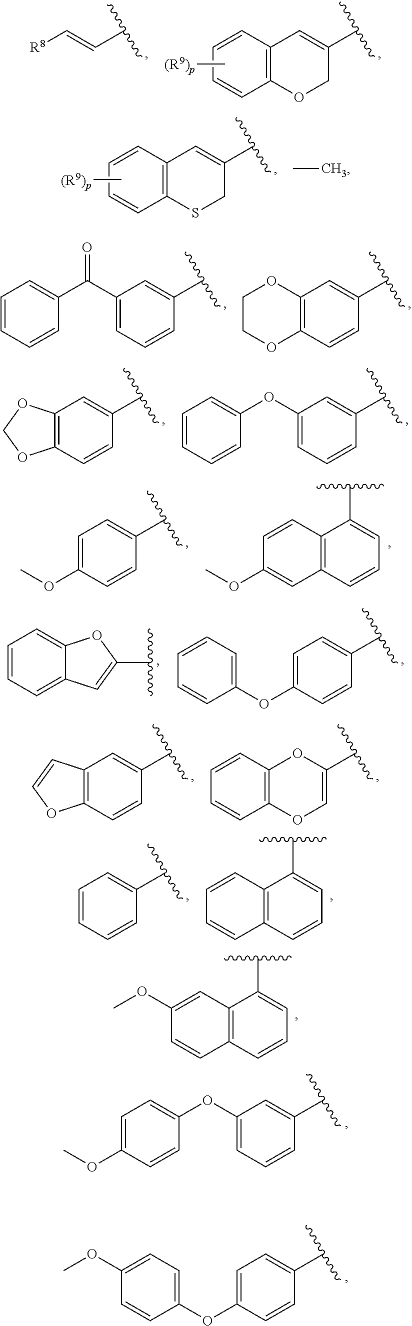

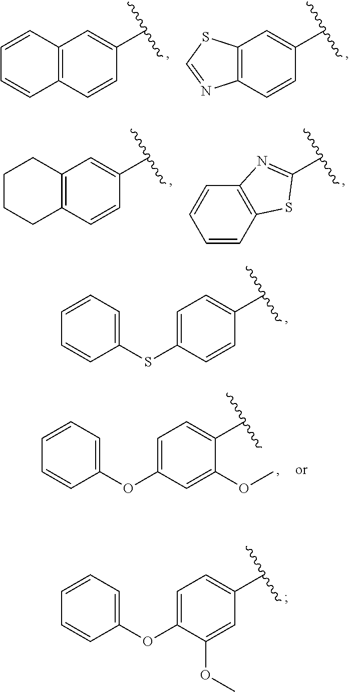

R⁸ is hydrogen, substituted or unsubstituted alkyl, substituted or unsubstituted heteroalkyl, substituted or unsubstituted carbocyclyl, substituted or unsubstituted heterocyclyl, substituted or unsubstituted aryl, or substituted or unsubstituted heteroaryl;
each R⁹ is independently halogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, substituted or unsubstituted heteroaliphatic, —OR^{A}, —N(R^{A})₂, —SR^{A}, —CN, —SCN, —C(═NR^{A})R^{A}, —C(═NR^{A})OR^{A}, —C(═NR^{A})N(R^{A})₂, C(═O)R^{A}, —C(═O)OR^{A}, —C(═O)N(R^{A})₂, —NO₂, —NR—C(═O)R^{A}, —NR^{A}C(═O)OR^{A}, —NR^{A}C(═O)N(R^{A})₂, —NR^{A}C(═NR^{A})N(R^{A})₂, —OC(═O)R^{A}, —OC(═O)OR^{A}, —OC(═O)N(R^{A})₂, —NR^{A}S(O)₂R^{A}, —OS(O)₂R^{A}, or —S(O)₂R^{A}; or two R⁹ groups are joined to form a substituted or unsubstituted heterocyclyl ring, or a substituted or unsubstituted carbocyclyl ring;
each R^{A} is independently hydrogen, substituted or unsubstituted acyl, substituted or unsubstituted alkyl, substituted or unsubstituted alkenyl, substituted or unsubstituted alkynyl, substituted or unsubstituted heteroaliphatic, substituted or unsubstituted carbocyclyl, substituted or unsubstituted carbocyclylalkyl, substituted or unsubstituted heterocyclyl, substituted or unsubstituted aryl, substituted or unsubstituted aralkyl, substituted or unsubstituted heteroaryl, substituted or unsubstituted hetaralkyl, a nitrogen protecting group when attached to a nitrogen atom, an oxygen protecting group when attached to an oxygen atom, or a sulfur protecting group when attached to a sulfur atom, or two R^{A} groups are joined to form a substituted or unsubstituted heterocyclyl ring, or a substituted or unsubstituted heteroaryl ring;
m is 0, 1, 2 or 3;
n is 0, 1, or 2;
t is 0, 1, or 2; and
p is 0, 1, 2, 3, or 4;
provided that the compound is not of formula:

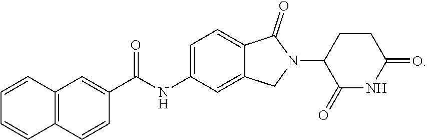 [SMILES: O=C1CCC(N2Cc3cc(NC(=O)c4ccc5ccccc5c4)ccc3C2=O)C(=O)N1]

### R¹

As described herein, each R¹ is, independently, halogen, —OR^{A}, or substituted or unsubstituted C₁-C₆ alkyl. In certain embodiments, each R¹ is, independently, halogen or —OR^{A}. In certain embodiments, each R¹ is independently halogen, or substituted or unsubstituted C₁-C₆ alkyl. In certain embodiments, each R¹ is independently —OR^{A}, or substituted or unsubstituted C₁-C₆ alkyl. In certain embodiments, each R¹ is independently halogen, or unsubstituted C₁-C₆ alkyl. In certain embodiments, each R¹ is independently halogen, or unsubstituted C₁₋₄ alkyl. In certain embodiments, each R¹ is independently unsubstituted C₁₋₄ alkyl. In certain embodiments, each R¹ is, independently, halogen (e.g., —F, —Cl, —Br, or —I).

As described herein, m is 0, 1, 2 or 3. In certain embodiments, m is 0, 1, or 2. In certain embodiments, m is 0 or 1. In certain embodiments, m is 0. In certain embodiments, m is 1. In certain embodiments, m is 2. In certain embodiments, m is 3.

### R²

As described herein, each R² is, independently, halogen or C₁-C₃ alkyl. In certain embodiments, each R² is, independently, —F, —Cl, —Br, —I, or C₁-C₃ alkyl. In certain embodiments, each R² is, independently, halogen (e.g., —F, —Cl, —Br, or —I). In certain embodiments, each R² is independently C₁-C₃ alkyl. In certain embodiments, each R² is independently C₁-C₂ alkyl. In certain embodiments, each R² is independently C₂-C₃ alkyl. In certain embodiments, each R² is independently methyl. In certain embodiments, each R² is independently ethyl. In certain embodiments, each R² is independently propyl. In certain embodiments, each R² is independently isopropyl.

As described herein, n is 0, 1, or 2. In certain embodiments, n is 0 or 1. In certain embodiments, n is 0. In certain embodiments, n is 1. In certain embodiments, n is 2.

### R³

As described herein, R³ is hydrogen or C₁-C₃ alkyl. In certain embodiments, R³ is C₁-C₃ alkyl. In certain embodiments, R³ is hydrogen.

### R⁴

As described herein, each R⁴ is, independently, hydrogen or C₁-C₃ alkyl; or two R⁴, together with the carbon atom to which they are attached, form a C═O, C₃-C₆ carbocycle, or a 4-6-membered heterocycle.

In certain embodiments, each R⁴ is, independently, hydrogen or C₁-C₃ alkyl; or two R⁴, together with the carbon atom to which they are attached, form a C═O. In certain embodiments, each R⁴, together with the carbon atom to which they are attached, form a C═O. In certain embodiments, each R⁴ is hydrogen.

In certain embodiments, each R⁴, together with the carbon atom to which they are attached, form a C₃-C₆ carbocycle or a 4-6-membered heterocycle. In certain embodiments, each R⁴, together with the carbon atom to which they are attached, form a C₃-C₆ carbocycle. In certain embodiments, each R⁴, together with the carbon atom to which they are attached, form a cyclopropyl. In certain embodiments, each R⁴, together with the carbon atom to which they are attached, form a cyclobutyl. In certain embodiments, each R⁴, together with the carbon atom to which they are attached, form a cyclopentyl. In certain embodiments, each R⁴, together with the carbon atom to which they are attached, form a cyclohexyl. In certain embodiments, each R⁴, together with the carbon atom to which they are attached, form a 4-6-membered heterocycle. In certain embodiments, each R⁴, together with the carbon atom to which they are attached, form a 4-membered heterocycle (e.g., oxetane, azetidine). In certain embodiments, each R⁴, together with the carbon atom to which they are attached, form a 5-membered heterocycle (e.g., pyrrolidine, tetrahydrofuran). In certain embodiments, each R⁴, together with the carbon atom to which they are attached, form a 6-membered heterocycle (e.g., piperidine, tetrahydropyran).

### R⁵

As described herein, R⁵ is hydrogen, halogen, or C₁-C₃ alkyl. In certain embodiments, R⁵ is hydrogen or halogen. In certain embodiments, R⁵ is halogen (e.g., —F, —Cl, —Br, or −I). In certain embodiments, R⁵ is hydrogen or C₁-C₃ alkyl. In certain embodiments, R⁵ is C₁-C₃ alkyl (e.g., methyl, ethyl, propyl, or isopropyl). In certain embodiments, R⁵ is hydrogen. In certain embodiments, R⁵ is deuterium.

### R⁶

As described herein R⁶ is hydrogen, substituted or unsubstituted acyl, substituted or unsubstituted alkyl, or a nitrogen protecting group.

In certain embodiments, R⁶ is hydrogen or substituted or unsubstituted alkyl. In certain embodiments, R⁶ is hydrogen or substituted or unsubstituted C₁-C₄ alkyl. In certain embodiments, R⁶ is hydrogen or unsubstituted alkyl. In certain embodiments, R⁶ is unsubstituted alkyl. In certain embodiments, R⁶ is unsubstituted C₁-C₄ alkyl. In certain embodiments, R⁶ is hydrogen.

### R⁷

As described herein, each R⁷ is, independently, hydrogen or C₁-C₃ alkyl; or each R⁷, together with the carbon atom to which they are attached, form a C═O. In certain embodiments, each R⁷ is, independently, C₁-C₃ alkyl; or each R⁷, together with the carbon atom to which they are attached, form a C═O. In certain embodiments, each R⁷ is, independently, hydrogen; or each R⁷, together with the carbon atom to which they are attached, form a C═O. In certain embodiments, each R⁷, together with the carbon atom to which they are attached, form a C═O. In certain embodiments, each R⁷ is hydrogen.

### A

As described herein, A is

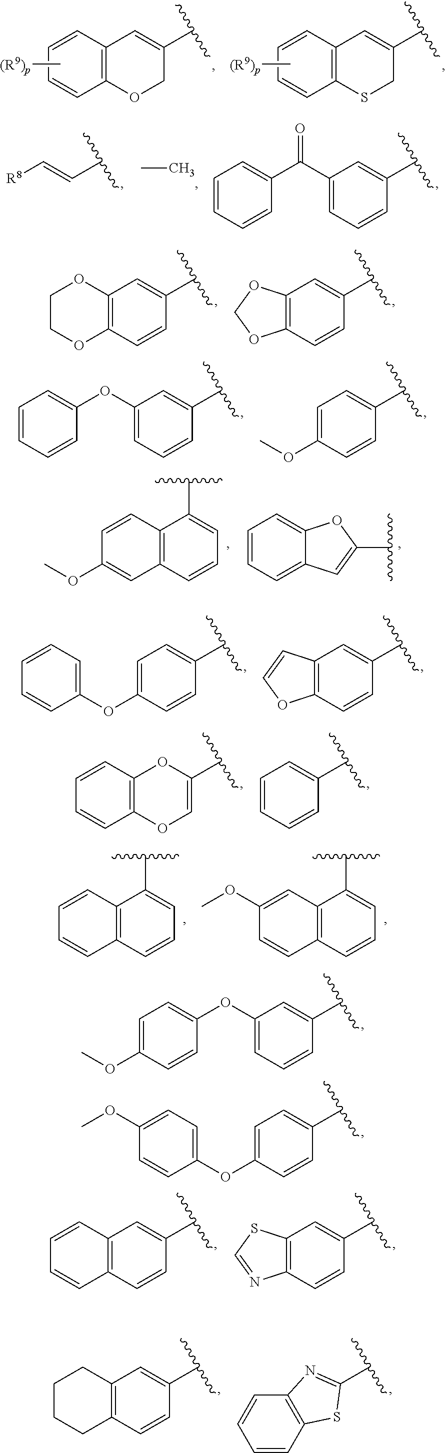

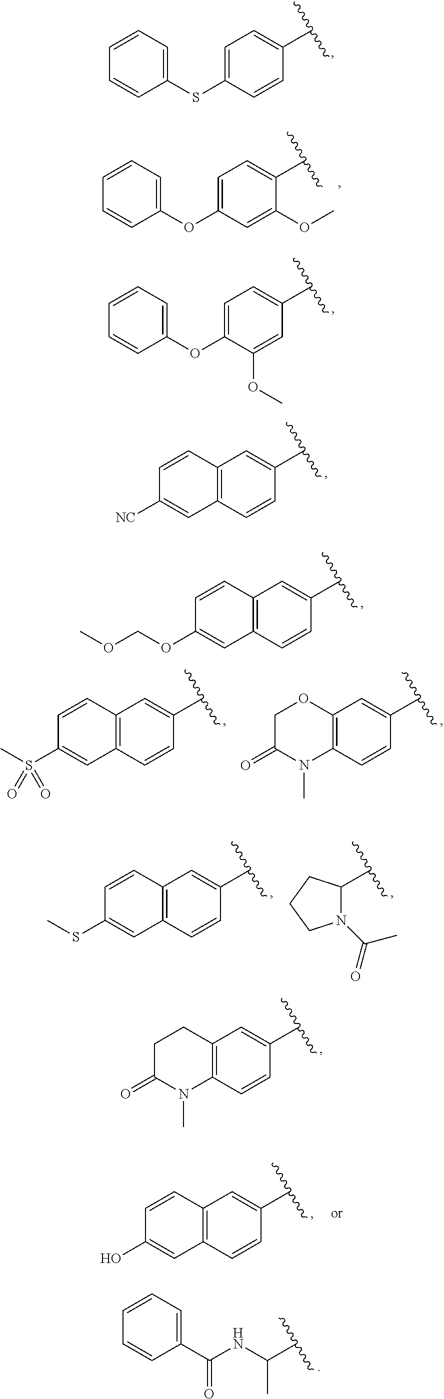

In certain embodiments, A is

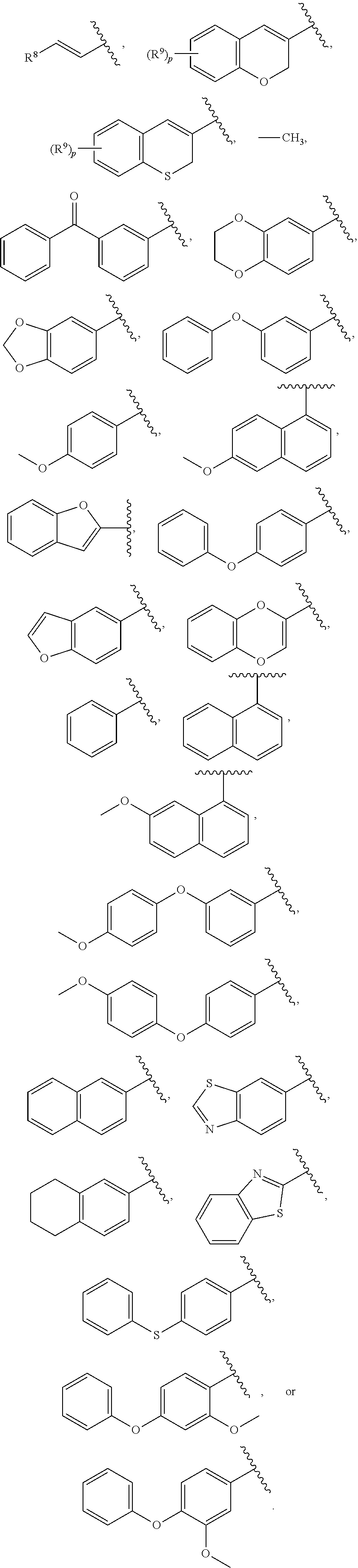

In certain embodiments, A is —CH₃.

In certain embodiments, A is

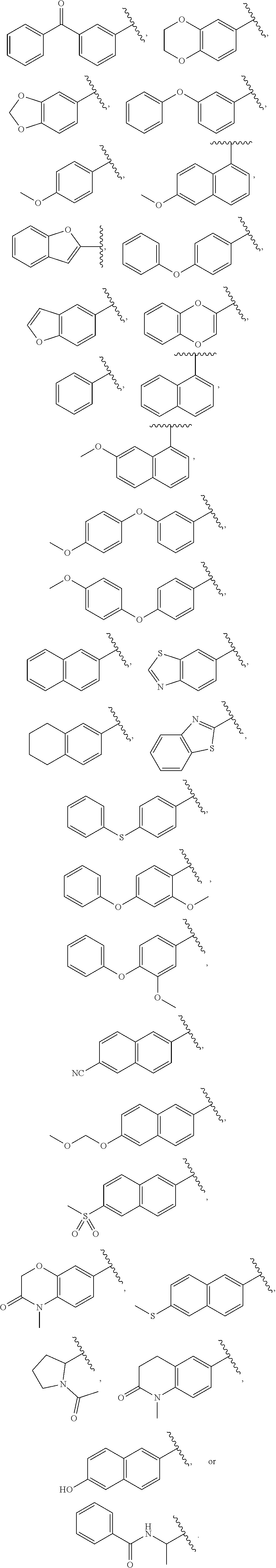

In certain embodiments, A is

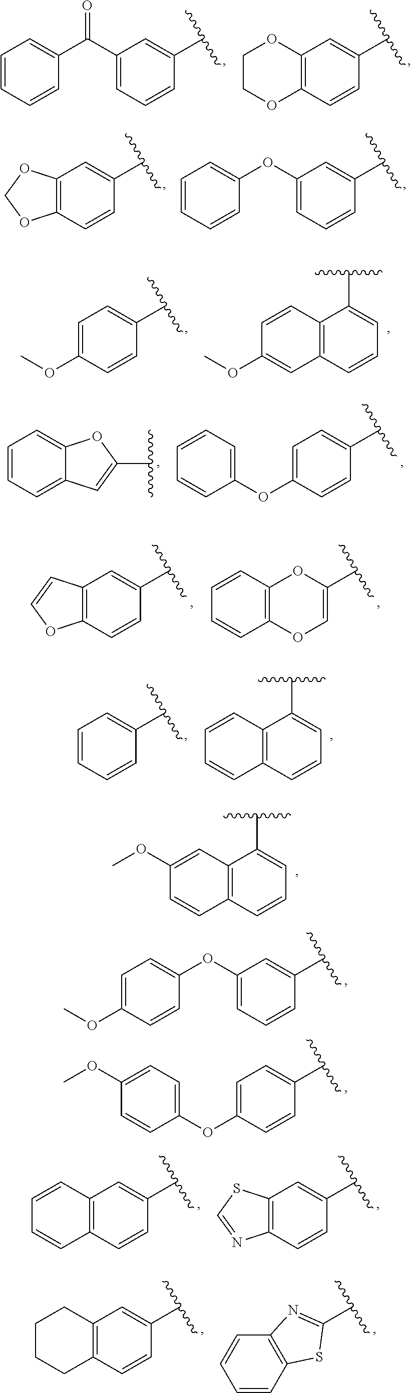

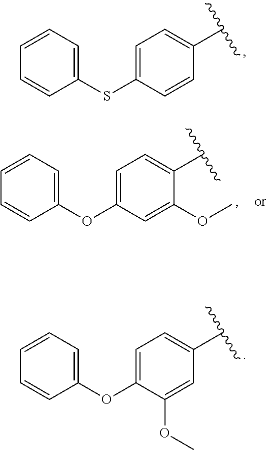 [SMILES: *c1ccc(Sc2ccccc2)cc1 ; *c1ccc(Oc2ccccc2)cc1OC ; *c1ccc(Oc2ccccc2)c(OC)c1]

In certain embodiments, A is

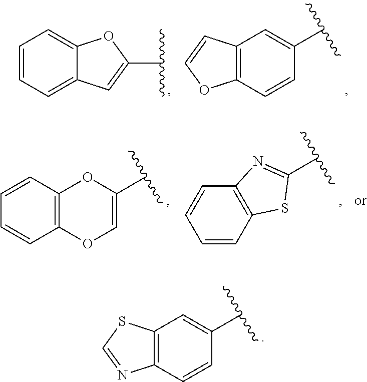

In certain embodiments, A is

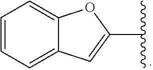 [SMILES: *c1cc2ccccc2o1]

In certain embodiments, A is

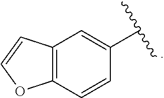 [SMILES: *c1ccc2occc2c1]

In certain embodiments, A is

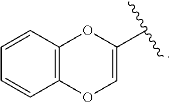 [SMILES: *C1=COc2ccccc2O1]

In certain embodiments, A is

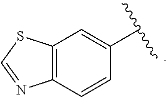 [SMILES: *c1ccc2ncsc2c1]

In certain embodiments, A is

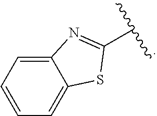 [SMILES: *c1nc2ccccc2s1]

In certain embodiments, A is

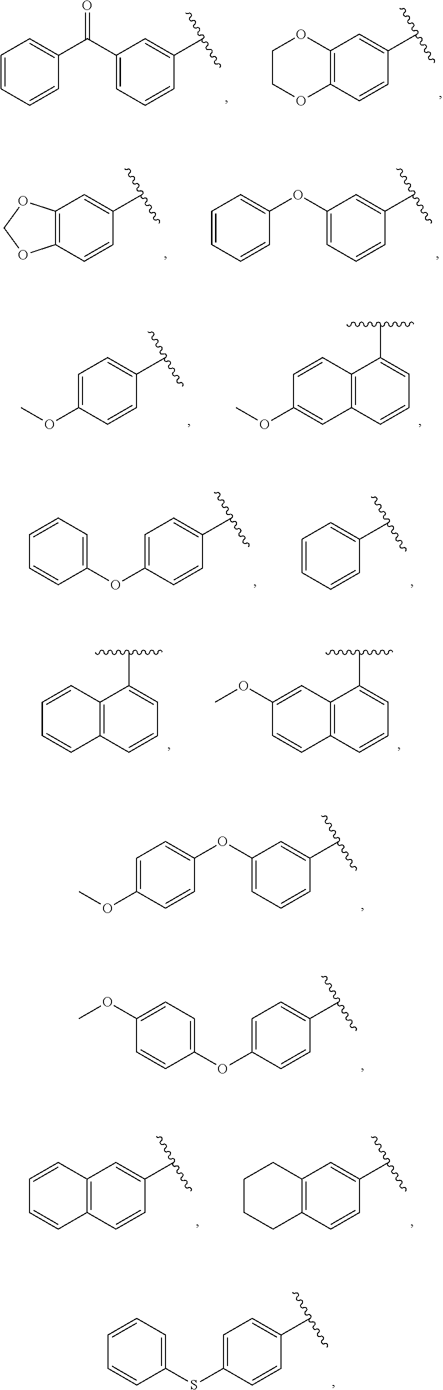

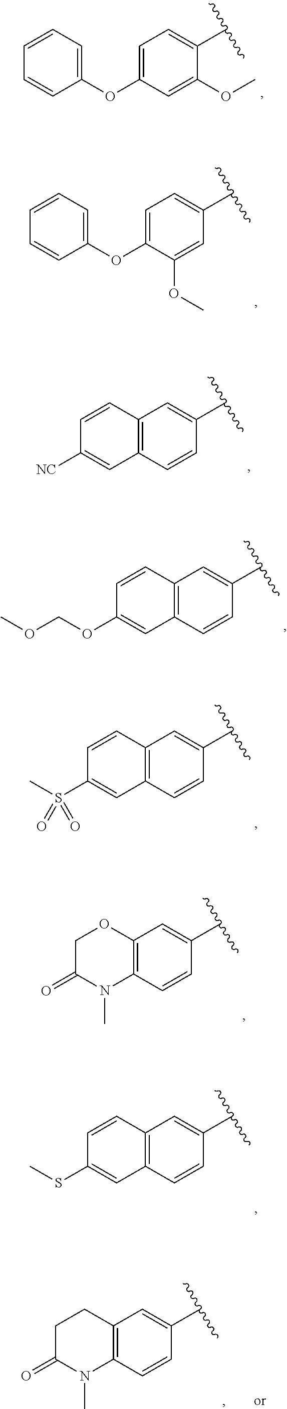

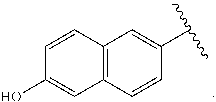 [SMILES: *c1ccc2cc(O)ccc2c1]

In certain embodiments, A is

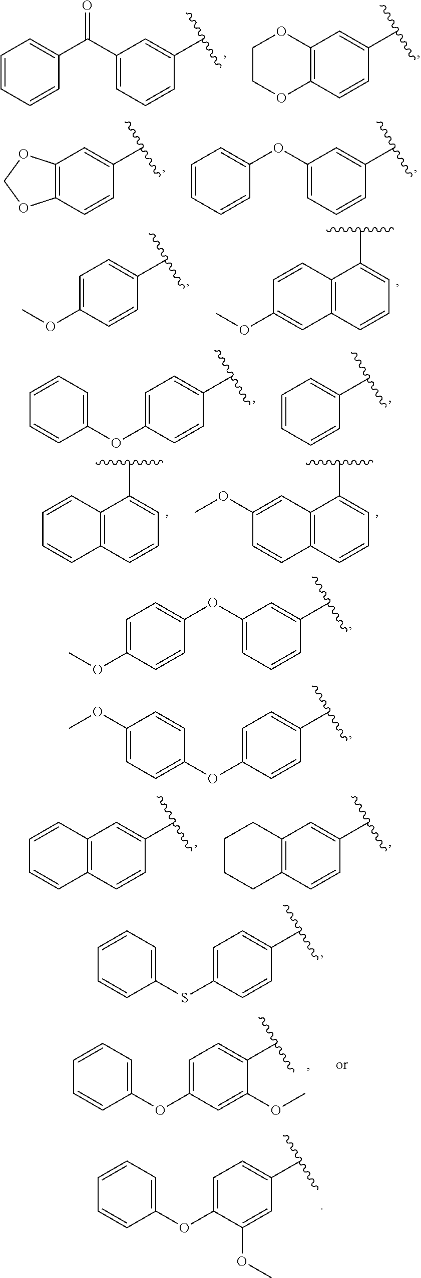

In certain embodiments, A is

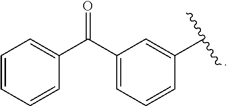 [SMILES: *c1cccc(C(=O)c2ccccc2)c1]

In certain embodiments, A is

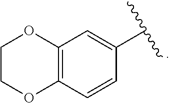 [SMILES: *c1ccc2c(c1)OCCO2]

In certain embodiments, A is

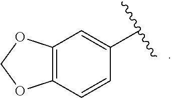 [SMILES: *c1ccc2c(c1)OCO2]

In certain embodiments, A is

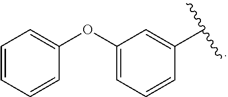 [SMILES: *c1cccc(Oc2ccccc2)c1]

In certain embodiments, A is

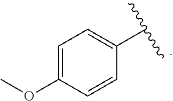 [SMILES: *c1ccc(OC)cc1]

In certain embodiments, A is

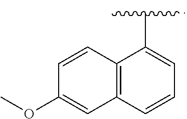 [SMILES: *c1cccc2cc(OC)ccc12]

In certain embodiments, A is

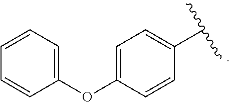 [SMILES: *c1ccc(Oc2ccccc2)cc1]

In certain embodiments, A is

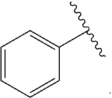 [SMILES: *c1ccccc1]

In certain embodiments, A is

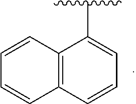 [SMILES: *c1cccc2ccccc12]

In certain embodiments, A is

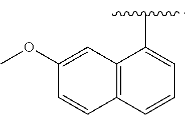 [SMILES: *c1cccc2ccc(OC)cc12]

In certain embodiments, A is

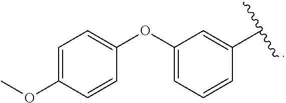 [SMILES: *c1cccc(Oc2ccc(OC)cc2)c1]

In certain embodiments, A is

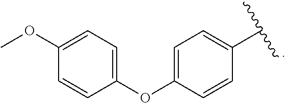 [SMILES: *c1ccc(Oc2ccc(OC)cc2)cc1]

In certain embodiments, A is

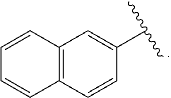 [SMILES: *c1ccc2ccccc2c1]

In certain embodiments, A is

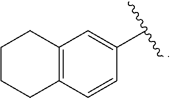 [SMILES: *c1ccc2c(c1)CCCC2]

In certain embodiments, A is

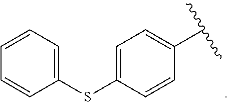 [SMILES: *c1ccc(Sc2ccccc2)cc1]

In certain embodiments, A is

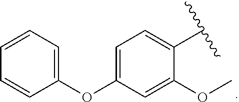 [SMILES: *c1ccc(Oc2ccccc2)cc1OC]

In certain embodiments, A is

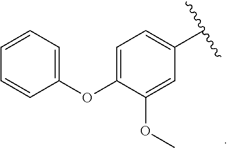 [SMILES: *c1ccc(Oc2ccccc2)c(OC)c1]

In certain embodiments, A is

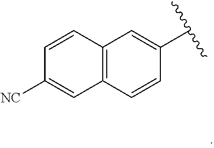 [SMILES: *c1ccc2cc(C#N)ccc2c1]

In certain embodiments, A is

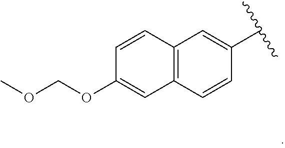 [SMILES: *c1ccc2cc(OCOC)ccc2c1]

In certain embodiments, A is

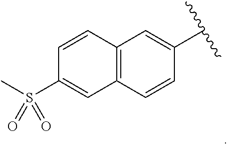 [SMILES: *c1ccc2cc(S(C)(=O)=O)ccc2c1]

In certain embodiments, A is

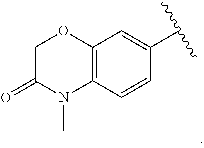 [SMILES: *c1ccc2c(c1)OCC(=O)N2C]

In certain embodiments, A is

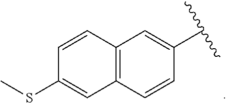 [SMILES: *c1ccc2cc(SC)ccc2c1]

In certain embodiments, A is

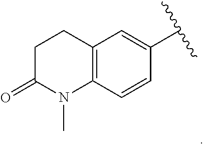 [SMILES: *c1ccc2c(c1)CCC(=O)N2C]

In certain embodiments, A is

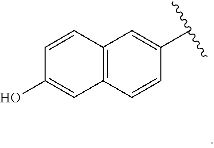 [SMILES: *c1ccc2cc(O)ccc2c1]

In certain embodiments, A is

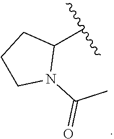 [SMILES: *C1CCCN1C(C)=O]

In certain embodiments, A is

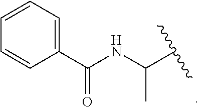 [SMILES: *C(C)NC(=O)c1ccccc1]

In certain embodiments, A is

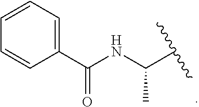 [SMILES: *[C@H](C)NC(=O)c1ccccc1]

In certain embodiments, A is

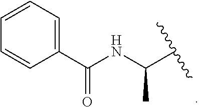 [SMILES: *[C@@H](C)NC(=O)c1ccccc1]

In certain embodiments, A is

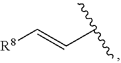

wherein R⁸ is hydrogen, substituted or unsubstituted alkyl, substituted or unsubstituted heteroalkyl, substituted or unsubstituted carbocyclyl, substituted or unsubstituted heterocyclyl, substituted or unsubstituted aryl, or substituted or unsubstituted heteroaryl. In certain embodiments, A is

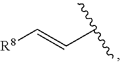

wherein R⁸ is substituted or unsubstituted alkyl, substituted or unsubstituted heteroalkyl, substituted or unsubstituted carbocyclyl, substituted or unsubstituted heterocyclyl, substituted or unsubstituted aryl, or substituted or unsubstituted heteroaryl. In certain embodiments, A is

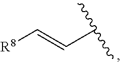

wherein R⁸ is substituted or unsubstituted carbocyclyl, substituted or unsubstituted heterocyclyl, substituted or unsubstituted aryl, or substituted or unsubstituted heteroaryl. In certain embodiments, A is

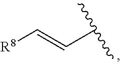

wherein R⁸ is substituted or unsubstituted aryl. In certain embodiments, A is

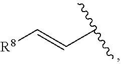

wherein R⁸ is substituted or unsubstituted phenyl. In certain embodiments, A is

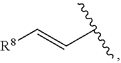

wherein R⁸ is unsubstituted phenyl or phenyl substituted with alkoxy. In certain embodiments, A

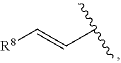

is wherein R⁸ is unsubstituted phenyl or phenyl substituted with C₁₋₄ alkoxy. In certain embodiments, A is

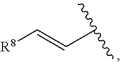

wherein R⁸ is phenyl substituted with C₁₋₄ alkoxy. In certain embodiments, A is

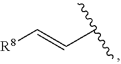

wherein R⁸ is phenyl substituted with 1-5 independent instances of C₁₋₄ alkoxy. In certain embodiments, A is

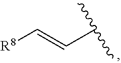

wherein R⁸ is phenyl substituted with 1-4 independent instances of C₁₋₄ alkoxy. In certain embodiments, A is

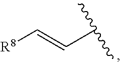

wherein R⁸ is phenyl substituted with 1-3 independent instances of C₁₋₄ alkoxy. In certain embodiments, A is

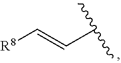

wherein R⁸ is phenyl substituted with 1-2 independent instances of C₁₋₄ alkoxy. In certain embodiments, A is

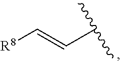

wherein R⁸ is phenyl substituted with 1 instance of C₁₋₄ alkoxy. In certain embodiments, A is

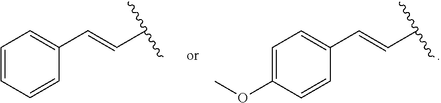 [SMILES: */C=C/c1ccccc1 ; */C=C/c1ccc(OC)cc1]

In certain embodiments, A is

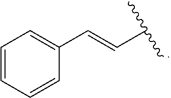 [SMILES: */C=C/c1ccccc1]

In certain embodiments, A is

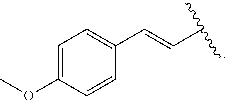 [SMILES: */C=C/c1ccc(OC)cc1]

In certain embodiments, A is

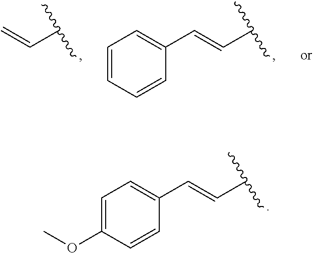

In certain embodiments, A is

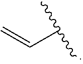 [SMILES: *C=C]

In certain embodiments, A is

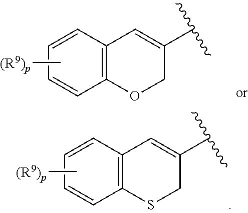

In certain embodiments, A is

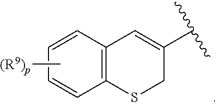

In certain embodiments, A is

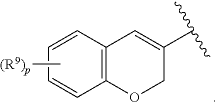

In certain embodiments, each R⁹ is independently halogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, substituted or unsubstituted heteroaliphatic, —OR^{A}, —N(R^{A})₂, —SR^{A}, —CN, —SCN, —C(═NR^{A})R^{A}, —C(═NR^{A})OR^{A}, —C(═NR^{A})N(R^{A})₂, —C(═O)R^{A}, —C(═O)OR^{A}, —C(═O)N(R^{A})₂, —NO₂, —NR^{A}C(═O)R^{A}, —NR^{A}C(═O)OR^{A}, —NR^{A}C(═O)N(R^{A})₂, —NR^{A}C(═NR^{A})N(R^{A})₂, —OC(═O)R^{A}, —OC(═O)OR^{A}, —OC(═O)N(R^{A})₂, —NR^{A}S(O)₂R^{A}, OS(O)₂R^{A}, or —S(O)₂R^{A}; or two R⁹ groups are joined to form a substituted or unsubstituted heterocyclyl ring, or a substituted or unsubstituted carbocyclyl ring. In certain embodiments, each R⁹ is independently halogen, substituted or unsubstituted alkyl, substituted or unsubstituted heteroaliphatic, —OR^{A}, —N(R^{A})₂, —SR^{A}, or —CN. In certain embodiments, each R⁹ is independently halogen, substituted or unsubstituted alkyl, or —OR^{A}. In certain embodiments, each R⁹ is independently halogen, unsubstituted alkyl, haloalkyl, or —OR^{A}. In certain embodiments, each R⁹ is independently halogen or —OR^{A}. In certain embodiments, each R⁹ is independently independently halogen, unsubstituted alkyl, haloalkyl, or —OR^{A}; and R^{A} is haloalkyl or unsubstituted alkyl. In certain embodiments, each R⁹ is independently halogen, unsubstituted C₁₋₄ alkyl, C₁₋₄ haloalkyl, or —OR^{A}; and R^{A} is C₁₋₄ haloalkyl or unsubstituted C₁₋₄ alkyl. In certain embodiments, each R⁹ is independently halogen, C₁₋₄ alkyl, C₁₋₄ haloalkyl, —OC₁₋₄ haloalkyl, or —OC₁₋₄ alkyl. In certain embodiments, each R⁹ is independently-OC₁₋₄ haloalkyl or —OC₁₋₄ alkyl. In certain embodiments, each R⁹ is independently halogen or —OC₁₋₄ alkyl. In certain embodiments, each R⁹ is independently halogen. In certain embodiments, each R⁹ is independently unsubstituted C₁₋₄ alkyl. In certain embodiments, each R⁹ is independently C₁₋₄ haloalkyl. In certain embodiments, each R⁹ is independently-OC₁₋₄ haloalkyl. In certain embodiments, each R⁹ is independently —OC₁₋₄ alkyl. In certain embodiments, each R⁹ is independently —Br, —CH₃, —CF₃, —OCF₃, or —OCH₃. In certain embodiments, each R⁹ is independently —Br, —CF₃, —OCF₃, or —OCH₃. In certain embodiments, each R⁹ is independently —Br, —OCF₃, or —OCH₃. In certain embodiments, each R⁹ is independently —Br or —OCH₃. In certain embodiments, each R⁹ is independently —OCF₃ or —OCH₃. In certain embodiments, each R⁹ is —OCH₃.

In certain embodiments, A is

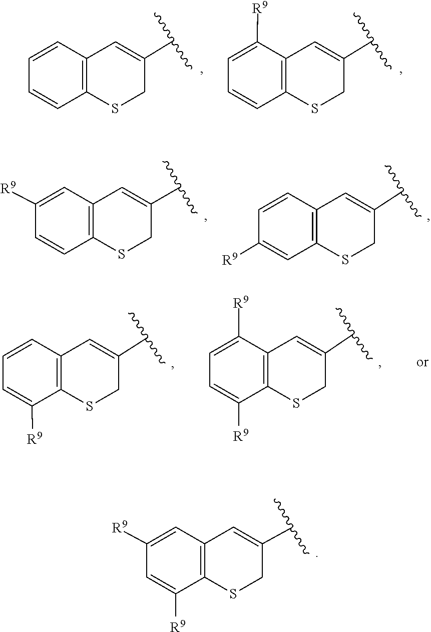

In certain embodiments, A is

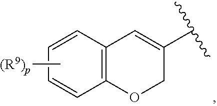

wherein each R⁹ is independently halogen, substituted or unsubstituted alkyl, substituted or unsubstituted heteroaliphatic, —OR^{A}, —N(R^{A})₂, —SR^{A}, or —CN. In certain embodiments, A is

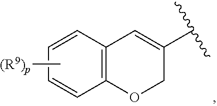

wherein each R⁹ is independently halogen, substituted or unsubstituted alkyl, substituted or unsubstituted heteroaliphatic, —OR^{A}, —N(R^{A})₂, —SR^{A}, or —CN; and p is 0, 1, or 2. In certain embodiments, A is

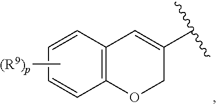

wherein R⁹ is halogen, substituted or unsubstituted alkyl, substituted or unsubstituted heteroaliphatic, —OR^{A}, —N(R^{A})₂, —SR^{A}, or —CN; and p is 1.

In certain embodiments, A is

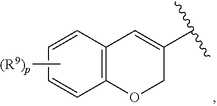

wherein each R⁹ is independently halogen, substituted or unsubstituted alkyl, or —OR^{A}. In certain embodiments, A is

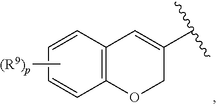

wherein each R⁹ is independently halogen, substituted or unsubstituted alkyl, or —OR^{A}; and p is 0, 1, or 2. In certain embodiments, A is

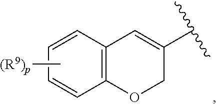

wherein R⁹ is halogen, substituted or unsubstituted alkyl, or —OR^{A}; and p is 1.

In certain embodiments, A is

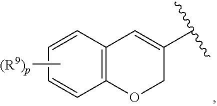

wherein each R⁹ is independently halogen, unsubstituted alkyl, haloalkyl, or —OR^{A}. In certain embodiments, A is

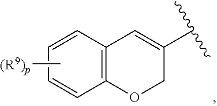

wherein each R⁹ is independently halogen, unsubstituted alkyl, haloalkyl, or —OR^{A}; and p is 0, 1, or 2. In certain embodiments, A is

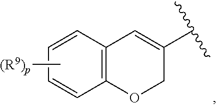

wherein each R⁹ is halogen, unsubstituted alkyl, haloalkyl, or —OR^{A}; and p is 1.

In certain embodiments, A is

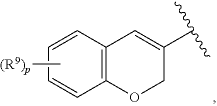

wherein each R⁹ is independently halogen, unsubstituted alkyl, haloalkyl, or —OR^{A}; and R^{A} is haloalkyl or unsubstituted alkyl. In certain embodiments, A is

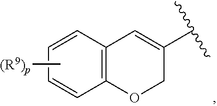

wherein each R⁹ is independently halogen, unsubstituted alkyl, haloalkyl, or —OR^{A}; R^{A} is haloalkyl or unsubstituted alkyl; and p is 0, 1, or 2. In certain embodiments, A is

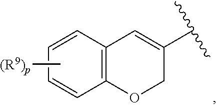

wherein each R⁹ is halogen, unsubstituted alkyl, haloalkyl, or —OR^{A}; R^{A} is haloalkyl or unsubstituted alkyl; and p is 1.

In certain embodiments, A is

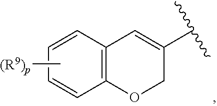

wherein each R⁹ is independently halogen, unsubstituted C₁₋₄ alkyl, C₁₋₄ haloalkyl, or —OR^{A}; and R^{A} is C₁₋₄ haloalkyl or unsubstituted C₁₋₄ alkyl. In certain embodiments, A is

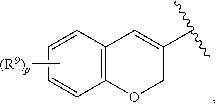

wherein each R⁹ is independently halogen, unsubstituted C₁₋₄ alkyl, C₁₋₄ haloalkyl, or —OR^{A}; R^{A} is C₁₋₄ haloalkyl or unsubstituted C₁₋₄ alkyl; and p is 0, 1, or 2. In certain embodiments, A is

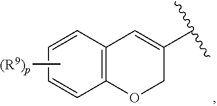

wherein R⁹ is halogen, unsubstituted C₁₋₄ alkyl, C₁₋₄ haloalkyl, or —OR^{A}; R^{A} is C₁₋₄ haloalkyl or unsubstituted C₁₋₄ alkyl; and p is 1.

In certain embodiments, A is

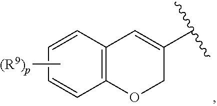

wherein each R⁹ is independently halogen, C₁₋₄ alkyl, C₁₋₄ haloalkyl, —OC₁₋₄ haloalkyl, or —OC₁₋₄ alkyl. In certain embodiments, A is

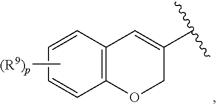

wherein each R⁹ is independently halogen, C₁₋₄ alkyl, C₁₋₄ haloalkyl, —OC₁₋₄ haloalkyl, or —OC₁₋₄ alkyl; and p is 0, 1, or 2. In certain embodiments, A is

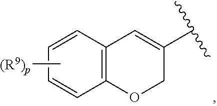

wherein R⁹ is halogen, C₁₋₄ alkyl, C₁₋₄ haloalkyl, —OC₁₋₄ haloalkyl, or —OC₁₋₄ alkyl; and p is 1.

In certain embodiments, A is

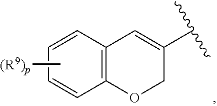

wherein each R⁹ is independently —Br, —CH₃, —CF₃, —OCF₃, or —OCH₃. In certain embodiments, A is

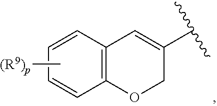

wherein each R⁹ is independently —Br, —CH₃, —CF₃, —OCF₃, or —OCH₃; and p is 0, 1, or 2. In certain embodiments, A is

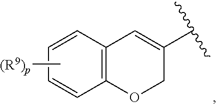

wherein R⁹ is —Br, —CH₃, —CF₃, —OCF₃, or —OCH₃; and p is 1.

In certain embodiments, A is

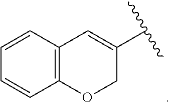 [SMILES: *C1=Cc2ccccc2OC1]

In certain embodiments, A is

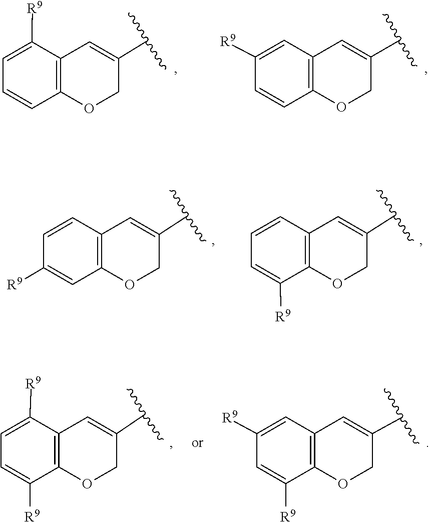

In certain embodiments, A is

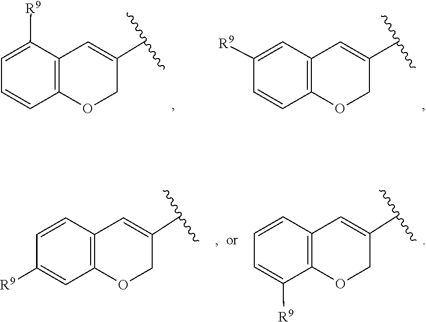

In certain embodiments, A is

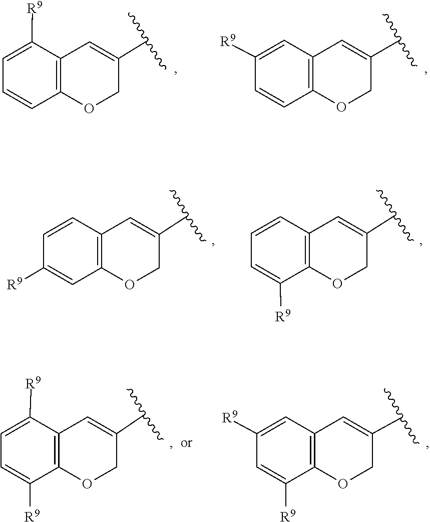

wherein each R⁹ is independently halogen, substituted or unsubstituted alkyl, substituted or unsubstituted heteroaliphatic, —OR^{A}, —N(R^{A})₂, —SR^{A}, or —CN.

In certain embodiments, A is

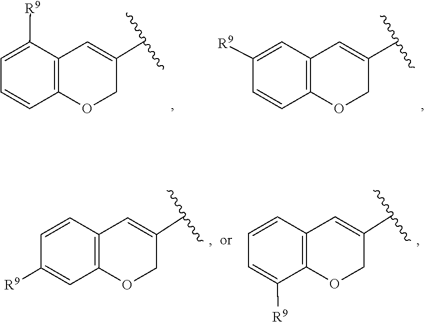

wherein R⁹ is halogen, substituted or unsubstituted alkyl, substituted or unsubstituted heteroaliphatic, —OR^{A}, —N(R^{A})₂, —SR^{A}, or —CN.

In certain embodiments, A is

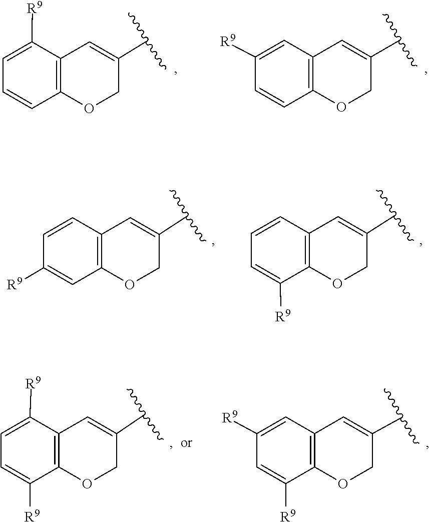

wherein each R⁹ is independently halogen, substituted or unsubstituted alkyl, or —OR^{A}.

In certain embodiments, A is

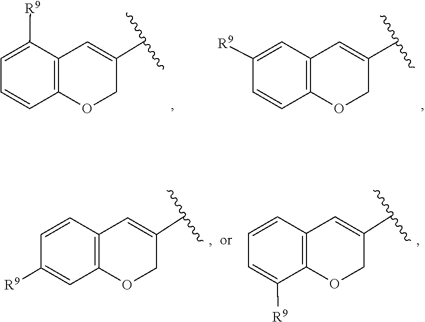

wherein R⁹ is halogen, substituted or unsubstituted alkyl, or —OR^{A}.

In certain embodiments, A is

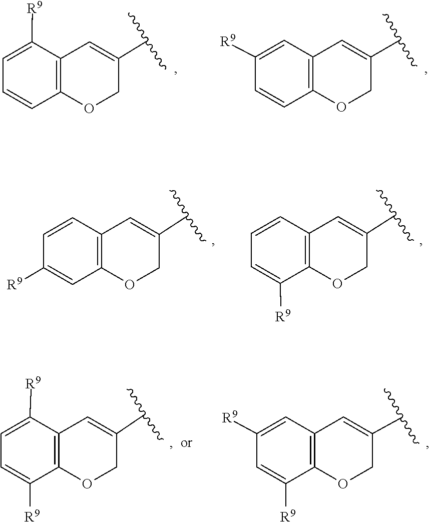

wherein each R⁹ is independently halogen, unsubstituted alkyl, haloalkyl, or —OR^{A}.

In certain embodiments, A is

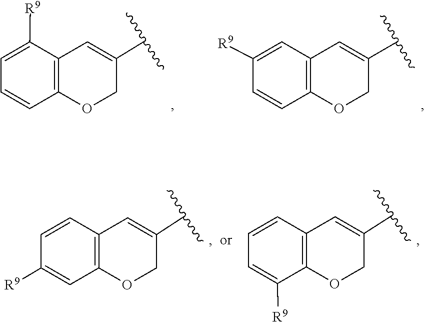

wherein R⁹ is halogen, unsubstituted alkyl, haloalkyl, or —OR^{A}.

In certain embodiments, A is

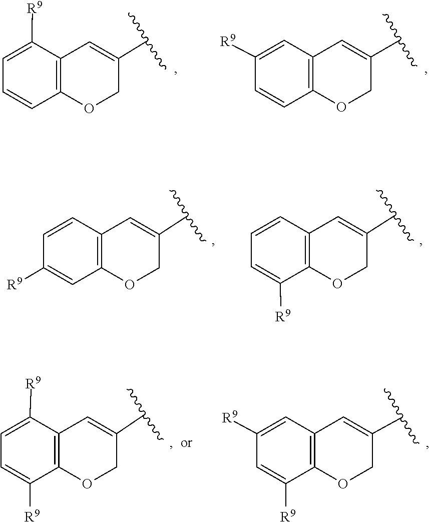

wherein each R⁹ is independently halogen, unsubstituted alkyl, haloalkyl, or —OR^{A}; and R^{A} is haloalkyl or unsubstituted alkyl.

In certain embodiments, A is

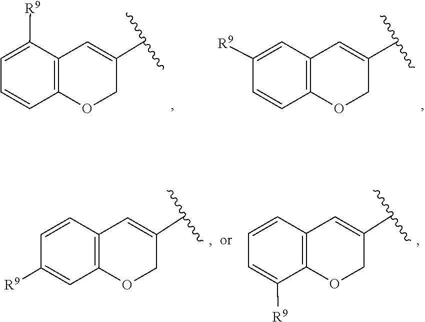

wherein R⁹ is halogen, unsubstituted alkyl, haloalkyl, or —OR^{A}; and R^{A} is haloalkyl or unsubstituted alkyl.

In certain embodiments, A is

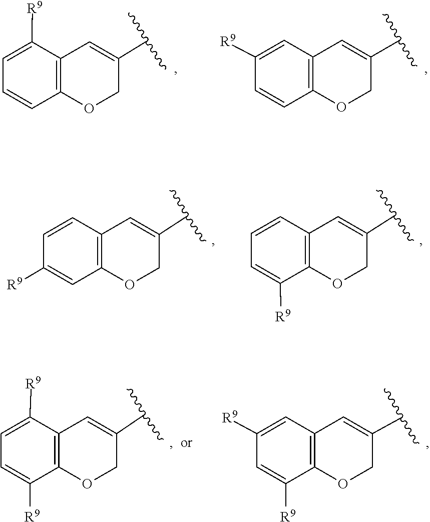

wherein each R⁹ is independently halogen, unsubstituted C₁₋₄ alkyl, C₁₋₄ haloalkyl, or —OR^{A}; and R^{A} is C₁₋₄ haloalkyl or unsubstituted C₁₋₄ alkyl.

In certain embodiments, A is

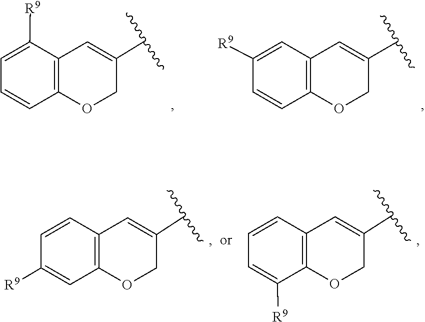

wherein R⁹ is halogen, unsubstituted C₁₋₄ alkyl, C₁₋₄ haloalkyl, or —OR^{A}; and R^{A} is C₁₋₄ haloalkyl or unsubstituted C₁₋₄ alkyl.

In certain embodiments, A is

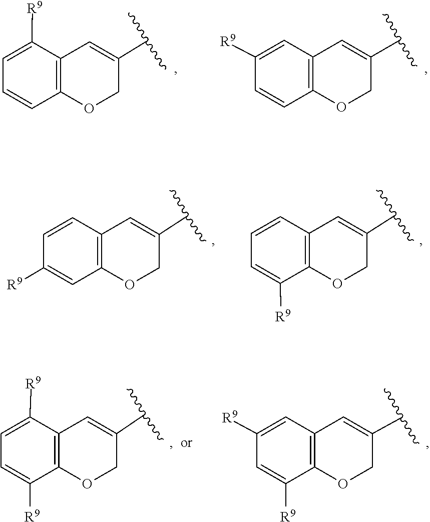

wherein each R⁹ is independently halogen, C₁₋₄ alkyl, C₁₋₄ haloalkyl, —OC₁₋₄ haloalkyl, or —OC₁₋₄ alkyl.

In certain embodiments, A is

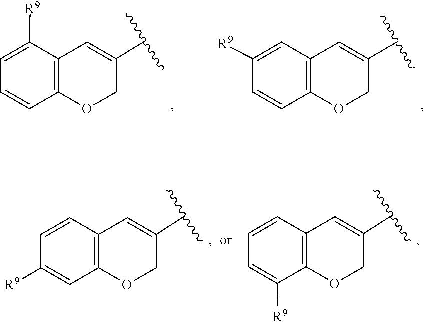

wherein R⁹ is halogen, C₁₋₄ alkyl, C₁₋₄ haloalkyl, —OC₁₋₄ haloalkyl, or —OC₁₋₄ alkyl.

In certain embodiments, A is

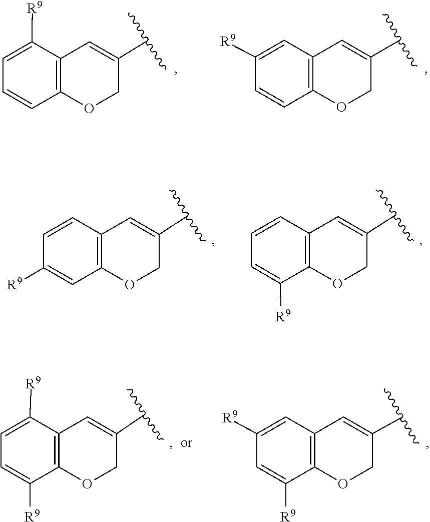

wherein each R⁹ is independently —Br, —CH₃, —CF₃, —OCF₃, or —OCH₃.

In certain embodiments, A is

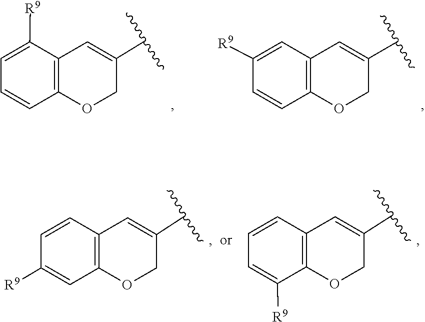

wherein R⁹ is —Br, —CH₃, —CF₃, —OCF₃, or —OCH₃.

In certain embodiments, A is

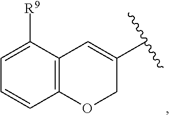

wherein R⁹ is halogen, substituted or unsubstituted alkyl, substituted or unsubstituted heteroaliphatic, —OR^{A}, —N(R^{A})₂, —SR^{A}, or —CN. In certain embodiments, A is

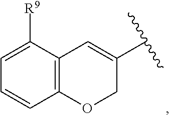

wherein R⁹ is halogen, substituted or unsubstituted alkyl, or —OR^{A}. In certain embodiments, A is

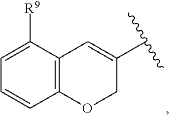

wherein R⁹ is halogen, unsubstituted alkyl, haloalkyl, or —OR^{A}. In certain embodiments, A is

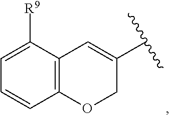

wherein R⁹ is halogen, unsubstituted alkyl, haloalkyl, or —OR^{A}; and R^{A} is haloalkyl or unsubstituted alkyl. In certain embodiments, A is

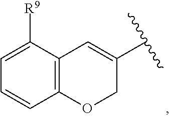

wherein R⁹ is halogen, unsubstituted C₁₋₄ alkyl, C₁₋₄ haloalkyl, or —OR^{A}; and R^{A} is C₁₋₄ haloalkyl or unsubstituted C₁₋₄ alkyl. In certain embodiments, A is

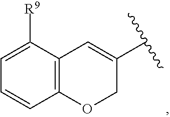

wherein R⁹ is halogen, C₁₋₄ alkyl, C₁₋₄ haloalkyl, —OC₁₋₄ haloalkyl, or —OC₁₋₄ alkyl. In certain embodiments, A is

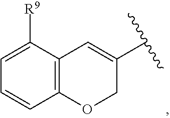

wherein R⁹ is —Br, —CH₃, —CF₃, —OCF₃, or —OCH₃.

In certain embodiments, A is

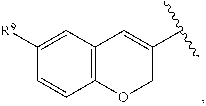

wherein R⁹ is halogen, substituted or unsubstituted alkyl, substituted or unsubstituted heteroaliphatic, —OR^{A}, —N(R^{A})₂, —SR^{A}, or —CN. In certain embodiments, A is

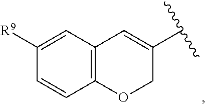

wherein R⁹ is halogen, substituted or unsubstituted alkyl, or —OR^{A}. In certain embodiments, A is

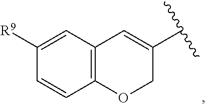

wherein R⁹ is halogen, unsubstituted alkyl, haloalkyl, or —OR^{A}. In certain embodiments, A is

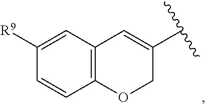

wherein R⁹ is halogen, unsubstituted alkyl, haloalkyl, or —OR^{A}; and R^{A} is haloalkyl or unsubstituted alkyl. In certain embodiments, A is

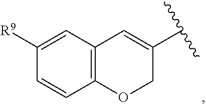

wherein R⁹ is halogen, unsubstituted C₁₋₄ alkyl, C₁₋₄ haloalkyl, or —OR^{A}; and R^{A} is C₁₋₄ haloalkyl or unsubstituted C₁₋₄ alkyl. In certain embodiments, A is

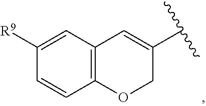

wherein R⁹ is halogen, C₁₋₄ alkyl, C₁₋₄ haloalkyl, —OC₁₋₄ haloalkyl, or —OC₁₋₄ alkyl. In certain embodiments, A is

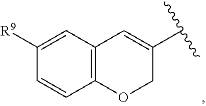

wherein R⁹ is —Br, —CH₃, —CF₃, —OCF₃, or —OCH₃.

In certain embodiments, A is

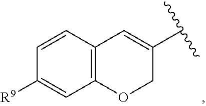

wherein R⁹ is halogen, substituted or unsubstituted alkyl, substituted or unsubstituted heteroaliphatic, —OR^{A}, —N(R^{A})₂, —SR^{A}, or —CN. In certain embodiments, A is

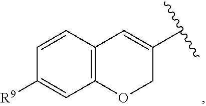

wherein R⁹ is halogen, substituted or unsubstituted alkyl, or —OR^{A}. In certain embodiments, A is

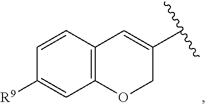

wherein R⁹ is halogen, unsubstituted alkyl, haloalkyl, or —OR^{A}. In certain embodiments, A is

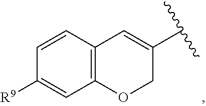

wherein R⁹ is halogen, unsubstituted alkyl, haloalkyl, or —OR^{A}; and R^{A} is haloalkyl or unsubstituted alkyl. In certain embodiments, A is

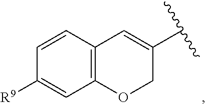

wherein R⁹ is halogen, unsubstituted C₁₋₄ alkyl, C₁₋₄ haloalkyl, or —OR^{A}; and R^{A} is C₁₋₄ haloalkyl or unsubstituted C₁₋₄ alkyl. In certain embodiments, A is

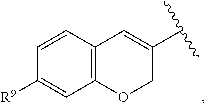

wherein R⁹ is halogen, C₁₋₄ alkyl, C₁₋₄ haloalkyl, —OC₁₋₄ haloalkyl, or —OC₁₋₄ alkyl. In certain embodiments, A is

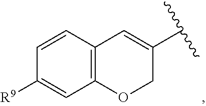

wherein R⁹ is —Br, —CH₃, —CF₃, —OCF₃, or —OCH₃.

In certain embodiments, A is

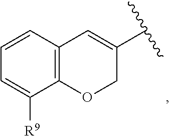

wherein R⁹ is halogen, substituted or unsubstituted alkyl, substituted or unsubstituted heteroaliphatic, —OR^{A}, —N(R^{A})₂, —SR^{A}, or —CN. In certain embodiments, A is

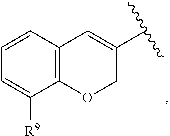

wherein R⁹ is halogen, substituted or unsubstituted alkyl, or —OR^{A}. In certain embodiments, A is

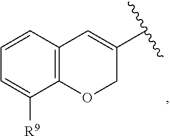

wherein R⁹ is halogen, unsubstituted alkyl, haloalkyl, or —OR^{A}. In certain embodiments, A is

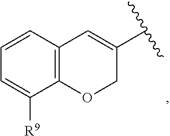

wherein R⁹ is halogen, unsubstituted alkyl, haloalkyl, or —OR^{A}; and R^{A} is haloalkyl or unsubstituted alkyl. In certain embodiments, A is

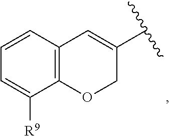

wherein R⁹ is halogen, unsubstituted C₁₋₄ alkyl, C₁₋₄ haloalkyl, or —OR^{A}; and R^{A} is C₁₋₄ haloalkyl or unsubstituted C₁₋₄ alkyl. In certain embodiments, A is

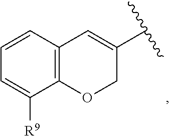

wherein R⁹ is halogen, C₁₋₄ alkyl, C₁₋₄ haloalkyl, —OC₁₋₄ haloalkyl, or —OC₁₋₄ alkyl. In certain embodiments, A is

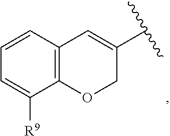

wherein R⁹ is —Br, —CH₃, —CF₃, —OCF₃, or —OCH₃.

In certain embodiments, A is

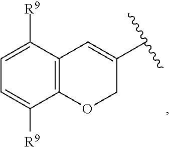

wherein each R⁹ is independently halogen, substituted or unsubstituted alkyl, substituted or unsubstituted heteroaliphatic, —OR^{A}, —N(R^{A})₂, —SR^{A}, or —CN. In certain embodiments, A is

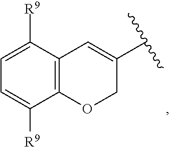

wherein each R⁹ is independently halogen, substituted or unsubstituted alkyl, or —OR^{A}. In certain embodiments, A is

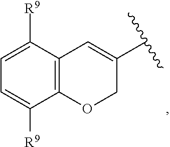

wherein each R⁹ is independently halogen, unsubstituted alkyl, haloalkyl, or —OR^{A}. In certain embodiments, A is

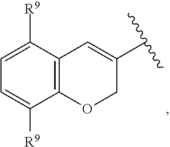

wherein each R⁹ is independently halogen, unsubstituted alkyl, haloalkyl, or —OR^{A}; and R^{A} is haloalkyl or unsubstituted alkyl. In certain embodiments, A is

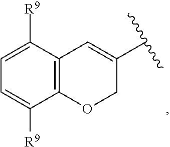

wherein each R⁹ is independently halogen, unsubstituted C₁₋₄ alkyl, C₁₋₄ haloalkyl, or —OR^{A}; and R^{A} is C₁₋₄ haloalkyl or unsubstituted C₁₋₄ alkyl. In certain embodiments, A is

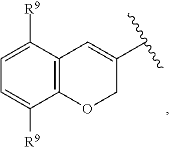

wherein each R⁹ is independently halogen, C₁₋₄ alkyl, C₁₋₄ haloalkyl, —OC₁₋₄ haloalkyl, or —OC₁₋₄ alkyl. In certain embodiments, A is

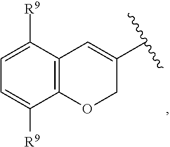

wherein each R⁹ is independently —Br, —CH₃, —CF₃, —OCF₃, or —OCH₃.

In certain embodiments, A is

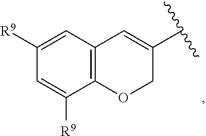

wherein each R⁹ is independently halogen, substituted or unsubstituted alkyl, substituted or unsubstituted heteroaliphatic, —OR^{A}, —N(R^{A})₂, —SR^{A}, or —CN. In certain embodiments, A is

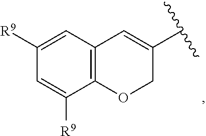

wherein each R⁹ is independently halogen, substituted or unsubstituted alkyl, or —OR^{A}. In certain embodiments, A is

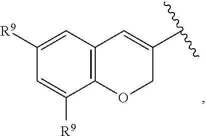

wherein each R⁹ is independently halogen, unsubstituted alkyl, haloalkyl, or —OR^{A}. In certain embodiments, A is

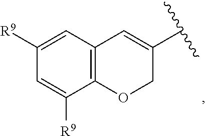

wherein each R⁹ is independently halogen, unsubstituted alkyl, haloalkyl, or —OR^{A}; and R^{A} is haloalkyl or unsubstituted alkyl. In certain embodiments, A is

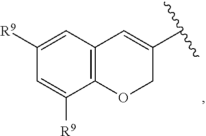

wherein each R⁹ is independently halogen, unsubstituted C₁₋₄ alkyl, C₁₋₄ haloalkyl, or —OR^{A}; and R^{A} is C₁₋₄ haloalkyl or unsubstituted C₁₋₄ alkyl. In certain embodiments, A is

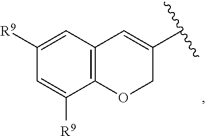

wherein each R⁹ is independently halogen, C₁₋₄ alkyl, C₁₋₄ haloalkyl, —OC₁₋₄ haloalkyl, or —OC₁₋₄ alkyl. In certain embodiments, A is

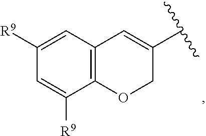

wherein each R⁹ is independently —Br, —CH₃, —CF₃, —OCF₃, or —OCH₃.

In certain embodiments, A is

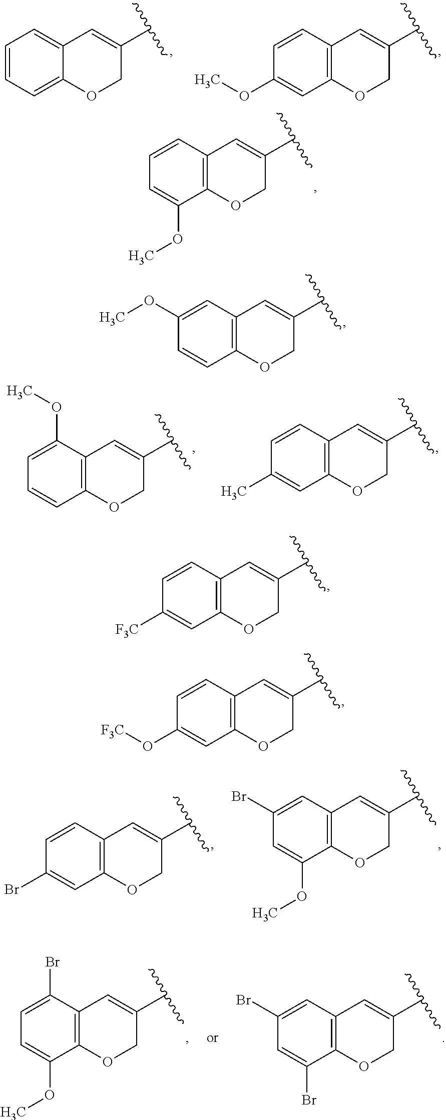

In certain embodiments, A is

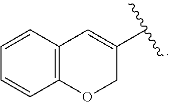 [SMILES: *C1=Cc2ccccc2OC1]

In certain embodiments, A is

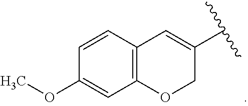 [SMILES: *C1=Cc2ccc(OC)cc2OC1]

In certain embodiments, A is

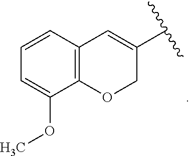 [SMILES: *C1=Cc2cccc(OC)c2OC1]

In certain embodiments, A is

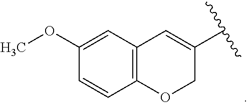 [SMILES: *C1=Cc2cc(OC)ccc2OC1]

In certain embodiments, A is

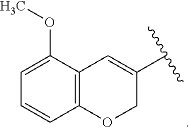 [SMILES: *C1=Cc2c(OC)cccc2OC1]

In certain embodiments, A is

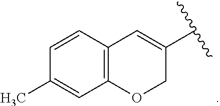 [SMILES: *C1=Cc2ccc(C)cc2OC1]

In certain embodiments, A is

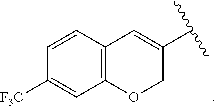 [SMILES: *C1=Cc2ccc(C(F)(F)F)cc2OC1]

In certain embodiments. A is

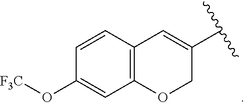 [SMILES: *C1=Cc2ccc(OC(F)(F)F)cc2OC1]

In certain embodiments. A is

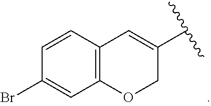 [SMILES: *C1=Cc2ccc(Br)cc2OC1]

In certain embodiments. A is

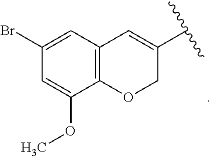 [SMILES: *C1=Cc2cc(Br)cc(OC)c2OC1]

In certain embodiments, A is

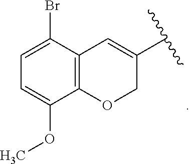 [SMILES: *C1=Cc2c(Br)ccc(OC)c2OC1]

In certain embodiments. A is

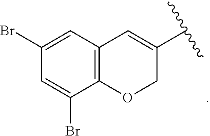 [SMILES: *C1=Cc2cc(Br)cc(Br)c2OC1]

In certain embodiments. A is —CH₃,

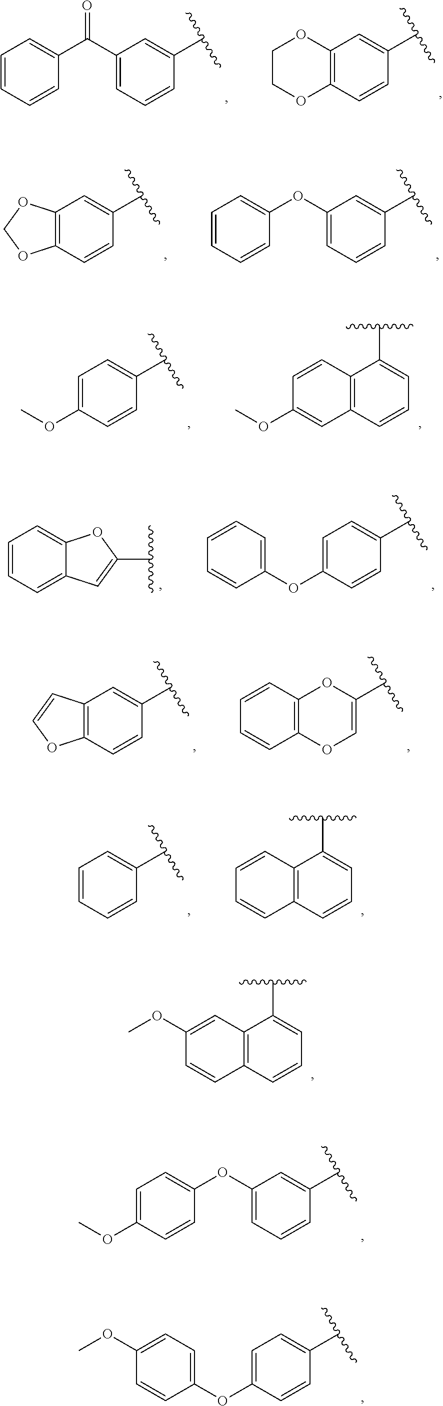

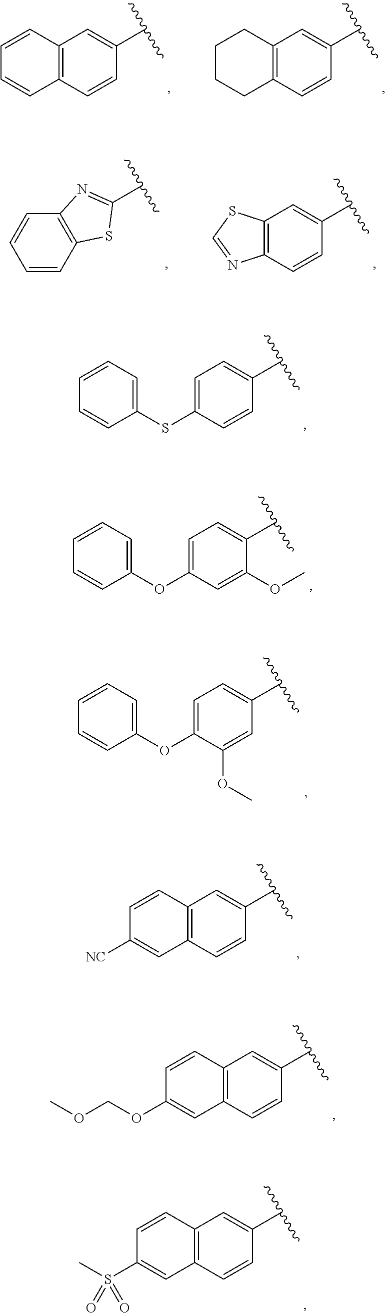

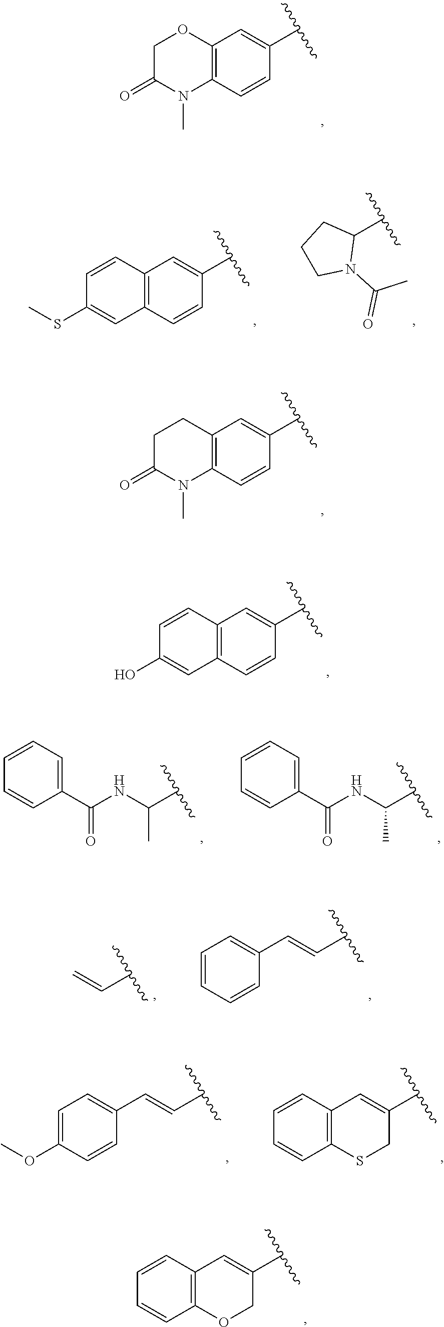

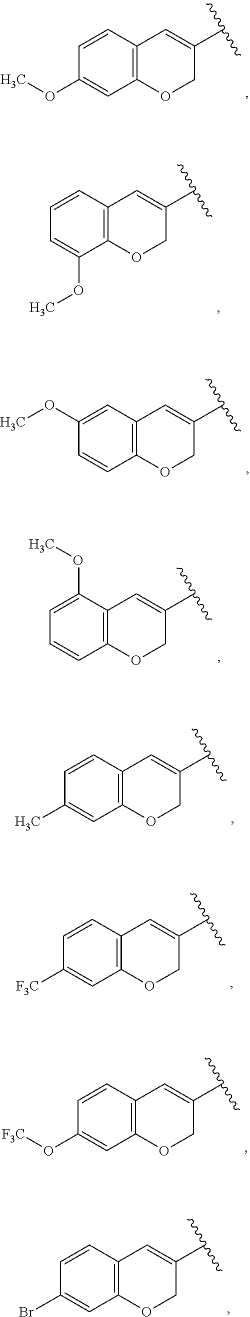

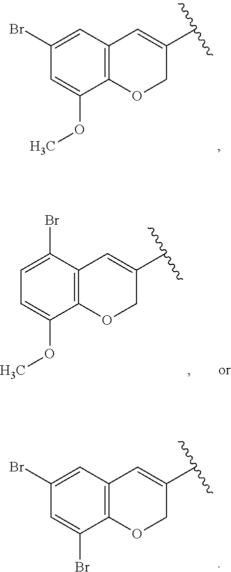 [SMILES: *C1=Cc2cc(Br)cc(OC)c2OC1 ; *C1=Cc2c(Br)ccc(OC)c2OC1 ; *C1=Cc2cc(Br)cc(Br)c2OC1]

In certain embodiments, A is —CH₃,

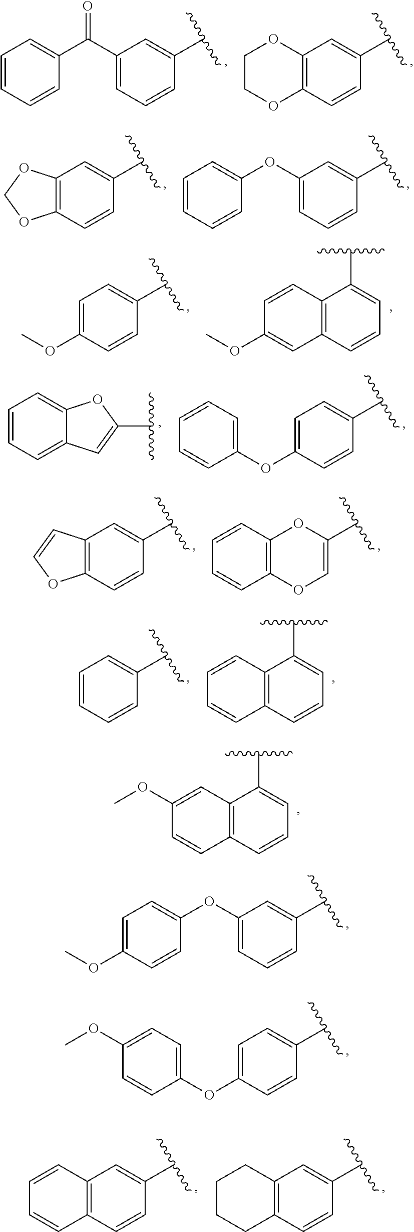

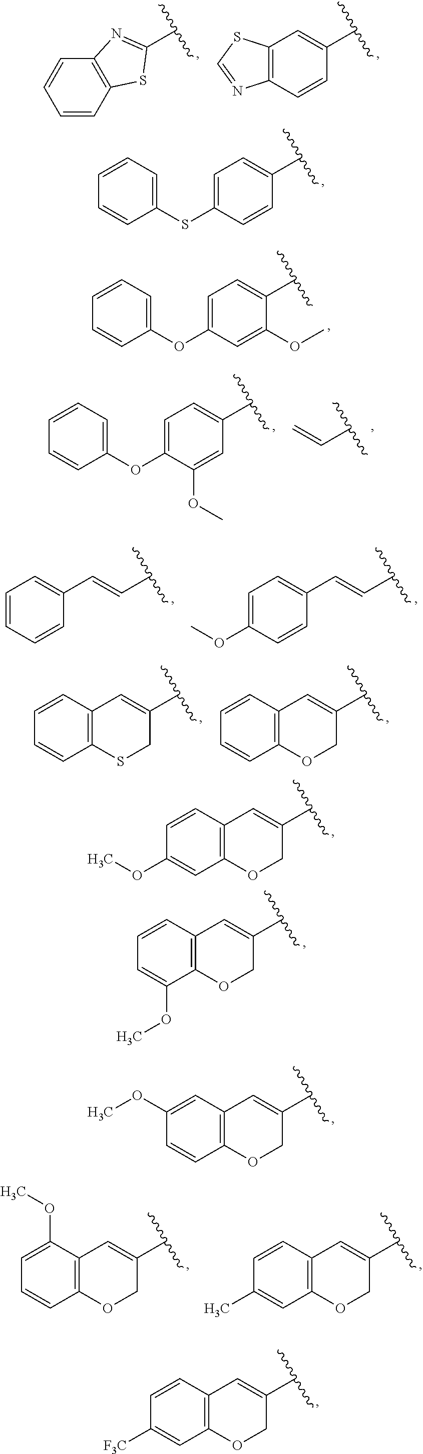

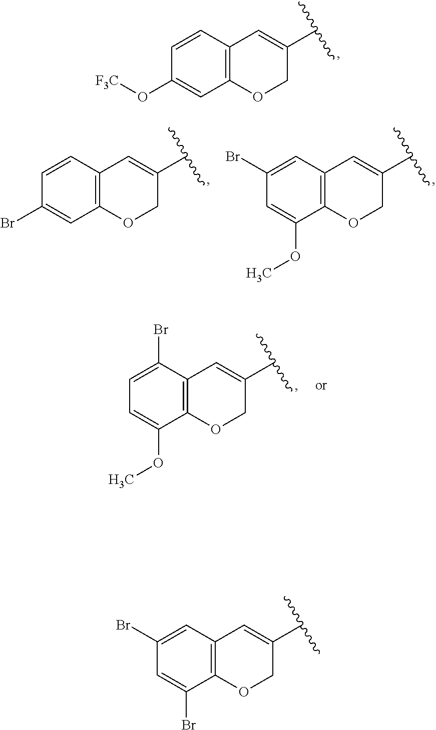 [SMILES: *C1=Cc2ccc(OC(F)(F)F)cc2OC1 ; *C1=Cc2ccc(Br)cc2OC1 ; *C1=Cc2cc(Br)cc(OC)c2OC1 ; *C1=Cc2c(Br)ccc(OC)c2OC1 ; *C1=Cc2cc(Br)cc(Br)c2OC1]

In certain embodiments. A is —CH₃,

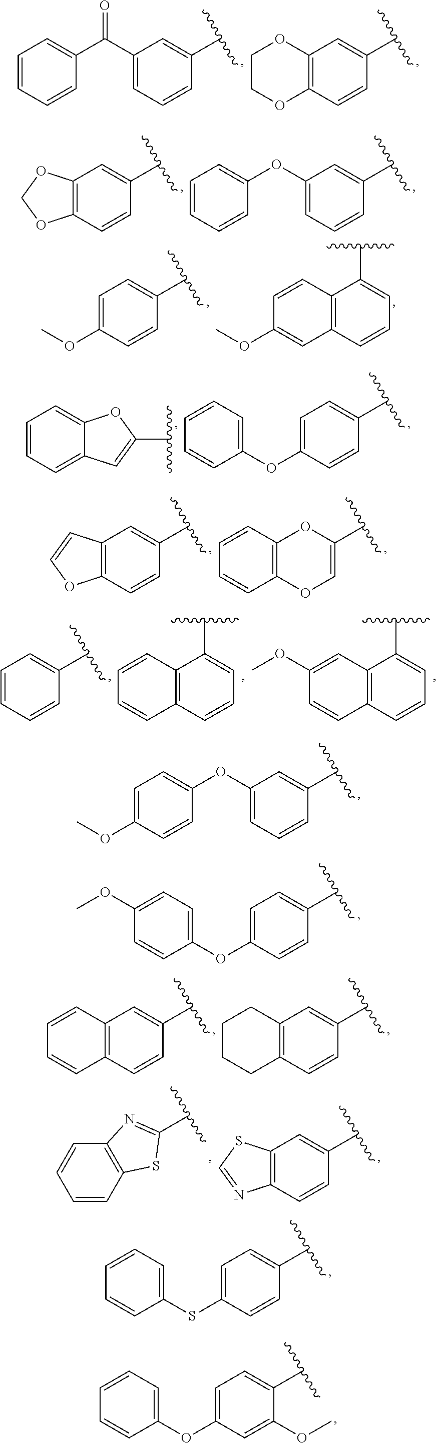

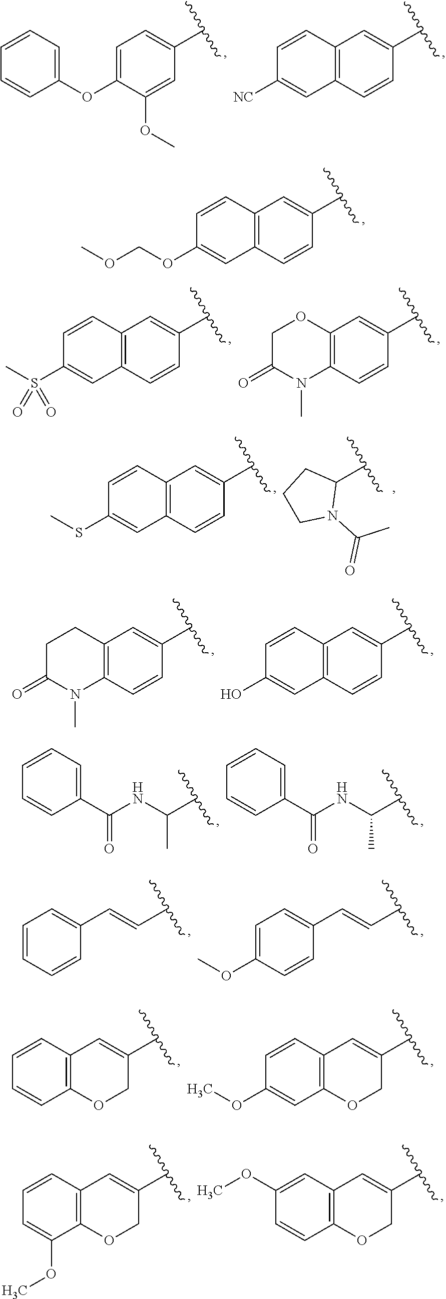

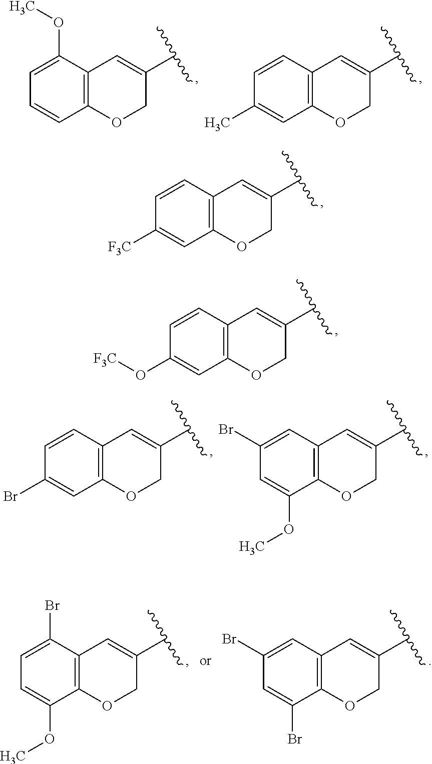

In certain embodiments. A is —CH₃,

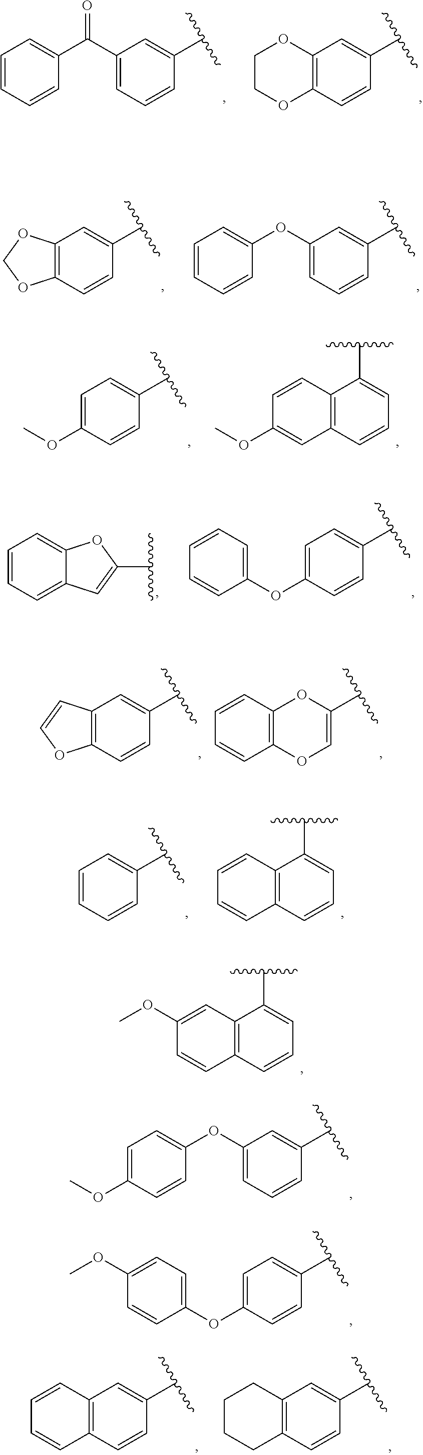

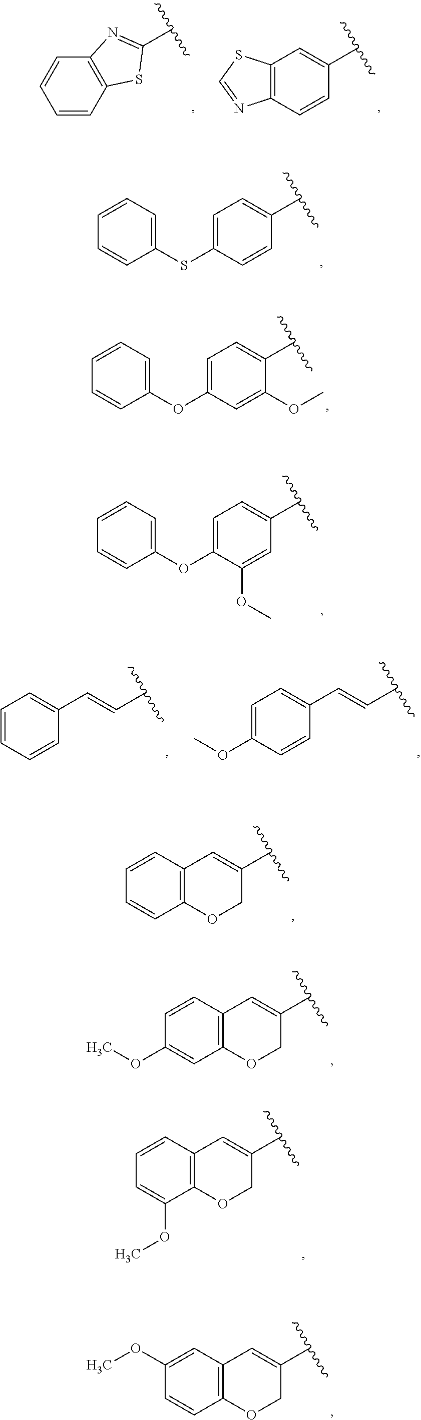

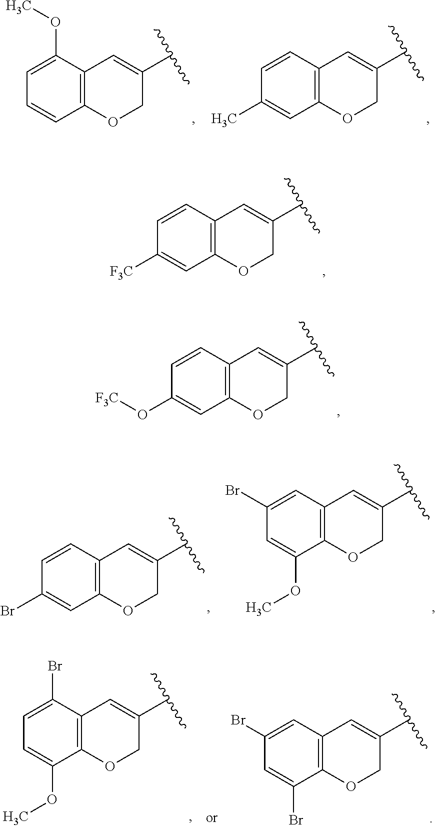

In certain embodiments, A is not one or more of the following:

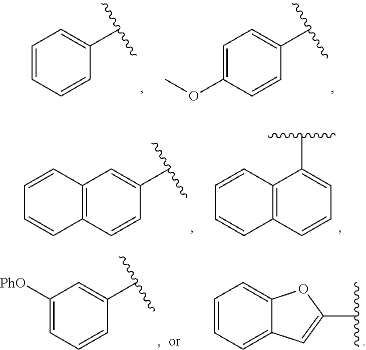

### R^{A}

As described herein, each occurrence of R^{A} is, independently, hydrogen, substituted or unsubstituted acyl, substituted or unsubstituted alkyl, substituted or unsubstituted alkenyl, substituted or unsubstituted alkynyl, substituted or unsubstituted heteroaliphatic, substituted or unsubstituted carbocyclyl, substituted or unsubstituted carbocyclylalkyl, substituted or unsubstituted heterocyclyl, substituted or unsubstituted aryl, substituted or unsubstituted aralkyl, substituted or unsubstituted heteroaryl, substituted or unsubstituted hetaralkyl, a nitrogen protecting group when attached to a nitrogen atom, an oxygen protecting group when attached to an oxygen atom, or a sulfur protecting group when attached to a sulfur atom, or two R^{A} groups are joined to form a substituted or unsubstituted heterocyclyl ring, or a substituted or unsubstituted heteroaryl ring.

In certain embodiments, each occurrence of R^{A} is, independently, hydrogen, substituted or unsubstituted acyl, substituted or unsubstituted alkyl, an oxygen protecting group when attached to an oxygen atom, or a nitrogen protecting group when attached to a nitrogen atom.

In certain embodiments, each occurrence of R^{A} is, independently, hydrogen, or substituted or unsubstituted alkyl. In certain embodiments, each occurrence of R^{A} is, independently, hydrogen or unsubstituted alkyl. In certain embodiments, R^{A} is hydrogen.

In certain embodiments, each occurrence of R^{A} is, independently, haloalkyl or unsubstituted alkyl. In certain embodiments, each occurrence of R^{A} is, independently, haloalkyl. In certain embodiments, each occurrence of R^{A} is, independently, unsubstituted alkyl. In certain embodiments, each R^{A} is independently C₁₋₄ haloalkyl or unsubstituted C₁₋₄ alkyl. In certain embodiments, each R^{A} is independently C₁₋₄ haloalkyl. In certain embodiments, each R^{A} is independently unsubstituted C₁₋₄ alkyl. In certain embodiments, each R^{A} is independently —CF₃ or —CH₃. In certain embodiments, each R^{A} is independently —CF₃. In certain embodiments, each R^{A} is independently —CH₃.

m

As described herein, m is 0, 1, 2 or 3. In certain embodiments, m is 0, 1, or 2. In certain embodiments, m is 0 or 1. In certain embodiments, m is 0. In certain embodiments, m is 1. In certain embodiments, m is 2. In certain embodiments, m is 3.

n

As described herein, n is 0, 1, or 2. In certain embodiments, n is 1 or 2. In certain embodiments, n is 0 or 1. In certain embodiments, n is 0. In certain embodiments, n is 1. In certain embodiments, n is 2.

p

As described herein, p is 0, 1, 2, 3, or 4. In certain embodiments, p is 0, 1, 2, or 3. In certain embodiments, p is 0, 1, or 2. In certain embodiments, p is 1 or 2. In certain embodiments, p is 0 or 2. In certain embodiments, p is 0 or 1. In certain embodiments, p is 0. In certain embodiments, p is 1. In certain embodiments, p is 2. In certain embodiments, p is 3.

t

As described herein, t is 0, 1, or 2. In certain embodiments, t is 1 or 2. In certain embodiments, t is 0 or 1. In certain embodiments, t is 0. In certain embodiments, t is 1. In certain embodiments, t is 2.

In certain embodiments, p is 0, 1, 2, or 3; and t is 1 or 2. In certain embodiments, p is 0, 1, 2, or 3; and t is 0 or 1. In certain embodiments, p is 0, 1, 2, or 3; and t is 0. In certain embodiments, p is 0, 1, 2, or 3; and t is 1. In certain embodiments, p is 0, 1, 2, or 3; and t is 2.

In certain embodiments, p is 0, 1, or 2; and t is 1 or 2. In certain embodiments, p is 0, 1, or 2; and t is 0 or 1. In certain embodiments, p is 0, 1, or 2; and t is 0. In certain embodiments, p is 0, 1, or 2; and t is 1. In certain embodiments, p is 0, 1, or 2; and t is 2.

In certain embodiments, p is 1 or 2; and t is 1 or 2. In certain embodiments, p is 1 or 2; and t is 0 or 1. In certain embodiments, p is 1 or 2; and t is 0. In certain embodiments, p is 1 or 2; and t is 1. In certain embodiments, p is 1 or 2; and t is 2.

In certain embodiments, p is 0 or 2; and t is 1 or 2. In certain embodiments, p is 0 or 2; and t is 0 or 1. In certain embodiments, p is 0 or 2; and t is 0. In certain embodiments, p is 0 or 2; and t is 1. In certain embodiments, p is 0 or 2; and t is 2.

In certain embodiments, p is 0 or 1; and t is 1 or 2. In certain embodiments, p is 0 or 1; and t is 0 or 1. In certain embodiments, p is 0 or 1; and t is 0. In certain embodiments, p is 0 or 1; and t is 1. In certain embodiments, p is 0 or 1; and t is 2.

In certain embodiments, p is 0; and t is 1 or 2. In certain embodiments, p is 0; and t is 0 or 1. In certain embodiments, p is 0; and t is 0. In certain embodiments, p is 0; and t is 1. In certain embodiments, p is 0; and t is 2.

In certain embodiments, p is 1; and t is 1 or 2. In certain embodiments, p is 1; and t is 0 or 1. In certain embodiments, p is 1; and t is 0. In certain embodiments, p is 1; and t is 1. In certain embodiments, p is 1; and t is 2.

In certain embodiments, p is 2; and t is 1 or 2. In certain embodiments, p is 2; and t is 0 or 1. In certain embodiments, p is 2; and t is 0. In certain embodiments, p is 2; and t is 1. In certain embodiments, p is 2; and t is 2.

### Further Embodiments of Formula (1)

In certain embodiments, the compound of Formula (I) is a compound of Formula (I-a):

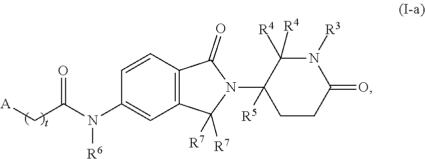

or a pharmaceutically acceptable salt, co-crystal, tautomer, stereoisomer, solvate, hydrate, polymorph, isotopically enriched derivative, or prodrug thereof, wherein A, R³, R⁴, R⁵, R⁶, R⁷, and t are as defined herein.

In certain embodiments, the compound of Formula (I) is a compound of Formula (I-b):

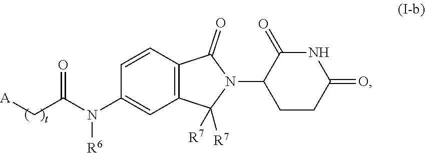

or a pharmaceutically acceptable salt, co-crystal, tautomer, stereoisomer, solvate, hydrate, polymorph, isotopically enriched derivative, or prodrug thereof, wherein A, R⁶, R⁷, and t are as defined herein.

In certain embodiments, the compound of Formula (I) is a compound of Formula (I-c):

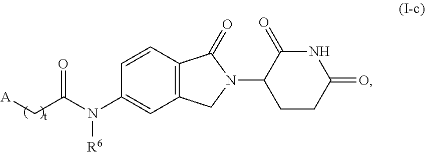

or a pharmaceutically acceptable salt, co-crystal, tautomer, stereoisomer, solvate, hydrate, polymorph, isotopically enriched derivative, or prodrug thereof, wherein A, R⁶, and t are as defined herein.

In certain embodiments, the compound of Formula (I) is a compound of Formula (I-d):

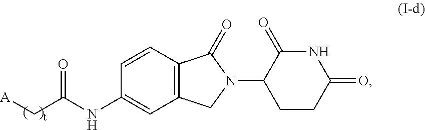

or a pharmaceutically acceptable salt, co-crystal, tautomer, stereoisomer, solvate, hydrate, polymorph, isotopically enriched derivative, or prodrug thereof, wherein A and t are as defined herein.

In certain embodiments, the compound of Formula (I) is a compound of Formula (I-e):

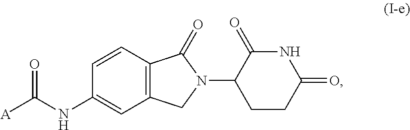

or a pharmaceutically acceptable salt, co-crystal, tautomer, stereoisomer, solvate, hydrate, polymorph, isotopically enriched derivative, or prodrug thereof, wherein A is as defined herein.

In certain embodiments, the compound of Formula (I) is a compound of Formula (I-f):

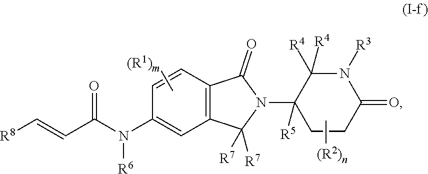

or a pharmaceutically acceptable salt, co-crystal, tautomer, stereoisomer, solvate, hydrate, polymorph, isotopically enriched derivative, or prodrug thereof, wherein R¹, R², R³, R⁴, R⁵, R⁶, R⁷, R⁸, m, n, and t are as defined herein.

In certain embodiments, the compound of Formula (I) is a compound of Formula (I-g):

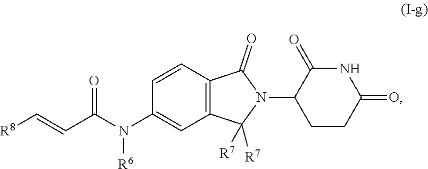

or a pharmaceutically acceptable salt, co-crystal, tautomer, stereoisomer, solvate, hydrate, polymorph, isotopically enriched derivative, or prodrug thereof, wherein R⁶, R⁷, and R⁸ are as defined herein.

In certain embodiments, the compound of Formula (I) is a compound of Formula (I-h):

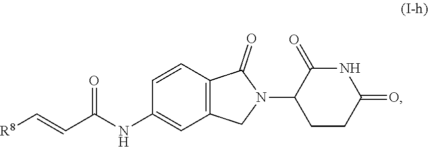

or a pharmaceutically acceptable salt, co-crystal, tautomer, stereoisomer, solvate, hydrate, polymorph, isotopically enriched derivative, or prodrug thereof, wherein R⁸ is as defined herein.

In certain embodiments, the compound of Formula (I) is a compound of Formula (I-i):

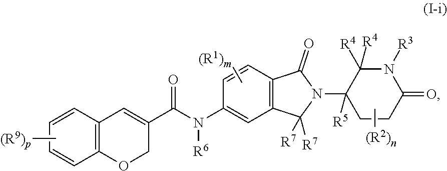

or a pharmaceutically acceptable salt, co-crystal, tautomer, stereoisomer, solvate, hydrate, polymorph, isotopically enriched derivative, or prodrug thereof, wherein R¹, R², R³, R⁴, R⁵, R⁶, R⁷, R⁹, m, n, and p are as defined herein.

In certain embodiments, the compound of Formula (I) is a compound of Formula (I-j):

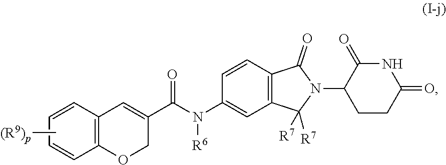

or a pharmaceutically acceptable salt, co-crystal, tautomer, stereoisomer, solvate, hydrate, polymorph, isotopically enriched derivative, or prodrug thereof, wherein R⁶, R⁷, R⁹, and p are as defined herein.

In certain embodiments, the compound of Formula (I) is a compound of Formula (I-k):

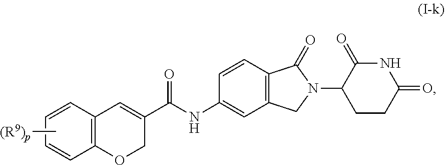

or a pharmaceutically acceptable salt, co-crystal, tautomer, stereoisomer, solvate, hydrate, polymorph, isotopically enriched derivative, or prodrug thereof, wherein R⁹ and p are as defined herein.

In certain embodiments, the compound of Formula (I) is a compound of Formula (I-l):

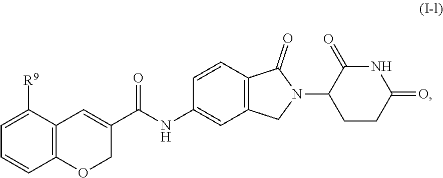

or a pharmaceutically acceptable salt, co-crystal, tautomer, stereoisomer, solvate, hydrate, polymorph, isotopically enriched derivative, or prodrug thereof, wherein R⁹ is as defined herein.

In certain embodiments, the compound of Formula (I) is a compound of Formula (I-m):

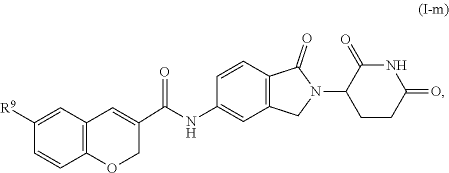

or a pharmaceutically acceptable salt, co-crystal, tautomer, stereoisomer, solvate, hydrate, polymorph, isotopically enriched derivative, or prodrug thereof, wherein R⁹ is as defined herein.

In certain embodiments, the compound of Formula (I) is a compound of Formula (I-n):

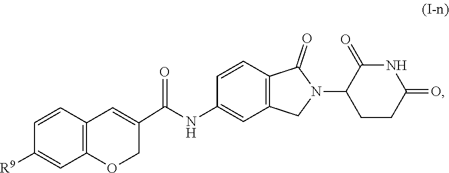

or a pharmaceutically acceptable salt, co-crystal, tautomer, stereoisomer, solvate, hydrate, polymorph, isotopically enriched derivative, or prodrug thereof, wherein R⁹ is as defined herein.

In certain embodiments, the compound of Formula (I) is a compound of Formula (I-o):

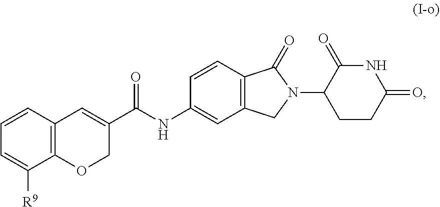

or a pharmaceutically acceptable salt, co-crystal, tautomer, stereoisomer, solvate, hydrate, polymorph, isotopically enriched derivative, or prodrug thereof, wherein R⁹ is as defined herein.

In certain embodiments, the compound of Formula (I) is a compound of Formula (I-p):

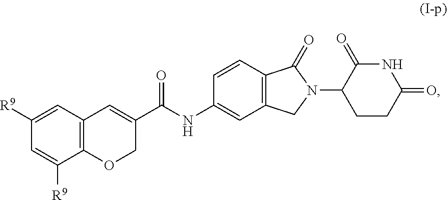

or a pharmaceutically acceptable salt, co-crystal, tautomer, stereoisomer, solvate, hydrate, polymorph, isotopically enriched derivative, or prodrug thereof, wherein R⁹ is as defined herein.

In certain embodiments, the compound of Formula (I) is a compound of Formula (I-q):

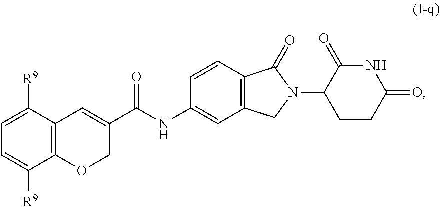

or a pharmaceutically acceptable salt, co-crystal, tautomer, stereoisomer, solvate, hydrate, polymorph, isotopically enriched derivative, or prodrug thereof, wherein R⁹ is as defined herein.

In certain embodiments, the compound of Formula (I) is a compound of the formula:

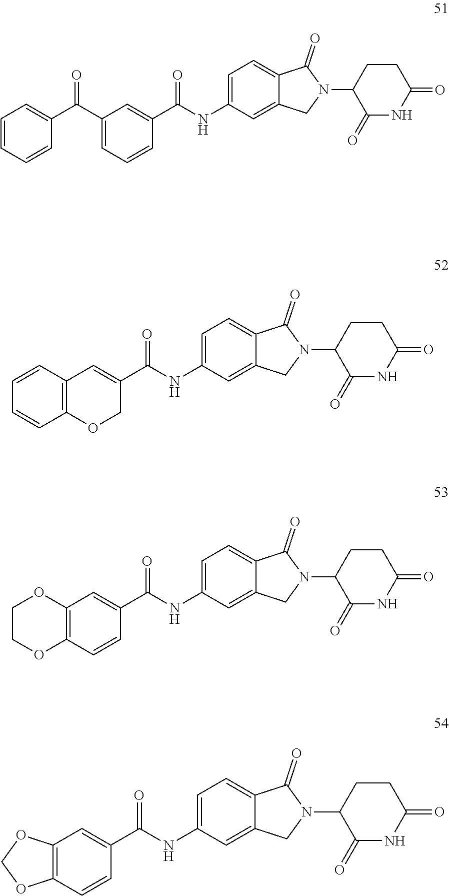 [SMILES: O=C1CCC(N2Cc3cc(NC(=O)c4cccc(C(=O)c5ccccc5)c4)ccc3C2=O)C(=O)N1 ; O=C1CCC(N2Cc3cc(NC(=O)C4=Cc5ccccc5OC4)ccc3C2=O)C(=O)N1 ; O=C1CCC(N2Cc3cc(NC(=O)c4ccc5c(c4)OCCO5)ccc3C2=O)C(=O)N1 ; O=C1CCC(N2Cc3cc(NC(=O)c4ccc5c(c4)OCO5)ccc3C2=O)C(=O)N1]

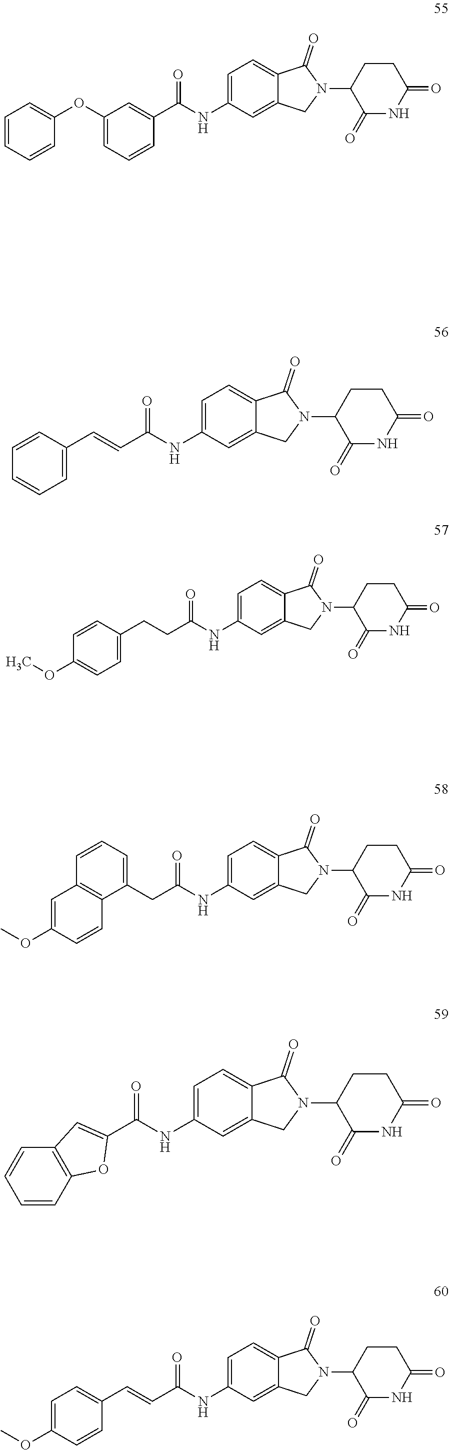 [SMILES: O=C1CCC(N2Cc3cc(NC(=O)c4cccc(Oc5ccccc5)c4)ccc3C2=O)C(=O)N1 ; O=C(/C=C/c1ccccc1)Nc1ccc2c(c1)CN(C1CCC(=O)NC1=O)C2=O ; COc1ccc(CCC(=O)Nc2ccc3c(c2)CN(C2CCC(=O)NC2=O)C3=O)cc1 ; COc1ccc2c(CC(=O)Nc3ccc4c(c3)CN(C3CCC(=O)NC3=O)C4=O)cccc2c1 ; O=C1CCC(N2Cc3cc(NC(=O)c4cc5ccccc5o4)ccc3C2=O)C(=O)N1 ; COc1ccc(/C=C/C(=O)Nc2ccc3c(c2)CN(C2CCC(=O)NC2=O)C3=O)cc1]

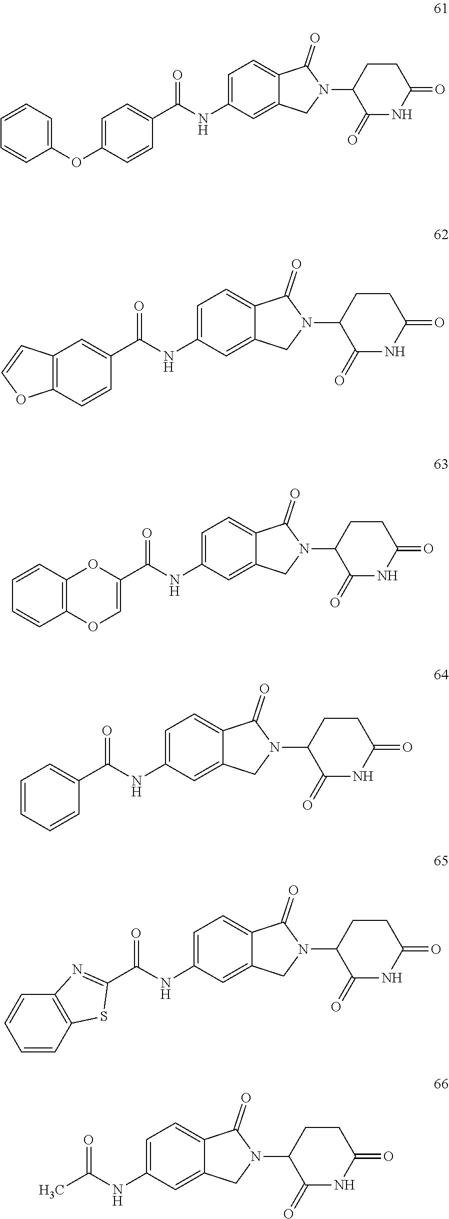 [SMILES: O=C1CCC(N2Cc3cc(NC(=O)c4ccc(Oc5ccccc5)cc4)ccc3C2=O)C(=O)N1 ; O=C1CCC(N2Cc3cc(NC(=O)c4ccc5occc5c4)ccc3C2=O)C(=O)N1 ; O=C1CCC(N2Cc3cc(NC(=O)C4=COc5ccccc5O4)ccc3C2=O)C(=O)N1 ; O=C1CCC(N2Cc3cc(NC(=O)c4ccccc4)ccc3C2=O)C(=O)N1 ; O=C1CCC(N2Cc3cc(NC(=O)c4nc5ccccc5s4)ccc3C2=O)C(=O)N1 ; CC(=O)Nc1ccc2c(c1)CN(C1CCC(=O)NC1=O)C2=O]

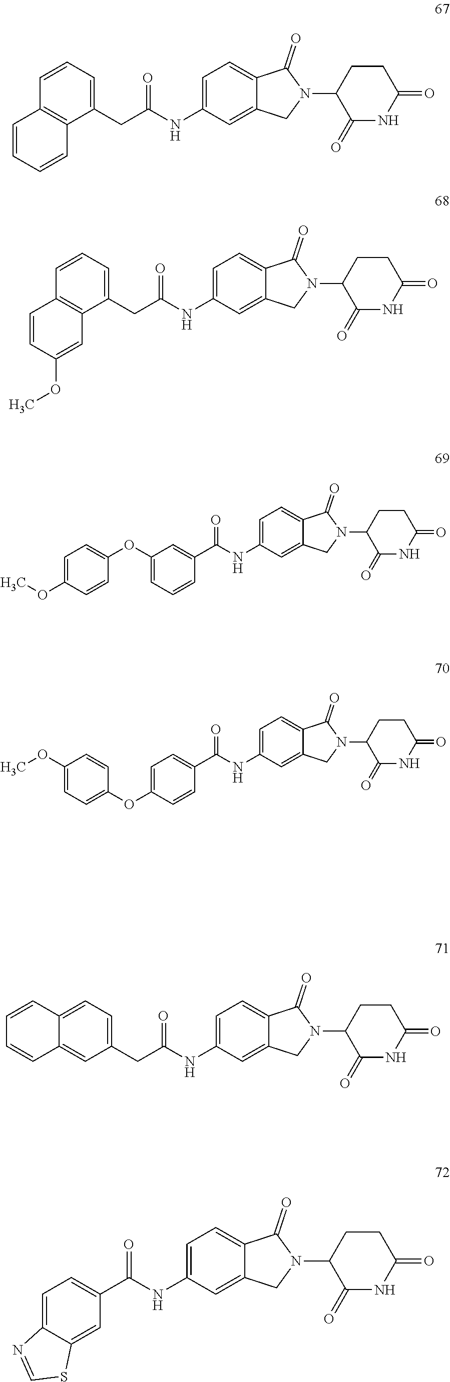 [SMILES: O=C1CCC(N2Cc3cc(NC(=O)Cc4cccc5ccccc45)ccc3C2=O)C(=O)N1 ; COc1ccc2cccc(CC(=O)Nc3ccc4c(c3)CN(C3CCC(=O)NC3=O)C4=O)c2c1 ; COc1ccc(Oc2cccc(C(=O)Nc3ccc4c(c3)CN(C3CCC(=O)NC3=O)C4=O)c2)cc1 ; COc1ccc(Oc2ccc(C(=O)Nc3ccc4c(c3)CN(C3CCC(=O)NC3=O)C4=O)cc2)cc1 ; O=C1CCC(N2Cc3cc(NC(=O)Cc4ccc5ccccc5c4)ccc3C2=O)C(=O)N1 ; O=C1CCC(N2Cc3cc(NC(=O)c4ccc5ncsc5c4)ccc3C2=O)C(=O)N1]

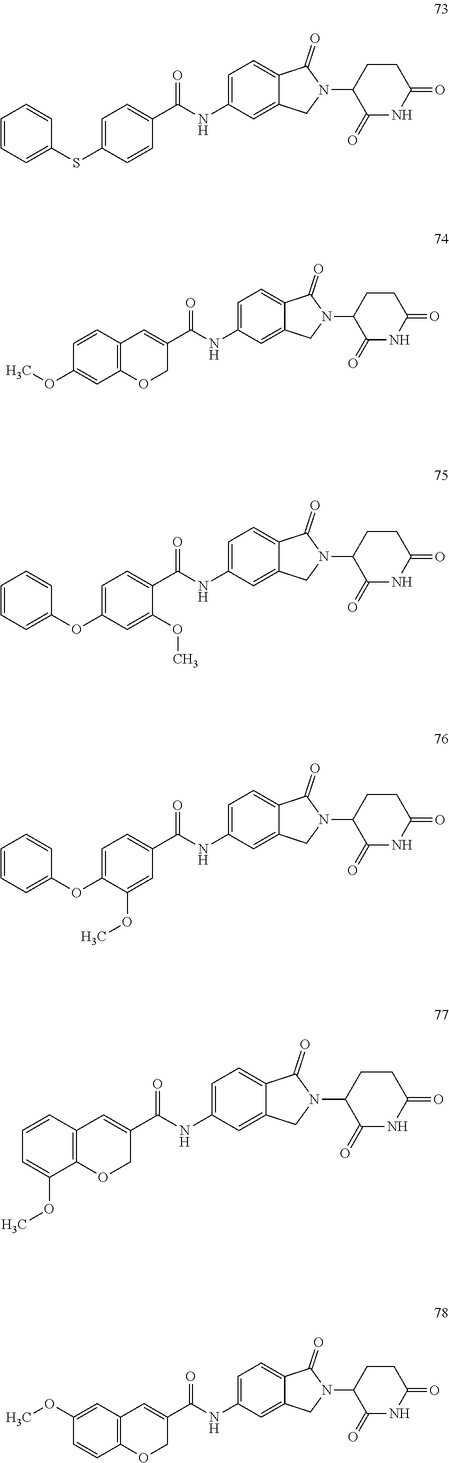 [SMILES: O=C1CCC(N2Cc3cc(NC(=O)c4ccc(Sc5ccccc5)cc4)ccc3C2=O)C(=O)N1 ; COc1ccc2c(c1)OCC(C(=O)Nc1ccc3c(c1)CN(C1CCC(=O)NC1=O)C3=O)=C2 ; COc1cc(Oc2ccccc2)ccc1C(=O)Nc1ccc2c(c1)CN(C1CCC(=O)NC1=O)C2=O ; COc1cc(C(=O)Nc2ccc3c(c2)CN(C2CCC(=O)NC2=O)C3=O)ccc1Oc1ccccc1 ; COc1cccc2c1OCC(C(=O)Nc1ccc3c(c1)CN(C1CCC(=O)NC1=O)C3=O)=C2 ; COc1ccc2c(c1)C=C(C(=O)Nc1ccc3c(c1)CN(C1CCC(=O)NC1=O)C3=O)CO2]

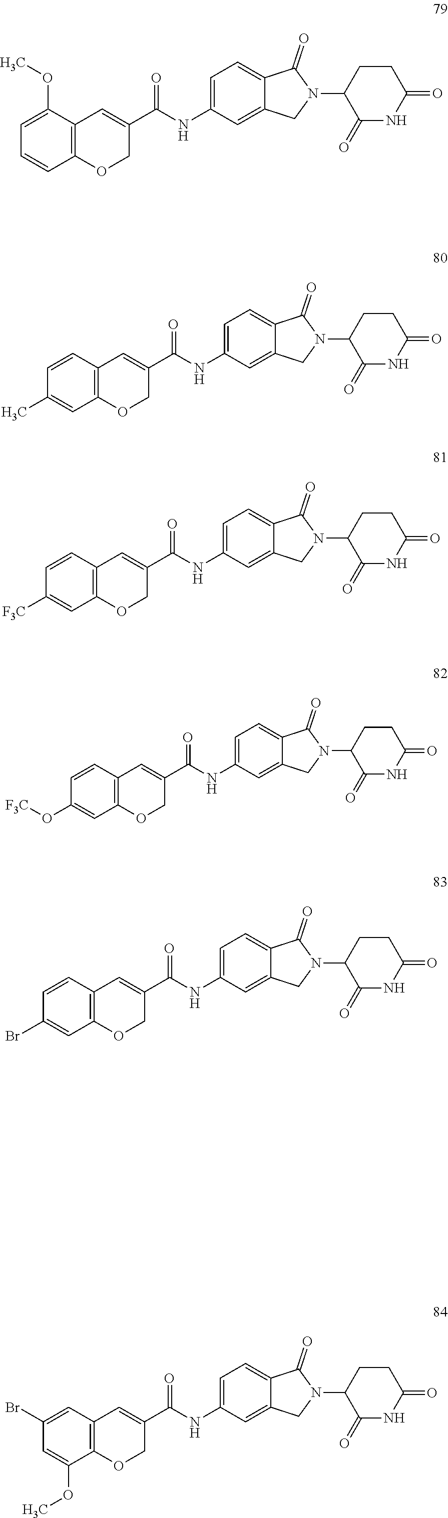 [SMILES: COc1cccc2c1C=C(C(=O)Nc1ccc3c(c1)CN(C1CCC(=O)NC1=O)C3=O)CO2 ; Cc1ccc2c(c1)OCC(C(=O)Nc1ccc3c(c1)CN(C1CCC(=O)NC1=O)C3=O)=C2 ; O=C1CCC(N2Cc3cc(NC(=O)C4=Cc5ccc(C(F)(F)F)cc5OC4)ccc3C2=O)C(=O)N1 ; O=C1CCC(N2Cc3cc(NC(=O)C4=Cc5ccc(OC(F)(F)F)cc5OC4)ccc3C2=O)C(=O)N1 ; O=C1CCC(N2Cc3cc(NC(=O)C4=Cc5ccc(Br)cc5OC4)ccc3C2=O)C(=O)N1 ; COc1cc(Br)cc2c1OCC(C(=O)Nc1ccc3c(c1)CN(C1CCC(=O)NC1=O)C3=O)=C2]

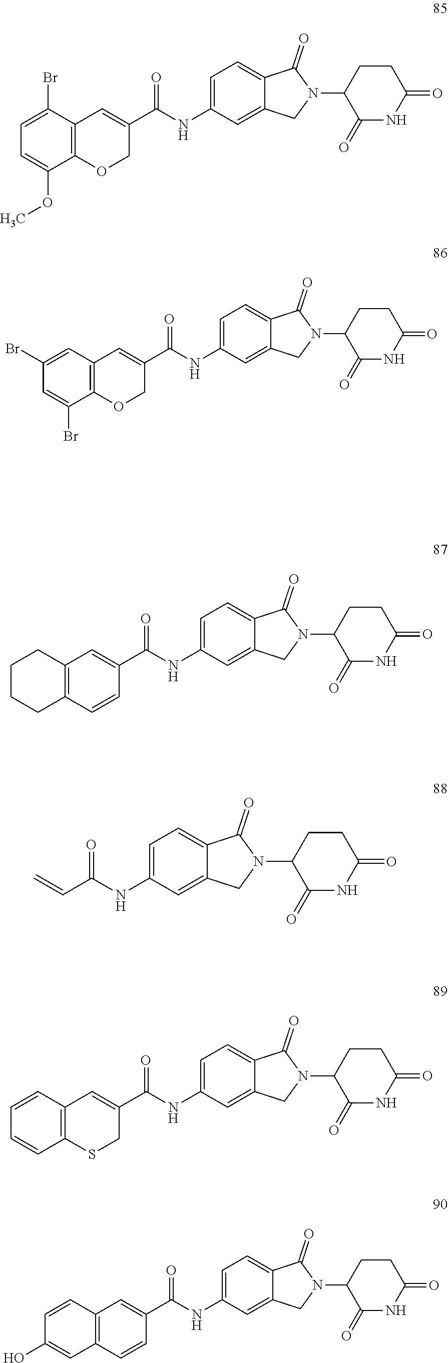 [SMILES: COc1ccc(Br)c2c1OCC(C(=O)Nc1ccc3c(c1)CN(C1CCC(=O)NC1=O)C3=O)=C2 ; O=C1CCC(N2Cc3cc(NC(=O)C4=Cc5cc(Br)cc(Br)c5OC4)ccc3C2=O)C(=O)N1 ; O=C1CCC(N2Cc3cc(NC(=O)c4ccc5c(c4)CCCC5)ccc3C2=O)C(=O)N1 ; C=CC(=O)Nc1ccc2c(c1)CN(C1CCC(=O)NC1=O)C2=O ; O=C1CCC(N2Cc3cc(NC(=O)C4=Cc5ccccc5SC4)ccc3C2=O)C(=O)N1 ; O=C1CCC(N2Cc3cc(NC(=O)c4ccc5cc(O)ccc5c4)ccc3C2=O)C(=O)N1]

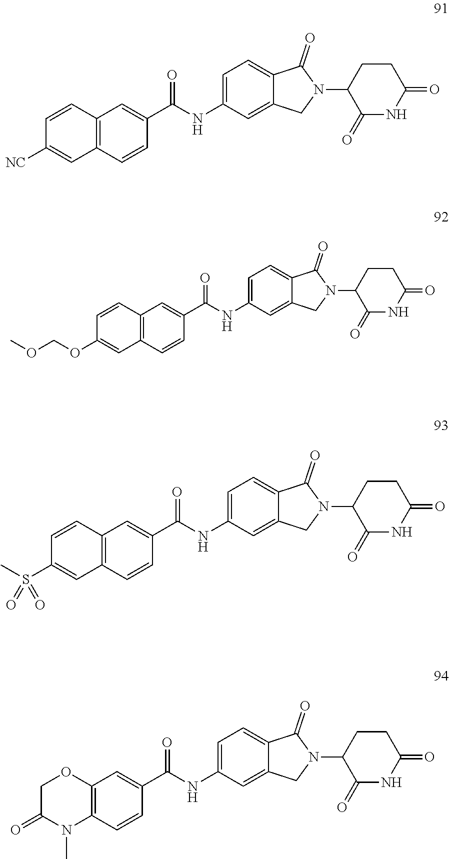 [SMILES: N#Cc1ccc2cc(C(=O)Nc3ccc4c(c3)CN(C3CCC(=O)NC3=O)C4=O)ccc2c1 ; COCOc1ccc2cc(C(=O)Nc3ccc4c(c3)CN(C3CCC(=O)NC3=O)C4=O)ccc2c1 ; CS(=O)(=O)c1ccc2cc(C(=O)Nc3ccc4c(c3)CN(C3CCC(=O)NC3=O)C4=O)ccc2c1 ; CN1C(=O)COc2cc(C(=O)Nc3ccc4c(c3)CN(C3CCC(=O)NC3=O)C4=O)ccc21]

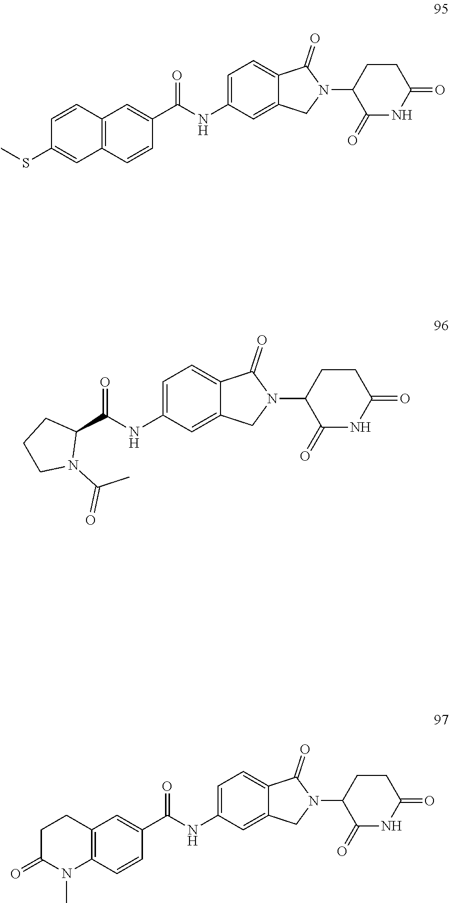 [SMILES: CSc1ccc2cc(C(=O)Nc3ccc4c(c3)CN(C3CCC(=O)NC3=O)C4=O)ccc2c1 ; CC(=O)N1CCC[C@H]1C(=O)Nc1ccc2c(c1)CN(C1CCC(=O)NC1=O)C2=O ; CN1C(=O)CCc2cc(C(=O)Nc3ccc4c(c3)CN(C3CCC(=O)NC3=O)C4=O)ccc21]

or a pharmaceutically acceptable salt, co-crystal, tautomer, stereoisomer, solvate, hydrate, polymorph, isotopically enriched derivative, or prodrug thereof.

In certain embodiments, the compound of Formula (I) is a compound of the formula:

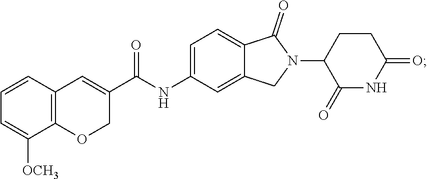 [SMILES: COc1cccc2c1OCC(C(=O)Nc1ccc3c(c1)CN(C1CCC(=O)NC1=O)C3=O)=C2]

or a pharmaceutically acceptable salt, co-crystal, tautomer, stereoisomer, solvate, hydrate, polymorph, isotopically enriched derivative, or prodrug thereof.

In certain embodiments, the compound of Formula (I) binds IKZF1 with a K_{d} of less than 100,000 nM, less than 50,000 nM, less than 20,000 nM, less than 10,000 nM, less than 5,000 nM, less than 2,500 nM, less than 1,000 nM, less than 900 nM, less than 800 nM, less than 700 nM, less than 600 nM, less than 500 nM, less than 400 nM, less than 300 nM, less than 200 nM, less than 100 nM, less than 90 nM, less than 80 nM, less than 70 nM, less than 60 nM, less than 50 nM, less than 40 nM, less than 30 nM, less than 20 nM, less than 10 nM, less than 5 nM, less than 4 nM, less than 3 nM, less than 2 nM, or less than 1 nM.

In certain embodiments, the compound of Formula (I) inhibits IKZF1 with an IC₅₀ of less than 100,000 nM, less than 50,000 nM, less than 20,000 nM, less than 10,000 nM, less than 5,000 nM, less than 2,500 nM, less than 1,000 nM, less than 900 nM, less than 800 nM, less than 700 nM, less than 600 nM, less than 500 nM, less than 400 nM, less than 300 nM, less than 200 nM, less than 100 nM, less than 90 nM, less than 80 nM, less than 70 nM, less than 60 nM, less than 50 nM, less than 40 nM, less than 30 nM, less than 20 nM, less than 10 nM, less than 5 nM, less than 4 nM, less than 3 nM, less than 2 nM, or less than 1 nM.

In certain embodiments, the compound of Formula (I) selectively binds and/or inhibits IKZF1 over another protein. In some embodiments, the compound of Formula (I) selectively binds and/or inhibits IKZF1 over a different IKAROS family protein (e.g., IKZF2, IKZF3, IKZF4, IKZF5). In some embodiments, the compound of Formula (I) selectively binds and/or inhibits IKZF1 over one or more of IKZF2, IKZF3, IKZF4, and IKZF5. In certain embodiments, the selectivity is between about 2-fold and about 5-fold. In certain embodiments, the selectivity is between about 5-fold and about 10-fold. In certain embodiments, the selectivity is between about 10-fold and about 20-fold. In certain embodiments, the selectivity is between about 20-fold and about 50-fold. In certain embodiments, the selectivity is between about 50-fold and about 100-fold. In certain embodiments, the selectivity is between about 100-fold and about 200-fold. In certain embodiments, the selectivity is between about 200-fold and about 500-fold. In certain embodiments, the selectivity is between about 500-fold and about 1000-fold. In certain embodiments, the selectivity is at least about 1000-fold.

In certain embodiments, the compound of Formula (I) promotes the degradation of up to 10%, up to 15%, up to 20%, up to 25%, up to 30%, up to 35%, up to 40%, up to 45%, up to 50%, up to 55%, up to 60%, up to 65%, up to 70%, up to 75%, up to 80%, up to 85%, up to 90%, up to 95%, up to 99%, or up to 100% of IKZF1 at a concentration of 100,000 nM or less, 50,000 nM or less, 20,000 nM or less, 10,000 nM or less, 5,000 nM or less, 3,500 nM or less, 2,500 nM or less, 1,000 nM or less, 900 nM or less, 800 nM or less, 700 nM or less, 600 nM or less, 500 nM or less, 400 nM or less, 300 nM or less, 200 nM or less, 100 nM or less, 90 nM or less, 80 nM or less, 70 nM or less, 60 nM or less, 50 nM or less, 40 nM or less, 30 nM or less, 20 nM or less, 10 nM or less, 5 nM or less, 4 nM or less, 3 nM or less, 2 nM or less, or 1 nM or less. In certain embodiments, the degradation is in a cell.

In certain embodiments, the compound of Formula (I) increases the rate of IKZF1 degradation of up to 10%, up to 15%, up to 20%, up to 25%, up to 30%, up to 35%, up to 40%, up to 45%, up to 50%, up to 55%, up to 60%, up to 65%, up to 70%, up to 75%, up to 80%, up to 85%, up to 90%, up to 95%, up to 99%, or up to 100% at a concentration of 100,000 nM or less, 50,000 nM or less, 20,000 nM or less, 10,000 nM or less, 5,000 nM or less, 3,500 nM or less, 2,500 nM or less, 1,000 nM or less, 900 nM or less, 800 nM or less, 700 nM or less, 600 nM or less, 500 nM or less, 400 nM or less, 300 nM or less, 200 nM or less, 100 nM or less, 90 nM or less, 80 nM or less, 70 nM or less, 60 nM or less, 50 nM or less, 40 nM or less, 30 nM or less, 20 nM or less, 10 nM or less, 5 nM or less, 4 nM or less, 3 nM or less, 2 nM or less, or 1 nM or less.

In certain embodiments, the compound of Formula (I) binds IKZF2 with a K_{d} of less than 100,000 nM, less than 50,000 nM, less than 20,000 nM, less than 10,000 nM, less than 5,000 nM, less than 2,500 nM, less than 1,000 nM, less than 900 nM, less than 800 nM, less than 700 nM, less than 600 nM, less than 500 nM, less than 400 nM, less than 300 nM, less than 200 nM, less than 100 nM, less than 90 nM, less than 80 nM, less than 70 nM, less than 60 nM, less than 50 nM, less than 40 nM, less than 30 nM, less than 20 nM, less than 10 nM, less than 5 nM, less than 4 nM, less than 3 nM, less than 2 nM, or less than 1 nM.

In certain embodiments, the compound of Formula (I) inhibits IKZF2 with an IC₅₀ of less than 100,000 nM, less than 50,000 nM, less than 20,000 nM, less than 10,000 nM, less than 5,000 nM, less than 2,500 nM, less than 1,000 nM, less than 900 nM, less than 800 nM, less than 700 nM, less than 600 nM, less than 500 nM, less than 400 nM, less than 300 nM, less than 200 nM, less than 100 nM, less than 90 nM, less than 80 nM, less than 70 nM, less than 60 nM, less than 50 nM, less than 40 nM, less than 30 nM, less than 20 nM, less than 10 nM, less than 5 nM, less than 4 nM, less than 3 nM, less than 2 nM, or less than 1 nM.

In certain embodiments, the compound of Formula (I) selectively binds and/or inhibits IKZF2 over another protein. In some embodiments, the compound of Formula (I) selectively binds and/or inhibits IKZF2 over a different IKAROS family protein (e.g., IKZF1, IKZF3, IKZF4, IKZF5). In some embodiments, the compound of Formula (I) selectively binds and/or inhibits IKZF2 over one or more of IKZF1, IKZF3, IKZF4, and IKZF5. In certain embodiments, the selectivity is between about 2-fold and about 5-fold. In certain embodiments, the selectivity is between about 5-fold and about 10-fold. In certain embodiments, the selectivity is between about 10-fold and about 20-fold. In certain embodiments, the selectivity is between about 20-fold and about 50-fold. In certain embodiments, the selectivity is between about 50-fold and about 100-fold. In certain embodiments, the selectivity is between about 100-fold and about 200-fold. In certain embodiments, the selectivity is between about 200-fold and about 500-fold. In certain embodiments, the selectivity is between about 500-fold and about 1000-fold. In certain embodiments, the selectivity is at least about 1000-fold.

In certain embodiments, the compound of Formula (I) promotes the degradation of up to 10%, up to 15%, up to 20%, up to 25%, up to 30%, up to 35%, up to 40%, up to 45%, up to 50%, up to 55%, up to 60%, up to 65%, up to 70%, up to 75%, up to 80%, up to 85%, up to 90%, up to 95%, up to 99%, or up to 100% of IKZF2 at a concentration of 100,000 nM or less, 50,000 nM or less, 20,000 nM or less, 10,000 nM or less, 5,000 nM or less, 3,500 nM or less, 2,500 nM or less, 1,000 nM or less, 900 nM or less, 800 nM or less, 700 nM or less, 600 nM or less, 500 nM or less, 400 nM or less, 300 nM or less, 200 nM or less, 100 nM or less, 90 nM or less, 80 nM or less, 70 nM or less, 60 nM or less, 50 nM or less, 40 nM or less, 30 nM or less, 20 nM or less, 10 nM or less, 5 nM or less, 4 nM or less, 3 nM or less, 2 nM or less, or 1 nM or less. In certain embodiments, the degradation is in a cell.

In certain embodiments, the compound of Formula (I) increases the rate of IKZF2 degradation of up to 10%, up to 15%, up to 20%, up to 25%, up to 30%, up to 35%, up to 40%, up to 45%, up to 50%, up to 55%, up to 60%, up to 65%, up to 70%, up to 75%, up to 80%, up to 85%, up to 90%, up to 95%, up to 99%, or up to 100% at a concentration of 100,000 nM or less, 50,000 nM or less, 20,000 nM or less, 10,000 nM or less, 5,000 nM or less, 3,500 nM or less, 2,500 nM or less, 1,000 nM or less, 900 nM or less, 800 nM or less, 700 nM or less, 600 nM or less, 500 nM or less, 400 nM or less, 300 nM or less, 200 nM or less, 100 nM or less, 90 nM or less, 80 nM or less, 70 nM or less, 60 nM or less, 50 nM or less, 40 nM or less, 30 nM or less, 20 nM or less, 10 nM or less, 5 nM or less, 4 nM or less, 3 nM or less, 2 nM or less, or 1 nM or less.

In certain embodiments, the compound of Formula (I) binds CK1α with a K_{d} of less than 100,000 nM, less than 50,000 nM, less than 20,000 nM, less than 10,000 nM, less than 5,000 nM, less than 2,500 nM, less than 1,000 nM, less than 900 nM, less than 800 nM, less than 700 nM, less than 600 nM, less than 500 nM, less than 400 nM, less than 300 nM, less than 200 nM, less than 100 nM, less than 90 nM, less than 80 nM, less than 70 nM, less than 60 nM, less than 50 nM, less than 40 nM, less than 30 nM, less than 20 nM, less than 10 nM, less than 5 nM, less than 4 nM, less than 3 nM, less than 2 nM, or less than 1 nM.

In certain embodiments, the compound of Formula (I) inhibits CK1α with an IC₅₀ of less than 100,000 nM, less than 50,000 nM, less than 20,000 nM, less than 10,000 nM, less than 5,000 nM, less than 2,500 nM, less than 1,000 nM, less than 900 nM, less than 800 nM, less than 700 nM, less than 600 nM, less than 500 nM, less than 400 nM, less than 300 nM, less than 200 nM, less than 100 nM, less than 90 nM, less than 80 nM, less than 70 nM, less than 60 nM, less than 50 nM, less than 40 nM, less than 30 nM, less than 20 nM, less than 10 nM, less than 5 nM, less than 4 nM, less than 3 nM, less than 2 nM, or less than 1 nM.

In certain embodiments, the compound of Formula (I) selectively binds and/or inhibits CK1α over another protein. In some embodiments, the compound of Formula (I) selectively binds and/or inhibits CK1α over a different CK1 family protein (e.g., CK1β, CK1γ, CK1δ, CK1ε). In some embodiments, the compound of Formula (I) selectively binds and/or inhibits CK1α over one or more of CK1β, CK1γ, CK1δ, and CK1ε. In certain embodiments, the selectivity is between about 2-fold and about 5-fold. In certain embodiments, the selectivity is between about 5-fold and about 10-fold. In certain embodiments, the selectivity is between about 10-fold and about 20-fold. In certain embodiments, the selectivity is between about 20-fold and about 50-fold. In certain embodiments, the selectivity is between about 50-fold and about 100-fold. In certain embodiments, the selectivity is between about 100-fold and about 200-fold. In certain embodiments, the selectivity is between about 200-fold and about 500-fold. In certain embodiments, the selectivity is between about 500-fold and about 1000-fold. In certain embodiments, the selectivity is at least about 1000-fold.

In certain embodiments, the compound of Formula (I) promotes the degradation of up to 10%, up to 15%, up to 20%, up to 25%, up to 30%, up to 35%, up to 40%, up to 45%, up to 50%, up to 55%, up to 60%, up to 65%, up to 70%, up to 75%, up to 80%, up to 85%, up to 90%, up to 95%, up to 99%, or up to 100% of CK1α at a concentration of 100,000 nM or less, 50,000 nM or less, 20,000 nM or less, 10,000 nM or less, 5,000 nM or less, 3,500 nM or less, 2,500 nM or less, 1,000 nM or less, 900 nM or less, 800 nM or less, 700 nM or less, 600 nM or less, 500 nM or less, 400 nM or less, 300 nM or less, 200 nM or less, 100 nM or less, 90 nM or less, 80 nM or less, 70 nM or less, 60 nM or less, 50 nM or less, 40 nM or less, 30 nM or less, 20 nM or less, 10 nM or less, 5 nM or less, 4 nM or less, 3 nM or less, 2 nM or less, or 1 nM or less. In certain embodiments, the degradation is in a cell.

In certain embodiments, the compound of Formula (I) increases the rate of CK1α degradation of up to 10%, up to 15%, up to 20%, up to 25%, up to 30%, up to 35%, up to 40%, up to 45%, up to 50%, up to 55%, up to 60%, up to 65%, up to 70%, up to 75%, up to 80%, up to 85%, up to 90%, up to 95%, up to 99%, or up to 100% at a concentration of 100,000 nM or less, 50,000 nM or less, 20,000 nM or less, 10,000 nM or less, 5,000 nM or less, 3,500 nM or less, 2,500 nM or less, 1,000 nM or less, 900 nM or less, 800 nM or less, 700 nM or less, 600 nM or less, 500 nM or less, 400 nM or less, 300 nM or less, 200 nM or less, 100 nM or less, 90 nM or less, 80 nM or less, 70 nM or less, 60 nM or less, 50 nM or less, 40 nM or less, 30 nM or less, 20 nM or less, 10 nM or less, 5 nM or less, 4 nM or less, 3 nM or less, 2 nM or less, or 1 nM or less.

In certain embodiments, the compound of Formula (I) binds GSPT1 with a K_{d} of less than 100,000 nM, less than 50,000 nM, less than 20,000 nM, less than 10,000 nM, less than 5,000 nM, less than 2,500 nM, less than 1,000 nM, less than 900 nM, less than 800 nM, less than 700 nM, less than 600 nM, less than 500 nM, less than 400 nM, less than 300 nM, less than 200 nM, less than 100 nM, less than 90 nM, less than 80 nM, less than 70 nM, less than 60 nM, less than 50 nM, less than 40 nM, less than 30 nM, less than 20 nM, less than 10 nM, less than 5 nM, less than 4 nM, less than 3 nM, less than 2 nM, or less than 1 nM.

In certain embodiments, the compound of Formula (I) inhibits GSPT1 with an IC₅₀ of less than 100,000 nM, less than 50,000 nM, less than 20,000 nM, less than 10,000 nM, less than 5,000 nM, less than 2,500 nM, less than 1,000 nM, less than 900 nM, less than 800 nM, less than 700 nM, less than 600 nM, less than 500 nM, less than 400 nM, less than 300 nM, less than 200 nM, less than 100 nM, less than 90 nM, less than 80 nM, less than 70 nM, less than 60 nM, less than 50 nM, less than 40 nM, less than 30 nM, less than 20 nM, less than 10 nM, less than 5 nM, less than 4 nM, less than 3 nM, less than 2 nM, or less than 1 nM.

In certain embodiments, the compound of Formula (I) selectively binds and/or inhibits GSPT1 over another protein. In some embodiments, the compound of Formula (I) selectively binds and/or inhibits GSPT1 over another protein (e.g., CK1α, IKZF1, IKZF2). In certain embodiments, the selectivity is between about 2-fold and about 5-fold. In certain embodiments, the selectivity is between about 5-fold and about 10-fold. In certain embodiments, the selectivity is between about 10-fold and about 20-fold. In certain embodiments, the selectivity is between about 20-fold and about 50-fold. In certain embodiments, the selectivity is between about 50-fold and about 100-fold. In certain embodiments, the selectivity is between about 100-fold and about 200-fold. In certain embodiments, the selectivity is between about 200-fold and about 500-fold. In certain embodiments, the selectivity is between about 500-fold and about 1000-fold. In certain embodiments, the selectivity is at least about 1000-fold.

In certain embodiments, the compound of Formula (I) promotes the degradation of up to 10%, up to 15%, up to 20%, up to 25%, up to 30%, up to 35%, up to 40%, up to 45%, up to 50%, up to 55%, up to 60%, up to 65%, up to 70%, up to 75%, up to 80%, up to 85%, up to 90%, up to 95%, up to 99%, or up to 100% of GSPT1 at a concentration of 100,000 nM or less, 50,000 nM or less, 20,000 nM or less, 10,000 nM or less, 5,000 nM or less, 3,500 nM or less, 2,500 nM or less, 1,000 nM or less, 900 nM or less, 800 nM or less, 700 nM or less, 600 nM or less, 500 nM or less, 400 nM or less, 300 nM or less, 200 nM or less, 100 nM or less, 90 nM or less, 80 nM or less, 70 nM or less, 60 nM or less, 50 nM or less, 40 nM or less, 30 nM or less, 20 nM or less, 10 nM or less, 5 nM or less, 4 nM or less, 3 nM or less, 2 nM or less, or 1 nM or less. In certain embodiments, the degradation is in a cell.

In certain embodiments, the compound of Formula (I) increases the rate of GSPT1 degradation of up to 10%, up to 15%, up to 20%, up to 25%, up to 30%, up to 35%, up to 40%, up to 45%, up to 50%, up to 55%, up to 60%, up to 65%, up to 70%, up to 75%, up to 80%, up to 85%, up to 90%, up to 95%, up to 99%, or up to 100% at a concentration of 100,000 nM or less, 50,000 nM or less, 20,000 nM or less, 10,000 nM or less, 5,000 nM or less, 3,500 nM or less, 2,500 nM or less, 1,000 nM or less, 900 nM or less, 800 nM or less, 700 nM or less, 600 nM or less, 500 nM or less, 400 nM or less, 300 nM or less, 200 nM or less, 100 nM or less, 90 nM or less, 80 nM or less, 70 nM or less, 60 nM or less, 50 nM or less, 40 nM or less, 30 nM or less, 20 nM or less, 10 nM or less, 5 nM or less, 4 nM or less, 3 nM or less, 2 nM or less, or 1 nM or less.

In certain embodiments, the compound of Formula (I) selectively degrades CK1α over GSPT1 and IKZF1. In certain embodiments, the selectivity is between about 2-fold and about 5-fold. In certain embodiments, the selectivity is between about 5-fold and about 10-fold. In certain embodiments, the selectivity is between about 10-fold and about 20-fold. In certain embodiments, the selectivity is between about 20-fold and about 50-fold. In certain embodiments, the selectivity is between about 50-fold and about 100-fold. In certain embodiments, the selectivity is between about 100-fold and about 200-fold. In certain embodiments, the selectivity is between about 200-fold and about 500-fold. In certain embodiments, the selectivity is between about 500-fold and about 1000-fold. In certain embodiments, the selectivity is at least about 1000-fold.

In certain embodiments, the compound of Formula (I) selectively degrades IKZF2 over GSPT1 and IKZF1. In certain embodiments, the selectivity is between about 2-fold and about 5-fold. In certain embodiments, the selectivity is between about 5-fold and about 10-fold. In certain embodiments, the selectivity is between about 10-fold and about 20-fold. In certain embodiments, the selectivity is between about 20-fold and about 50-fold. In certain embodiments, the selectivity is between about 50-fold and about 100-fold. In certain embodiments, the selectivity is between about 100-fold and about 200-fold. In certain embodiments, the selectivity is between about 200-fold and about 500-fold. In certain embodiments, the selectivity is between about 500-fold and about 1000-fold. In certain embodiments, the selectivity is at least about 1000-fold.

In certain embodiments, the compound of Formula (I) selectively degrades CK1α and IKZF2 over GSPT1 and IKZF1. In certain embodiments, the selectivity is between about 2-fold and about 5-fold. In certain embodiments, the selectivity is between about 5-fold and about 10-fold. In certain embodiments, the selectivity is between about 10-fold and about 20-fold. In certain embodiments, the selectivity is between about 20-fold and about 50-fold. In certain embodiments, the selectivity is between about 50-fold and about 100-fold. In certain embodiments, the selectivity is between about 100-fold and about 200-fold. In certain embodiments, the selectivity is between about 200-fold and about 500-fold. In certain embodiments, the selectivity is between about 500-fold and about 1000-fold. In certain embodiments, the selectivity is at least about 1000-fold.

### Pharmaceutical Compositions, Kits, and Administration

The present disclosure provides pharmaceutical compositions comprising a compound of Formula (I), or a pharmaceutically acceptable salt, co-crystal, tautomer, stereoisomer, solvate, hydrate, polymorph, isotopically enriched derivative, or prodrug thereof, and optionally a pharmaceutically acceptable excipient. In certain embodiments, the pharmaceutical composition described herein comprises a compound of Formula (I), or a pharmaceutically acceptable salt thereof, and a pharmaceutically acceptable excipient.

In certain embodiments, the compound of Formula (I) is provided in an effective amount in the pharmaceutical composition. In certain embodiments, the effective amount is a therapeutically effective amount. In certain embodiments, the effective amount is a prophylactically effective amount. In certain embodiments, the effective amount is an amount effective for treating a proliferative disorder in a subject in need thereof. In certain embodiments, the effective amount is an amount effective for treating cancer in a subject in need thereof. In certain embodiments, the effective amount is an amount effective for preventing cancer in a subject in need thereof. In certain embodiments, the effective amount is an amount effective for treating a pediatric cancer (e.g., childhood acute leukemia (AL) or medulloblastoma (MB)), lung cancer, breast cancer, colon cancer, colorectal cancer, ovarian cancer, gastric cancer, or a hematological cancer in a subject in need thereof. In certain embodiments, the effective amount is an amount effective for treating ovarian cancer, gastric cancer, or a hematological cancer in a subject in need thereof. In certain embodiments, the effective amount is an amount effective for treating ovarian cancer in a subject in need thereof. In certain embodiments, the effective amount is an amount effective for treating a hematological cancer in a subject in need thereof. In certain embodiments, the effective amount is an amount effective for treating a leukemia, a lymphoma, or multiple myeloma in a subject in need thereof. In certain embodiments, the effective amount is an amount effective for treating multiple myeloma in a subject in need thereof. In certain embodiments, the effective amount is an amount effective for treating a leukemia or a lymphoma in a subject in need thereof. In certain embodiments, the effective amount is an amount effective for treating primary effusion lymphoma (PEL), del(5q) myelodysplastic syndrome (MDS), chronic myeloid leukemia (CML), chronic lymphoid leukemia (CLL), acute myeloid leukemia (AML), hairy cell leukemia, chronic myelomonocytic leukemia (CMML), juvenile myelomonocyte leukemia (JMML), large granular lymphocytic leukemia (LGL), acute lymphoblastic leukemia (ALL), B-cell acute lymphoblastic leukemia (B-ALL), or T-cell acute lymphoblastic leukemia (T-ALL), B-cell lymphoma, diffuse large B-cell lymphoma (DLBCL), T-cell-lymphoma, Hodgkin's lymphoma, non-Hodgkin's lymphoma, hairy cell lymphoma, or Burkett's lymphoma in a subject in need thereof. In certain embodiments, the effective amount is an amount effective for treating primary effusion lymphoma (PEL), del(5q) myelodysplastic syndrome (MDS), or acute myeloid leukemia (AML) in a subject in need thereof. In certain embodiments, the effective amount is an amount effective for treating acute myeloid leukemia (AML) in a subject in need thereof. In certain embodiments, the effective amount is an amount effective for treating diffuse large B-cell lymphoma (DLBCL) in a subject in need thereof. In certain embodiments, the effective amount is an amount effective for treating T-cell acute lymphoblastic leukemia (T-ALL) in a subject in need thereof.

In certain embodiments, the subject is an animal. The animal may be of either sex and may be at any stage of development. In certain embodiments, the subject described herein is a human. In certain embodiments, the subject is a non-human animal. In certain embodiments, the subject is a mammal. In certain embodiments, the subject is a non-human mammal. In certain embodiments, the subject is a domesticated animal, such as a dog, cat, cow, pig, horse, sheep, or goat. In certain embodiments, the subject is a companion animal, such as a dog or cat. In certain embodiments, the subject is a livestock animal, such as a cow, pig, horse, sheep, or goat. In certain embodiments, the subject is a zoo animal. In another embodiment, the subject is a research animal, such as a rodent (e.g., mouse, rat), dog, pig, or non-human primate. In certain embodiments, the animal is a genetically engineered animal. In certain embodiments, the animal is a transgenic animal (e.g., transgenic mice and transgenic pigs). In certain embodiments, the subject is a fish or reptile.

In certain embodiments, the effective amount is an amount effective for promoting the degradation of at least about 10%, at least about 20%, at least about 30%, at least about 40%, at least about 50%, at least about 60%, at least about 70%, at least about 80%, at least about 90%, at least about 95%, at least about 98%, or at least about 99% of a protein.

In certain embodiments, the effective amount is an amount effective for promoting the degradation of at least about 10%, at least about 20%, at least about 30%, at least about 40%, at least about 50%, at least about 60%, at least about 70%, at least about 80%, at least about 90%, at least about 95%, at least about 98%, or at least about 99% of IKZF1. In certain embodiments, the effective amount is an amount effective for promoting the degradation of IKZF1 by a range between a percentage described in this paragraph and another percentage described in this paragraph, inclusive.

In certain embodiments, the effective amount is an amount effective for promoting the degradation of at least about 10%, at least about 20%, at least about 30%, at least about 40%, at least about 50%, at least about 60%, at least about 70%, at least about 80%, at least about 90%, at least about 95%, at least about 98%, or at least about 99% of IKZF2. In certain embodiments, the effective amount is an amount effective for promoting the degradation of IKZF2 by a range between a percentage described in this paragraph and another percentage described in this paragraph, inclusive.

In certain embodiments, the effective amount is an amount effective for promoting the degradation of at least about 10%, at least about 20%, at least about 30%, at least about 40%, at least about 50%, at least about 60%, at least about 70%, at least about 80%, at least about 90%, at least about 95%, at least about 98%, or at least about 99% of CK1α. In certain embodiments, the effective amount is an amount effective for promoting the degradation of CK1α by a range between a percentage described in this paragraph and another percentage described in this paragraph, inclusive.

In certain embodiments, the effective amount is an amount effective for promoting the degradation of at least about 10%, at least about 20%, at least about 30%, at least about 40%, at least about 50%, at least about 60%, at least about 70%, at least about 80%, at least about 90%, at least about 95%, at least about 98%, or at least about 99% of GSPT1. In certain embodiments, the effective amount is an amount effective for promoting the degradation of GSPT1 by a range between a percentage described in this paragraph and another percentage described in this paragraph, inclusive.

The present disclosure provides pharmaceutical compositions comprising a compound that interacts with CK1α, IKZF1, IKZF2, and/or GSPT1 for use in treating a proliferative disorder in a subject in need thereof. In certain embodiments, the composition is for use in treating cancer. In certain embodiments, the composition is for use in treating a pediatric cancer (e.g., childhood acute leukemia (AL) or medulloblastoma (MB)), lung cancer, breast cancer, colon cancer, colorectal cancer, ovarian cancer, gastric cancer, or a hematological cancer. In certain embodiments, the composition is for use in treating ovarian cancer, gastric cancer, or a hematological cancer. In certain embodiments, the composition is for use in treating ovarian cancer. In certain embodiments, the composition is for use in treating a hematological cancer. In certain embodiments, the composition is for use in treating a leukemia, a lymphoma, or multiple myeloma. In certain embodiments, the composition is for use in treating a leukemia or a lymphoma. In certain embodiments, the composition is for use in treating multiple myeloma. In certain embodiments, the composition is for use in treating primary effusion lymphoma (PEL), del(5q) myelodysplastic syndrome (MDS), chronic myeloid leukemia (CML), chronic lymphoid leukemia (CLL), acute myeloid leukemia (AML), hairy cell leukemia, chronic myelomonocytic leukemia (CMML), juvenile myelomonocyte leukemia (JMML), large granular lymphocytic leukemia (LGL), acute lymphoblastic leukemia (ALL), B-cell acute lymphoblastic leukemia (B-ALL), or T-cell acute lymphoblastic leukemia (T-ALL), B-cell lymphoma, diffuse large B-cell lymphoma (DLBCL), T-cell-lymphoma, Hodgkin's lymphoma, non-Hodgkin's lymphoma, hairy cell lymphoma, or Burkett's lymphoma. In certain embodiments, the composition is for use in treating primary effusion lymphoma (PEL), del(5q) myelodysplastic syndrome (MDS), or acute myeloid leukemia (AML) in a subject in need thereof. In certain embodiments, the composition is for use in treating acute myeloid leukemia (AML) in a subject in need thereof. In certain embodiments, the composition is for use in treating diffuse large B-cell lymphoma (DLBCL). In certain embodiments, the composition is for use in treating T-cell acute lymphoblastic leukemia (T-ALL).

A compound or composition, as described herein, can be administered in combination with one or more additional pharmaceutical agents (e.g., therapeutically and/or prophylactically active agents). The compounds or compositions can be administered in combination with additional pharmaceutical agents that improve their activity (e.g., activity (e.g., potency and/or efficacy) in treating a disease in a subject in need thereof, in preventing a disease in a subject in need thereof, and/or in reducing the risk to develop a disease in a subject in need thereof), improve bioavailability, improve their ability to cross the blood-brain barrier, improve safety, reduce drug resistance, reduce and/or modify metabolism, inhibit excretion, and/or modify distribution in a subject or cell. It will also be appreciated that the therapy employed may achieve a desired effect for the same disorder, and/or it may achieve different effects. In certain embodiments, a pharmaceutical composition described herein including a compound described herein and an additional pharmaceutical agent exhibit a synergistic effect that is absent in a pharmaceutical composition including one of the compound and the additional pharmaceutical agent, but not both.

The compound or composition can be administered concurrently with, prior to, or subsequent to one or more additional pharmaceutical agents, which may be useful as, e.g., combination therapies. Pharmaceutical agents include therapeutically active agents. Pharmaceutical agents also include prophylactically active agents. Pharmaceutical agents include small organic molecules such as drug compounds (e.g., compounds approved for human or veterinary use by the U.S. Food and Drug Administration as provided in the Code of Federal Regulations (CFR)), peptides, proteins, carbohydrates, monosaccharides, oligosaccharides, polysaccharides, nucleoproteins, mucoproteins, lipoproteins, synthetic polypeptides or proteins, small molecules linked to proteins, glycoproteins, steroids, nucleic acids, DNAs, RNAs, nucleotides, nucleosides, oligonucleotides, antisense oligonucleotides, lipids, hormones, vitamins, and cells. In certain embodiments, the additional pharmaceutical agent is a pharmaceutical agent useful for treating and/or preventing a disease (e.g., cancer). Each additional pharmaceutical agent may be administered at a dose and/or on a time schedule determined for that pharmaceutical agent. The additional pharmaceutical agents may also be administered together with each other and/or with the compound or composition described herein in a single dose or administered separately in different doses. The particular combination to employ in a regimen will take into account compatibility of the compound described herein with the additional pharmaceutical agent(s) and/or the desired therapeutic and/or prophylactic effect to be achieved. In general, it is expected that the additional pharmaceutical agent(s) in combination be utilized at levels that do not exceed the levels at which they are utilized individually. In some embodiments, the levels utilized in combination will be lower than those utilized individually.

In certain embodiments, the compound or pharmaceutical composition is a solid. In certain embodiments, the compound or pharmaceutical composition is a powder. In certain embodiments, the compound or pharmaceutical composition can be dissolved in a liquid to make a solution. In certain embodiments, the compound or pharmaceutical composition is dissolved in water to make an aqueous solution. In certain embodiments, the pharmaceutical composition is a liquid for parental injection. In certain embodiments, the pharmaceutical composition is a liquid for oral administration (e.g., ingestion). In certain embodiments, the pharmaceutical composition is a liquid (e.g., aqueous solution) for intravenous injection. In certain embodiments, the pharmaceutical composition is a liquid (e.g., aqueous solution) for subcutaneous injection.

After formulation with an appropriate pharmaceutically acceptable excipient in a desired dosage, the pharmaceutical compositions of this invention can be administered to humans and other animals orally, parenterally, intracisternally, intraperitoneally, topically, bucally, or the like, depending on the disease or condition being treated.

In certain embodiments, a pharmaceutical composition comprising a compound of Formula (I) is administered, orally or parenterally, at dosage levels of each pharmaceutical composition sufficient to deliver from about 0.001 mg/kg to about 200 mg/kg in one or more dose administrations for one or several days (depending on the mode of administration). In certain embodiments, the effective amount per dose varies from about 0.001 mg/kg to about 200 mg/kg, about 0.001 mg/kg to about 100 mg/kg, about 0.01 mg/kg to about 100 mg/kg, from about 0.01 mg/kg to about 50 mg/kg, preferably from about 0.1 mg/kg to about 40 mg/kg, preferably from about 0.5 mg/kg to about 30 mg/kg, from about 0.01 mg/kg to about 10 mg/kg, from about 0.1 mg/kg to about 10 mg/kg, of subject body weight per day, one or more times a day, to obtain the desired therapeutic and/or prophylactic effect. In certain embodiments, the compounds described herein may be at dosage levels sufficient to deliver from about 0.001 mg/kg to about 200 mg/kg, from about 0.001 mg/kg to about 100 mg/kg, from about 0.01 mg/kg to about 100 mg/kg, from about 0.01 mg/kg to about 50 mg/kg, preferably from about 0.1 mg/kg to about 40 mg/kg, preferably from about 0.5 mg/kg to about 30 mg/kg, from about 0.01 mg/kg to about 10 mg/kg, from about 0.1 mg/kg to about 10 mg/kg, and more preferably from about 1 mg/kg to about 25 mg/kg, of subject body weight per day, one or more times a day, to obtain the desired therapeutic and/or prophylactic effect. The desired dosage may be delivered three times a day, two times a day, once a day, every other day, every third day, every week, every two weeks, every three weeks, or every four weeks. In certain embodiments, the desired dosage may be delivered using multiple administrations (e.g., two, three, four, five, six, seven, eight, nine, ten, eleven, twelve, thirteen, fourteen, or more administrations). In certain embodiments, the composition described herein is administered at a dose that is below the dose at which the agent causes non-specific effects.

In certain embodiments, the pharmaceutical composition is administered at a dose of about 0.001 mg to about 1000 mg per unit dose. In certain embodiments, the pharmaceutical composition is administered at a dose of about 0.01 mg to about 200 mg per unit dose. In certain embodiments, the pharmaceutical composition is administered at a dose of about 0.01 mg to about 100 mg per unit dose. In certain embodiments, pharmaceutical composition is administered at a dose of about 0.01 mg to about 50 mg per unit dose. In certain embodiments, the pharmaceutical composition is administered at a dose of about 0.01 mg to about 10 mg per unit dose. In certain embodiments, the pharmaceutical composition is administered at a dose of about 0.1 mg to about 10 mg per unit dose.

Pharmaceutical compositions described herein can be prepared by any method known in the art of pharmacology. In general, such preparatory methods include the steps of bringing the composition comprising a compound of Formula (I) into association with a carrier and/or one or more other accessory ingredients, and then, if necessary and/or desirable, shaping and/or packaging the product into a desired single- or multi-dose unit.

Pharmaceutical compositions can be prepared, packaged, and/or sold in bulk, as a single unit dose, and/or as a plurality of single unit doses. As used herein, a “unit dose” is a discrete amount of the pharmaceutical composition comprising a predetermined amount of the active ingredient. The amount of the active ingredient is generally equal to the dosage of the active ingredient which would be administered to a subject and/or a convenient fraction of such a dosage, such as, for example, one-half or one-third of such a dosage.

Relative amounts of the active ingredient, the pharmaceutically acceptable excipient, and/or any additional ingredients in a pharmaceutical composition of the invention will vary, depending upon the identity, size, and/or condition of the subject treated and further depending upon the route by which the composition is to be administered. By way of example, the composition may comprise between 0.1% and 100% (w/w) active ingredient.

Pharmaceutically acceptable excipients used in the manufacture of provided pharmaceutical compositions include inert diluents, dispersing and/or granulating agents, surface active agents and/or emulsifiers, disintegrating agents, binding agents, preservatives, buffering agents, lubricating agents, and/or oils. Excipients, such as cocoa butter and suppository waxes, coloring agents, coating agents, sweetening, flavoring, and perfuming agents, may also be present in the composition.

Exemplary diluents include calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, powdered sugar, and mixtures thereof.

Exemplary granulating and/or dispersing agents include potato starch, corn starch, tapioca starch, sodium starch glycolate, clays, alginic acid, guar gum, citrus pulp, agar, bentonite, cellulose, and wood products, natural sponge, cation-exchange resins, calcium carbonate, silicates, sodium carbonate, cross-linked poly(vinyl-pyrrolidone) (crospovidone), sodium carboxymethyl starch (sodium starch glycolate), carboxymethyl cellulose, cross-linked sodium carboxymethyl cellulose (croscarmellose), methylcellulose, pregelatinized starch (starch 1500), microcrystalline starch, water insoluble starch, calcium carboxymethyl cellulose, magnesium aluminum silicate (Veegum), sodium lauryl sulfate, quaternary ammonium compounds, and mixtures thereof.

Exemplary surface active agents and/or emulsifiers include natural emulsifiers (e.g. acacia, agar, alginic acid, sodium alginate, tragacanth, chondrux, cholesterol, xanthan, pectin, gelatin, egg yolk, casein, wool fat, cholesterol, wax, and lecithin), colloidal clays (e.g. bentonite (aluminum silicate) and Veegum (magnesium aluminum silicate)), long chain amino acid derivatives, high molecular weight alcohols (e.g. stearyl alcohol, cetyl alcohol, oleyl alcohol, triacetin monostearate, ethylene glycol distearate, glyceryl monostearate, and propylene glycol monostearate, polyvinyl alcohol), carbomers (e.g. carboxy polymethylene, polyacrylic acid, acrylic acid polymer, and carboxyvinyl polymer), carrageenan, cellulosic derivatives (e.g. carboxymethylcellulose sodium, powdered cellulose, hydroxymethyl cellulose, hydroxypropyl cellulose, hydroxypropyl methylcellulose, methylcellulose), sorbitan fatty acid esters (e.g. polyoxyethylene sorbitan monolaurate (Tween 20), polyoxyethylene sorbitan (Tween 60), polyoxyethylene sorbitan monooleate (Tween 80), sorbitan monopalmitate (Span 40), sorbitan monostearate (Span 60), sorbitan tristearate (Span 65), glyceryl monooleate, sorbitan monooleate (Span 80)), polyoxyethylene esters (e.g. polyoxyethylene monostearate (Myrj 45), polyoxyethylene hydrogenated castor oil, polyethoxylated castor oil, polyoxymethylene stearate, and Solutol), sucrose fatty acid esters, polyethylene glycol fatty acid esters (e.g. Cremophor™), polyoxyethylene ethers, (e.g. polyoxyethylene lauryl ether (Brij 30)), poly(vinyl-pyrrolidone), diethylene glycol monolaurate, triethanolamine oleate, sodium oleate, potassium oleate, ethyl oleate, oleic acid, ethyl laurate, sodium lauryl sulfate, Pluronic F-68, Poloxamer-188, cetrimonium bromide, cetylpyridinium chloride, benzalkonium chloride, docusate sodium, and/or mixtures thereof.

Exemplary binding agents include starch (e.g. cornstarch and starch paste), gelatin, sugars (e.g. sucrose, glucose, dextrose, dextrin, molasses, lactose, lactitol, mannitol, etc.), natural and synthetic gums (e.g. acacia, sodium alginate, extract of Irish moss, panwar gum, ghatti gum, mucilage of isapol husks, carboxymethylcellulose, methylcellulose, ethylcellulose, hydroxyethylcellulose, hydroxypropyl cellulose, hydroxypropyl methylcellulose, microcrystalline cellulose, cellulose acetate, poly(vinyl-pyrrolidone), magnesium aluminum silicate (Veegum), and larch arabogalactan), alginates, polyethylene oxide, polyethylene glycol, inorganic calcium salts, silicic acid, polymethacrylates, waxes, water, alcohol, and/or mixtures thereof.

Exemplary preservatives include antioxidants, chelating agents, antimicrobial preservatives, antifungal preservatives, alcohol preservatives, acidic preservatives, and other preservatives. In certain embodiments, the preservative is an antioxidant. In other embodiments, the preservative is a chelating agent.

Exemplary antioxidants include alpha tocopherol, ascorbic acid, acorbyl palmitate, butylated hydroxyanisole, butylated hydroxytoluene, monothioglycerol, potassium metabisulfite, propionic acid, propyl gallate, sodium ascorbate, sodium bisulfite, sodium metabisulfite, and sodium sulfite.

Exemplary chelating agents include ethylenediaminetetraacetic acid (EDTA) and salts and hydrates thereof (e.g., sodium edetate, disodium edetate, trisodium edetate, calcium disodium edetate, dipotassium edetate, and the like), citric acid and salts and hydrates thereof (e.g., citric acid monohydrate), fumaric acid and salts and hydrates thereof, malic acid and salts and hydrates thereof, phosphoric acid and salts and hydrates thereof, and tartaric acid and salts and hydrates thereof. Exemplary antimicrobial preservatives include benzalkonium chloride, benzethonium chloride, benzyl alcohol, bronopol, cetrimide, cetylpyridinium chloride, chlorhexidine, chlorobutanol, chlorocresol, chloroxylenol, cresol, ethyl alcohol, glycerin, hexetidine, imidurea, phenol, phenoxyethanol, phenylethyl alcohol, phenylmercuric nitrate, propylene glycol, and thimerosal.

Exemplary antifungal preservatives include butyl paraben, methyl paraben, ethyl paraben, propyl paraben, benzoic acid, hydroxybenzoic acid, potassium benzoate, potassium sorbate, sodium benzoate, sodium propionate, and sorbic acid.

Exemplary alcohol preservatives include ethanol, polyethylene glycol, phenol, phenolic compounds, bisphenol, chlorobutanol, hydroxybenzoate, and phenylethyl alcohol.

Exemplary acidic preservatives include vitamin A, vitamin C, vitamin E, beta-carotene, citric acid, acetic acid, dehydroacetic acid, ascorbic acid, sorbic acid, and phytic acid.

Other preservatives include tocopherol, tocopherol acetate, deteroxime mesylate, cetrimide, butylated hydroxyanisol (BHA), butylated hydroxytoluened (BHT), ethylenediamine, sodium lauryl sulfate (SLS), sodium lauryl ether sulfate (SLES), sodium bisulfite, sodium metabisulfite, potassium sulfite, potassium metabisulfite, Glydant Plus, Phenonip, methylparaben, Germall 115, Germaben II, Neolone, Kathon, and Euxyl.

Exemplary buffering agents include citrate buffer solutions, acetate buffer solutions, phosphate buffer solutions, ammonium chloride, calcium carbonate, calcium chloride, calcium citrate, calcium glubionate, calcium gluceptate, calcium gluconate, D-gluconic acid, calcium glycerophosphate, calcium lactate, propanoic acid, calcium levulinate, pentanoic acid, dibasic calcium phosphate, phosphoric acid, tribasic calcium phosphate, calcium hydroxide phosphate, potassium acetate, potassium chloride, potassium gluconate, potassium mixtures, dibasic potassium phosphate, monobasic potassium phosphate, potassium phosphate mixtures, sodium acetate, sodium bicarbonate, sodium chloride, sodium citrate, sodium lactate, dibasic sodium phosphate, monobasic sodium phosphate, sodium phosphate mixtures, tromethamine, magnesium hydroxide, aluminum hydroxide, alginic acid, pyrogen-free water, isotonic saline, Ringer's solution, ethyl alcohol, and mixtures thereof.

Exemplary lubricating agents include magnesium stearate, calcium stearate, stearic acid, silica, talc, malt, glyceryl behanate, hydrogenated vegetable oils, polyethylene glycol, sodium benzoate, sodium acetate, sodium chloride, leucine, magnesium lauryl sulfate, sodium lauryl sulfate, and mixtures thereof.

Exemplary natural oils include almond, apricot kernel, avocado, babassu, bergamot, black current seed, borage, cade, camomile, canola, caraway, carnauba, castor, cinnamon, cocoa butter, coconut, cod liver, coffee, corn, cotton seed, emu, eucalyptus, evening primrose, fish, flaxseed, geraniol, gourd, grape seed, hazelnut, hyssop, isopropyl myristate, jojoba, kukui nut, lavandin, lavender, lemon, Litsea cubeba, macademia nut, mallow, mango seed, meadowfoam seed, mink, nutmeg, olive, orange, orange roughy, palm, palm kernel, peach kernel, peanut, poppy seed, pumpkin seed, rapeseed, rice bran, rosemary, safflower, sandalwood, sasquana, savoury, sea buckthorn, sesame, shea butter, silicone, soybean, sunflower, tea tree, thistle, tsubaki, vetiver, walnut, and wheat germ oils. Exemplary synthetic oils include, but are not limited to, butyl stearate, caprylic triglyceride, capric triglyceride, cyclomethicone, diethyl sebacate, dimethicone 360, isopropyl myristate, mineral oil, octyldodecanol, oleyl alcohol, silicone oil, and mixtures thereof.

Liquid dosage forms for oral and parenteral administration include, but are not limited to, pharmaceutically acceptable emulsions, microemulsions, solutions, suspensions, syrups, and elixirs. In addition to the active agents, the liquid dosage forms may contain inert diluents commonly used in the art such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, oral compositions can also include adjuvants such as wetting agents, emulsifying and suspending agents, sweetening, flavoring, and perfuming agents. In certain embodiments for parenteral administration, agents of the invention are mixed with solubilizing agents such CREMOPHOR EL® (polyethoxylated castor oil), alcohols, oils, modified oils, glycols, polysorbates, cyclodextrins, polymers, and combinations thereof.

Injectable preparations, for example, sterile injectable aqueous or oleaginous suspensions, may be formulated according to the known art using suitable dispersing or wetting agents and suspending agents. Sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose, any bland fixed oil can be employed including synthetic mono- or diglycerides. In addition, fatty acids such as oleic acid are used in the preparation of injectables.

Injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

Solid dosage forms for oral administration include capsules, tablets, pills, powders, and granules. In such solid dosage forms, the active agent is mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

Solid compositions of a similar type may also be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions which can be used include polymeric substances and waxes. Solid compositions of a similar type may also be employed as fillers in soft and hard-filled gelatin capsules using such excipients as lactose or milk sugar as well as high molecular weight polyethylene glycols and the like.

The active agents can also be in micro-encapsulated form with one or more excipients as noted above. The solid dosage forms of tablets, dragees, capsules, pills, and granules can be prepared with coatings and shells such as enteric coatings, release controlling coatings and other coatings well known in the pharmaceutical formulating art. In such solid dosage forms the active agent may be admixed with at least one inert diluent such as sucrose, lactose or starch. Such dosage forms may also comprise, as is normal practice, additional substances other than inert diluents, e.g., tableting lubricants and other tableting aids such a magnesium stearate and microcrystalline cellulose. In the case of capsules, tablets, and pills, the dosage forms may also comprise buffering agents. They may optionally contain opacifying agents and can also be of a composition that they release the active ingredient(s) only, or preferentially, in a certain part of the intestinal tract, optionally, in a delayed manner. Examples of embedding compositions which can be used include polymeric substances and waxes.

Formulations suitable for topical administration include liquid or semi-liquid preparations such as liniments, lotions, gels, applicants, oil-in-water or water-in-oil emulsions such as creams, ointments, or pastes; or solutions or suspensions such as drops. Formulations for topical administration to the skin surface can be prepared by dispersing the drug with a dermatologically acceptable carrier such as a lotion, cream, ointment, or soap. Useful carriers are capable of forming a film or layer over the skin to localize application and inhibit removal. For topical administration to internal tissue surfaces, the agent can be dispersed in a liquid tissue adhesive or other substance known to enhance adsorption to a tissue surface. For example, hydroxypropylcellulose or fibrinogen/thrombin solutions can be used to advantage. Alternatively, tissue-coating solutions, such as pectin-containing formulations can be used. Ophthalmic formulation, car drops, and eye drops are also contemplated as being within the scope of this invention. Additionally, the present disclosure contemplates the use of transdermal patches, which have the added advantage of providing controlled delivery of an agent to the body. Such dosage forms can be made by dissolving or dispensing the agent in the proper medium. Absorption enhancers can also be used to increase the flux of the agent across the skin. The rate can be controlled by either providing a rate controlling membrane or by dispersing the agent in a polymer matrix or gel.

Additionally, the carrier for a topical formulation can be in the form of a hydroalcoholic system (e.g., quids and gels), an anhydrous oil or silicone based system, or an emulsion system, including, but not limited to, oil-in-water, water-in-oil, water-in-oil-in-water, and oil-in-water-in-silicone emulsions. The emulsions can cover a broad range of consistencies including thin lotions (which can also be suitable for spray or aerosol delivery), creamy lotions, light creams, heavy creams, and the like. The emulsions can also include microemulsion systems. Other suitable topical carriers include anhydrous solids and semisolids (such as gels and sticks); and aqueous based mousse systems.

Also encompassed by the disclosure are kits (e.g., pharmaceutical packs). The kits provided may comprise a pharmaceutical composition or compound described herein and a container (e.g., a vial, ampule, bottle, syringe, and/or dispenser package, or other suitable container). In some embodiments, provided kits may optionally further include a second container comprising a pharmaceutical excipient for dilution or suspension of a pharmaceutical composition or compound described herein. In some embodiments, the pharmaceutical composition or compound described herein provided in the first container and the second container are combined to form one unit dosage form.

Thus, in one aspect, provided are kits including a first container comprising a compound or pharmaceutical composition described herein. In certain embodiments, the kits are useful for treating a proliferative disorder (e.g., a cancer) in a subject in need thereof. In certain embodiments, the kits are useful for treating cancer (e.g., a hematological cancer) in a subject in need thereof. In certain embodiments, the kits are useful for preventing cancer (e.g., a hematological cancer) in a subject in need thereof. In certain embodiments, the kits are useful for reducing the risk of developing cancer (e.g., a hematological cancer) in a subject in need thereof. In certain embodiments, the kits are useful for promoting the degradation of a protein (e.g., a target protein) in a subject or cell. In certain embodiments, the kits are useful for promoting the degradation of IKZF1 in a subject or cell. In certain embodiments, the kits are useful for promoting the degradation of IKZF2 in a subject or cell. In certain embodiments, the kits are useful for promoting the degradation of CK1α in a subject or cell. In certain embodiments, the kits are useful for promoting the degradation of GSPT1 in a subject or cell.

In certain embodiments, a kit described herein further includes instructions for using the kit. A kit described herein may also include information as required by a regulatory agency such as the U.S. Food and Drug Administration (FDA). In certain embodiments, the information included in the kits is prescribing information. In certain embodiments, a kit described herein may include one or more additional pharmaceutical agents described herein as a separate composition.

### Methods of Treatment

Acute myeloid leukemia (AML) is a genetically complex and heterogeneous set of diseases characterized by a diverse set of mutations. The 5-year overall survival in adult AML is less than 20% and has only improved modestly in the past 30 years, and approximately one third of AML cases are unresponsive to standard treatments. Acquisition of a gene expression program that corresponds to a gain of self-renewal properties imparts a worse clinical outcome in AML patients and correlates with therapy resistance and relapse. Therefore, there is an unmet need to develop new therapies for AML patients. Targeting the mechanisms that contribute to self-renewal and disease progression can provide a new strategy for therapeutic intervention in leukemia.

Recently, IKZF2 was established as a viable target in acute myeloid leukemia (AML) through the key role of IKZF2 in maintenance of leukemia stem cell (LSC) function. As such, degradation of IKZF2 is an attractive method for the development of new anticancer therapy. Accordingly, use of a compound that degrades IKZF2 provides a method of treating cancers that rely on IKZF2 activity.

In addition, CK1α was discovered to be a promising autophagy regulator in cancer cells (e.g., lung, breast, multiple myeloma, colon). CK1α regulates diverse signaling processes such as Wnt-signaling, cell cycle, and apoptosis. CK1α may also be involved in tumor progression and be overexpressed in colorectal cancer. Recent studies show that CK1α inactivation is associated with autophagy inhibition that correlates with the suppression of cancer cell growth. Autophagy is an important factor influencing chemotherapy resistance to 5-FU in colorectal cancer. Since autophagy is regulated by CK1α, its inhibition or degradation provides a therapeutic option for treatment of 5-FU resistant colorectal cancer.

As such, degradation of CK1α is an attractive method for the development of new anticancer therapies. Accordingly, use of a compound that degrades CK1α provides a method of treating cancers that rely on CK1α activity.

IKZF1 has attracted attention as a target due to its role in hematopoiesis, immune function, and tumor suppression, as well as its complex role in the regulation of transcription and chromatin remodeling. IKZF1 has been established as one of the most clinically relevant tumor suppressors in high-risk acute lymphoblastic leukemia (ALL). Its dysfunction has also been linked to the development of chronic lymphocytic leukemia (CLL).

GSPT1 is a small GTPase initially found essential for the G1 to S phase transition of the cell cycle and later reported to function as a polypeptide chain release factor 3 (eRF3). In this role, GSPT1 associates with eRF1 to mediate stop codon recognition and nascent protein release from the ribosome. GSPT1 has also been shown to play several additional roles in critical cellular processes such as cell cycle regulation, cytoskeleton organization, and apoptosis. Changes in the expression pattern of translation factors can lead to several changes in the tumor cells, such as an increase in the overall rate of protein synthesis and/or overexpression of proteins involved in cell growth and proliferation. Indeed, GSPT1 has been found to be overexpressed and oncogenic in a number of cancers, including gastric, colorectal, lung, and breast cancers. GSPT1 is overexpressed in many pediatric cancers, but mostly in hematopoietic malignancies including B-ALL, T-ALL, and AML. Acute leukemia cells are highly sensitive to GSPT1 degradation, strongly supporting this mechanism as a therapeutic approach to the treatment of hematological cancers.

The present disclosure provides methods for treating proliferative disease. In certain embodiments, the present disclosure provides methods for treating cancer. In certain embodiments, the application provides a method of treating a pediatric cancer (e.g., childhood acute leukemia (AL) or medulloblastoma (MB)), lung cancer, breast cancer, colon cancer, colorectal cancer, ovarian cancer, gastric cancer, or a hematological cancer. In certain embodiments, the application provides a method of treating ovarian cancer, gastric cancer, or a hematological cancer. In certain embodiments, the application provides a method of treating ovarian cancer. In certain embodiments, the application provides a method of treating a hematological cancer. In certain embodiments, the application provides a method of treating a leukemia, a lymphoma, or multiple myeloma. In certain embodiments, the application provides a method of treating a leukemia or a lymphoma. In certain embodiments, the application provides a method of treating primary effusion lymphoma (PEL), del(5q) myelodysplastic syndrome (MDS), chronic myeloid leukemia (CML), chronic lymphoid leukemia (CLL), acute myeloid leukemia (AML), hairy cell leukemia, chronic myelomonocytic leukemia (CMML), juvenile myelomonocyte leukemia (JMML), large granular lymphocytic leukemia (LGL), acute lymphoblastic leukemia (ALL), B-cell acute lymphoblastic leukemia (B-ALL), or T-cell acute lymphoblastic leukemia (T-ALL), B-cell lymphoma, diffuse large B-cell lymphoma (DLBCL), T-cell-lymphoma, Hodgkin's lymphoma, non-Hodgkin's lymphoma, hairy cell lymphoma, or Burkett's lymphoma. In certain embodiments, the application provides a method of treating primary effusion lymphoma (PEL), del(5q) myelodysplastic syndrome (MDS), or acute myeloid leukemia (AML). In certain embodiments, the application provides a method of treating acute myeloid leukemia (AML). In certain embodiments, the application provides a method of treating acute myeloid leukemia (AML) in patients having wild-type p53 cells. In certain embodiments, the application provides a method of treating acute myeloid leukemia (AML) in patients having mutations or genetic alterations such as DNMT3A, NPM1, Flt3, CEBPA, MLL-fusions, del-5q, IDH1 and SF3B1. In certain embodiments, the application provides a method of treating primary effusion lymphoma (PEL). In certain embodiments, the application provides a method of treating del(5q) myelodysplastic syndrome (MDS). In certain embodiments, the application provides a method of treating chronic myeloid leukemia (CML). In certain embodiments, the application provides a method of treating hairy cell leukemia. In certain embodiments, the application provides a method of treating chronic myelomonocytic leukemia (CMML). In certain embodiments, the application provides a method of treating juvenile myelomonocyte leukemia (JMML). In certain embodiments, the application provides a method of treating large granular lymphocytic leukemia (LGL). In certain embodiments, the application provides a method of treating B-cell lymphoma. In certain embodiments, the application provides a method of treating diffuse large B-cell lymphoma (DLBCL). In certain embodiments, the application provides a method of treating chronic lymphocytic leukemia (CLL). In certain embodiments, the application provides a method of treating acute lymphoblastic leukemia (ALL). In certain embodiments, the application provides a method of treating B-cell acute lymphoblastic leukemia (B-ALL). In certain embodiments, the application provides a method of treating T-cell acute lymphoblastic leukemia (T-ALL). In certain embodiments, the application provides a method of treating T-cell-lymphoma. In certain embodiments, the application provides a method of treating Hodgkin's lymphoma. In certain embodiments, the application provides a method of treating non-Hodgkin's lymphoma. In certain embodiments, the application provides a method of treating hairy cell lymphoma. In certain embodiments, the application provides a method of treating Burkett's lymphoma. In certain embodiments, the application provides a method of treating multiple myeloma. In certain embodiments, the application provides a method of treating ovarian cancer. In certain embodiments, the application provides a method of treating gastric cancer. In certain embodiments, the application provides a method of promoting the degradation of a protein, e.g., a target protein. In certain embodiments, the application provides a method of promoting the degradation of IKZF1. In certain embodiments, the application provides a method of promoting the degradation of IKZF2. In certain embodiments, the application provides a method of promoting the degradation of CK1α. In certain embodiments, the application provides a method of promoting the degradation of GSPT1.

In certain embodiments, the methods comprise administering to a subject in need thereof (e.g., a subject with a cancer) a compound that interacts with IKZF1, for example, a compound that is an inhibitor of IKZF1, a modulator of IKZF1, a binder of IKZF1, a compound that modifies IKZF1, or a compound that promotes the degradation of IKZF1. In certain embodiments, the methods comprise administering to a subject in need thereof (e.g., a subject with a cancer) a compound that interacts with IKZF2, for example, a compound that is an inhibitor of IKZF2, a modulator of IKZF2, a binder of IKZF2, a compound that modifies IKZF2, or a compound that promotes the degradation of IKZF2. In certain embodiments, the methods comprise administering to a subject in need thereof (e.g., a subject with a cancer) a compound that interacts with CK1α, for example, a compound that is an inhibitor of CK1α, a modulator of CK1α, a binder of CK1α, a compound that modifies CK1α, or a compound that promotes the degradation of CK1α. In certain embodiments, the methods comprise administering to a subject in need thereof (e.g., a subject with a cancer) a compound that interacts with GSPT1, for example, a compound that is an inhibitor of GSPT1, a modulator of GSPT1, a binder of GSPT1, a compound that modifies GSPT1, or a compound that promotes the degradation of GSPT1.

In certain embodiments, the methods comprise administering a compound of Formula (I), or a pharmaceutically acceptable salt, co-crystal, tautomer, stereoisomer, solvate, hydrate, polymorph, isotopically enriched derivative, or prodrug, or composition thereof, to a subject in need thereof. In some embodiments, the method comprises administering a pharmaceutical composition comprising a compound of Formula (I), or a pharmaceutically acceptable salt, co-crystal, tautomer, stereoisomer, solvate, hydrate, polymorph, isotopically enriched derivative, or prodrug, or composition thereof, to a subject in need thereof.

In certain embodiments, the methods of the disclosure comprise administering to the subject an effective amount of a compound of Formula (I), or a pharmaceutically acceptable salt, co-crystal, tautomer, stereoisomer, solvate, hydrate, polymorph, isotopically enriched derivative, or prodrug, or composition thereof. In some embodiments, the effective amount is a therapeutically effective amount. In some embodiments, the effective amount is a prophylactically effective amount.

In certain embodiments, the subject being treated is an animal. The animal may be of either sex and may be at any stage of development. In certain embodiments, the subject is a mammal. In certain embodiments, the subject being treated is a human. In certain embodiments, the subject is a domesticated animal, such as a dog, cat, cow, pig, horse, sheep, or goat. In certain embodiments, the subject is a companion animal, such as a dog or cat. In certain embodiments, the subject is a livestock animal, such as a cow, pig, horse, sheep, or goat. In certain embodiments, the subject is a zoo animal. In another embodiment, the subject is a research animal such as a rodent (e.g., mouse, rat), dog, pig, or non-human primate. In certain embodiments, the animal is a genetically engineered animal. In certain embodiments, the animal is a transgenic animal.

Certain methods described herein may comprise administering one or more additional pharmaceutical agent(s) in combination with the compounds described herein. The additional pharmaceutical agent(s) may be administered at the same time as the compound of Formula (I), or at different times than the compound of Formula (I). For example, the compound of Formula (I) and any additional pharmaceutical agent(s) may be on the same dosing schedule or different dosing schedules. All or some doses of the compound of Formula (I) may be administered before all or some doses of an additional pharmaceutical agent, after all or some does an additional pharmaceutical agent, within a dosing schedule of an additional pharmaceutical agent, or a combination thereof. The timing of administration of the compound of Formula (I) and additional pharmaceutical agents may be different for different additional pharmaceutical agents.

In certain embodiments, the additional pharmaceutical agent comprises an agent useful in the treatment of proliferative disease. In certain embodiments, the additional pharmaceutical agent is useful in the treatment of cancer. In certain embodiments, the additional pharmaceutical agent is useful in the treatment of a pediatric cancer (e.g., childhood acute leukemia (AL) or medulloblastoma (MB)), lung cancer, breast cancer, colon cancer, colorectal cancer, ovarian cancer, gastric cancer, or a hematological cancer. In certain embodiments, the additional pharmaceutical agent is useful in the treatment of ovarian cancer, gastric cancer, or a hematological cancer. In certain embodiments, the additional pharmaceutical agent is useful in the treatment of ovarian cancer. In certain embodiments, the additional pharmaceutical agent is useful in the treatment of a hematological cancer. In certain embodiments, the additional pharmaceutical agent cancer is useful in the treatment of multiple myeloma, a leukemia, or a lymphoma. In certain embodiments, the additional pharmaceutical agent cancer is useful in the treatment of multiple myeloma. In certain embodiments, the additional pharmaceutical agent cancer is useful in the treatment of a leukemia or a lymphoma. In certain embodiments, the additional pharmaceutical agent is useful in the treatment of primary effusion lymphoma (PEL), del(5q) myelodysplastic syndrome (MDS), chronic myeloid leukemia (CML), chronic lymphoid leukemia (CLL), acute myeloid leukemia (AML), hairy cell leukemia, chronic myelomonocytic leukemia (CMML), juvenile myelomonocyte leukemia (JMML), large granular lymphocytic leukemia (LGL), acute lymphoblastic leukemia (ALL), B-cell acute lymphoblastic leukemia (B-ALL), or T-cell acute lymphoblastic leukemia (T-ALL), B-cell lymphoma, diffuse large B-cell lymphoma (DLBCL), T-cell-lymphoma, Hodgkin's lymphoma, non-Hodgkin's lymphoma, hairy cell lymphoma, or Burkett's lymphoma. In certain embodiments, the additional pharmaceutical agent is useful in the treatment of acute myeloid leukemia (AML).

In another aspect, the present disclosure provides methods for promoting the degradation of a protein (e.g., a target protein), the method comprising contacting the protein with a compound of Formula (I), or a pharmaceutically acceptable salt, co-crystal, tautomer, stereoisomer, solvate, hydrate, polymorph, isotopically enriched derivative, or prodrug, or composition thereof. In certain embodiments, the degradation is in a cell. In certain embodiments, the degradation is in a subject. In certain embodiments, the degradation is in a biological sample.

In another aspect, the present disclosure provides methods for promoting the degradation of IKZF1, the method comprising contacting IKZF1 with a compound of Formula (I), or a pharmaceutically acceptable salt, co-crystal, tautomer, stereoisomer, solvate, hydrate, polymorph, isotopically enriched derivative, or prodrug, or composition thereof. In certain embodiments, the degradation is in a cell. In certain embodiments, the degradation is in a subject. In certain embodiments, the degradation is in a biological sample.

In another aspect, the present disclosure provides methods for promoting the degradation of IKZF2, the method comprising contacting IKZF2 with a compound of Formula (I), or a pharmaceutically acceptable salt, co-crystal, tautomer, stereoisomer, solvate, hydrate, polymorph, isotopically enriched derivative, or prodrug, or composition thereof. In certain embodiments, the degradation is in a cell. In certain embodiments, the degradation is in a subject. In certain embodiments, the degradation is in a biological sample.

In another aspect, the present disclosure provides methods for promoting the degradation of CK1α, the method comprising contacting CK1α with a compound of Formula (I), or a pharmaceutically acceptable salt, co-crystal, tautomer, stereoisomer, solvate, hydrate, polymorph, isotopically enriched derivative, or prodrug, or composition thereof. In certain embodiments, the degradation is in a cell. In certain embodiments, the degradation is in a subject. In certain embodiments, the degradation is in a biological sample.

In another aspect, the present disclosure provides methods for promoting the degradation of GSPT1, the method comprising contacting GSPT1 with a compound of Formula (I), or a pharmaceutically acceptable salt, co-crystal, tautomer, stereoisomer, solvate, hydrate, polymorph, isotopically enriched derivative, or prodrug, or composition thereof. In certain embodiments, the degradation is in a cell. In certain embodiments, the degradation is in a subject. In certain embodiments, the degradation is in a biological sample.

In another aspect, the application provides a method of promoting the ubiquitination of IKZF1, the method comprising contacting IKZF1 with a compound of Formula (I), or a pharmaceutically acceptable salt, co-crystal, tautomer, stereoisomer, solvate, hydrate, polymorph, isotopically enriched derivative, or prodrug, or composition thereof. In certain embodiments, the ubiquitination takes place in a cell. In certain embodiments, the ubiquitination takes place in a subject. In certain embodiments, the ubiquitination takes place in a biological sample.

In another aspect, the application provides a method of promoting the ubiquitination of IKZF2, the method comprising contacting IKZF2 with a compound of Formula (I), or a pharmaceutically acceptable salt, co-crystal, tautomer, stereoisomer, solvate, hydrate, polymorph, isotopically enriched derivative, or prodrug, or composition thereof. In certain embodiments, the ubiquitination takes place in a cell. In certain embodiments, the ubiquitination takes place in a subject. In certain embodiments, the ubiquitination takes place in a biological sample.

In another aspect, the application provides a method of promoting the ubiquitination of CK1α, the method comprising contacting CK1α with a compound of Formula (I), or a pharmaceutically acceptable salt, co-crystal, tautomer, stereoisomer, solvate, hydrate, polymorph, isotopically enriched derivative, or prodrug, or composition thereof. In certain embodiments, the ubiquitination takes place in a cell. In certain embodiments, the ubiquitination takes place in a subject. In certain embodiments, the ubiquitination takes place in a biological sample.

In another aspect, the application provides a method of promoting the ubiquitination of GSPT1, the method comprising contacting GSPT1 with a compound of Formula (I), or a pharmaceutically acceptable salt, co-crystal, tautomer, stereoisomer, solvate, hydrate, polymorph, isotopically enriched derivative, or prodrug, or composition thereof. In certain embodiments, the ubiquitination takes place in a cell. In certain embodiments, the ubiquitination takes place in a subject. In certain embodiments, the ubiquitination takes place in a biological sample.

### EXAMPLES

In order that the invention described herein may be more fully understood, the following examples are set forth. The examples described in this application are offered to illustrate the compounds, pharmaceutical compositions, and methods provided herein and are not to be construed in any way as limiting their scope.

### Compound Synthesis

Commercial solvents and reagents were used as received. N,N-Dimethylformamide (DMF, Beantown Chemical, catalog no. BT138690) and dichloromethane (DCM, Fisher Chemical, catalog no. D138-4) were vigorously purged with argon for 1 h, followed by passage under argon pressure through two packed columns of neutral alumina (Pure Process Technology).

Reactions were performed in a single-neck, flame-dried, round-bottom flask fitted with a rubber septum under argon unless otherwise noted. Analytical thin-layer chromatography (TLC) was performed using glass plates pre-coated with silica gel (0.25 mm, 60 Å pore size) impregnated with a fluorescent indicator (254 nm). TLC plates were visualized by exposure to ultraviolet light (UV) or submersion in ninhydrin or p-anisaldehyde followed by brief heating with a heat gun (10-15 s). Organic solutions were concentrated by rotary evaporation at 30° C. to 35° C. For purification, normal phase flash-column chromatography was performed. Some normal phase column chromatography purifications were performed using a Teledyne ISCO CombiFlash NextGen 300+ instrument. Normal phase purification used silica gel (60 Å, 40-63 μm particle size) purchased from Silicycle. Purification with the CombiFlash was performed using silica gel (60 Å, 40-63 μm particle size) purchased from Silicycle and 12-gram RediSep Rf silica flash columns (60 Å, 40-63 μm particle size) purchased from Teledyne ISCO. All analogs tested in cells were purified by preparative HPLC on a Waters Prep 150 LC system and Agilent Infinity II system with a XBridge BEH C18 OBD prep column (130 Å, 5 mm, 19 mm 3 100 mm) and an Agilent C18 prep column (100 Å, 5 mm, 30 mm 3 100 mm).

¹H and ¹³C NMR spectra were recorded on Bruker AVANCE NEO 400, Bruker AVANCE NEO 400B, JEOL ECZ400S, Varian Unity/Inova500, or Agilent 600 MHZ instruments. Chemical shifts are expressed in parts per million (ppm, δ scale) and are referenced to residual protium in the NMR solvent (¹H: CDCl₃, δ 7.26; CD₃OD δ 3.31, DMSO-d₆ δ 2.50; ¹³C: CDCl₃, δ 77.0; CD₃OD, δ 49.0; (CD₃)₂SO, δ 39.0). The purity of all compounds was determined by ¹H NMR and was confirmed to be greater than 95%.

Infrared (IR) spectra experiments were performed on a Bruker ALPHA FT-IR instrument. Small molecule high-resolution mass spectra (HRMS) was performed at the Harvard Center for Mass Spectrometry using a Thermo q-Exactive Plus.

Compounds of Formula (I) may be prepared using the synthetic schemes and procedures described in detail below.

### N-(2-(2,6-dioxopiperidin-3-yl)-1-oxoisoindolin-5-yl)-6-methoxy-2-naphthamide (DEG-35)

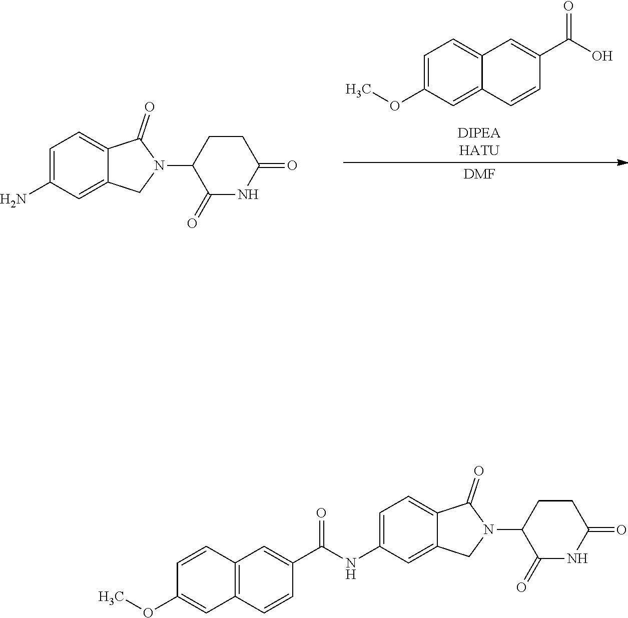

In a flame-dried round-bottom flask, 6-methoxy-2-naphthoic acid (23 mg, 1.5 equiv) was dissolved in DMF (771 μL). 5-NH₂-lenalidomide (20 mg, 1.0 equiv) was added followed by HATU (88 mg, 3.0 equiv). Diisopropylethylamine (40 μL) was added and the solution was stirred for 12 h at 24° C. The reaction was quenched with the addition of a saturated solution of sodium bicarbonate (2 mL) and diluted with ethyl acetate (1 mL). The biphasic mixture was transferred to a separatory funnel and the organic layer was collected. The aqueous layer was extracted with ethyl acetate (2×2 mL) and the combined organic layers were dried over sodium sulfate. The dried solution was filtered through cotton and the filtrate was concentrated by rotary evaporation. The solid obtained was purified by flash gel chromatography (SiO₂, 2-4% methanol-dichloromethane, 2 steps) followed by reversed phase HPLC to afford DEG-35 as a white solid (7 mg, 21%). HRMS-ESI (m/z): [M+H]⁺ calculated for C₂₅H₂₄N₃O₄, 430.1761; found, 430.1765.

### 6-methoxy-N-(2-(1-methyl-2,6-dioxopiperidin-3-yl)-1-oxoisoindolin-5-yl)-2-naphthamide (Me-DEG-35)

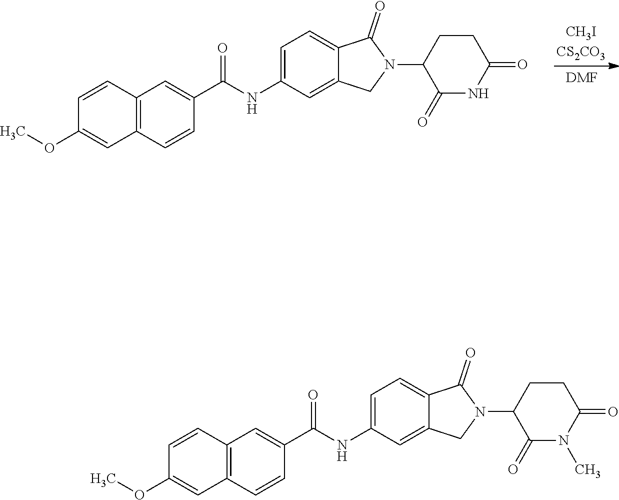

Methyl iodide (8.0 μL, 0.12 mmol, 1.00 equiv) was added to a solution of DEG-35 (50.0 mg, 0.113 mmol, 1.00 equiv) and cesium carbonate (40.0 mg, 0.12 mmol, 1.00 equiv) in DMF (1.13 mL, 0.1 M) under argon and stirred for 5 h at 24° C. The reaction mixture was diluted with water (5 mL) and transferred to a separatory funnel. The solution was diluted with brine (30 mL) and the aqueous layer was extracted with ethyl acetate (4×20 mL). The combined organic layers were dried over sodium sulfate and filtered through cotton. The filtrate was concentrated by rotary evaporation. The residue obtained was purified by high performance liquid chromatography (reverse phase, 95% to 5% water/acetonitrile) to afford Me-DEG-35 as a white solid (41.4 mg, 80% yield). ¹H NMR (400 MHZ, DMSO-d6) δ 10.67 (s, 1H, H9), 8.54 (s, 1H, H16), 8.19 (s, 1H, H6), 8.03-7.94 (m, 3H, H10/H11/H15), 7.88 (d, J=9.0 Hz, 1H, H7), 7.74 (d, J=8.3 Hz, 1H, H8), 7.43 (d, J=2.1 Hz, 1H, H12), 7.27 (dd, J=8.9, 2.4 Hz, 1H, H14), 5.18 (dd, J=13.4, 5.0 Hz, 1H, H4), 4.49 (d, J=17.2 Hz, 1H, H5), 4.33 (d, J=17.1 Hz, 1H, H5), 3.92 (s, 3H, H13), 3.05-2.94 (m, 4H, H1/H2), 2.80-2.73 (m, 1H, H2), 2.47-2.33 (m, 1H, H3), 2.07-1.98 (m, 1H, H3). HRMS ESI (m/z): [M+H]⁺ calculated for C26H24N3O5, 458.1710; found, 458.1713.

### N-(2-(2,6-dioxopiperidin-3-yl)-1-oxoisoindolin-5-yl)-8-methoxy-2H-chromene-3-carboxamide (DEG-77)

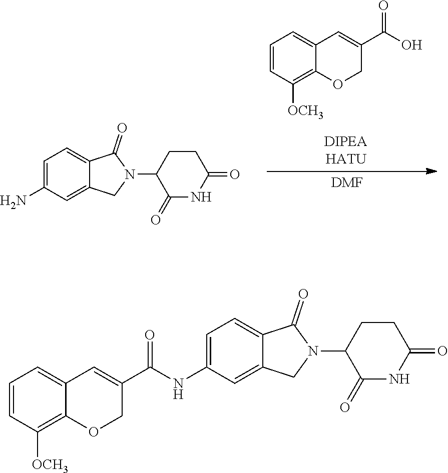

HATU (154.0 mg, 0.40 mmol, 3.00 equiv), and diisopropylamine (71 μL, 0.40 mmol, 3.00 equiv) were added sequentially to a solution of 8-methoxy-2H-chromene-3-carboxylic acid (55.7 mg, 0.27 mmol, 2.00 equiv) and 5-NH₂-lenalidomide (35.0 mg, 0.13 mg, 1.00 equiv) in dimethylformamide (1.35 mL, 0.1 M) and stirred for 18 h at 24° C. The product mixture was diluted with water (5 mL) and transferred to a separatory funnel. The aqueous layer was extracted with ethyl acetate (2×5 mL) and the organic layers were combined. The combined organic layers were dried over sodium sulfate and filtered through cotton. The filtrate was concentrated by rotary evaporation. The residue obtained was purified by flash column chromatography (SiO₂, 1%-4% methanol-dichloromethane, 3 steps) and further purified by high performance liquid chromatography to afford DEG-77 as a yellow solid (27.9 mg, 46% yield). ¹H NMR (400 MHZ, DMF-d₇) δ 10.96 (s, 1H), 10.44 (s, 1H), 8.20 (s, 1H), 7.89 (dd, J=8.3, 1.5 Hz, 1H), 7.74 (d, J=8.3 Hz, 1H), 7.61 (s, 1H), 7.09 (dd, J=8.0, 1.3 Hz, 1H), 6.97 (t, J=7.8 Hz, 1H), 6.91 (dd, J=7.6, 1.3 Hz, 1H), 5.24 (dd, J=13.3, 5.1 Hz, 1H), 5.08 (d, J=1.0 Hz, 1H), 4.57 (d, J=17.0 Hz, 1H), 4.45 (d, J=17.0 Hz, 1H), 3.87 (s, 3H), 3.10-3.00 (m, 1H), 2.78-2.72 (m, 1H), 2.61-2.50 (m, 1H), 2.22-2.14 (m, 1H). ¹³C NMR (101 MHz, DMF-d₇) δ 173.08, 171.48, 168.47, 164.28, 148.54, 144.19, 143.89, 143.09, 129.17, 127.47, 127.31, 123.88, 122.46, 121.98, 120.90, 120.00, 115.16, 114.51, 64.77, 55.97, 52.33, 47.66, 31.81, 23.35. IR (ATR-FTIR), cm⁻¹: 1692 (m), 1531 (s), 1422 (s), 1224 (m). HRMS-ESI (m/z): [M+H]⁺ calculated for C₂₄H₂₂N₃O₆, 448.1503; found, 448.1504.

Additional compounds of Formula (I), shown below, were prepared in an analogous manner to the methods used to prepare DEG-35 and DEG-77.

| | Compound structure | Characterization |
|---|---|---|
| DEG-51 | 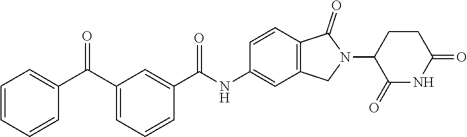 [SMILES: O=C1CCC(N2Cc3cc(NC(=O)c4cccc(C(=O)c5ccccc5)c4)ccc3C2=O)C(=O)N1] 3-benzoyl-N-(2-(2,6-dioxopiperidin-3-yl)-1- oxoisoindolin-5-yl)benzamide | ¹H NMR (400 MHz, DMSO): δ 10.99 (s, 1H), 10.74 (s, 1H₉), 8.33 (s, 1H, H₁₃), 8.28 (d, J = 7.9 Hz, 1H), 8.14 (s, 1H), 7.96 (d, J = 7.7 Hz, 1H), 7.86-7.69 (m, 6H), 7.60 (t, J = 7.6 Hz, 2H,), 5.11 (dd, J = 13.3, 5.1 Hz, 1H), 4.48 (d, J = 17.3 Hz, 1H), 4.34 (d, J = 17.3 Hz, 1H), 2.97-2.87 (m, 1H), 2.64-2.57 (m, 1H), 2.45-2.34 (m, 1H), 2.05-1.98 (m, 1H). ¹³C NMR (101 MHz, DMSO): δ 195.30, 172.90, 171.10, 167.79, 165.10, 143.10, 142.20, 137.27, 136.63, 134.88, 133.07, 132.76, 131.76, |
| | | 129.76, 128.92, 128.75, 128.69, 126.99, |
| | | 123.55, 120.09, 114.61, 51.57, 47.18, |
| | | 31.23, 22.53. HRMS-ESI (m/z): [M + H]⁺ |
| | | calculated for C₂₇H₂₂N₃O₅, 468.1554; |
| | | found, 468.1555. |
| DEG-52 | 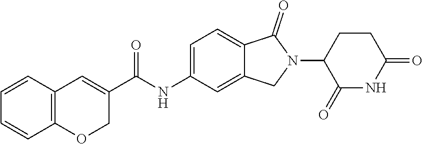 [SMILES: O=C1CCC(N2Cc3cc(NC(=O)C4=Cc5ccccc5OC4)ccc3C2=O)C(=O)N1] N-(2-(2,6-dioxopiperidin-3-yl)-1-oxoisoindolin-5- yl)-2H-chromene-3-carboxamide | ¹H NMR (400 MHz, DMSO) δ 10.98 (s, 1H), 10.37 (s, 1H), 8.06 (s, 1H), 7.77 (d, J = 9.4 Hz, 1H), 7.70 (d, J = 8.3 Hz, 1H), 7.54 (s, 1H), 7.33-7.26 (m, 2H), 7.00 (t, J = 7.6 Hz, 1H), 6.90 (d, J = 8.0 Hz, 1H), 5.10 (dd, J = 13.3, 5.1 Hz, 1H), 5.00 (s, 2H), 4.44 (d, J = 17.2 Hz, 1H), 4.32 (d, J = 17.2 Hz, 1H), 2.97-2.87 (m, 1H), 2.64- 2.56 (m, 1H), 2.43-2.36 (m, 1H), 2.03- 1.98 (m, 1H). ¹³C NMR (101 MHz, DMSO) δ 172.91, 171.12, 167.80, 163.73, 154.32, 143.12, 142.16, 131.50, 128.75, |
| | | 128.59, 126.68, 126.58, 123.55, 121.96, |
| | | 121.01, 119.68, 115.80, 114.15, 64.30, |
| | | 51.55, 47.16, 31.23, 22.53. HRMS-ESI |
| | | (m/z): [M + H]⁺ calculated for C₂₃H₂₀N₃O₅, |
| | | 418.1397; found, 418.1397. |
| DEG-53 | 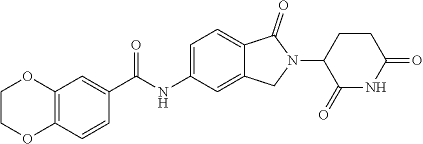 [SMILES: O=C1CCC(N2Cc3cc(NC(=O)c4ccc5c(c4)OCCO5)ccc3C2=O)C(=O)N1] N-(2-(2,6-dioxopiperidin-3-yl)-1-oxoisoindolin-5- yl)-2,3-dihydrobenzo[b]dioxine-6- carboxamide | ¹H NMR (400 MHz, DMSO-d₆) δ 10.98 (s, 1H), 10.35 (s, 1H), 8.13 (s, 1H), 7.82 (dd, J = 8.4, 1.5 Hz, 1H), 7.69 (d, J = 8.3 8.4 Hz, 1H), 5.10 (dd, J = 13.2, 5.0 Hz, 1H), 4.46 (d, J = 17.2 Hz, 1H), 4.39-4.25 (m, 5H), 2.97-2.86 (m, 1H), 2.63-2.57 (m, 1H), 2.45-2.33 (m, 1H), 2.04-1.98 (m, 1H). HRMS-ESI (m/z): [M + H]+ calculated for C22H20N3O6, 422.1347; found, 422.1347. |
| DEG-54 | 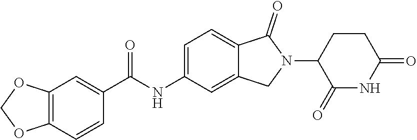 [SMILES: O=C1CCC(N2Cc3cc(NC(=O)c4ccc5c(c4)OCO5)ccc3C2=O)C(=O)N1] N-(2-(2,6-dioxopiperidin-3-yl)-1-oxoisoindolin-5- yl)benzo[d][1,3]dioxole-5-carboxamide | ¹H NMR (400 MHz, DMSO) δ 10.98 (s, 1H), 10.37 (s, 1H), 8.11 (s, 1H), 7.81 (d, J = 8.5 Hz, 1H), 7.69 (d, J = 8.1 Hz, 1H), 7.60 (dd, J = 8.1, 1.7 Hz, 1H), 7.53 (d, J = 1.8 Hz, 1H), 7.07 (d, J = 9.1, 1H), 6.14 (s, 2H), 5.09 (dd, J = 13.4, 5.4 Hz, 1H), 4.45 (d, J = 17.0 Hz, 1H), 4.31 (d, J = 17.0 Hz, 1H), 2.97-2.85 (m, 1H), 2.67-2.55 (m, 1H), 2.45-2.30 (m, 1H), 2.05-1.96 (m, 1H). ¹³C NMR (101 MHz, DMSO) δ 172.92, 171.13, 167.87, 164.89, 150.31, 147.44, 143.08, 142.54, 128.37, 126.62, |
| | | 123.47, 123.12, 119.93, 114.36, 108.02, |
| | | 107.82, 101.91, 51.57, 47.18, 31.23, |
| | | 22.54. HRMS-ESI (m/z): [M + H]⁺ |
| | | calculated for C₂₁H₁₈N₃O₆, 408.1190; |
| | | found, 408.1190. |
| DEG-55 | 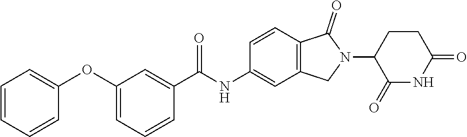 [SMILES: O=C1CCC(N2Cc3cc(NC(=O)c4cccc(Oc5ccccc5)c4)ccc3C2=O)C(=O)N1] N-(2-(2,6-dioxopiperidin-3-yl)-1-oxoisindolin-5- yl)-3-phenoxybenzamide | ¹H NMR (400 MHz, DMSO) δ 10.98 (s, 1H), 10.58 (s, 1H), 8.12 (s, 1H), 7.84-7.76 (m, 2H), 7.71 (d, J = 8.3 Hz, 1H), 7.61 (t, J = 2.0 Hz, 1H), 7.57 (t, J = 7.9 Hz, 1H), 7.46-7.41 (m, 2H), 7.27-7.17 (m, 2H), 7.10-7.07 (m, 2H), 5.10 (dd, J = 13.2, 5.1, Hz, 1H), 4.46 (d, J = 17.4 Hz, 1H), 4.32 (d, J = 17.2 Hz, 1H), 2.97-2.87 (m, 1H), 2.64-2.57 (m, 1H), 2.45-2.33 (m, 1H), 2.04-1.96 (m, 1H). ¹³C NMR (101 MHz, DMSO) δ 173.38, 171.58, 168.27, 165.54, 157.29, 156.70, 143.55, 142.71, 136.95, |
| | | 130.75, 130.71, 127.38, 124.40, 123.98, |
| | | 123.23, 122.33, 120.54, 119.38, 118.14, |
| | | 115.04, 52.04, 47.64, 31.70, 23.00. |
| | | HRMS-ESI (m/z): [M + H]⁺ calculated for |
| | | C₂₆H₂₂N₃O₅, 456.1554; found, 456.1556. |
| DEG-56 | 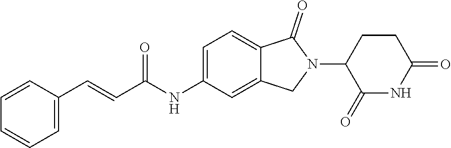 [SMILES: O=C(/C=C/c1ccccc1)Nc1ccc2c(c1)CN(C1CCC(=O)NC1=O)C2=O] N-(2-(2,6-dioxopiperidin-3-yl)-1-oxoisoindolin-5- yl)cinnamamide | ¹H NMR (400 MHz, DMSO) δ 11.00 (s, 1H), 10.57 (s, 1H), 8.09 (s, 1H), 7.72-7.60 (m, 5H), 7.49-7.41 (m, 3H), 6.87 (d, J = 15.7 Hz, 1H), 5.10 (dd, J = 13.2, 4.9 Hz, 1H), 4.46 (d, J = 17.2 Hz, 1H), 4.32 (d, J = 17.3 Hz, 1H), 2.97-2.87 (m, 1H), 2.63- 2.57 (m, 1H), 2.42-2.33 (m, 1H), 2.04- 1.96 (m, 1H). ¹³C NMR (101 MHz, DMSO) δ 173.38, 171.59, 168.29, 164.40, 143.83, 142.97, 141.35, 135.06, 130.46, 129.54, 128.31, 126.98, 124.23, 122.41, 119.39, 113.85, 52.03, 47.66, 31.71, |
| | | 23.02. HRMS-ESI (m/z): [M + H]⁺ |
| | | calculated for C₂₂H₂₀N₃O₄, 390.1448; |
| | | found, 390.1448. |
| DEG-57 | 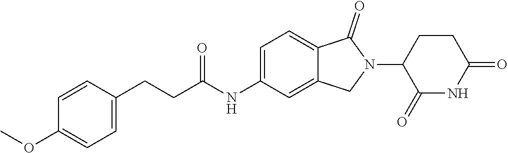 [SMILES: COc1ccc(CCC(=O)Nc2ccc3c(c2)CN(C2CCC(=O)NC2=O)C3=O)cc1] N-(2-(2,6-dioxopiperidin-3-yl)-1-oxoisoindolin-5- yl)-3-(4-methoxyphenyl)propenamide | ¹H NMR (400 MHz, DMSO) δ 10.99 (s, 1H), 10.25 (s, 1H), 7.98 (s, 1H), 7.65 (d, J = 8.3 Hz, 1H), 7.57 (d, J = 8.0 Hz, 1H), 7.16 (d, J = 8.4 Hz, 2H), 6.84 (d, J = 8.5 Hz, 2H), 5.08 (dd, J = 13.2, 5.0 Hz, 1H), 4.42 (d, J = 17.2 Hz, 1H), 4.28 (d, J = 17.2 Hz, 1H), 3.70 (s, 3H), 2.96-2.83 (m, 3H), 2.66-2.55 (m, 3H), 2.43-2.30 (m, 1H), 2.01-1.95 (m, 1H). ¹³C NMR (101 MHz, DMSO) δ 173.40, 171.61, 171.49, 168.31, 158.02, 143.78, 142.93, 133.35, 129.68, 126.63, 124.13, 119.10, 114.20, 113.55, 55.42, 51.97, 47.59, 38.88, 31.69, |
| | | 30.33, 23.00. HRMS-ESI (m/z): |
| | | [M + NH₄]⁺ calculated for C₂₂H₂₅N₄O₄, |
| | | 409.1870; found, 409.1850 |
| DEG-58 | 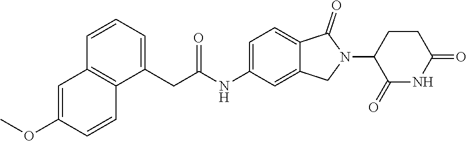 [SMILES: COc1ccc2c(CC(=O)Nc3ccc4c(c3)CN(C3CCC(=O)NC3=O)C4=O)cccc2c1] N-(2-(2,6-dioxopipperidin-3-yl)-1-oxoisoindolin-5- yl)-2-(6-methoxynaphthalen-1-yl)acetamide | ¹H NMR (400 MHz, DMSO) δ 10.97 (s, 1H), 10.64 (s, 1H), 8.04 (d, J = 9.2 Hz, 1H), 7.97 (s, 1H), 7.75 (d, J = 8.2 Hz, 1H), 7.69-7.60 (m, 2H), 7.42 (t, J = 7.6 Hz, 1H), 7.36-7.32 (m, 2H), 7.21 (dd, J = 9.2, 2.6 Hz, 1H), 5.08 (dd, J = 13.2, 5.1 Hz, 1H), 4.40 (d, J = 17.4 Hz, 1H), 4.26 (d, J = 17.4 Hz, 1H), 4.15 (s, 2H), 3.88 (s, 3H), 2.96-2.84 (m, 1H), 2.62-2.55 (m, 1H), 2.42-2.30 (m, 1H), 2.01-1.94 (m, 1H), ¹³C NMR (101 MHz, DMSO) δ 173.36, 171.57, 170.08, 168.29, 157.46, 143.81, |
| | | 142.91, 135.30, 132.62, 127.86, 126.85, |
| | | 126.79, 126.58, 126.32, 125.99, 124.17, |
| | | 119.25, 118.90, 113.73, 107.14, 55.67, |
| | | 52.00, 47.60, 41.29, 31.69, 22.98. HRMS- |
| | | ESI (m/z): [M + H]⁺ calculated for |
| | | C₂₆H₂₄N₃O₅, 458.1710; found, 458.1711. |
| DEG-59 | 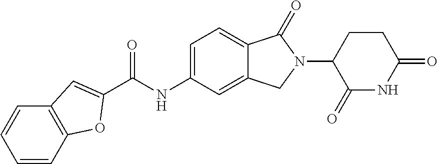 [SMILES: O=C1CCC(N2Cc3cc(NC(=O)c4cc5ccccc5o4)ccc3C2=O)C(=O)N1] N-(2-(2,6-dioxopiperidin-3-yl)-1-oxoisoindolin-5- yl)benzofuran-2-carboxamide | ¹H NMR (400 MHz, DMSO) δ 11.06- 10.78 (m, 2H) 8.17 (s, 1H), 7.91 (d, J = 9.0 Hz, 1H), 7.87-7.83 (m, 2H), 7.77-7.72 (m, 2H), 7.53 (t, J = 7.6 Hz, 1H), 7.39 (t, J = 7.52 Hz, 1H), 5.12 (dd, J = 13.3, 5.0 Hz, 1H), 4.49 (d, J = 17.3 Hz, 1H), 4.34 (d, J = 17.3 Hz, 1H), 2.98-2.86 (m, 1H), 2.65- 2.58 (m, 1H), 2.46-2.33 (m, 1H), 2.05- 1.97 (m, 1H). ¹³C NMR (101 MHz, DMSO) δ 172.90, 171.10, 167.75, 156.95, 154.54, 148.42, 143.12, 141.63, 127.38, 127.14, 127.07, 123.95, 123.60, 123.04, 120.14, 114.66, 112.01, 111.25, 51.58, |
| | | 47.19, 31.23, 22.53. HRMS-ESI (m/z): |
| | | [M + H]⁺ calculated for C₂₂H₁₈N₃O₅, |
| | | 404.1241; found, 404.1239. |
| DEG-60 | 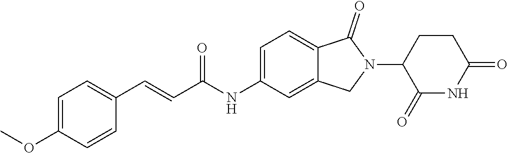 (E)-N-(2-(2,6-dioxopiperidin-3-yl)-1- oxoisoindolin-5-yl)-3-(4- methoxyphenyl)acrylamide | ¹H NMR (400 MHz, DMSO-d₆) δ 10.99 (s, 1H), 10.48 (s, 1H), 8.08 (s, 1H), 7.69 (s, 2H), 7.63-7.56 (m, 3H), 7.02 (d, J = 8.4 Hz, 2H), 6.72 (d, J = 15.6 Hz, 1H), 5.10 (dd, J = 13.1, 4.8 Hz), 4.45 (d, J = 17.2 Hz, 1H), 4.31 (d, J = 17.2 Hz, 1H), 3.80 (s, 3H), 2.97-2.87 (m, 1H), 2.60 (d, J = 16.3 Hz, 1H), 2.44-2.32 (m, 1H), 2.03- 1.96 (m, 1H). ¹³C NMR (101 MHz, DMSO) δ 172.91, 171.13, 167.85, 164.26, 160.75, 143.35, 142.66, 140.65, 129.52, 127.15, 126.32, 123.73, 119.32, 118.83, 114.52, 113.26, 55.32, 51.33, 47.17, 31.23, 22.55. HRMS-ESI (m/z): [M + H]⁺ |
| | | calculated for C₂₃H₂₂N₃O₅, 420.1554; |
| | | found, 420.1552. |
| DEG-61 | 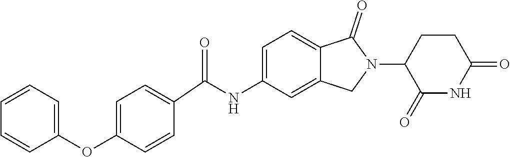 [SMILES: O=C1CCC(N2Cc3cc(NC(=O)c4ccc(Oc5ccccc5)cc4)ccc3C2=O)C(=O)N1] N-(2-(2,6-dioxopiperidin-3-yl)-1-oxoisoindolin-5- yl)-4-phenoxybenzamide | ¹H NMR (400 MHz, DMSO-d₆) δ 11.00 (s, 1H, H₁), 10.51 (s, 1H, H₉), 8.14 (s, 1H), H₆), 8.02 (d, J = 8.7 Hz, 1H), H₁₀), 7.83 (d, J = 8.4 Hz, 1H, H₇), 7.71 (d, J = 8.3 Hz, 1H, H₈), 7.46 (t, J = 7.9 Hz, 2H, H₁₃), 7.24 (t, J = 7.4 Hz, 1H, H₁₄), 7.14-7.09 (m, 4H, H₁₁/H₁₂), 5.11 (dd, J = 13.2, 5.0 Hz, 1H, H₄), 4.47 (d, J = 17.2 Hz, 1H, H₅), 4.32 (d, J = 17.3 Hz, 1H, H₅), 2.97-2.87 (m, 1H, H₂), 2.64-2.57 (m, 1H, H₂), 2.45- 2.33 (m, 1H, H₃), 2.05-1.98 (m, 1H, H₃). HRMS-ESI (m/z): [M + H]⁺ calculated |
| | | for C₂₆H₂₂N₃O₅, 456.1554; found, |
| | | 456.1552. |
| DEG-62 | 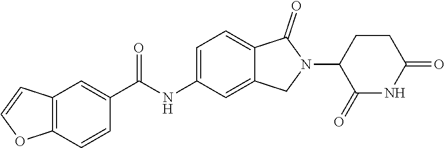 [SMILES: O=C1CCC(N2Cc3cc(NC(=O)c4ccc5occc5c4)ccc3C2=O)C(=O)N1] N-(2-(2,6-dioxopiperidin-3-yl)-1-oxoisoindolin-5- yl)benzofuran-5-carboxamide | ¹H NMR (400 MHz, DMSO-d₆) δ 10.99 (s, 1H), 10.60 (s, 1H), 8.35-8.33 (m, 1H), 8.17 (s, 1H), 8.14 (d, J = 2.1 Hz, 1H), 7.96 (dd, J = 8.6, 1.6 Hz, 1H), 7.86 (d, J = 8.4 Hz, 1H), 7.78-7.70 (m, 2H), 7.13 (d, J = 1.5 Hz, 1H), 5.11 (dd, J = 13.2, 5.1 Hz, 1H), 4.48 (d, J = 17.2 Hz, 1H), 4.33 (d, J = 17.2 Hz, 1H), 2.97-2.87 (m, 1H), 2.65- 2.57 (m, 1H), 2.45-2.34 (m 1H), 2.05- 1.97 (m, 1H). ¹³C NMR (101 MHz, DMSO) δ 173.39, 171.60, 168.34, 166.49, 156.54, 148.00, 143.60, 143.12, 130.29, 127.62, 127.12, 124.97, 123.99, 122.06, |
| | | 120.38, 114.81, 111.74, 107.73, 52.04, |
| | | 47.66, 31.71, 23.02. HRMS-ESI (m/z): |
| | | [M + H]⁺ calculated for C₂₂H₁₈N₃O₅, |
| | | 404.1241; found, 404.1242. |
| DEG-63 | 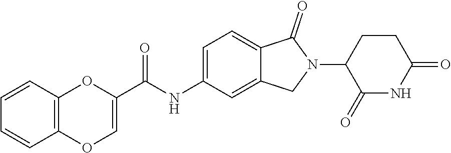 [SMILES: O=C1CCC(N2Cc3cc(NC(=O)C4=COc5ccccc5O4)ccc3C2=O)C(=O)N1] N-(2-(2,6-dioxopiperidin-3-yl)-1-oxoisoindolin-5- yl)benzo[b][1,4]dioxine-2-carboxamide | ¹H NMR (400 MHz, DMSO) δ 10.99 (s, 1H), 10.01 (s, 1H), 8.04 (s, 1H), 7.81 (d, J = 8.0 Hz, 1H), 7.71 (d, J = 8.2 Hz, 1H), 7.21 (s, 1H), 7.06-6.95 (m, 3H), 6.89 (d, J = 7.4 Hz, 1H), 5.10 (dd, J = 13.0, 4.4 Hz, 1H), 4.46 (d, J = 17.2 Hz, 1H), 4.32 (d, J = 17.2 Hz, 1H), 2.98-2.85 (m, 1H), 2.61 (d, J = 16.8 Hz, 1H), 2.45-2.33 (m, 1H), 2.05-1.96 (m, 1H). ¹³C NMR (101 MHz, DMSO) δ 172.90, 171.09, 167.73, 158.45, 143.00, 141.48, 141.21, 140.51, 133.10, 131.13, 127.21, 125.57, 125.12, 123.49, 120.41, 116.78, 116.55, 114.96, 51.58, |
| | | 47.15, 31.22, 22.52. HRMS-ESI (m/z): |
| | | [M + H]⁺ calculated for C₂₂H₁₈N₃O₆, |
| | | 420.1190; found, 420.1189. |
| DEG-64 | 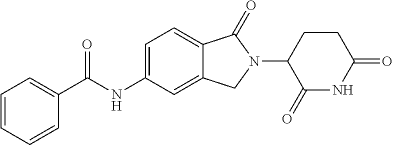 [SMILES: O=C1CCC(N2Cc3cc(NC(=O)c4ccccc4)ccc3C2=O)C(=O)N1] N-(2-(2,6-dioxopiperidin-3-yl)-1-oxoisoindolin-5- yl)benzamide | ¹H NMR (400 MHz, DMSO-d₆) δ 10.97 (s, 1H), 10.58 (s, 1H), 8.15 (s, 1H), 7.97 (d, J = 7.5 Hz, 2H), 7.84 (d, J = 8.3 Hz, 1H), 7.71 (d, J = 8.3 Hz, 1H), 7.64-7.59 (m, 1H), 7.58-7.52 (m, 2H), 5.10 (dd, J = 13.0, 5.1 Hz, 1H), 4.47 (d, J = 17.3 Hz, 1H), 4.33 (d, J = 17.3 Hz, 1H), 2.97-2.86 (m, 1H), 2.65-2.56 (m, 1H), 2.45-2.31 (m, 1H), 2.05-1.96 (m, 1H). ¹³C NMR (101 MHz, DMSO) δ 172.95, 171.14, 167.85, 165.99, 143.11, 142.47, 134.68, 131.85, 128.47, 127.79, 126.76, 123.52, 119.96, 114.41, 51.58, 47.19, 31.24, |
| | | 22.55. HRMS-ESI (m/z): [M + H]⁺ |
| | | calculated for C₂₀H₁₈N₃O₄, 364.1292; |
| | | found, 364.1292. |
| DEG-65 | 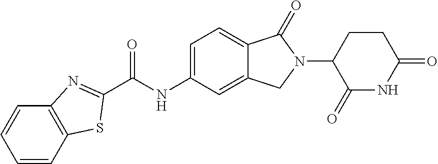 [SMILES: O=C1CCC(N2Cc3cc(NC(=O)c4nc5ccccc5s4)ccc3C2=O)C(=O)N1] N-(2-(2,6-dioxopiperidin-3-yl)-1-oxoisoindolin-5- yl)benzo[d]thiazole-2-carboxamide | δ 11.44 (s, 1H), 10.99 (s, 1H), 8.29 (d, J = 7.7 Hz 1H), 8.24 (d, J = 7.8 Hz, 2H), 8.01 (d, J = 8.3 Hz, 1H), 7.75 (d, J = 8.4 Hz, 1H), 7.71-7.60 (m, 2H), 5.11 (dd, J = 13.2, 5.1 Hz, 1H), 4.50 (d, J = 17.5 Hz, 1H), 4.34 (d, J = 17.5 Hz, 1H), 2.98-2.85 (m, 1H), 2.68-2.56 (m, 1H), 2.46-2.34 (m, 1H), 2.05-1.97 (m, 1H). ¹³C NMR (101 MHz, DMSO) δ 172.93, 171.09, 167.71, 164.35, 158.60, 152.66, 143.08, 141.12, 136.47, 127.61, 127.37, 127.30, 124.29, 123.62, 123.18, 120.60, 115.11, 51.61, 47.24, 31.24, 22.52. HRMS-ESI |
| | | (m/z): [M + H]⁺ calculated for C₂₁H₁₇N₄O₄, |
| | | 421.0965; found, 421.0965. |
| DEG-66 | 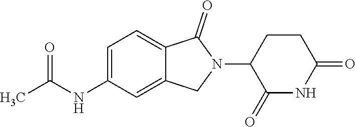 [SMILES: CC(=O)Nc1ccc2c(c1)CN(C1CCC(=O)NC1=O)C2=O] N-(2-(2,6-dioxopiperidin-3-yl)-1-oxoisoindolin-5- yl)acetamide | ¹H NMR (400 MHz, DMSO-d₆) δ 10.94 (s, 1H), 10.27 (s, 1H), 7.95 (s, 1H), 7.64 (d, J = 8.2 Hz, 1H), 7.97 (d, J = 8.1 Hz, 1H), 5.07 (dd, J = 13.3, 5.1 Hz, 1H), 4.42 (d, J = 17.2 Hz, 1H), 4.27 (d, J = 17.2 Hz, 1H), 2.96-2.83 (m, 1H), 2.64-2.55 (m, 1H), 2.43-2.29 (m, 1H), 2.09 (s, 3H), 2.03-1.93 (m, 1H). ¹³C NMR (101 MHz, DMSO) δ 172.92, 171.13, 168.85, 167.86, 143.29, 142.55, 126.15, 123.64, 118.59, 113.02, 51.53, 47.15, 31.23, 24.18, 22.54. |
| | | HRMS-ESI (m/z): [M + H]⁺ calculated for |
| | | C₁₅H₁₆N₃O₄, 302.1135; found, 302.1139. |
| DEG-67 | 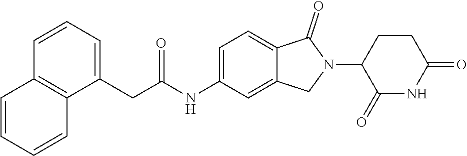 [SMILES: O=C1CCC(N2Cc3cc(NC(=O)Cc4cccc5ccccc45)ccc3C2=O)C(=O)N1] N-(2-(2,6-dioxopiperidin-3-yl)-1-oxoisoindolin-5- yl)-2-(naphthalen-1-yl)acetamide | ¹H NMR (400 MHz, DMSO-d₆) δ 10.95 (s, 1H), 10.67 (s, 1H), 8.12 (d, J = 8.0 Hz, 1H), 7.97 (s, 1H), 7.94 (d, J = 7.2 Hz, 1H), 7.85 (d, J = 8.2 Hz, 1H), 7.66 (d, J = 8.3 Hz, 1H), 7.62 (d, J = 8.3 Hz, 1H), 7.59- 7.45 (m, 4H), 5.07 (dd, J = 13.2, 5.2 Hz, 1H), 4.40 (d, J = 17.5 Hz, 1H), 4.26 (d, J = 17.5 Hz, 1H), 4.20 (s, 2H), 2.95-2.83 (m, 1H), 2.63-2.54 (m, 1H), 2.42-2.29 (m, 1H), 2.02-1.93 (m, 1H). ¹³C NMR (101 MHz, DMSO) δ 172.89, 171.09, 169.60, 167.81, 143.34, 142.43, 133.37, 132.19, 131.98, 128.46, 127.90, 127.33, |
| | | 126.38, 126.16, 125.72, 125.55, 124.15, |
| | | 123.70, 118.78, 113.26, 51.53, 47.13, |
| | | 40.72, 31.21, 22.51. HRM-ESI (m/z): |
| | | [M + H]⁺ calculated for C₂₅H₂₂N₃O₄, |
| | | 428.1605; found, 428.1606. |
| DEG-68 | 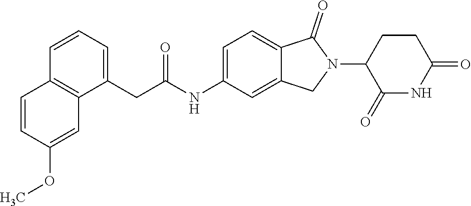 [SMILES: COc1ccc2cccc(CC(=O)Nc3ccc4c(c3)CN(C3CCC(=O)NC3=O)C4=O)c2c1] N-(2-(2,6-dioxopiperidin-3-yl)-1-oxoisoindolin-5- yl)-2-(7-methoxynaphthalen-1-yl)acetamide | ¹H NMR (400 MHz, DMSO-d₆) δ 10.93 (s, 1H), 10.73 (s, 1H), 7.98 (s, 1H), 7.86 (d, J = 9.0 Hz, 1H), 7.77 (d, J = 8.0 Hz, 1H), 7.67 (d, J = 8.2 Hz, 1H), 7.62 (d, J = 8.4 Hz, 1H), 7.46 (d, J = 5.9 Hz, 2H), 7.32 (t, J = 7.6 Hz, 1H), 7.18 (dd, J = 8.9, 2.5 Hz, 1H), 5.07 (dd, J = 13.4, 5.1 Hz, 1H), 4.41 (d, J = 17.5 Hz, 1H), 4.26 (d, J = 17.5 Hz, 1H), 4.15 (s, 2H), 3.85 (s, 3H), 2.95-2.83 (m, 1H), 2.62-2.53 (m, 1H), 2.42-2.29 (m, 1H), 2.02-1.93 (m, 1H), ¹³C NMR (101 MHz, DMSO) δ 172.91, 171.11, 169.72, 167.81, 157.42, 143.36, 142.40, 133.15, 131.06, 130.09, 128.77, 128.39, 127.05, 126.43, 123.72, 123.15, 118.81, 117.84, 113.30, 103.17, 55.22, |
| | | 51.54, 47.15, 41.16, 31.22, 22.52. HRMS- |
| | | ESI (m/z): [M + H]⁺ calculated for |
| | | C₂₆H₂₄N₃O₅, 458.1710; found, 458.1712. |
| DEG-69 | 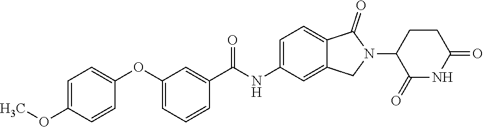 [SMILES: COc1ccc(Oc2cccc(C(=O)Nc3ccc4c(c3)CN(C3CCC(=O)NC3=O)C4=O)c2)cc1] N-(2-(2,6-dioxopiperidin-3-yl)-1-oxoisoindolin-5- yl)-3-(4-methoxyphenoxy)benzamide | ¹H NMR (400 MHz, DMSO-d₆) δ 10.98 (s, 1H), 10.56 (s, 1H), 8.11 (s, 1H), 7.81 (d, J = 8.3 Hz, 1H), 7.71 (s, 1H), 7.69 (s, 1H), 7.54-7.49 (m, 2H), 7.15 (dd, J = 8.5, 2.9 Hz, 1H), 7.09-7.04 (m, 2H), 7.02-6.97 (m, 2H), 5.09 (dd, J = 13.2, 5.1 Hz, 1H), 4.45 (d, J = 17.5 Hz, 1H), 4.31 (d, J = 17.5 Hz, 1H), 3.76 (s, 3H), 2.97-2.85 (m, 1H), 2.64-2.55 (m, 1H), 2.45-2.31 (m, 1H), 2.05-1.95 (m, 1H). ¹³C NMR (101 MHz, DMSO) δ 172.91, 171.11, 167.81 |
| | | 165.21, 158.18, 155.94, 148.93, 143.07, |
| | | 142.25, 136.36, 130.09, 126.88, 123.50, |
| | | 121.85, 120.98, ,120.52, 120.05, 116.36, |
| | | 115.23, 114.54, 55.44, 51.57, 47.17, |
| | | 31.22, 22.53. HRMS-ESI (m/z): [M + H]⁺ |
| | | calculated for C₂₇H₂₄N₃O₆, 486.1660; |
| | | found, 486.1662. |
| DEG-70 | 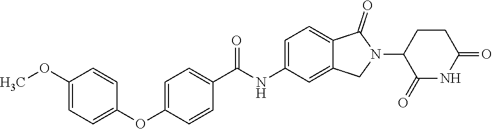 [SMILES: COc1ccc(Oc2ccc(C(=O)Nc3ccc4c(c3)CN(C3CCC(=O)NC3=O)C4=O)cc2)cc1] N-(2-(2,6-dioxopiperidin-3-yl)-1-oxoisoindolin-5- yl)-4-(4-methoxyphenoxy)benzamide | ¹H NMR (400 MHz, DMSO) δ 10.99 (s, 1H), 10.47 (s, 1H), 8.12 (s, 1H), 8.98 (d, J = 8.9 Hz, 2H), 7.81 (d, J = 8.3 Hz, 1H), 7.70 (d, J = 8.4 Hz, 1H), 7.12-7.06 (m, 2H), 7.05-6.99 (m, 4H), 5.10 (dd, J = 13.2, 5.1 Hz, 1H), 4.46 (d, J = 17.5 Hz, 1H), 4.32 (d, J = 17.5 Hz, 1H), 3.77 (s, 3H), 2.98-2.85 (m, 1H), 2.67-2.55 (m, 1H), 2.45-2.31 (m, 1H), 2.05-1.95 (m, 1H). ¹³C NMR (101 MHz, DMSO) δ 172.95, 171.16, 167.87, 165.19, 161.21, 156.24, 148.26, 143.12, 142.56, 130.07, 128.45, 126.64, 123.52, 121.48, 119.92, |
| | | 116.30, 115.30, 114.36, 55.47, 51.56, |
| | | 47.18, 31.25, 22.55. HRMS-ESI (m/z): |
| | | [M + H]⁺ calculated for C₂₇H₂₄N₃O₆, |
| | | 486.1660; found, 486.1660. |
| DEG-71 | 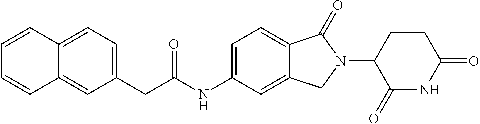 [SMILES: O=C1CCC(N2Cc3cc(NC(=O)Cc4ccc5ccccc5c4)ccc3C2=O)C(=O)N1] N-(2-(2,6-dioxopiperidin-3-yl)-1-oxoisoindolin-5- yl)-2-(naphthalen-2-yl)acetamide | ¹H NMR (400 MHz, DMF-d₇) δ 10.96 (s, 1H), 10.59 (s, 1H), 7.99 (s, 1H), 7.88 (d, J = 8.2 Hz, 3H), 7.84 (s, 1H), 7.66 (d, J = 8.2 Hz, 1H), 7.62 (d, J = 8.4 Hz, 1H), 7.54- 7.45 (m, 3H), 5.07 (dd, J = 13.2, 5.1 Hz, 1H), 4.41 (d, J = 17.4 Hz, 1H), 4.27 (d, J = 17.4 Hz, 1H), 3.88 (s, 2H), 2.95-2.83 (m, 1H), 2.63-2.54 (m, 1H), 2.43-2.29 (m, 1H), 2.02-1.93 (m, 1H). ¹³C NMR (101 MHz, DMSO) δ 172.90, 171.10, 169.61, 167.81, 143.32, 142.42, 133.38, 133.00, 131.88, 127.77, 127.68, 127.51, |
| | | 127.45, 126.40, 126.17, 125.67, 123.69, |
| | | 118.83, 113.31, 51.53, 47.14, 43.47, |
| | | 31.21, 22.52. HRMS-ESI (m/z): [M + H]⁺ |
| | | calculated for C₂₅H₂₂N₃O₄, 428.1605; |
| | | found, 428.1604. |
| DEG-72 | 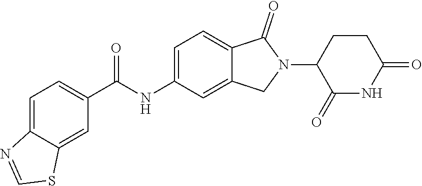 [SMILES: O=C1CCC(N2Cc3cc(NC(=O)c4ccc5ncsc5c4)ccc3C2=O)C(=O)N1] N-(2-(2,6-dioxopiperidin-3-yl)-1-oxoisoindolin-5- yl)benzo[d]thiazole-6-carboxamide | δ 11.00 (s, 1H, H₁), 10.74 (s, 1H, H₉), 9.59 (s, 1H, H₁₁), 8.82 (s, 1H, H₁₀), 8.24 (d, J = 7.9 Hz, 1H, H₁₂), 8.18 (s, 1H, H₆), 8.12 (d, J = 8.8, 1H, H₁₃), 7.85 (d, J = 7.9 Hz, 1H, H₇), 7.73 (d, J = 8.8 Hz, 1H, H₈), 5.11 (dd, J = 13.2, 5.1 Hz, 1H, H₄), 4.49 (d, J = 17.4 Hz, 1H, H₅), 4.34 (d, J = 17.4 Hz, 1H, H₅), 2.98-2.87 (m, 1H, H₂), 2.67-2.56 (m, 1H, H₂), 2.46-2.34 (m, 1H, H₃), 2.05-1.97 (m, 1H, H₃). ¹³C NMR (101 MHz, DMSO) δ 172.95, 171.15, 167.84, 165.53, 159.44, 155.00, 143.18, 142.45, 133.73, 131.84, 126.87, 125.90, 123.61, 122.86, 122.83, 119.94, 114.40, 51.57, 47.21, 31.25, 22.55. HRMS-ESI (m/z): [M + H]⁺ |
| | | calculated for C₂₁H₁₇N₄O₄S₁, 421.0965; |
| | | found, 421.0964 |
| DEG-73 | 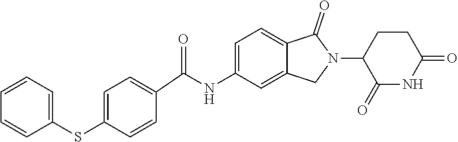 [SMILES: O=C1CCC(N2Cc3cc(NC(=O)c4ccc(Sc5ccccc5)cc4)ccc3C2=O)C(=O)N1] N-(2-(2,6-dioxopiperidin-3-yl)-1-oxoisoindolin-5- yl)-4-(phenylthio)benzamide | ¹H NMR (400 MHz, DMF-d₇) δ 10.98 (s, 1H), 10.54 (s, 1H), 8.12 (s, 1H), 7.92 (d, J = 8.4 Hz, 2H), 7.81 (d, J = 8.3 Hz, 1H), 7.70 (d, J = 8.3 Hz, 1H), 7.51-7.40 (m, 5H), 7.34 (d, J = 8.4 Hz, 2H), 5.09 (dd, J = 13.2, 5.1 Hz, 1H), 4.46 (d, J = 17.4 Hz, 1H), 4.32 (d, J = 17.4 Hz, 1H), 2.97-2.85 (m, 1H), 2.65-2.54 (m, 1H), 2.45-2.30 (m, 1H), 2.05-1.95 (m, 1H). ¹³C NMR (101 MHz, DMSO) δ 172.94, 171.13, 167.86, 165.29, 143.13, 142.39, 141.11, 132.75 (2 × CH), 132.57, 132.36, 129.98 (2 × CH), 128.83 (2 × CH), 128.70, |
| | | 128.27 (2 × CH), 126.80, 123.55, 119.96, |
| | | 114.42, 51.60, 47.21, 31.24, 22.55. |
| | | HRMS-ESI (m/z): [M + H]⁺ calculated for |
| | | C₂₆H₂₂N₃O₄S₁, 472.1326; found, |
| | | 472.1326. |
| DEG-74 | 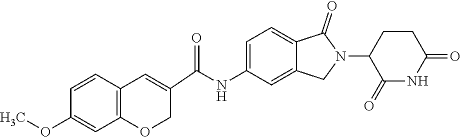 [SMILES: COc1ccc2c(c1)OCC(C(=O)Nc1ccc3c(c1)CN(C1CCC(=O)NC1=O)C3=O)=C2] N-(2-(2,6-dioxopiperidin-3-yl)-1-oxoisoindolin-5- yl)-7-methoxy-2H-chromene-3-carboxamide | ¹H NMR (400 MHz, DMSO-d₆) δ 10.99 (s, 1H, H₁), 10.39 (s, 1H, H₉), 8.06 (s, 1H, H₆), 7.76 (d, J = 9.5 Hz, 1H, H₇), 7.70 (d, J = 8.3 Hz, 1H, H₈), 7.50 (s, 1H, H₁₅), 6.92-6.82 (m, 3H, H₁₁/H₁₂/H₁₄), 5.10 (dd, J = 13.2, 5.1 Hz, 1H, H₄), 4.93 (s, 2H, H₁₀), 4.46 (d, J = 17.3 Hz, 1H, H₅), 4.31 (d, J = 17.3 Hz, 1H, H₅), 3.73 (s, 3H, H₁₃), 2.96-2.86 (m, 1H, H₂), 2.68-2.54 (m, 1H, H₂), 2.44-2.31 (m, 1H, H₃), 2.03- 1.97 (m, 1H, H₃). HRMS-ESI (m/z): [M + H]⁺ calculated for C₂₄H₂₂N₃O₆, |
| | | 448.1503; found, 448.1504. |
| DEG-75 | 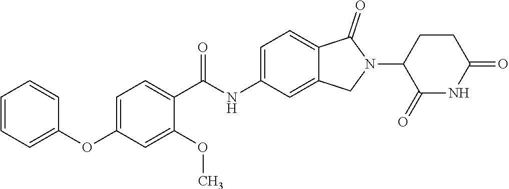 N-(2-(2,6-dioxopiperidin-3-yl)-1-oxoisoindolin-5- yl)-2-methoxy-4-phenoxybenzamide | 10.96 (s, 1H), 10.33 (s, 1H), 8.12 (s, 1H), 7.75 (d, J = 6.4 Hz, 1H), 7.69 (t, J = 7.7 Hz, 2H), 7.46 (t, J = 7.9 Hz, 2H), 7.22 (t, J = 7.4 Hz, 1H), 7.11 (d, J = 7.8 Hz, 2H), 6.88 (d, J = 2.1 Hz, 1H), 6.57 (dd, J = 8.5, 2.2 Hz, 1H), 5.09 (dd, J = 13.2, 5.1 Hz, 1H), 4.45 (d, J = 17.3 Hz, 1H), 4.31 (d, J = 17.3 Hz, 1H), 3.89 (s, 3H), 2.97-2.85 (m, 1H), 2.64-2.55 (m, 1H), 2.46-2.30 (m, 1H), 2.04-1.95 (m, 1H). ¹³C NMR (101 MHz, DMSO) δ 172.92, 171.12, 167.85, 164.14, 160.51, 158.41, 155.50, 143.29, 142.22, 131.77, 130.27 (2 × CH), 126.60, 124.39, 123.62, 119.46 (3 × CH), 119.15, 113.81, 109.29, 102.46, 56.27, 51.58, 47.18, 31.23, 22.53. HRMS-ESI |
| | | (m/z): [M + H]⁺ calculated for C₂₇H₂₄N₃O₆, |
| | | 486.1660; found, 486.1662. |
| DEG-76 | 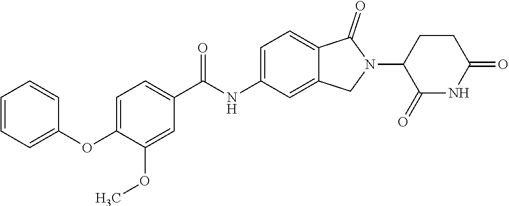 [SMILES: COc1cc(C(=O)Nc2ccc3c(c2)CN(C2CCC(=O)NC2=O)C3=O)ccc1Oc1ccccc1] N-(2-(2,6-dioxopiperidin-3-yl)-1-oxoisoindolin-5- yl)-3-methoxy-4-phenoxybenzamide | ¹H NMR (400 MHz, DMF-d₇) δ 10.99 (s, 1H), 10.53 (s, 1H), 8.12 (s, 1H), 7.83 (d, J = 8.3 Hz, 1H), 7.72 (d, J = 8.4 Hz, 2H), 7.62 (d, J = 8.3 Hz, 1H), 7.37 (t, J = 7.9 Hz, 2H), 7.14-7.07 (m, 2H), 6.93 (d, J = 8.1 Hz, 2H), 5.10 (dd, J = 13.3, 5.0 Hz, 1H), 4.47 (d, J = 17.2 Hz, 1H), 4.33 (d, J = 17.2 Hz, 1H), 3.86 (s, 3H), 2.98-2.85 (m, 1H), 2.65-2.56 (m, 1H), 2.46-2.30 (m, 1H), 2.05-1.96 (m, 1H). ¹³C NMR (101 MHz, DMSO) δ 172.97, 171.17, 167.87, 165.20, 156.92, 150.72, 147.17, 143.14, 142.43, 131.26, 129.97 (2 × CH), 126.82, 123.57, 123.04, 121.14, 120.22, 120.09, 117.04 (2 × CH), 114.56, 112.76, 55.95, 51.58, 47.20, 31.26, 22.56. HRMS- |
| | | ESI (m/z): [M + H]⁺ calculated for |
| | | C₂₇H₂₄N₃O₆, 486.1660; found, 486.1662. |
| DEG-78 | 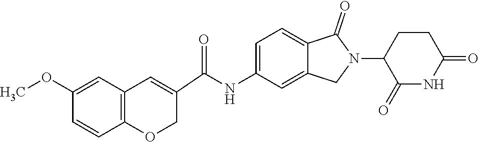 [SMILES: COc1ccc2c(c1)C=C(C(=O)Nc1ccc3c(c1)CN(C1CCC(=O)NC1=O)C3=O)CO2] N-(2-(2,6-dioxopiperidin-3-yl)-1-oxoisoindolin-5- yl)-6-methoxy-2H-chromene-3-carboxamide | ¹H NMR (400 MHz, DMSO-d₆) δ 10.98 (s, 1H), 10.37 (s, 1H), 8.06 (s, 1H), 7.76 (d, J = 8.4 Hz, 1H), 7.70 (d, J = 8.3 Hz, 1H), 7.49 (s, 1H), 6.91-6.82 (m, 3H), 5.10 (dd, J = 13.2, 5.0 Hz, 1H), 4.93 (s, 2H), 4.46 (d, J = 17.2 Hz, 1H), 4.31 (d, J = 17.2 Hz, 1H), 3.73 (s, 3H), 2.97-2.87 (m, 1H), 2.64-2.56 (m, 1H), 2.45-2.32 (m, 1H), 2.03-1.97 (m, 1H). ¹³C NMR (101 MHz, DMSO) δ 173.38, 171.59, 168.27, 164.25, 154.50, 148.62, 143.60, 142.65, 129.26, 127.93, 127.17, 124.04, 122.17, |
| | | 120.13, 117.37, 116.95, 114.61, 113.53, |
| | | 64.77, 55.98, 52.03, 47.63, 31.70, 23.01. |
| | | HRMS-ESI (m/z): [M + H]⁺ calculated for |
| | | C₂₄H₂₂N₃O₆, 448.1503; found, 448.1504. |
| DEG-79 | 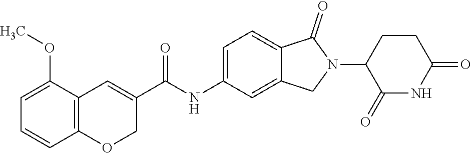 [SMILES: COc1cccc2c1C=C(C(=O)Nc1ccc3c(c1)CN(C1CCC(=O)NC1=O)C3=O)CO2] N-(2-(2,6-dioxopiperidin-3-yl)-1-oxoisoindolin-5- yl)-5-methoxy-2H-chromene-3-carboxamide | ¹H NMR (400 MHz, DMSO-d₆) δ 10.98 (s, 1H), 10.41 (s, 1H), 8.06 (s, 1H), 7.80 (dd, J = 8.4, 1.4 Hz, 1H), 7.76 (s, 1H), 7.69 (d, J = 8.3 Hz, 1H), 7.26 (t, J = 8.2 Hz, 1H), 6.67 (d, J = 8.4 Hz, 1H), 6.53 (d, J = 8.2 Hz, 1H), 5.10 (d, J = 13.2, 5.0 Hz, 1H), 4.92 (s, 2H), 4.45 (d, J = 17.2 Hz, 1H), 4.31 (d, J = 17.2 Hz, 1H), 3.88 (s, 3H), 2.95-2.87 (m, 1H), 2.64-2.56 (m, 1H), 2.44-2.32 (m, 1H), 2.03-1.98 (m, 1H). ¹³C NMR (101 MHz, DMSO) δ 173.38, 171.60, 168.30, 164.21, 156.94, 155.63, 143.53, 142.76, 132.54, 127.04, |
| | | 124.86, 124.15, 123.94, 120.22, 114.71, |
| | | 110.81, 109.00, 104.84, 64.31, 56.32, |
| | | 52.02, 47.62, 31.70, 23.01. HRMS-ESI |
| | | (m/z): [M + H]⁺ calculated for C₂₄H₂₂N₃O₆, |
| | | 448.1503; found, 448.1503. |
| DEG-80 | 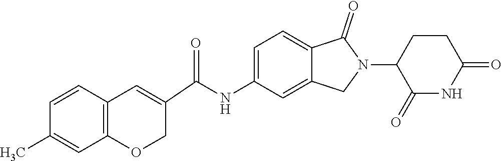 [SMILES: Cc1ccc2c(c1)OCC(C(=O)Nc1ccc3c(c1)CN(C1CCC(=O)NC1=O)C3=O)=C2] N-(2-(2,6-dioxopiperidin-3-yl)-1-oxoisoindolin-5- yl)-7-methyl-2H-chromene-3-carboxamide | ¹H NMR (400 MHz, DMSO-d₆) δ 10.98 (s, 1H), 10.32 (s, 1H), 8.05 (s, 1H), 7.76 (d, J = 8.3 Hz, 1H), 7.69 (d, J = 8.3 Hz, 1H), 7.51 (s, 1H), 7.19 (d, J = 7.6 Hz, 1H), 6.82 (d, J = 7.6 Hz, 1H), 6.72 (s, 1H), 5.90 (dd, J = 13.2, 5.0 Hz, 1H), 4.96 (s, 2H), 4.46 (d, J = 17.2 Hz, 1H), 4.31 (d, J = 17.2 Hz, 1H), 2.97-2.86 (m, 1H), 2.63- 2.57 (m, 1H), 2.44-2.35 (m, 1H), 2.28 (s, 3H), 2.04-1.99 (m, 1H). ¹³C NMR (101 MHz, DMSO) δ 173.38, 171.60, 168.30, 164.29, 154.80, 143.59, 142.71, 142.36, 129.21, 129.03, 127.07, 125.83, 124.01 |
| | | 123.20, 120.11, 118.88, 116.73, 114.57, |
| | | 64.78, 52.03, 47.63, 31.70, 23.01, 21.69. |
| | | HRMS-ESI (m/z): [M + H]⁺ calculated for |
| | | C₂₄H₂₂N₃O₅, 432.1554; found, 432.1553. |
| DEG-81 | 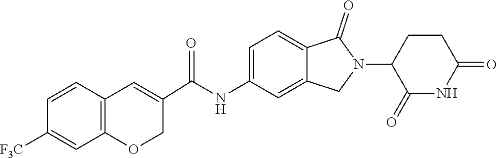 [SMILES: O=C1CCC(N2Cc3cc(NC(=O)C4=Cc5ccc(C(F)(F)F)cc5OC4)ccc3C2=O)C(=O)N1] N-(2-(2,6-dioxopiperidin-3-yl)-1-oxoisoindolin-5- yl)-7-(trifluoromethyl)-2H-chromene-3- carboxamide | ¹H NMR (400 MHz, DMSO-d₆) δ 10.98 (s, 1H, H₁), 10.48 (s, 1H, H₉), 8.06 (s, 1H, H₆), 7.77 (d, J = 8.4 Hz, 1H, H₇), 7.71 (d, J = 8.3 Hz, 1H, H₈), 7.57 (s, 1H, H₁₄), 7.53 (d, J = 7.8 Hz, 1H, H₁₄), 7.34 (d, J = 7.9 Hz, 1H, H₁₂), 7.22 (s, 1H, H₁₁), 5.13- 5.07 (m, 3H, H₄/H₁₀), 4.47 (d, J = 17.3 Hz, 1H, H₅), 4.32 (d, J = 17.3 Hz, 1H, H₅), 2.97-2.86 (m, 1H, H₂), 2.64-2.56 (m, 1H, H₂), 2.46-2.32 (m, 1H, H₃), 2.04- 1.97 (m, 1H, H₃). ¹⁹F NMR (376 MHz, DMSO) δ −61.46. HRMS-ESI (m/z): [M + H]⁺ calculated for C₂₄H₁₉F₃N₃O₅, 486.1271; found, 486.1273. |
| DEG-82 | 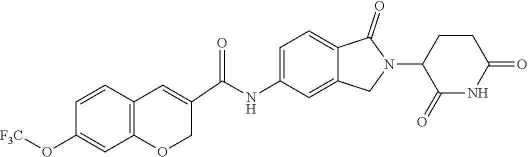 [SMILES: O=C1CCC(N2Cc3cc(NC(=O)C4=Cc5ccc(OC(F)(F)F)cc5OC4)ccc3C2=O)C(=O)N1] N-(2-(2,6-dioxopiperidin-3-yl)-1-oxoisoindolin-5- yl)-7-(trifluoromethoxy)-2H-chromene-3- carboxamide | ¹H NMR (400 MHz, DMSO-d₆) δ 10.99 (s, 1H, H₁), 10.42 (s, 1H, H₉), 8.05 (s, 1H, H₆), 7.76 (d, J = 8.2 Hz, 1H, H₇), 7.70 (d, J = 8.3 Hz, 1H, H₈), 7.55 (s, 1H, H₁₄), 7.44 (d, J = 8.4 Hz, 1H, H₁₃), 7.00 (d, J = 8.4 Hz, 1H, H₁₂), 6.93 (s, 1H, H₁₁), 5.13- 5.06 (m, 3H, H₄/H₁₀), 4.46 (d, J = 17.3 Hz, 1H, H₅), 4.31 (d, J = 17.3 Hz, 1H, H₅), 2.97-2.87 (m, 1H, H₂), 2.64-2.57 (m, 1H, H₂), 2.45-2.32 (m, 1H, H₃), 2.03- 1.97 (m, 1H, H₃). ¹⁹F NMR (376 MHz, DMSO-d₆) δ −56.67. HRMS-ESI (m/z): [M + H]⁺ calculated for C₂₄H₁₉F₃N₃O₆, 502.1220; found, 502.1221. |
| DEG-83 | 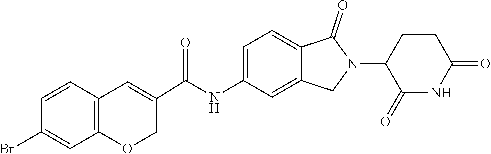 [SMILES: O=C1CCC(N2Cc3cc(NC(=O)C4=Cc5ccc(Br)cc5OC4)ccc3C2=O)C(=O)N1] 7-bromo-N-(2-(2,6-dioxopiperidin-3-yl)-1- oxoisoindolin-5-yl)-2H-chromene-3-carboxamide | 10.98 (s, 1H), 10.39 (s, 1H), 8.04 (s, 1H), 7.75 (dd, J = 8.3, 1.5 Hz, 1H), 7.70 (d, J = 8.3 Hz, 1H), 7.27 (d, J = 8.1 Hz, 1H), 7.19 (dd, J = 8.0, 1.8 Hz, 1H), 7.14 (d, J = 1.7 Hz, 1H), 5.09 (dd, J = 13.3, 5.1 Hz, 1H), 5.03 (d, J = 0.9 Hz, 2H), 4.45 (d, J = 17.2 Hz, 1H), 4.31 (d, J = 17.2 Hz, 1H), 2.96- 2.86 (m, 1H), 2.64-2.56 (m, 1H), 2.44- 2.33 (m, 1H), 2.04-1.97 (m, 1H). ¹³C NMR (101 MHz, DMSO) δ 173.38, 171.59, 168.26, 164.02, 155.53, 143.60, 142.53, 130.59, 128.11, 127.49, 127.23, 125.39, 124.05, 123.98, 120.78, 120.17, |
| | | 119.22, 114.65, 65.19, 52.03, 47.64, |
| | | 31.70, 23.00. HRMS-ESI (m/z): [M + H]⁺ |
| | | calculated for C₂₃H₁₉Br₁N₃O₅, 496.0503; |
| | | found, 496.0506. |
| DEG-84 | 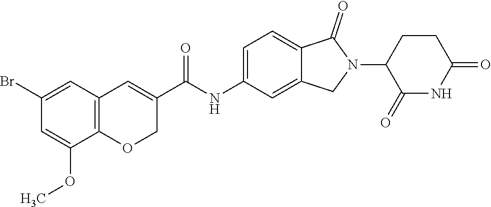 [SMILES: COc1cc(Br)cc2c1OCC(C(=O)Nc1ccc3c(c1)CN(C1CCC(=O)NC1=O)C3=O)=C2] 6-bromo-N-(2-(2,6-dioxopiperidin-3-yl)-1- oxoisoindolin-5-yl)-8-methoxy-2H-chromene-3- carboxamide | ¹H NMR (400 MHz, Acetone-d₆) δ 9.75 (s, 1H, H₁), 9.68 (s, 1H, H₉), 8.15 (s, 1H, H₆), 7.74 (dd, J = 8.4, 1.2 Hz, 1H, H₇), 7.70 (d, J = 10.8 Hz, 1H, H₈), 7.43 (s, 1H, H₁₄), 7.13 (d, J = 2.1 Hz, 1H, H₁₂), 7.02 (d, J = 2.1 Hz, 1H, H₁₃), 5.18 (dd, J = 13.4, 5.1 Hz, 1H, H₄), 5.06 (s, 2H, H₁₀), 4.52 (d, J = 16.7 Hz, 1H, H₅), 4.44 (d, J = 16.6 Hz, 1H, H₅), 3.87 (s, 3H, H₁₁), 3.04- 2.94 (m, 1H, H₂), 2.80-2.73 (m, 1H, H₂), 2.61-2.50 (m, 1H, H₃), 2.23-2.17 (m, 1H, H₃). HRMS-ESI (m/z): [M + H]⁺ calculated for C₂₄H₂₁Br₁N₃O₆, 526.0608; found, 526.0609. |
| DEG-85 | 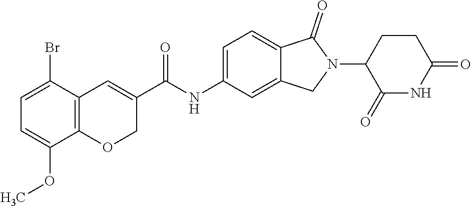 [SMILES: COc1ccc(Br)c2c1OCC(C(=O)Nc1ccc3c(c1)CN(C1CCC(=O)NC1=O)C3=O)=C2] 5-bromo-N-(2-(2,6-dioxopiperidin-3-yl)-1- oxoisoindolin-5-yl)-8-methoxy-2H-chromene-3- carboxamide | ¹H NMR (400 MHz, DMSO-d₆) δ 10.98 (s, 1H), 10.56 (s, 1H), 8.04 (s, 1H), 7.83 (d, J = 8.3 Hz, 1H), 7.78 (d, J = 8.3 Hz, 1H), 7.57 (s, 1H), 7.23 (d, J = 8.8 Hz, 1H), 7.00 (d, J = 8.8 Hz, 1H), 5.09 (dd, J = 8.2, 5.1 Hz, 1H), 4.96 (s, 2H), 4.46 (d, J = 17.3 Hz, 1H), 4.32 (d, J = 17.3 Hz, 1H), 3.79 (s, 3H), 2.97-2.84 (m, 1H), 2.64-2.55 (m, 1H), 2.46-2.30 (m, 1H), 2.05-1.98 (m, 1H). ¹³C NMR (101 MHz, DMSO) δ 172.92, 171.11, 167.79, 163.30, 147.55, 144.98, 143.09, 141.96, 128.35, 126.98, 126.90, 124.95, 123.55, 121.24, 119.99, 115.64, 114.54, 112.58, 64.16, 55.94, 51.57, 47.17, 31.23, 22.53. HRMS-ESI (m/z): [M + H]⁺ calculated for C₂₄H₂₁Br₁N₃O₆, 526.0608; found, 526.0609. |
| DEG-86 | 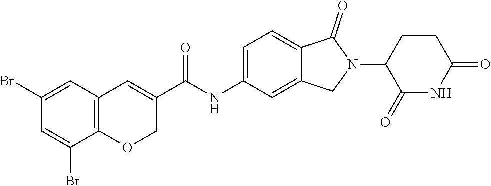 [SMILES: O=C1CCC(N2Cc3cc(NC(=O)C4=Cc5cc(Br)cc(Br)c5OC4)ccc3C2=O)C(=O)N1] 6,8-dibromo-N-(2-(2,6-dioxopiperidin-3-yl)-1- oxoisoindolin-5-yl)-2H-chromene-3-carboxamide | 10.80 (s, 2H, H₁/H₉), 8.05 (s, 1H, H₆), 7.75 (m, 2H, H₇/H₁₁), 7.69 (d, J = 6.0 Hz, 1H, H₈), 7.54 (d, J = 2.0 Hz, 1H, H₁₂), 7.47 (s, 1H, H₁₃), 5.15 (s, 2H, H₁₀), 5.09 (dd, J = 13.2, 4.8 Hz, 1H, H₄), 4.45 (d, J = 17.2 Hz, 1H, H₅), 4.30 (d, J = 17.2 Hz, 1H, H₅), 2.96-2.85 (m, 1H, H₂), 2.63-2.55 (m, 1H, H₂), 2.44-2.31 (m, 1H, H₃), 2.04- 1.95 (m, 1H, H₃). ¹³C NMR (101 MHz, DMSO) δ 173.02, 171.22, 167.81, 163.27, 150.34, 143.14, 142.32, 135.59, 130.14, 128.93, 126.79, 126.62, 124.27, 123.59, 119.89, 114.35, 113.17, 110.25, 65.65, 51.57, 47.17, 31.27, 22.57. HRMS-ESI (m/z): [M + H]⁺ calculated for |
| | | C₂₃H₁₈Br₂N₃O₅, 574.9641; found, 574.9644. |
| DEG-87 | 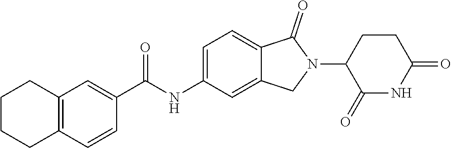 [SMILES: O=C1CCC(N2Cc3cc(NC(=O)c4ccc5c(c4)CCCC5)ccc3C2=O)C(=O)N1] N-(2-(2,6-dioxopiperidin-3-yl)-1-oxoisoindolin-5- yl)-5,6,7,8-tetrahydronaphthalene-2-carboxamide | ¹H NMR (400 MHz, DMSO-d₆) δ 10.97 (s, 1H), 10.42 (s, 1H), 8.13 (s, 1H), 7.83 (d, J = 8.3 Hz, 1H), 7.70 (t, J = 4.2 Hz, 3H), 7.21 (d, J = 8.6 Hz, 1H), 5.09 (dd, J = 13.3, 5.1 Hz, 1H), 4.46 (d, J = 17.2 Hz, 1H), 4.32 (d, J = 17.2 Hz, 1H), 2.98-2.86 (m, 1H), 2.79 (d, J = 8.6 Hz, 4H), 2.65- 2.55 (m, 1H), 2.46-2.30 (m, 1H), 2.05- 1.95 (m, 1H), 1.77 (t, J = 3.0 Hz, 4H). ¹³C NMR (101 MHz, DMSO) δ 172.92, 171.13, 167.87, 165.92, 143.09, 142.60, 141.05, 136.79, 131.73, 129.00, 128.36, |
| | | 126.59, 124.87, 123.47, 119.88, 114.33, |
| | | 51.56, 47.17, 31.23, 28.81, 22.55, 22.46. |
| | | HRMS-ESI (m/z): [M + H]⁺ calculated for |
| | | C₂₄H₂₄N₃O₄, 418.1761; found, 418.1761. |
| DEG-90 | 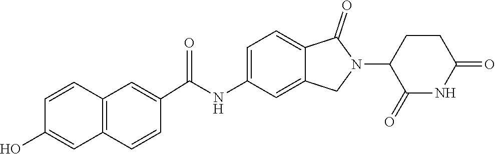 [SMILES: O=C1CCC(N2Cc3cc(NC(=O)c4ccc5cc(O)ccc5c4)ccc3C2=O)C(=O)N1] N-(2-(2,6-dioxopiperidin-3-yl)-1-oxoisoindolin-5- yl)-6-hydroxy-2-naphthamide | IR (ATR-FTIR), cm⁻¹: 2925 (w), 1707 (m), 1684 (m), 1612 (m), 1532 (m), 1233 (m), 1195 (m), 1164 (s), 766 (w). HRMS- ESI (m/z): [M + H]⁺ calculated for C24H20N3O5, 430.1397; found, 430.1397. |
| DEG-91 | 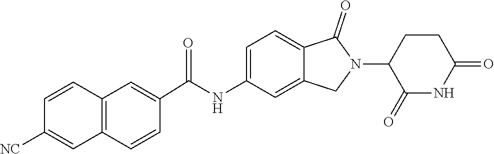 [SMILES: N#Cc1ccc2cc(C(=O)Nc3ccc4c(c3)CN(C3CCC(=O)NC3=O)C4=O)ccc2c1] 6-cyano-N-(2-(2,6-dioxopiperidin-3-yl)-1- oxoisoindolin-5-yl)-2-naphthamide | ¹H NMR (400 MHz, DMSO-d₆): δ 10.88 (s, 2H), 8.70 (s, 2H), 8.30 (d, J = 8.0 Hz, 1H), 8.23 (d, J = 8.0 Hz, 1H), 8.20-8.12 (m, 2H), 7.75 (dd, J = 16.0 Hz, 8.0 Hz, 2H), 7.75 (d, J = 8.0 Hz, 1H), 5.11 (dd, J = 16.0, 4.0 Hz, 1H), 4.50 (d, J = 16.0 Hz, 1H), 4.35 (d, J = 20.0 Hz, 1H), 2.99-2.85 (m, 1H), 2.68-2.56 (m, 1H), 2.46-2.33 (m, 1H), 2.06-1.97 (m, 1H). HRMS-ESI (m/z): [M + H]⁺ calculated for C₂₅H₁₈N₄, 439.1401; found, 439.1411. |
| DEG-92 | 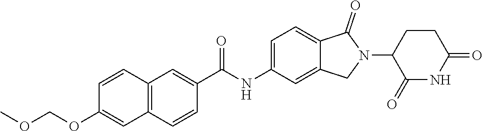 [SMILES: COCOc1ccc2cc(C(=O)Nc3ccc4c(c3)CN(C3CCC(=O)NC3=O)C4=O)ccc2c1] N-(2-(2,6-dioxopiperidin-3-yl)-1-oxoisoindolin-5- yl)-6-(methoxymethoxy)-2-naphthamide | ¹H NMR (400 MHz, DMSO-d₆): δ 10.99 (s, 1H), 10.67 (s, 1H), 8.56 (s, 1H), 8.19 (s, 1H), 8.05 (d, J = 8.0 Hz, 1H), 8.00 (d, J = 8.4 Hz, 1H), 7.94 (d, J = 8.4 Hz, 1H), 7.88 (d, J = 8.6 Hz, 1H), 7.73 (d, J = 8.4 Hz, 1H), 7.53 (d, J = 2.2 Hz, 1H), 7.35 (dd, J = 8.8, 2.8 Hz, 1H), 5.37 (s, 2H), 5.11 (dd, J = 13.4, 5.4 Hz, 1H), 4.49 (d, J = 16.6 Hz, 1H), 4.43 (d, J = 17.4 Hz, 1H), 3.44 (s, 3H), 2.98-2.86 (m, 1H), 2.65- 2.56 (m, 1H), 2.47-2.33 (m, 1H), 2.06- 1.97 (m, 1H). HRMS-ESI (m/z): [M + H]⁺ |
| | | calculated for C₂₆H₂₃N₃O₆, 474.1600; |
| | | found, 474.1667. |
| DEG-93 | 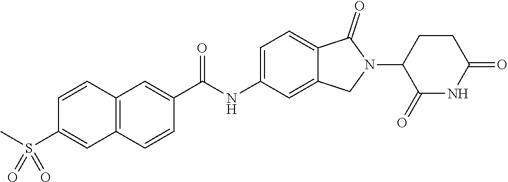 [SMILES: CS(=O)(=O)c1ccc2cc(C(=O)Nc3ccc4c(c3)CN(C3CCC(=O)NC3=O)C4=O)ccc2c1] N-(2-(2,6-dioxopiperidin-3-yl)-1-oxoisoindolin-5- yl)-6-(methylsulfonyl)-2-naphthamide | ¹H NMR (400 MHz, DMSO-d₆): δ 10.99 (s, 1H), 10.87 (s, 1H), 8.71 (d, J = 14.2 Hz, 2H), 8.37 (d, J = 9.0 Hz, 2H), 8.18 (d, J = 10.7 Hz, 2H), 8.06 (d, J = 9.0 Hz, 1H), 7.88 (d, J = 8.4 Hz, 1H), 7.75 (d, J = 8.7 Hz, 1H), 5.11 (dd, J = 12.8, 5.3 Hz, 1H), 4.49 (d, J = 16.8 Hz, 1H), 4.35 (d, J = 17.2 Hz, 1H), 3.34 (s, 3H), 3.00-2.86 (m, 1H), 2.68-2.56 (m, 1H), 2.47-2.33 (m, 1H), 2.06-1.98 (m, 1H). HRMS-ESI (m/z): [M + H]⁺ calculated for C₂₅H₂₁N₃O₆S, 492.1224; found, 492.1228. |
| DEG-94 | 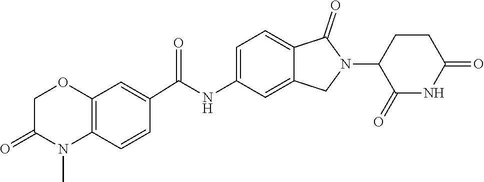 [SMILES: CN1C(=O)COc2cc(C(=O)Nc3ccc4c(c3)CN(C3CCC(=O)NC3=O)C4=O)ccc21] N-(2-(2,6-dioxopiperidin-3-yl)-1-oxoisoindolin-5- yl)-4-methyl-3-oxo-3,4-dihydro-2H- benzo[b][1,4]oxazine-7-carboxamide | ¹H NMR (400 MHz, DMF-d₇): δ 10.94 (s, 1H), 10.55 (s, 1H), 8.29 (s, 1H), 7.98 (d, J = 8.3 Hz, 1H), 7.87 (dd, J = 8.9, 2.4 Hz, 1H), 7.78-7.72 (m, 2H), 7.36 (d, J = 8.8 Hz, 1H), 5.23 (dd, J = 13.3, 5.4 Hz, 1H), 4.80 (s, 2H), 4.58 (d, J = 17.0 Hz, 1H), 4.47 (d, J = 16.4 Hz, 1H), 3.41 (s, 3H), 3.11-2.99 (m, 1H), 2.80-2.76 (m, 1H), 2.63-2.50 (m, 1H), 2.23-2.14 (m, 1H). HRMS-ESI (m/z): [M + H]⁺ calculated for C₂₃H₂₀N₄O₆, 449.1456; found, 449.1464. |
| DEG-95 | 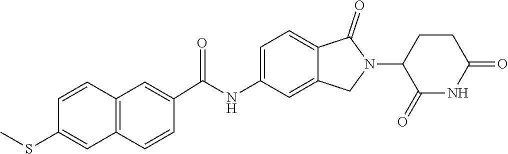 [SMILES: CSc1ccc2cc(C(=O)Nc3ccc4c(c3)CN(C3CCC(=O)NC3=O)C4=O)ccc2c1] N-(2-(2,6-dioxopiperidin-3-yl)-1-oxoisoindolin-5- yl)-6-(methylthio)-2-naphthamide | ¹H NMR (400 MHz, DMSO-d₆): δ 10.99 (s, 1H), 10.70 (s, 1H), 8.54 (s, 1H), 8.18 (s, 1H), 8.05-7.94 (m, 3H), 7.88 (d, J = 8.2 Hz, 1H), 7.79 (s, 1H), 7.73 (d, J = 8.8 Hz, 1H), 7.50 (dd, J = 8.6, 1.9 Hz, 1H), 5.11 (dd, J = 13.0, 5.5 Hz, 1H), 4.49 (d, J = 17.0 Hz, 1H), 4.34 (d, J = 17.8 Hz, 1H), 2.99-2.85 (m, 1H), 2.69-2.56 (m, 4H), 2.46-2.33 (m, 1H), 2.07-1.96 (m, 1H). HRMS-ESI (m/z): [M + H]⁺ calculated for C₂₅H₂₁N₃O₄S, 460.1326; found, 460.1330. |
| DEG-96 | 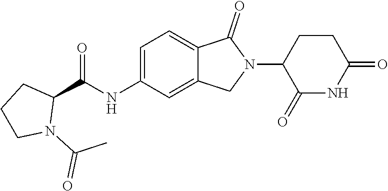 [SMILES: CC(=O)N1CCC[C@H]1C(=O)Nc1ccc2c(c1)CN(C1CCC(=O)NC1=O)C2=O] (2S)-1-acetyl-N-(2-(2,6-dioxopiperidin-3-yl)-1- oxoisoindolin-5-yl)pyrrolidine-2-carboxamide | ¹H NMR (mixture of rotamers, 400 MHz, DMSO-d₆): δ 10.96 (s, 1H), 10.51 (s, 0.2H), 10.30 (s, 0.8H), 7.98 (s, 0.2H), 7.95 (d, J = 6.2 Hz, 0.8H), 7.66 (t, J = 8.6 Hz, 1H), 7.63-7.56 (m, 1H), 5.08 (dd, J = 13.0, 5.4 Hz, 1H), 4.55 (dd, J = 8.6, 3.0 Hz, 0.2H), 4.47-4.37 (m, 1.8H), 4.28 (d, J = 17.2 Hz, 1H), 3.64-3.36 (m, 2H), 2.97- 2.84 (m, 1H), 2.64-2.55 (m, 1H), 2.44- 2.29 (m, 1.2H), 2.21-2.10 (m, 0.8H), 2.04-1.86 (m, 6H), 1.86-1.78 (m, 1H). HRMS-ESI (m/z): [M + H]⁺ calculated for C₂₀H₂₂N₄O₅, 399.1663; found, 399.1668. |
| DEG-97 | 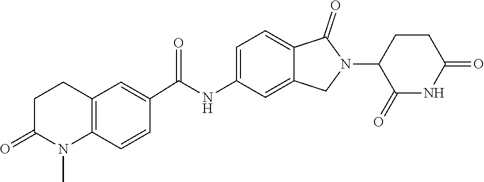 [SMILES: CN1C(=O)CCc2cc(C(=O)Nc3ccc4c(c3)CN(C3CCC(=O)NC3=O)C4=O)ccc21] | ¹H NMR (400 MHz, DMSO-d₆): δ 10.98 (s, 1H), 10.46 (s, 1H), 8.14 (s, 1H), 7.93 (d, J = 8.0 Hz, 1H), 7.87 (s, 1H), 7.84 (d, J = 8.4 Hz, 1H), 7.70 (d, J = 8.6 Hz, 1H), 7.23 (d, J = 8.8 Hz, 1H), 5.10 (dd, J = 12.7, 4.9 Hz, 1H), 4.47 (d, J = 17.4 Hz, 1H), 4.32 (d, J = 17.4 Hz, 1H), 3.30 (s, 3H), 3.02-2.85 (m, 3H), 2.66-2.56 (m, 1H). HRMS-ESI (m/z): [M + H]⁺ calculated for C₂₄H₂₂N₄O₅, 447.1663; found, 447.1671. |
| | N-(2-(2,6-dioxopiperidin-3-yl)-1-oxoisoindolin-5- | |
| | yl)-1-methyl-2-oxo-1,2,3,4-tetrahydroquinoline-6- | |
| | carboxamide | |
| DEG-98 | 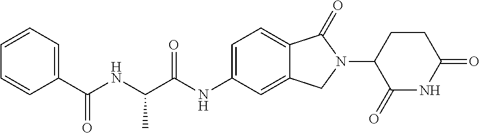 [SMILES: C[C@H](NC(=O)c1ccccc1)C(=O)Nc1ccc2c(c1)CN(C1CCC(=O)NC1=O)C2=O] N-((2S)-1-((2-(2,6-dioxopiperidin-3-yl)-1- oxoisoindolin-5-yl)amino)-1-oxopropan-2- yl)benzamide | ¹H NMR (400 MHz, DMSO-d₆): δ 10.96 (s, 1H), 10.43 (s, 1H), 8.71 (d, J = 6.6 Hz, 1H), 7.98 (d, J = 4.5 Hz, 1H), 7.92 (d, J = 6.6 Hz, 2H), 7.70-7.62 (m, 2H), 7.54 (t, J = 7.5 Hz, 1H), 7.47 (t, J = 7.5 Hz, 2H), 5.08 (dd, J = 13.0, 5.5 Hz, 1H), 4.63 (qu, J = 7.1 Hz, 1H), 4.43 (d, J = 17.6 Hz, 1H), 4.29 (d, J = 17.0 Hz, 1H), 2.97-2.84 (m, 1H), 2.64-2.54 (m, 1H), 2.44-2.29 (m, 1H), 2.04-1.94 (m, 1H), 1.45 (d, J = 6.9 Hz, 3H). HRMS-ESI (m/z): [M + H]⁺ calculated for C₂₃H₂₂N₄O₅, 435.1663; found, 435.1672. |

### N-(2-(2,6-dioxopiperidin-3-yl)-1-oxoisoindolin-5-yl)acrylamide (88)

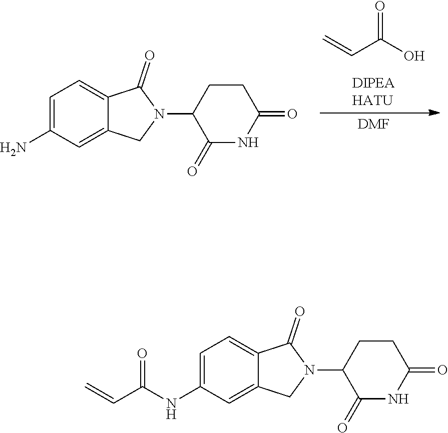

5-NH₂-Lenalidomide (30.0 mg, 116 μmol, 1.0 equiv.) was added in a single portion to a stirred solution of acrylic acid (500 μL, 63 equiv.) in a 20 mL vial. EDCl (26.7 mg, 139 μmol, 1.20 equiv.) was added into the mixture. The reaction mixture was stirred for 16 h at 24° C. TLC showed full conversion of the starting material. The product mixture was then loaded on silica gel directly and subjected to purification by flash column chromatography (ISCO 12 g gold column, 0 to 10% gradient MeOH in DCM over 20 min) to afford the product as a white solid (20.0 mg, 63.9 μmol, 55%). ¹H NMR (400 MHZ, DMSO) δ 10.98 (s, 1H), 10.47 (s, 1H), 8.05 (t, J=1.2 Hz, 1H), 7.74-7.55 (m, 2H), 6.48 (dd, J=17.0, 10.1 Hz, 1H), 6.31 (dd, J=17.0, 2.0 Hz, 1H), 5.81 (dd, J=10.1, 2.0 Hz, 1H), 5.09 (dd, J=13.3, 5.1 Hz, 1H), 4.45 (d, J=17.5 Hz, 1H), 4.31 (d, J=17.2 Hz, 1H), 2.91 (ddd, J=17.2, 13.6, 5.4 Hz, 1H), 2.64-2.55 (m, 1H), 2.43-2.31 (m, 1H), 1.99 (dtd, J=12.6, 5.2, 2.2 Hz, 1H). ¹³C NMR (101 MHz, DMSO) δ 172.9, 171.1, 167.8, 163.5, 143.3, 142.2, 131.6, 127.7, 126.6, 123.7, 119.0, 113.5, 51.5, 47.1, 31.2, 22.5. IR (ATR-FTIR), in DMSO, cm⁻¹: 3487 (br), 2996 (w), 2912 (w), 1686 (w), 1436 (m), 1407 (m), 1310 (m), 1235 (w), 1204 (w), 1042 (s), 1020 (s), 952 (s), 930 (m), 897 (w). HRMS-ESI (m/z): [M+H]+ calculated for C₁₆H₁₆N₃O₄ 314.1135; found, 314.1135.

### Selective Degradation of IKZF2

Evaluation of previously discovered cereblon ligands showed that degradation of IKZF2 was sensitive to the exact substitution pattern of the projection from the 5-position of the isoindolinone, while degradation of IKZF1 appeared to be more permissive. These compounds were without an amide moiety at the 5-position of the isoindolinone, and were not sterically inhibiting recruitment of IKZF1. Based on further modeling, it was hypothesized that rigidifying the core structure via an amide linkage would reduce the overall conformational flexibility, and thus potentially serve to fix the compound in a favorable conformation to recruit IKZF2 and block recruitment of IKZF1. Compounds of the present disclosure, which have an amide moiety, possess increased selectivity for IKZF2 over IKZF1.

### Diversification of the Compound Scaffold

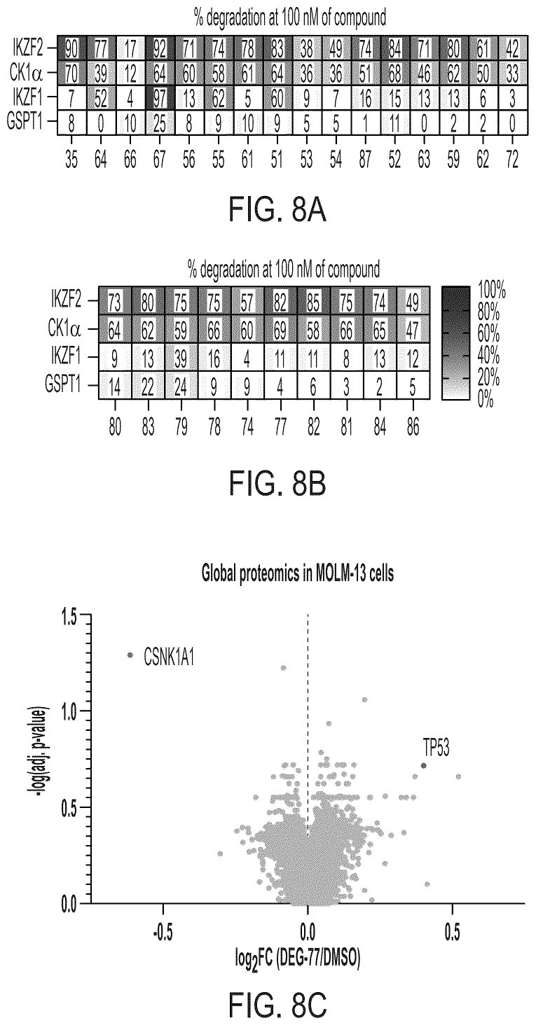

Known compounds such as DEG-35 suffered from limited solubility and additional scaffolds were explored. DEG-64 (phenylamide) and DEG-66 (acyl amide) indicated that a bicyclic substituent was useful for selective degradation of IKZF2 and CK1α. Additional analogs (DEG51-56, 59, 61-64, 66, 67, 72, 87) which contain heteroatoms, sp³-hybridized carbons, and non-continuous pi systems, were tested at 100 nM or 1 μM in the panel of reporter cell lines. The homonaphthalene 67, vinyl phenyl 56, diphenyl ethers 55 and 61, 2H-chromene 52, and benzofuran 59 showed good degradation activity for CK1α and/or IKZF2, while sparing IKZF1 and/or GSPT1 (FIG. 8A). All analogs were fully soluble in DMSO to at least 50 mM.

Diversification around the 2H-chromene 52 showed potency and selectivity for IKZF2 and CK1α (FIG. 8B). To probe substituent toleration around the 2H-chromene core, additional analogs (e.g., DEG-74, 77-84, and 86) were generated. The 2H-chromene analogs induced potent IKZF2 and CK1α degradation (DC₅₀ IKZF2=6-34 nM, DC₅₀ CK1α=1-10 nM, Tables 1-2), reflecting a remarkable tolerance to substitution about the 2H-chromene scaffold (FIG. 8B). Tables 1-3 show degradation data for compounds of the disclosure.

Based on these data, DEG-77, was further characterized. Global proteomics of DEG-77 after 2 h treatment in MOLM-13 cells reflected its high selectivity, rapid degradation of CK1α, and the response through elevated TP53 (FIG. 8C). Notably, degradation of IKZF2 is only observed after 24 h in MOLM-13 cells. DEG-77 additionally possessed improved in vitro metabolic stability (t_{1/2}>6 h) and in vivo half-life (t_{1/2}=8 h) which correlate with in vivo therapeutic efficacy leading to a significant increase in survival upon treatment in AML mouse models. Thus, DEG-77 is a potent and selective IKZF2 and CK1α degrader with favorable metabolic stability and solubility properties.

TABLE 1

| | IKZF1% | IKZF1% | IKZF2% | IKZF2% |
|---|---|---|---|---|
| | degradation | degradation | degradation | degradation |
| Cmpd | at 1 μM | at 100 nM | at 1 μM | at 100 nM |
| 64 | 85.4 ± 0.5 | 52.1 ± 0.1 | 91.9 ± 0 | 77.4 ± 0.7 |
| 66 | 10.9 ± 0.9 | 4.4 ± 0.8 | 48.5 ± 0.2 | 16.6 ± 0.1 |
| 67 | 98.1 ± 0.1 | 97.2 ± 0.1 | 94.6 ± 0 | 92.2 ± 0.1 |
| 56 | 29.4 ± 0.3 | 13.1 ± 0.8 | 89.6 ± 0.1 | 70.8 ± 0.3 |
| 55 | 86.8 ± 0.4 | 62.1 ± 0.2 | 91.1 ± 0.1 | 73.9 ± 0.4 |
| 61 | 9.6 ± 0.5 | 5.2 ± 1 | 91.4 ± 0.1 | 78.4 ± 0.8 |
| 51 | 84 ± 0.2 | 59.5 ± 0.2 | 93.9 ± 0 | 82.6 ± 0.3 |
| 53 | 8.8 ± 1.6 | 9.3 ± 0.8 | 76.5 ± 0.7 | 38.5 ± 0.2 |
| 54 | 9.7 ± 1.4 | 7.4 ± 0.8 | 84.1 ± 0.3 | 49.2 ± 0.2 |
| 87 | 27.2 ± 0.7 | 15.5 ± 1 | 89.1 ± 0.4 | 74 ± 0.5 |
| 52 | 29.2 ± 0.5 | 15.2 ± 0.2 | 93.8 ± 0.1 | 83.9 ± 0.4 |
| 63 | 35.2 ± 0.6 | 13.4 ± 0.7 | 90.7 ± 0.2 | 70.8 ± 0.6 |
| 59 | 26.5 ± 1.2 | 13.4 ± 0.6 | 92 ± 0.1 | 79.7 ± 0.1 |
| 62 | 22.3 ± 0.5 | 6.4 ± 0.3 | 90.6 ± 0.1 | 61.1 ± 0.5 |
| 72 | 6.5 ± 1.2 | 2.7 ± 1.2 | 82.5 ± 0.9 | 41.8 ± 2 |
| 58 | 98.4 ± 0 | 97.7 ± 0.1 | 95.8 ± 0.1 | 93 ± 0.2 |
| 68 | 97.6 ± 0.1 | 96.9 ± 0.1 | 94.4 ± 0.1 | 92.2 ± 0.1 |
| 71 | 98.4 ± 0.1 | 98.5 ± 0.1 | 88.5 ± 3 | 93.8 ± 0.5 |
| 60 | 40.3 ± 0.9 | 17.6 ± 0.8 | 89.5 ± 0.2 | 74.7 ± 0.3 |
| 57 | 92.5 ± 0.2 | 81.9 ± 0.7 | 88.6 ± 0.2 | 76.2 ± 0.4 |
| 69 | 98.6 ± 0.1 | 98.6 ± 0.1 | 93.2 ± 0.7 | 89.5 ± 0.1 |
| 70 | 16.2 ± 0.6 | 6.5 ± 1.4 | 84.5 ± 0.3 | 53.6 ± 0.6 |
| 75 | 7 ± 0.4 | 2.4 ± 1.1 | 83.3 ± 0.3 | 54.6 ± 1.1 |
| 76 | 17.2 ± 1.2 | 8.7 ± 1.8 | 92.8 ± 0 | 81.8 ± 0.4 |
| 73 | 26.3 ± 0.6 | 11.7 ± 0.3 | 87.8 ± 0.3 | 67.4 ± 1.2 |
| 80 | 24 ± 0.1 | 8.6 ± 0.1 | 91.5 ± 0.2 | 72.6 ± 0.9 |
| 83 | 47.5 ± 0.2 | 12.9 ± 1 | 94.4 ± 0.2 | 80.4 ± 0.5 |
| 79 | 70.3 ± 0.2 | 39.4 ± 0.8 | 93 ± 0.1 | 74.6 ± 0.3 |
| 78 | 33.9 ± 0.9 | 15.7 ± 1.1 | 90.3 ± 0 | 75 ± 0.8 |
| 74 | 32.5 ± 0.4 | 4.3 ± 0.5 | 96 ± 0 | 57.1 ± 1.1 |
| 77 | 29 ± 0.9 | 10.7 ± 1.1 | 94.4 ± 0.1 | 82.3 ± 0.6 |
| 82 | 19.9 ± 0.3 | 10.6 ± 1.2 | 93.3 ± 0.1 | 85.4 ± 0 |
| 81 | 17.4 ± 3.3 | 8 ± 0.5 | 91.8 ± 0.1 | 75.1 ± 0.5 |
| 84 | 26.1 ± 0.5 | 12.6 ± 0.5 | 87.8 ± 0.2 | 74.4 ± 0.4 |
| 86 | 22.6 ± 0.5 | 12.3 ± 0.9 | 62.6 ± 0.5 | 48.7 ± 1.1 |
| 90 | 6.3 ± 1.6 | 6 ± 0.1 | 89 ± 0.2 | 60 ± 1 |
| 91 | 14.4 | 8.5 | 95.3 | 89.4 |
| 92 | 8.3 | 6.7 | 94.5 | 87.6 |
| 93 | 7.0 | 4.9 | 87.7 | 56.0 |
| 94 | 20.8 | 8.7 | 85.2 | 51.8 |
| 95 | 9.1 | 7.7 | 94.1 | 86.7 |
| 96 | 5.0 | 3.6 | 57.0 | 21.4 |
| 97 | 4.1 | 2.4 | 87.1 | 52.6 |
| 98 | 79.7 | 50.9 | 68.1 | 35.8 |

TABLE 2

| | CK1α % | CK1α % | GSPT1% | GSPT1% |
|---|---|---|---|---|
| | degradation | degradation | degradation | degradation |
| Cmpd | at 1 μM | at 100 nM | at 1 μM | at 100 nM |
| 64 | 65.5 ± 0.7 | 38.7 ± 1.9 | 14.1 ± 0.5 | 0 ± 1 |
| 66 | 25.4 ± 0.8 | 12 ± 1.3 | 8.6 ± 0.8 | 9.9 ± 0.8 |
| 67 | 62.8 ± 0.5 | 64.4 ± 0.5 | 30.9 ± 1 | 25.3 ± 0.5 |
| 56 | 67 ± 1.1 | 60.2 ± 2.3 | 11.1 ± 1.3 | 7.6 ± 1.5 |
| 55 | 67.4 ± 1.4 | 57.6 ± 1.7 | 14 ± 1.8 | 8.8 ± 0.8 |
| 61 | 65.8 ± 1.2 | 60.9 ± 0.7 | 16.9 ± 2.3 | 10.2 ± 2.8 |
| 51 | 69.8 ± 0.5 | 63.7 ± 1.1 | 13.7 ± 0.3 | 9 ± 1.6 |
| 53 | 55.4 ± 3.9 | 36.2 ± 4.5 | 2.4 ± 0.9 | 5 ± 1.4 |
| 54 | 57.6 ± 3.5 | 33.5 ± 10 | 3.3 ± 1.7 | 5.2 ± 1.5 |
| 87 | 56.9 ± 0.8 | 51.1 ± 1.2 | 4 ± 0.6 | 1.2 ± 2.5 |
| 52 | 72.8 ± 0.1 | 68.4 ± 0.2 | 12.2 ± 1.9 | 10.5 ± 1.7 |
| 63 | 60.3 ± 1.2 | 46.4 ± 1.4 | 1.2 ± 0.3 | −0.1 ± 1.2 |
| 59 | 63.3 ± 3.9 | 61.7 ± 2.4 | −1 ± 3.4 | 2 ± 3.6 |
| 62 | 64.7 ± 0.4 | 50.2 ± 1.7 | 4.4 ± 1.9 | 2 ± 2.1 |
| 72 | 54.5 ± 3.8 | 33.2 ± 3.2 | −0.9 ± 2.6 | −1.7 ± 3.4 |
| 58 | 57 ± 0.1 | 63.4 ± 0.6 | 41.9 ± 0.8 | 29 ± 1.9 |
| 68 | 64.4 ± 1.6 | 59.7 ± 1.2 | 21.3 ± 1.8 | 16.6 ± 1.3 |
| 71 | −153 ± 2.3 | −190.2 ± 2.8 | 69.4 ± 0.6 | 63.1 ± 0.4 |
| 60 | 68.3 ± 1.2 | 64.3 ± 1.3 | 16.5 ± 2 | 11.6 ± 1.4 |
| 57 | 54.1 ± 2.1 | 40.3 ± 11.2 | 9.9 ± 0.8 | 11.7 ± 1.3 |
| 69 | 65.5 ± 2.3 | 64.8 ± 0.3 | 16.8 ± 1.9 | 12.5 ± 1.2 |
| 70 | 55.8 ± 5.6 | 44.9 ± 3.4 | 4.8 ± 0.4 | 4.3 ± 1.1 |
| 75 | 64 ± 1.5 | 48.1 ± 2.2 | 12.6 ± 1.2 | 0.4 ± 1.4 |
| 76 | 69.9 ± 0.5 | 64.1 ± 0.8 | 21.3 ± 1.7 | 12.1 ± 0.5 |
| 73 | 60.4 ± 0.6 | 56.6 ± 1.2 | 19.8 ± 0.8 | 11.1 ± 1.4 |
| 80 | 66.9 ± 1.1 | 63.5 ± 1.5 | 28 ± 0.8 | 14.3 ± 2.1 |
| 83 | 30.2 ± 2.3 | 61.9 ± 0.4 | 49 ± 0.8 | 21.8 ± 2.3 |
| 79 | 38.1 ± 3.4 | 59.4 ± 1 | 44.3 ± 1.3 | 24 ± 1 |
| 78 | 69.5 ± 1.4 | 65.5 ± 1.3 | 14.4 ± 0.5 | 9.2 ± 0.8 |
| 74 | 64.8 ± 0.6 | 59.5 ± 1.1 | 37.5 ± 2.1 | 9 ± 2.6 |
| 77 | 72.7 ± 0.8 | 68.6 ± 1 | 2.6 ± 1 | 3.7 ± 1.3 |
| 82 | 62.7 ± 1.4 | 58 ± 2.2 | 12.3 ± 0.9 | 6.1 ± 0.3 |
| 81 | 72 ± 0.3 | 66.3 ± 2 | 11.9 ± 1.9 | 2.8 ± 0.8 |
| 84 | 68 ± 1.8 | 64.7 ± 0.1 | 4.1 ± 2.6 | 1.6 ± 1.1 |
| 86 | 54.7 ± 1.2 | 46.5 ± 2.5 | 9.2 ± 0.7 | 5.3 ± 2 |
| 90 | 71.1 ± 0.6 | 54.5 ± 0.6 | 7.8 ± 1.4 | −1.9 ± 1.4 |
| 91 | 80.3 | 74.6 | 11.5 | 9.7 |
| 92 | 80.1 | 76.2 | 5.0 | 5.3 |
| 93 | 74.6 | 60.2 | 10.6 | 7.8 |
| 94 | 71.5 | 51.3 | 13.2 | 9.3 |
| 95 | 80.1 | 76.5 | 24.8 | 16.3 |
| 96 | 26.3 | 9.2 | 10.1 | 8.6 |
| 97 | 74.1 | 60.1 | −3.4 | 1.6 |
| 98 | 41.2 | 18.2 | 6.2 | 5.5 |

TABLE 3

| | DC₅₀ | Dₘₐₓ | DC₅₀ | Dₘₐₓ | DC₅₀ | Dₘₐₓ | DC₅₀ | Dₘₐₓ | In vitro |
|---|---|---|---|---|---|---|---|---|---|
| | CK1a | CK1a | IKZF2 | IKZF2 | IKZF1 | IKZF1 | GSPT1 | GSPT1 | solubility |
| Cmpd | (nM) | (%) | (nM) | (%) | (nM) | (%) | (nM) | (%) | in DMSO |
| 91 | 2.6 | 81.2 | 7 | 96.6 | N/A | 20.6 | N/A | 14.8 | >100 mM |
| 92 | 2.9 | 80.2 | 7.9 | 95.9 | N/A | 14.5 | N/A | 12.8 | >100 mM |
| 93 | 21.1 | 78.9 | 73.1 | 95.1 | N/A | 10.4 | N/A | 14.6 | 25 > x ≥ 10 mM |
| 94 | 42.5 | 76.7 | 78.8 | 92.8 | N/A | 30.5 | N/A | 13.7 | 100 > x ≥ 50 mM |
| 95 | 2.8 | 78.9 | 10.4 | 95.4 | N/A | 17.2 | 1187 | 33.5 | >100 mM |
| 96 | 502.9 | 40.3 | 339.7 | 79.5 | N/A | 8.3 | N/A | 14.2 | >100 mM |
| 97 | 32 | 78.0 | 83.1 | 95.9 | N/A | 13.1 | N/A | 5.6 | >100 mM |
| 98 | 175.2 | 54.1 | 142.8 | 80.5 | 72 | 88 | N/A | 0 | >100 mM |

### Therapeutic Potential of Degradation of CK1α and IKZF2

In addition to investigation of DEG-77 for efficacy in AML, DEG-77 was evaluated in other indications as activation of p53-dependent apoptosis in cancer cells has been an area of intense research for decades. Accordingly, there is still an unmet need for targeted therapeutics that reactivate the p53 pathway. CK1α degradation by DEG-77 represents a different but orthogonal mechanism of action for the activation of p53, potentially providing the opportunity to overcome past obstacles with therapies such as MDM2 inhibitors in various cancer indications.

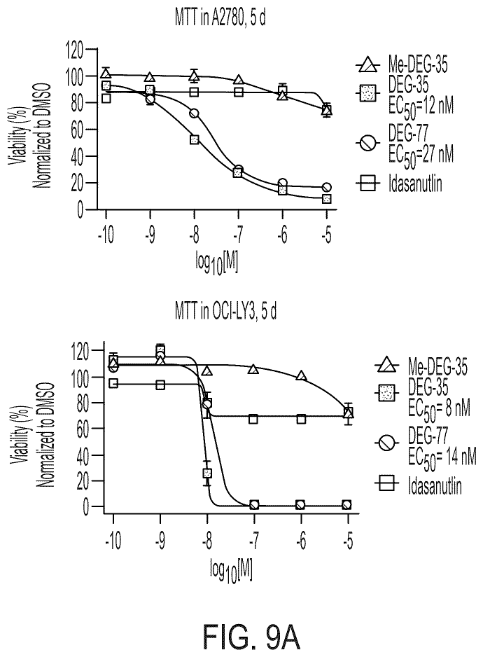

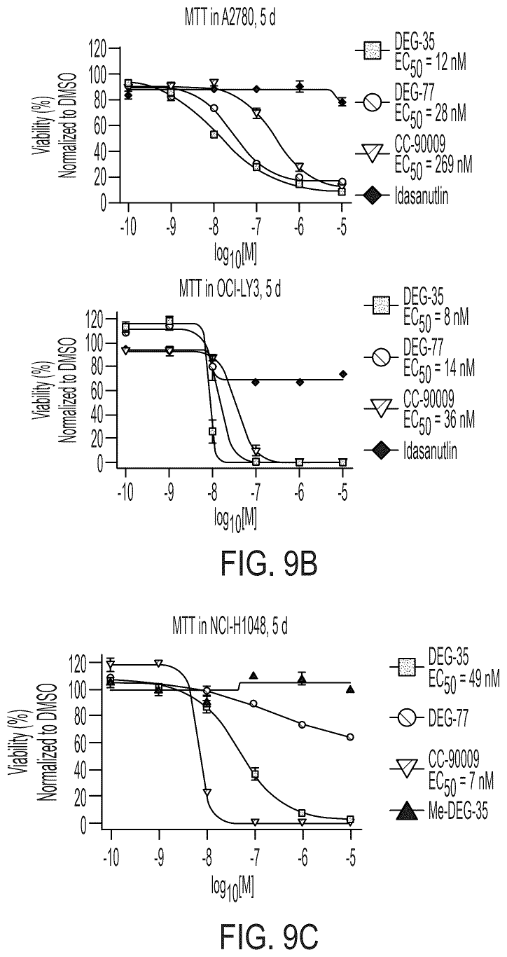

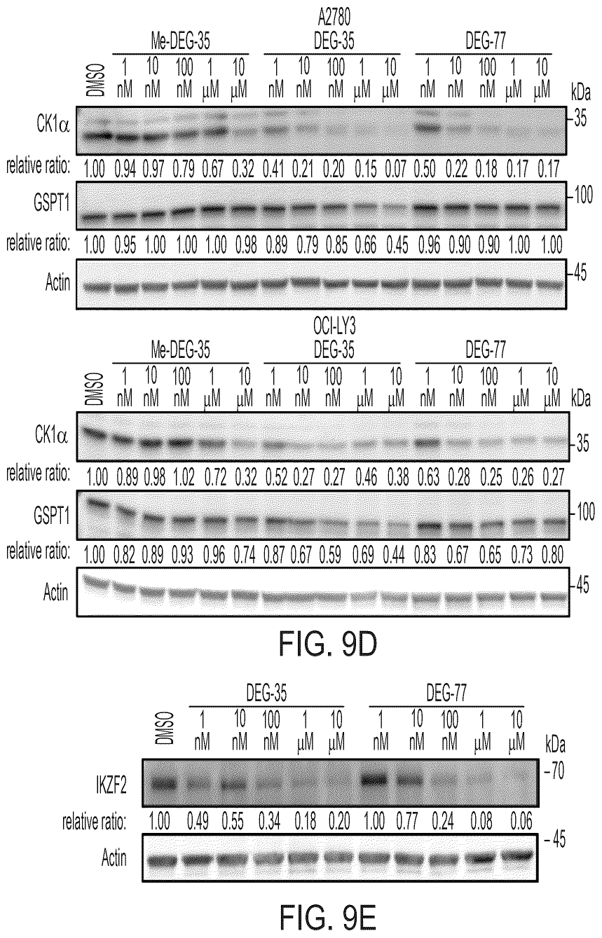

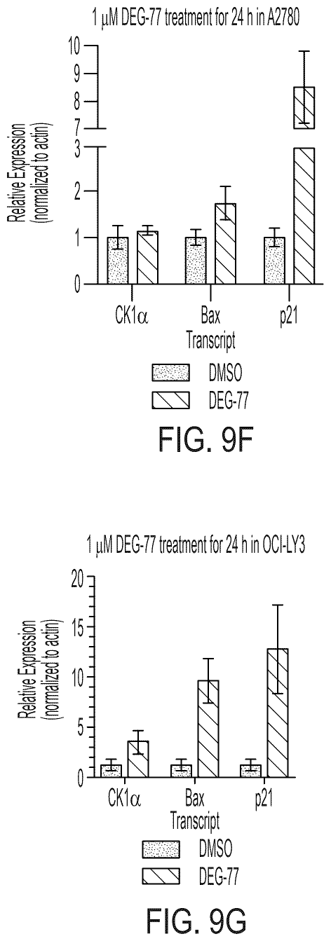

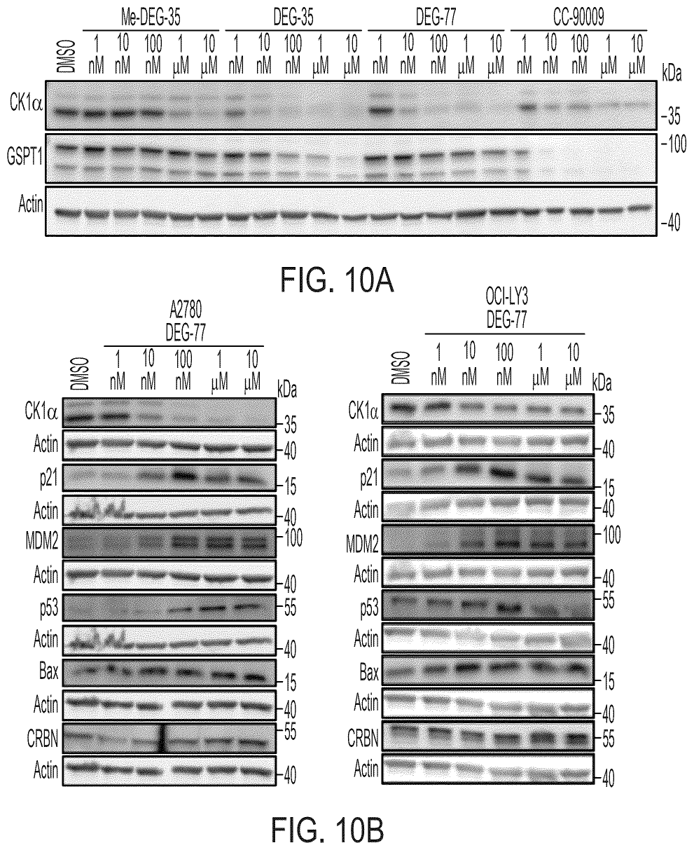

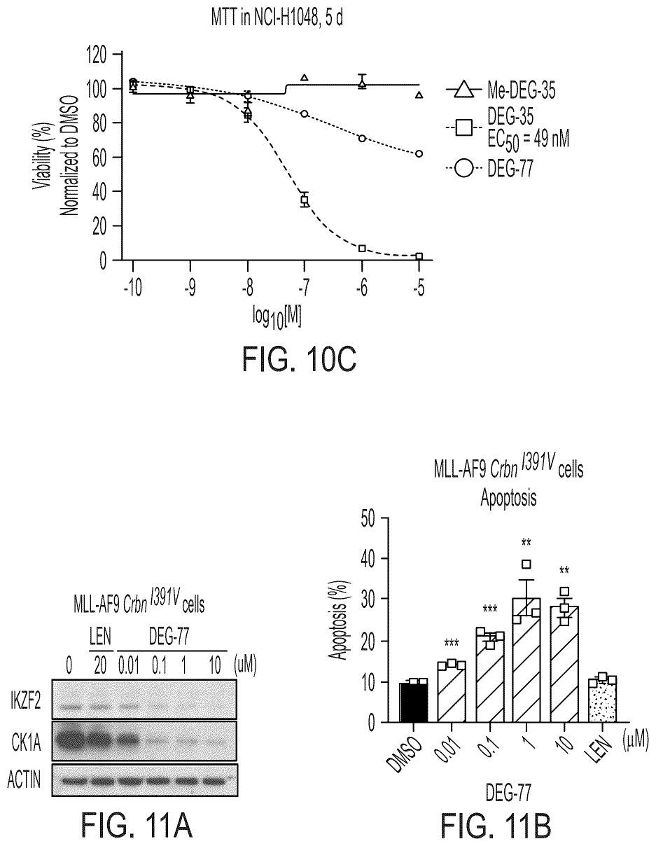

To examine additional cancers that may be sensitive to DEG-77, data generated from evaluating DEG-35 in a PRISM Screen was used. The endometrioid ovarian cancer cell line A2780, small cell lung cancer cell line NCI-H1048, and DLBCL cell line OCI-LY3 were evaluated for sensitivity to DEG-77 and the methylated negative control Me-DEG-35 via MTT assay (FIG. 9A, 9C). A2780 and OCI-LY3 both displayed exquisite sensitivity to DEG-77 with EC₅₀ values of 27 nM and 14 nM, respectively, while the negative control compound Me-DEG-35 showed limited activity. However, surprisingly, NCI-H1048 cells were selectively sensitive to DEG-35 and not DEG-77. Thus, all cell lines were treated with potent GSPT1 degrader CC-90009 to probe if they are sensitive to its degradation (FIG. 9B, 9C). A2780 and OCI-LY3 cells displayed higher EC₅₀ values of 269 nM and 36 nM respectively for CC-90009, suggesting their preferential sensitivity to DEG-35 (EC₅₀ values of 12 nM and 8 nM) and DEG-77 was due to the degradation of CK1α and not GSPT1. In contrast, NCI-H1048 cells were preferentially sensitive to CC-90009 and DEG-35 with EC₅₀ values of 7 nM and 49 nM, respectively. Additionally, when blotting for CK1α and GSPT1 degradation in NCI-H1048 cells treated with DEG-77 and DEG-35, DEG-35 led to significant GSPT1 degradation at nanomolar concentrations while DEG-77 did not affect protein levels (FIG. 10A). This suggests that A2780 and OCI-LY3 cells are sensitive to CK1α degradation while NCI-H1048 cells are not.

CK1α degradation upon DEG-77 treatment was validated by Western blot and demonstrated that GSPT1 is largely spared by DEG-77 but does begin to degrade at higher concentrations of DEG-35 in both cell lines (FIG. 9D). Additionally, p53 activation was demonstrated in both cell lines upon DEG-77 treatment via Western blot and real-time quantitative polymerase chain reaction (RT-qPCR), indicating that CK1α degradation activates p53 in indications beyond AML (FIGS. 9F, 9G, and 10B). Notably, A2780 cells express IKZF2 which was also degraded upon DEG-77 treatment, which may therefore contribute to the overall antiproliferative effects in this cell line (FIG. 9E). Due to apparent p53 activation upon CK1α degradation, the sensitivity of OCI-LY3 and A2780 cells to MDM2 inhibitor idasanutlin (which activates the p53 pathway in cells via a distinct mechanism) was evaluated. A2780 cells had no demonstrable sensitivity and OCI-LY3 had very limited sensitivity to idasanutlin, indicating that MDM2 inhibition is not sufficient to induce apoptosis in these cell lines (FIG. 9B). Collectively, these results demonstrate that DEG-77 has efficacy against ovarian cancer cell line A2780 and DLBCL cell line OCI-LY3 which are both largely insensitive to MDM2 inhibition, suggesting DEG-77 may overcome certain limitations of MDM2 inhibitors.

Compounds have Improved Efficacy Over DEG-35 and can Block Leukemic Stem Cell Activity

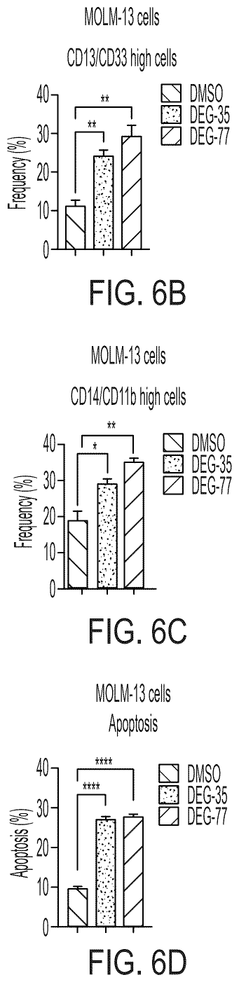

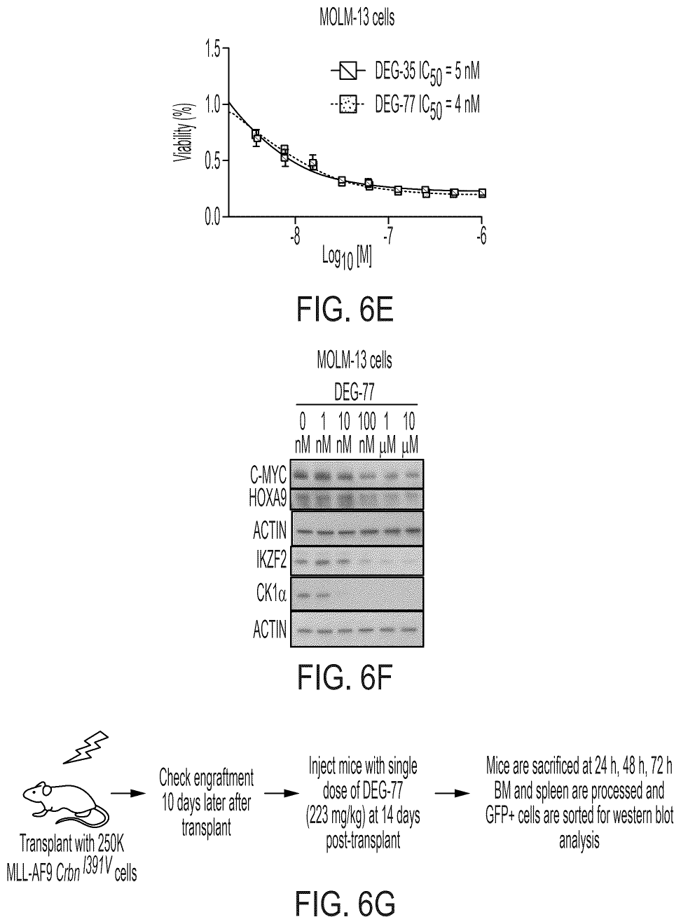

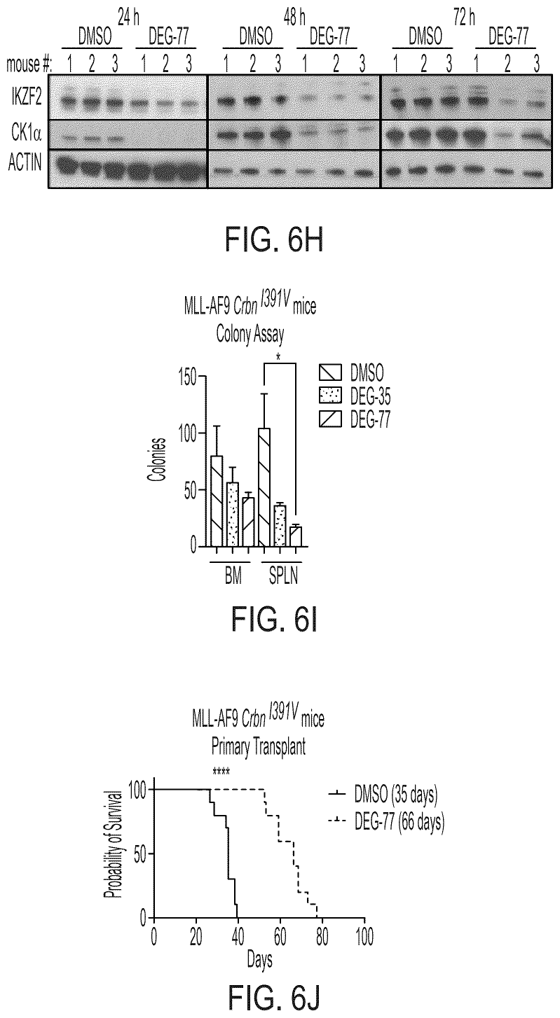

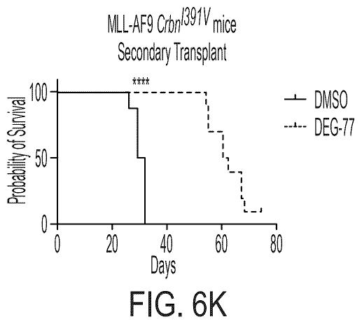

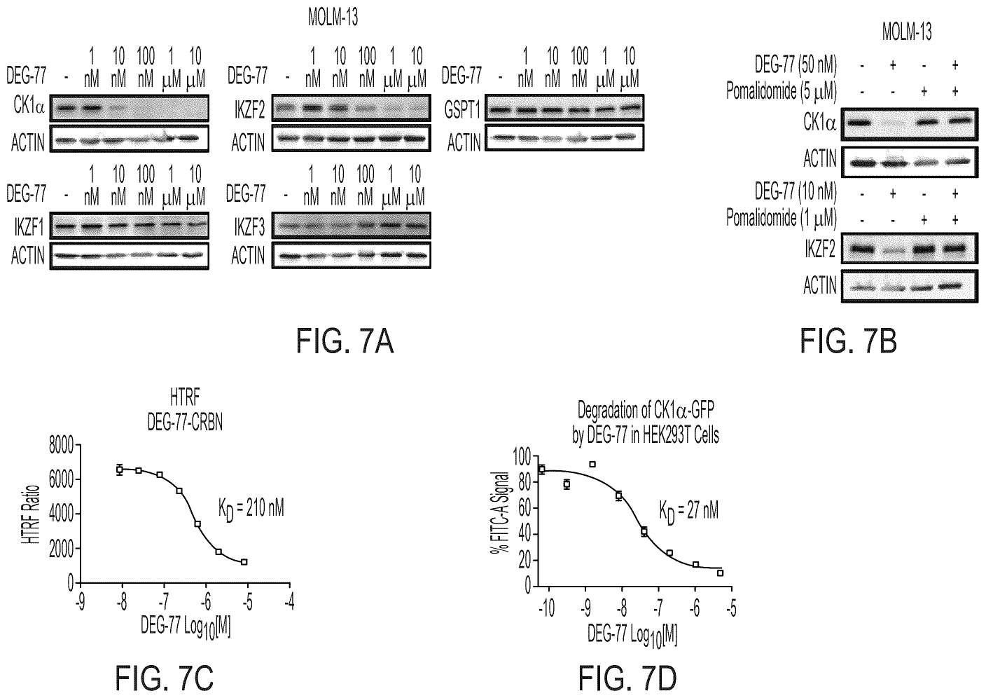

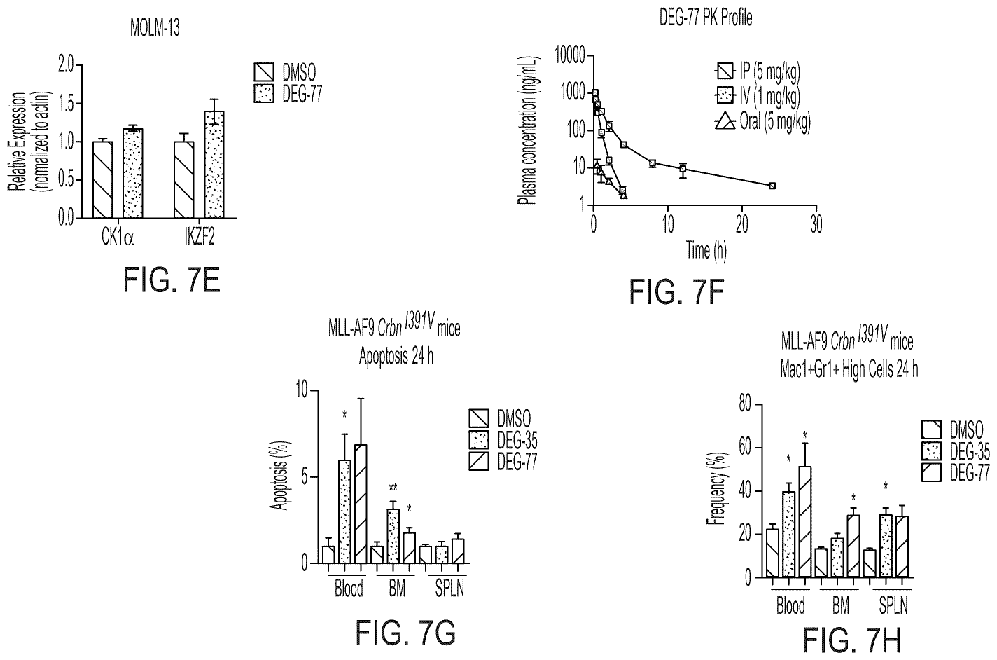

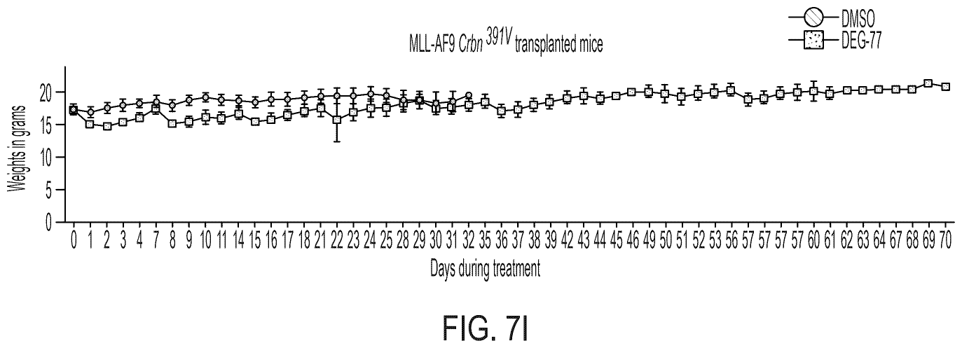

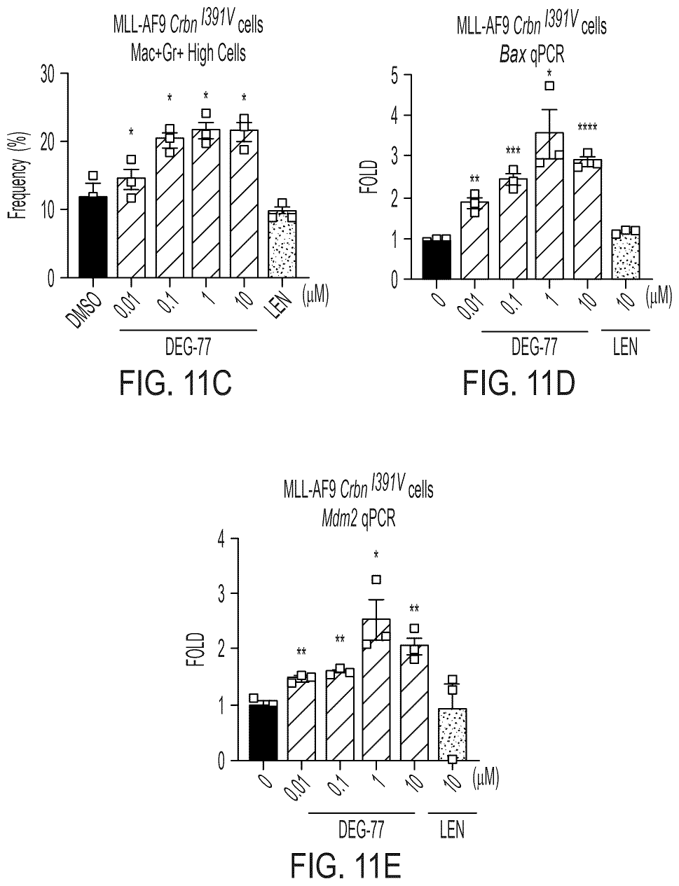

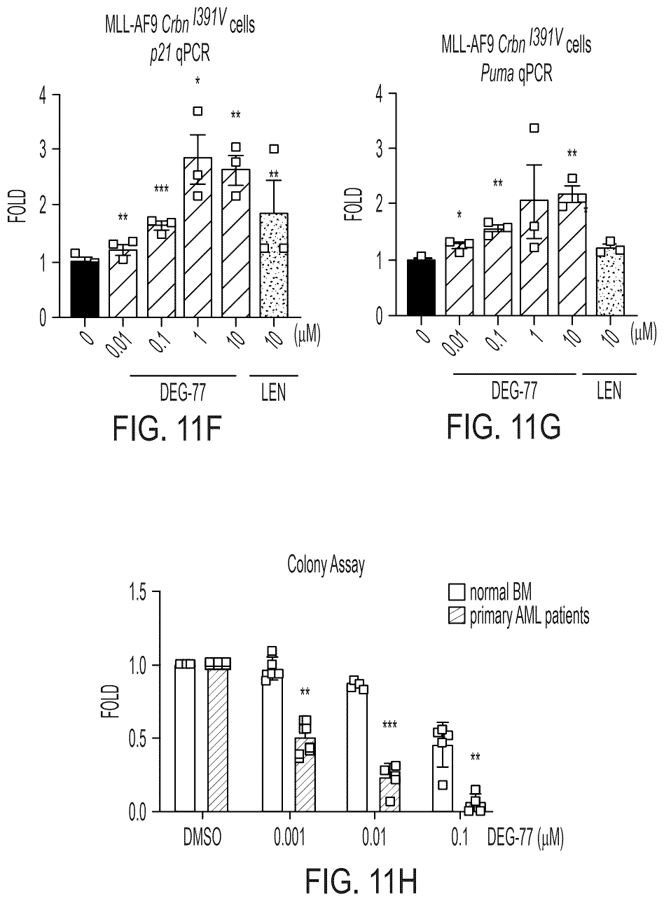

DEG-77 exhibited a 10-fold increase in solubility in DMSO compared to that of DEG-35 and maintained comparable activity against IKZF2 and CK1α in MOLM-13 cells at 24 hr. Similar potency in inducing myeloid differentiation, apoptosis, and cytotoxicity with comparable IC₅₀ values were also observed (IC₅₀ DEG-35=6 nM, IC₅₀ DEG-77=4 nM, FIG. 6A-E). Additionally, DEG-77 had increased selectivity against IKZF2 (DC₅₀=15.3 nM) and CK1α (DC₅₀=10 nM) over other known neosubstrates (e.g., GSPT1, IKZF1, IKZF3, FIG. 7A). Degradation of IKZF2 and CK1α by DEG-77 was CRBN-dependent and occurred post-translationally (FIG. 7B-E). Furthermore, DEG-77 treatment also reduced levels of HOXA9 and c-MYC as observed in DEG-35 treated MOLM-13 cells (FIG. 6F). Furthermore, DEG-77 had similar activity and kinetics as DEG-35 resulting in increased apoptosis, myeloid differentiation, and induction of the TP53 pathway, and comparable preferential sensitivity to leukemia versus healthy cells (FIGS. 11A-H and S7G-L).

To measure its efficacy in vivo, pharmacokinetic and pharmacodynamic assays were performed (FIG. 6G and FIG. 7F). Single treatment of DEG-77 at 223 mg/kg in BL6 mice transplanted with MLL-AF9 Crbn^{1391V} cells led to degradation of IKZF2 and CK1α in bone marrow leukemic cells at 24, 48, and 72 h (FIG. 6H). At the 24 h time point following DEG-77 injection, leukemic cells in blood, bone marrow, and spleen underwent increased apoptosis and myeloid differentiation (FIGS. 7G, 7H). Moreover, sorted leukemic cells from the bone marrow and spleen of DEG-77 treated mice had reduced colony forming ability suggesting reduced in vitro self-renewal (FIG. 6I).

To determine the in vivo efficacy of DEG-77 on survival of leukemic mice, the MLL-AF9 mice were treated with DEG-77 once per week at 223 mg/kg. Interestingly, DEG-77 treated mice had significantly prolonged survival of 66 days compared to 35 days of vehicle treated mice (FIG. 6J) without total body weight loss (FIG. 7I). Importantly, the secondary transplant of DMSO and DEG-77 treated mice maintained a significant survival difference in which DEG-77 mice treated cells in the primary had a pronounced delay in leukemia progression, indicating that DEG-77 can also reduce leukemic stem cell activity (FIG. 6K).

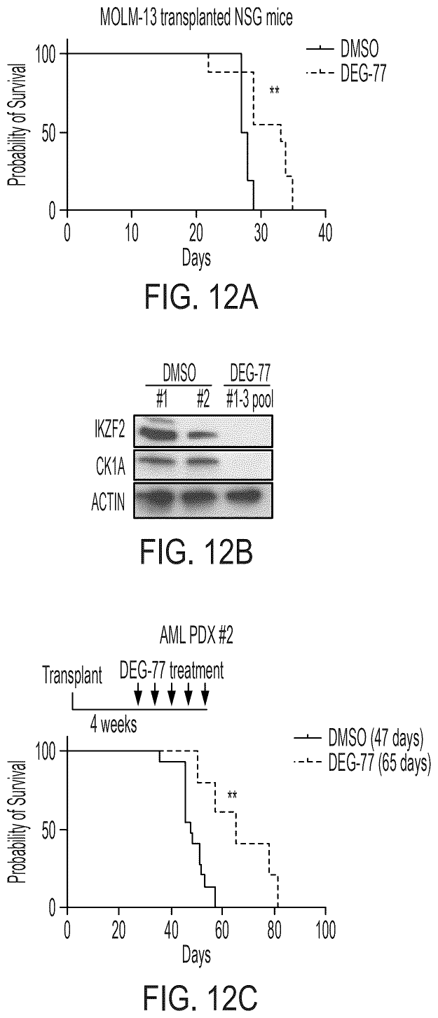

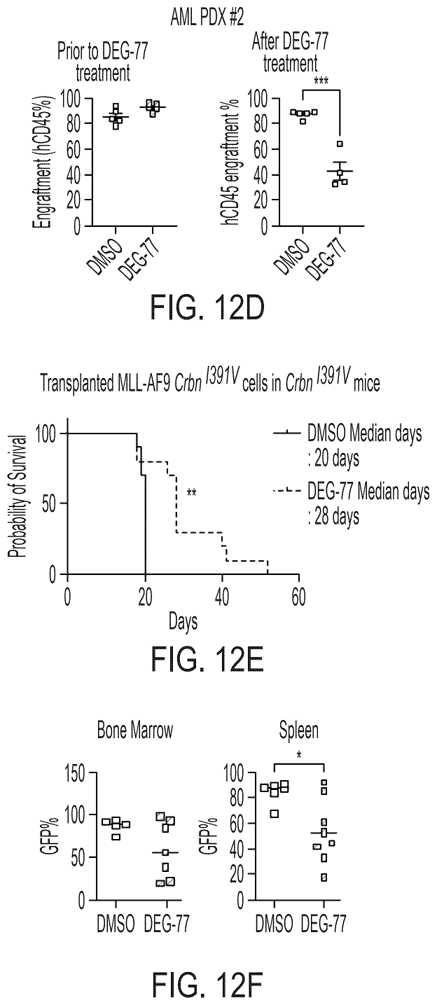

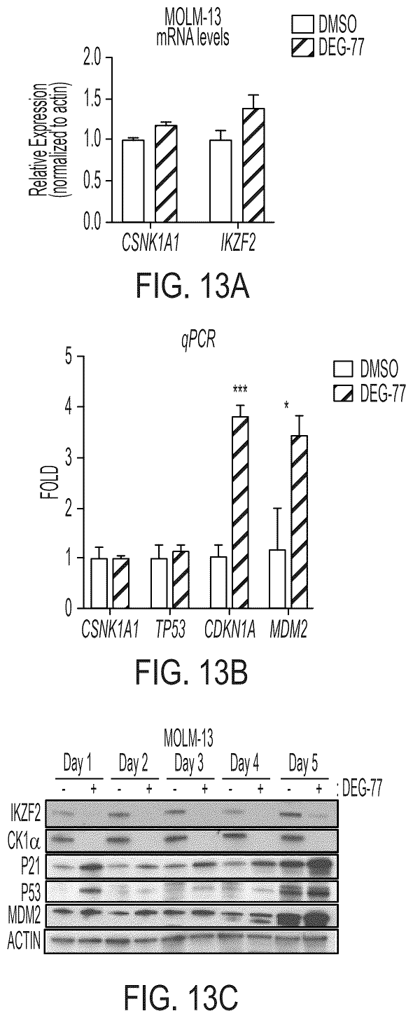

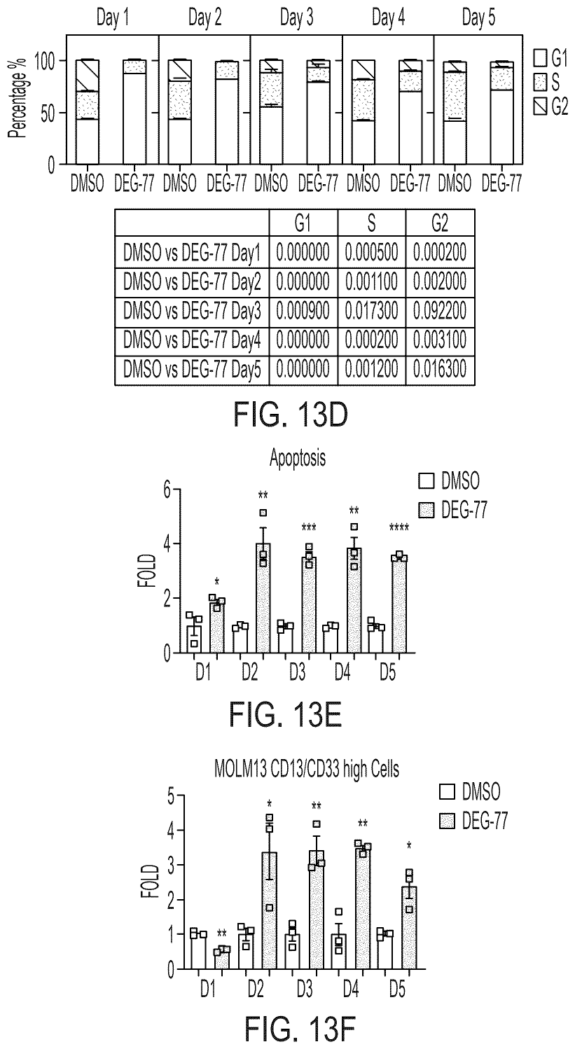

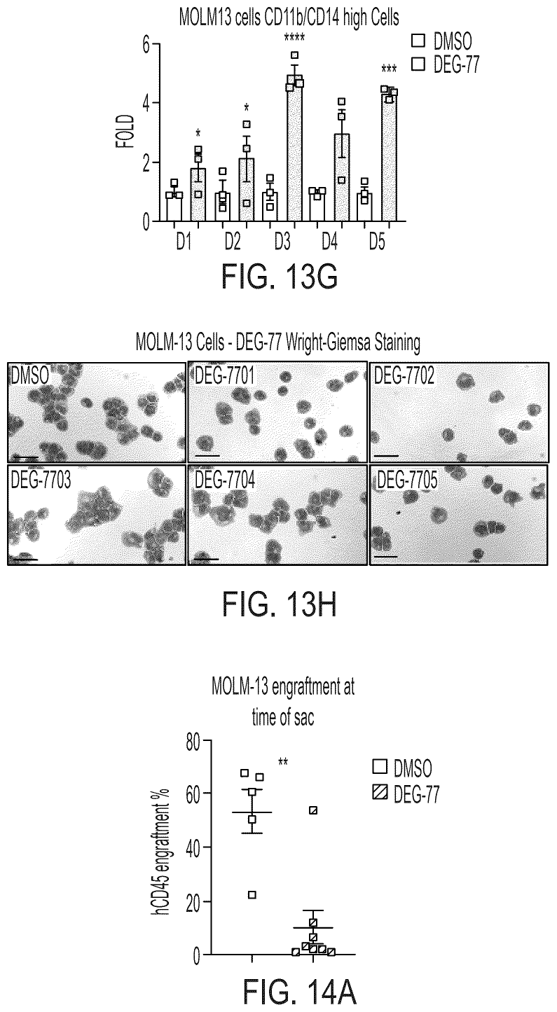

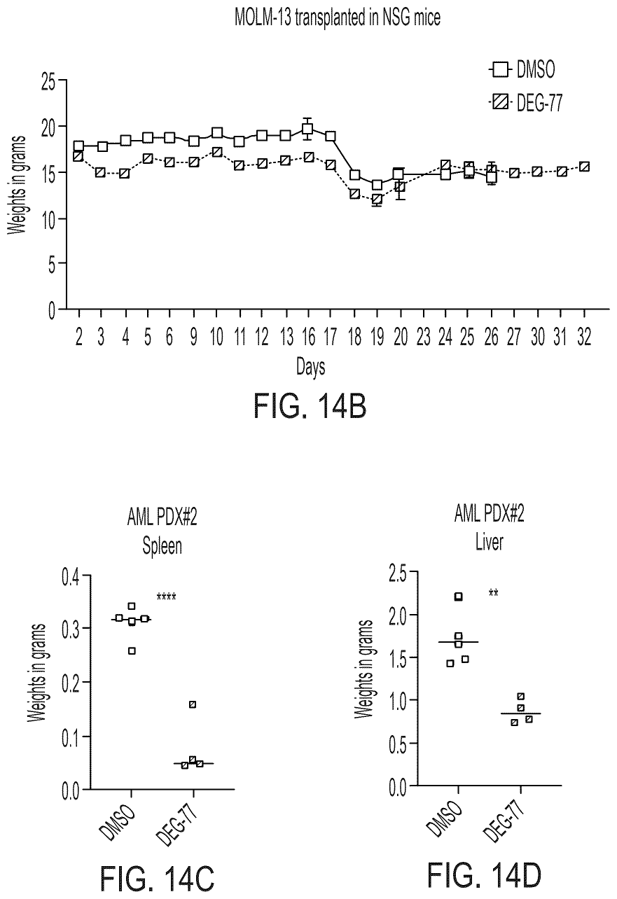

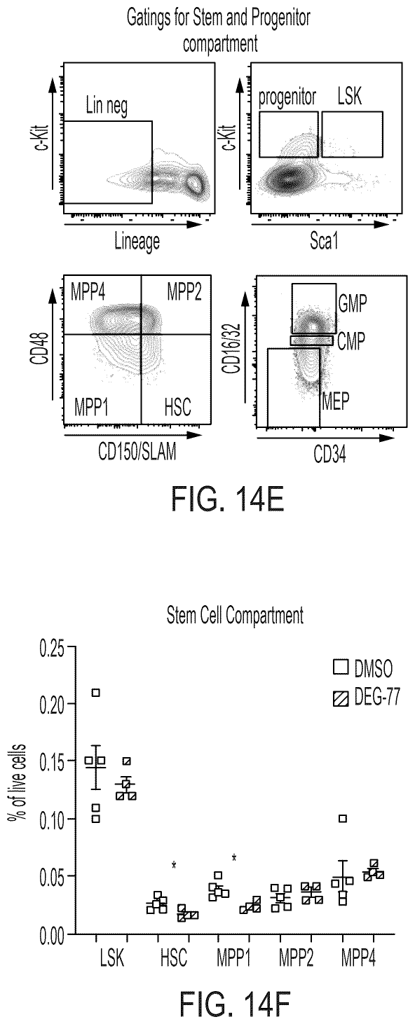

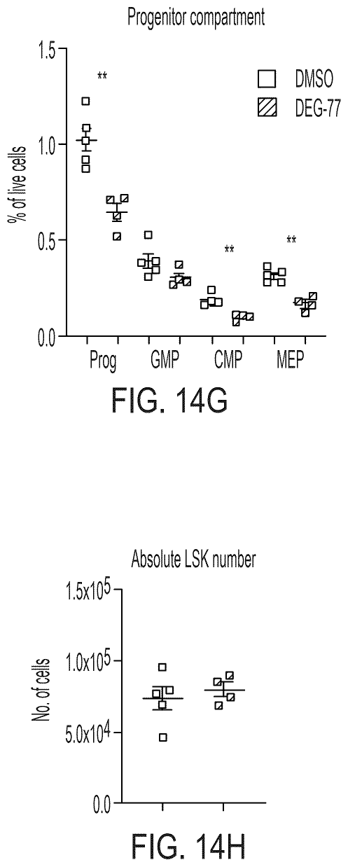

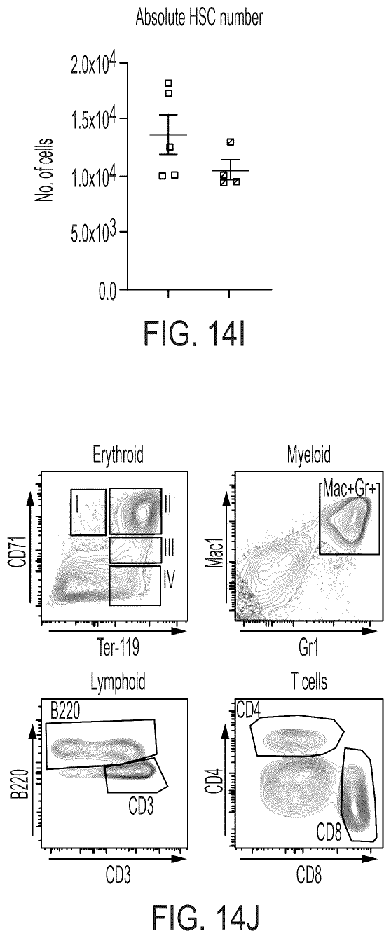

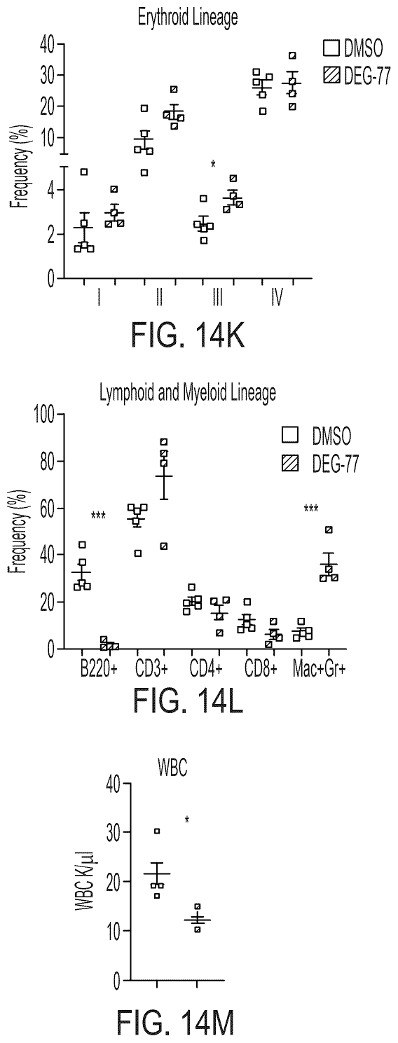

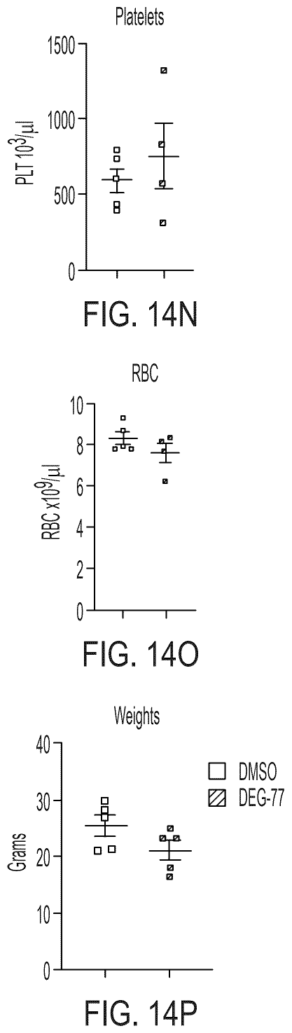

DEG-77 treatment also increased survival and reduced leukemia burden in bone marrow of MOLM-13 transplanted mice without gross weight loss (FIGS. 12A, 14A-B). Both IKZF2 and CK1α were degraded in bone marrow leukemic cells after DEG-77 treatment (FIG. 12B). In the PDX AML model, PDX cells were allowed to engraft for 4 weeks and then treated with DMSO or DEG-77 weekly. This resulted in a significant extension of survival and reduced disease burden for the animals treated with DEG-77 (FIGS. 12C, 14C-D). PDX cells exhibited >85% engraftment prior to DEG-77 treatment in the bone marrow and only around 40% engraftment after a single injection (FIG. 12D) indicating that DEG-77 can reduce leukemic cells in the bone marrow.

Toxicity was determined by testing Crbn^{1391V} mice with four rounds of DMSO or DEG-77 resulting in reduced HSC and MPP1 frequency in the stem cell compartment and CMP and MEP in the progenitor compartment (FIGS. 14E-G). However, we did not observe significant changes in absolute number of LSK and HSC in the DEG-77 treated mice (FIGS. 12H-I). A decrease in B cells and increase in myeloid cells was found which resulted in a modest reduction of WBC counts (FIGS. 12J-M). Importantly, platelets and RBC counts remained unchanged without a major reduction in total body weight (FIGS. 12N-P).

To further test the therapeutic index, a significant increase in survival was found along with reduction of leukemic cell burden in the spleen when DEG-77 was administered to Crbn^{1391V} mice transplanted with MLL-AF9 Crbn^{1391V} cells (FIGS. 12E-F). These data suggest that DEG-77 has efficiency in selectively killing leukemic cells in an in vivo model where all cells are sensitive to the degrader.

Taken together, the disclosure data provides compounds for multi-targeted degradation of IKZF2/CK1α to enhance efficacy against cancer (e.g., AML). In particular, the disclosure reports the design and synthesis of a series of molecular glue degraders that target IKZF2 and CK1α. Through a series of SAR studies, it was found that extension of lenalidomide with a naphthamide from the 5-position of the isooxindole ring promoted IKZF2 and CK1α degradation. It was further found that linkage of the naphthyl group to the isooxindole through an amide greatly increased degradation potency for IKZF2 and CK1α while preventing IKZF1 recruitment, as in DEG-35. The rigid amide bond may introduce additional hydrogen bonds to stabilize the ternary complex with CK1α and also rigidify the structure to adopt a preferential conformation for forming the ternary complex with IKZF2 over IKZF1. Further studies to develop degraders that exhibited the same activity as DEG-35, but with superior solubility and pharmacokinetic properties, were spurred by the finding that the methoxy group of DEG-35 was a metabolic liability. 2H-Chromene analogs of DEG-35 possessed similar degradation profiles, which were also sensitive to substituents and substitution patterns. These analogs showed potent IKZF2 and CK1α degradation, as well as high selectivity for the desired targets over other neosubstrates, namely IKZF1 and GSPT1, which highlights the difficulty in designing a molecular glue that both degrades the target of interest and avoids degradation of undesired neosubstrates. In addition, compounds of the disclosure (e.g., DEG-77) have anti-proliferative effects in cancer types beyond AML.

The development of potent degraders for CK1α additionally suggest that kinases may be a broad class of proteins amenable to degradation by molecular glues that ligand to CRBN.

Multicomponent and combination therapies are a common strategy to treat multigenic diseases and may extend to multi-targeting degraders. Multi-targeting degraders offer the unique opportunity to not only inhibit enzymatic activity but also ablate scaffolding functions of additional proteins. The ability to degrade targets belonging to multiple classes of proteins may broaden the number of pathways that can be targeted with one molecule and may have the potential to act synergistically. Additionally, the use of a single molecule to target multiple proteins may result in reduced toxicity and superior pharmacokinetic and safety profiles.

### Experimental Methods

Culture and transduction of human leukemia cell lines: MOLM-13, OCI-AML5, Kasumi-1, NB4, KG-1A, NOMO-1 and OCI-AML3 cells were cultured in RPMI media (Cell gro) supplemented with 10% FBS, glutamine and penicillin/streptomycin and grown at 37° C. with 5% CO₂. Cells were transduced with viruses expressing empty vector, CK1α wt, CK1α G40N, murine or human IKZF2 wt or IKZF2 mutants by spinning in RPMI with 10% FBS together with viral supernatant containing 10 μg/ml polybrene at 1600 RPM for 1 hr. Media was changed 24 hrs later, and after 72 hrs post transduction cells were sorted for either GFP or RFP and used for experiments.

Transduction of HEK293T cells for fluorescence assays: HEK293T cells were cultured in a 10 cm plate in antibiotic-free DMEM supplemented with 10% FBS and grown to between 70-90% confluence. VSV-G (4 μg), Gag-Pol (6 ug), desired vector (IKZF2-GFP, IKZF1-GFP, GFP-CK1a, 10 ug), and TransitPro (30 μL) were diluted into OptiMEM to a final volume of 1 mL. After tapping to mix and incubation at room temperature (15 min), the DNA/OptiMEM solution was added dropwise to HEK293T cells and swirled gently to mix. Cells were incubated at 37° C. and 5% CO₂ for 5-8 h. Media was replaced with antibiotic-free DMEM supplemented with 10% FBS (10 mL). Cells were incubated for an additional 24-48 h prior to harvesting virus. To harvest, media was collected and centrifuged (500×g, 5 min). The supernatant was removed, passed through a 0.45 μm filter, and aliquoted prior to being snap frozen in liquid nitrogen and stored at −80° C. prior to use. An aliquot of lentivirus (1 mL) was thawed and used to create a 6-well viral dilution plate containing 0, 1:3, 1:5, 1:10, 1:50, and 1:100 viral dilutions in antibiotic-free DMEM supplemented with 10% FBS and polybrene (10 ug/mL) for a final volume of 500 μL per well. 1×105 HEK293T cells in antibiotic-free DMEM (1 mL) supplemented with 10% FBS and polybrene (10 ug/mL) were added to each well and rocked gently to mix. Cells were incubated with virus at 37° C. and 5% CO2 for 48 h. The media was changed and cells were incubated for an additional 24 h. To begin the selection, media was changed to DMEM supplemented with 10% FBS, 10% penicillin/streptomycin, and puromycin (2 μg/mL). Media was periodically refreshed until cells in control well (no virus) appeared dead, generally about 4-5 days after beginning selection. Healthy cells from all wells of virus were harvested and sorted for GFP expression using fluorescence-activated cell sorting (FACS) on the Fortessa flow cytometer at the Harvard Bauer Core and binned into “high”, “medium”, and “low” expression levels as compared to a non-transduced HEK293T cell line. Each cell pool was then evaluated for POI-GFP degradation via Western blot and flow cytometry and subsequently used for experiments.

Site-directed mutagenesis of plasmids: Site-directed mutagenesis of pMSCV-IRES-RFP15-mouse IKZF2, pHFUW-human IKZF2-IRES-GFP and pLC-Flag-CSNK1A1 constructs was performed to obtain the mutants. QuikChange Lightning and Multi Site-Directed Mutagenesis Kits (#210513 and #210518. Agilent Technologies) were used.

Isolation of cord blood-derived CD34+ HSPC cells: Five mixed cord blood units (each unit is from a healthy donor) were used for purifying cord blood CD34+ HSPCs. Mononuclear cells were purified from cord blood by using Hespan and Ficoll-Hypaque Plus density centrifugation, followed by positive selection using the Auto MACS Pro Separator and isolation Kit (Miltenyi).

Culture of primary AML patient cells: Culture of all primary AML patient samples, unless otherwise noted, were collected under the Biospecimen collection/banking study 09-141. The use of the samples for research purposes is covered under the Biospecimen research protocol 16-354. Frozen AML patient cells were thawed in warm RPMI containing 20% FBS and penicillin/streptomycin, washed, counted and cultured in StemSpan SFEMII containing StemSpan CD34+ expansion supplement (Stem Cell tech) at 1×106 cells/ml.

Generation of MLL-AF9 Crbn^{1391V} leukemic cells: Lin-Sca+ Kit+ (LSK) cells were isolated from bone marrow cells of wildtype or Crbn^{1391V} mice that were 6 to 8 weeks old. Briefly, bone marrow cells were enriched for c-Kit positive cells by incubating with CD117/c-Kit microbeads and then processed through the autoMACS Pro Separator (Miltenyi Biotec), following the manufacturer's instructions. CKit enriched cells were stained with Lineage antibody cocktail (CD3, CD4, CD8, Gr1, B220, CD19, TER119 conjugated with PeCy5), Scal-Pac Blue, CD34-FITC, SLAM-APC, CD48-PE, and c-KIT-APC-Cy7. After staining, cells were sorted for LSK cells using a BD FACS Aria II instrument. Sorted LSK cells were incubated overnight in Stemspan SFEM medium (Stem Cell Technologies) with 10 ng/ml IL-3, 10 ng/ml IL-6, 50 ng/ml SCF. 10 ng/ml thrombopoietin (TPO), and 20 ng/ml FLT-3 ligand. Cells were plated on retronectin-coated plates together with supernatant containing retrovirus expressing MLL-AF9 together with GFP (a gift from Scott Armstrong, Dana Farber Cancer Institute) and spun for 1 hr at 2500 RPM. After two cycles of spinfection, the cells were grown in M3434 methylcellulose medium (Stem Cell Technologies) for a week. Cells were sorted for GFP positivity and sorted 200,000 GFP positive cells were injected retro-orbitally with 250,000 bone marrow support cells into lethally irradiated 6-week-old female C57BL/6 mice.

Culture of murine MLL-AF9 leukemic cells: Mouse leukemic bone marrow cells from MLL-AF9 wt and MLL-AF9 Crbn^{1391V} mice were grown in RPMI (Cellgro) medium containing 10% FBS, 6 ng/ml IL-3, 10 ng/ml SCF. 10 ng/ml IL-6 and 10 ng/ml GM-CSF.

Western blot analysis: One million human AML cell lines or murine leukemic cells were counted and treated with drugs at the indicated concentrations for 24 hr. Cells were pelleted, washed with PBS and lysed in Laemmli buffer. Boiled lysates were processed through Qia Shredder and electrophoresed on 4%-15% gradient SDS-PAGE gels. Proteins were transferred onto PVDF membrane and blotted with specified Abs.

1.0×10⁶ MOLM-13 cells were suspended in 1 mL of media and treated with a thousand-fold stock solution in DMSO (1 μL). Cells were incubated at 37° C. for 24 h. Cells were harvested into a microcentrifuge tube and centrifuged (21,130 g) for 2 min. Media was aspirated and cells were washed with PBS (2×1 mL). Cells were then lysed in Laemmeli buffer (150 μL) by vigorous vortexing for 10 min. Lysates were boiled at 95° C. for 5 min. Lysates were then run on a 4%-15% gradient SDS-PAGE gel.

2.0×10⁶ A2780 and TOV-21G cells were suspended in 2 mL of media and treated with a thousand-fold stock solution in DMSO (2 μL). Cells were incubated at 37° C. for 24 h. Media was harvested into a microcentrifuge tube and centrifuged (21,130 g) for 3 min. Media was aspirated. Cells were trypsinized with 750 μL of 0.25% trypsin for 3 min. Trypsin was quenched with 750 μL of media. Media was harvested into the microcentrifuge tubes and centrifuged (21,130 g) for 3 min. Media was aspirated and cells were washed with PBS (2×1 mL). Cells were then lysed in Laemmeli buffer (200 μL) by vigorous vortexing for 20 min. Lysates were boiled at 95° C. for 10 min. Lysates were then run on a 4%-15% gradient SDS-PAGE gel.

Viability Assay: 10,000 cells (e.g., MOLM-13 cells, MLL-AF9 leukemic cells or normal BM cells) were plated onto flat-bottom 96-well plates in media with increasing concentration of compound up to 100 M. Cells were cultured for 72 h at 37° C. in a 5% CO₂ incubator. For measuring viability, Cell-Titer Glo kit (Promega) or MTT kit was used following manufacturer's instructions. For Cell-Titer Glo assay, cells were cooled to room temperature for 20-30 min, 100 μL of the cultured cells were transferred to opaque-white bottom 96-well plates and mixed with 100 μL of Cell-Titer Glo reagent. The mixture was incubated for 15 min at room temperature and read using a Synergy H1 Hybrid reader (BioTek) for luminescence. Data was normalized as percentage viability and graphed by non-linear regression curves in Graph Pad PRISM 7.0.

Flow cytometry for apoptosis and differentiation: For measuring differentiation, MOLM-13 cells treated with compound for 48 hrs were stained with anti-CD13-FITC, anti-CD33-APC, anti-CD14-FITC or anti-CD11b/Mac1-APC. For murine MLL-AF9 leukemic cells, cells treated with compound for 24 hrs were stained with anti-Mac1-PB, anti-Ly-6G (Gr1)-PE, anti-CD115-APC and anti-F4/80-Pe-Cy7. Samples were stained with 2% FBS RPMI with diluted antibodies for 20 min in the dark, washed and analyzed with LSRII or LSR Fortessa (BD Biosciences) and data was graphed by using FlowJo™ version 10.4. For apoptosis assessment, MOLM-13 cells treated with compound for 48 hrs or MLL-AF9 cells treated with compound for 24 hrs, were washed with cold PBS and incubated with anti-Annexin V-APC (BD Biosciences) in the Annexin-V binding buffer (10 mM HEPES, pH 7.4, 140 mM NaCl, 4 mM KCl, 0.75 mM MgCl₂, 1 mM CaCl₂)) together with DAPI in the reaction volume of 100 mL for 15 minutes as recommended by the manufacturer.

Flow cytometry for measuring degradation: 4.5×10⁴ HEK293T GFP-POI (CK1α, GSPT1, IKZF1, IKZF2) cells were seeded in a U-bottom 96-well plate in 200 μL of media and allowed to adhere for 2 h at 37° C. and 5% CO₂. Cells were treated with 2 μL of a one-hundred-fold stock solution of compound in DMSO and incubated at 37° C. and 5% CO₂ for 24 h. The media was aspirated and 100 AL of a 50/50 0.5% trypsin-EDTA/PBS solution were added to each well and incubated at 37° C. for 4 min. Media (100 μL) and propidium iodide/PBS (0.5 mg/mL, 4 μL) were added to each well and cells were resuspended via pipetting prior to analysis with a BD LSR Fortessa flow cytometer at the Harvard University Bauer Core Facility. Raw FITC sample averages were exported to Excel and normalized to the DMSO treated average representing 100% fluorescence. These values were graphed using GraphPad Prism 7.0 and the nonlinear regression curve fit model was used to generate best-fit curves, error bars, Dₘₐₓ and DC₅₀ values.

Cell Cycle Analysis: For cell cycle analysis, MOLM-13 cells treated with 1 μM DEG-35 or DEG-77 were washed with PBS and fixed in ice-cold 70% ethanol for few hours. Fixed cells were pelleted and washed twice with PBS and incubated in PBS containing 3.8 mM Sodium Citrate, propidium iodide (50 ug/ml) and RNase A (100 ug/ml) for 30 min at 4° C. in the dark. Nuclei were then analyzed by running at low flow rate in LSRII for 10,000 events.

Wright-Giemsa Staining: 50,000-100,000 MOLM-13 cells were cytospun onto poly-lysine coated glass slides. Slides were then processed for Wright-Giemsa Staining by dipping slides into fixative, Solution A and Solution B of the Three-Step Stain Kit (Richard-Allan Scientific) for 30 seconds each. Dried slides were mounted with cover slides.

Colony forming Unit Assay: Colony Assay for human CD34+ HSPCs, normal bone marrow cells or AML patient cells were performed by plating 5000 cells on methylcellulose (MethoCult H4434 Classic-Stem Cell Technologies). Colonies were assessed and counted 10 to 14 days after seeding. 500 sorted murine LSK cells from Wt or Crbn^{1391V} mice and 1000 bone marrow cells from MLL-AF9 Wt or Crbn^{1391V} mice were plated on MethoCult M3434 and counted 5-10 days after plating.

Mass spectrometry data analysis: Analysis was performed in Thermo Scientific Proteome Discoverer version 2.4.1.15. The raw data was searched against a protein sequence database including all entries from the Human Uniprot database (Aug. 19, 2016; 20,156 entries, SwissProt) and a list of common contaminant proteins. Search parameters included a 10-ppm precursor ion tolerance and 0.02 Da (HCD) or 0.6 Da (CID) fragment ion tolerance, full tryptic protease specificity with up to two missed cleavages, a static modification of TMTpro 16-plex at lysine residues and N-termini (+304.2071 Da), static carboxyamidomethylation of cysteine residues (+57.0214 Da), and variable oxidation on methionine residues (+15.9949 Da). Peptide spectral matches (PSMs) were filtered for a false discovery rate (FDR) of 1% using Percolator. TMT reporter ions were quantified using the Reporter Ions Quantifier node and the total intensity was normalized for each TMT channel. Quantified proteins were required to have at least three unique peptides and four separate PSMs. All TMT-based experiments were performed with four replicates.

In cellulo co-immunoprecipitation: HEK-293T cells stably transfected with Flag-tagged CRBN were plated in a 10 cm dish and allowed to grow to full confluency. Cells were treated with 10 μM MG132 and incubated for 1 h at 37° C. The cells were then treated with either 10 μM DEG35 or DMSO and incubated for 3 h at 37° C. Cells were washed with PBS (2×10 mL) and Cell Lysis Buffer (CST, 400 μL) containing MG132 (10 μM) and 1× protease inhibitors was added to each plate. The cells were incubated for 5 min on ice and then harvested by scraping. Lysate was incubated on ice for 5 min and then cleared by centrifugation (21,130×g) for 10 min at 4° C. Solubilized protein concentration was determined by BCA assay and adjusted to 1 mg/mL in lysis buffer. Adjusted lysate (200 μL) was added to anti-Flag M2 magnetic beads (30 μL) previously washed with TBS (3× 300 μL) and incubated for 1 h at 4° C. The supernatant was removed, and the beads were washed with lysis buffer (3×300 μL). 1× Laemmli sample buffer (50 μL) was added to the beads and boiled for 5 min at 95° C. Results were analyzed by Western blot.

HTR-FRET: PerkinElmer's Cereblon binding assay kit was used. A series of 4-fold serial dilutions of lenalidomide, DEG-35, and DEG-77 in 4% DMSO/dilution buffer were prepared fresh for each experiment. Compound (5 μL) was added to each well of a 384-well microplate. A solution of CRBN/1× PROTAC binding buffer (5 μL) and a solution of premixed thalidomide red glutathione-europium cryptate antibody/1× PROTAC binding buffer (10 μL) were added to each well. The plate was sealed with TopSeal-A PLUS and incubated at 25° C. for 3 h. The seal was removed, and the plate was analyzed.

In vivo drug administration in MLL-AF9 and PDX mouse model: A total of 250,000 of MLL-AF9 BM secondary mouse leukemia cells were injected retroorbitally into 6-8 weeks-old female C57BL/6 recipient mice that has been sublethally irradiated at 450 cGy. For AML PDX model, 1 million PDX cells were transplanted by intravenous injection into 8-10 weeks old female NSG mice that had been sublethally irradiated at 200 rads 24 hr before transplant. For both models, 3 days after transplant, DEG-35 or vehicle was administrated by intraperitoneal injections at 50 mg/kg in DMSO in 50 ul volume for in vivo long-term studies. DEG-77 was treated at 223 mg/kg in DMSO in 50 ul volume. Mice weight was monitored every day to check for toxicity. Mice were sacrificed when moribund, and organ weights were measured for disease burden. Spleen and bone marrow cells were isolated and processed for western blot analysis. All animal studies were performed on animal protocols approved by the Institutional Animal Care and Use Committee (IACUC) at Memorial Sloan Kettering Cancer Center. In the case for pharmacodynamic experiments, DEG-35 or DEG-77 were administrated 2 weeks post-transplant of MLL-AF9 BM secondary mouse leukemia cells in C57BL/6 recipient mice. DEG-35, DEG-77 or DMSO was administrated by intraperitoneal injections at 50 mg/kg or 223 mg/kg. After single dose treatment of DEG-35 or DEG-77, mice were sacrificed at either 24 hr, 48 hr or 72 hr later and, spleen and bone marrow cells were processed for analysis. For Pharmacokinetics experiments, a single dose of DEG-35 at 5 mg/kg or 50 mg/kg and DEG-77 at 5 mg/kg in DMSO were administered intraperitoneally into male CD1 mice. Plasma was extracted from each mouse after indicated time and DEG-35 in plasma was measured using HPLC. For examining the toxicity of DEG-77 in the hematopoietic system of Crbn^{1391V} mice, four rounds of DMSO or 223 mg/kg DEG-77 was treated in 8-12 weeks old female Crbn^{1391V} mice. Mice were injected once a week and were sacrificed 4 weeks later for blood analysis using the Element HT5 Hematology Analyzer (HESKA) and analysis of the hematopoietic cells by flow cytometry.

Reverse-transcriptase quantitative polymerase chain reaction (RT-qPCR): A2780 or TOV-21G cells were seeded in a 6-well plate at a density of 2.0×10⁶ cells/well in 2 mL media and allowed to adhere for 2 h at 37° C. and 5% CO₂. Cells were treated with 2 μL of a one-thousand-fold stock solution of D77 in DMSO to a final concentration of 1 μM or DMSO and incubated for 24 h at 37° C. and 5% CO₂. Media was harvested into a microcentrifuge tube and centrifuged (21,130 g) for 3 min. Media was aspirated. Cells were trypsinized with 750 μL of 0.25% trypsin for 3 min. Trypsin was quenched with 750 μL of media. Media was harvested into the microcentrifuge tubes and centrifuged (21,130 g) for 3 min. Media was aspirated and cells were washed with PBS (2×1 mL). The Monarch Total RNA Miniprep Kit was used to isolate and purify RNA from the cells. Resulting RNA extracts were diluted to 56.8 ng/μL and the NEB Luna Universal One-Step RT-qPCR Kit was used to prepare the samples for RT-qPCR. 20 μL of combined RNA/enzyme/primer mixes were aliquoted into a 96-well plate and centrifuged (1000×g) for 1 min at room temperature. Samples were run in biological triplicate. RT-qPCR was performed in a BioRad iQ5 Real-Time PCR Detection System and results were exported to Microsoft Excel for analysis following the ΔΔCt model (Livak K. J. and Schmittgen T. D.). Processed gene expression values were then graphed using GraphPad Prism 7.0.

Quantitative global proteomics: MOLM-13 cells were seeded at 2.3×10⁶ cells in 1 mL of RPMI. Cells were treated with 50 nM DEG-77 or DMSO (5 replicates each) and incubated at 37° C. for 2 h. Cells were harvested and pelleted by centrifugation (500×g, 2 min) at 24° C. and washed with PBS (2×1 mL). The washed cells were suspended in 5% SDS/50 mM TEAB (110 μL) containing protease inhibitor cocktail and sonicated on ice using a probe tip sonicator (11% amplitude, 5 s on, 3 s off, 30 s total). The sonicated lysates were cleared by centrifugation (13,000×g, 10 min) at 4° C. The soluble protein concentration was determined by BCA assay and adjusted to 1.1 mg/mL with lysis buffer. Lysates (100 μL) were reduced with dithiothreitol (11 μL, 50 mM, final concentration 5 mM) at 24° C. for 30 min and subsequently alkylated with iodoacetamide (9 μL, 200 mM, final concentration 15 mM) at 24° C. for 30 min in the dark. The samples were acidified with 12% phosphoric acid (12 μL) and diluted with S-trap buffer (90% methanol, 100 mM TEAB, pH 7.55, 720 μL). The diluted samples were transferred to an S-trap micro attached to a vacuum manifold and the supernatant was drained. The samples were washed with S-trap buffer (3×800 μL) and dried by centrifugation (4000×g, 1 min) at 24° C. Sequencing-grade trypsin (11 μg) in 50 mM TEAB (125 μL) was added to each S-trap and the samples were incSubated at 37° C. for 16 h. 50 mM TEAB (80 μL) was added to each sample and incubated at 24° C. for 30 min. The S-traps were centrifuged (4000×g, 1 min) at 24° C. and the digested peptides were sequentially eluted with 0.2% formic acid/water (80 μL) and 50% acetonitrile/water (80 μL). The combined eluents were combined and dried by vacufuge. The dried peptides were resuspended in water (25 μL) and TMT 10-plex reagent (10 μL) was added and incubated at 24° C. for 1 h. To quench excess TMT reagent, 5% hydroxylamine (1.2 μL) was added and incubated at 24° C. for 15 min. The labeled samples were combined and dried by vacufuge. The dried sample was resuspended in 0.1% TFA/water (300 μL), loaded on to a Pierce peptide desalting spin column (catalog #89851), desalted according to manufacturer instructions, and dried by vacufuge.

The desalted peptides were resuspended in 0.1% TFA/water (450 μL) and fractionated by high pH reverse-phase HPLC. The sample was injected onto an Aeris 3.6 μm Peptide XB-C18 column with mobile phase A containing 10 mM NH₄HCO₃ in 5% acetonitrile/water, pH 8.0 and mobile phase B containing 10 mM NH₄HCO₃ in 90% acetonitrile water, pH 8.0. The sample was run through the column over 60 min with a flow rate of 1 mL/min with the following gradient: 1% B for 4 min, 35% B in 50 min, 60% B in 4 min, 80% B in 2 min. 0.7 mL fractions were collected between 4 min and 60 min. The fractions were dried, resuspended in 0.1% TFA/water (50 μL) and combined non-continuously into 20 samples and dried by vacufuge. The samples were then resuspended in 0.1% TFA/water (40 μL) for injection on to the mass spectrometer.

Proteomics mass spectrometry acquisition procedures for protein level quantification: The LC-MS/MS experiment was performed on an Orbitrap Eclipse Tribrid Mass Spectrometer coupled with a Vanquish Neo HPLC system (Thermo scientific) at the Harvard Center for Mass Spectrometry. The TMT labelled peptides were first trapped on a trapping cartridge (300 μm×5 mm PepMap™ Neo C18 Trap Cartridge, Thermo scientific) prior to separation on a silica-chip-based micropillar column (μPAC, C18 pillar surface, 50 cm bed, Thermo scientific). The column oven temperature was maintained at 35° C. Peptides were eluted using a multi-step gradient at a flow rate of 300 nL/min over 180 min. The mobile phase A and weak wash liquid was water with 0.1% formic acid (FA), and mobile phase B was acetonitrile with 0.1% FA, and strong wash liquid was 80% acetonitrile with 0.1% FA. The mobile phase gradient consisted of a linear 125 min gradient from 2% to 20% mobile phase B, followed by a 24 min increase to 35% B, a further 10-min increase to 95% B, a 20 min plateau phase at 95% B. The autosampler temperature was 7° C. The column “Fast equilibration” was enabled. The Orbitrap Eclipse MS was operated in DDA mode with 3 s cycle time. The electrospray ionization was in positive mode with voltage of 2.1 kV and the capillary temperature at 275° C. Dynamic exclusion was enabled with a mass tolerance of 10 ppm and exclusion duration of 60 s. Full scan was performed in the range of 400-1,600 m/z at a resolution of 120,000, RF lens 30%, normalized AGC target 200% and maximum injection time set to auto. Precursors were isolated in a window of 0.7 m/z and charge states from two to six. Fragmentation was performed by HCD using normalized collision energy (NCE) of 38% at the resolution of 50,000. The normalized AGC target was set to 250%, and a maximum ion injection time set to 200 ms.

QUANTIFICATION AND STATISTICAL ANALYSIS: Statistical parameters for each data are reported in Figure descriptions. The 2-tailed Student's t test was used for significance testing in the bar graphs, except where stated otherwise. P values less than 0.05 were considered significant. Error bars reflect the SEM, except where stated otherwise. Survival probabilities were estimated using the Kaplan-Meier method and compared with the log-rank test. All statistical analyses were carried out using GraphPad Prism 7.0 and the R statistical environment.

### EQUIVALENTS AND SCOPE

In the claims articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or all of the group members are present in, employed in, or otherwise relevant to a given product or process.

Furthermore, the invention encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group. It should it be understood that, in general, where the invention, or aspects of the invention, is/are referred to as comprising particular elements and/or features, certain embodiments of the invention or aspects of the invention consist, or consist essentially of, such elements and/or features. For purposes of simplicity, those embodiments have not been specifically set forth in haec verba herein. It is also noted that the terms “comprising” and “containing” are intended to be open and permits the inclusion of additional elements or steps. Where ranges are given, endpoints are included. Furthermore, unless otherwise indicated or otherwise evident from the context and understanding of one of ordinary skill in the art, values that are expressed as ranges can assume any specific value or sub-range within the stated ranges in different embodiments of the invention, to the tenth of the unit of the lower limit of the range, unless the context clearly dictates otherwise.

This application refers to various issued patents, published patent applications, journal articles, and other publications, all of which are incorporated herein by reference. If there is a conflict between any of the incorporated references and the instant specification, the specification shall control. In addition, any particular embodiment of the present invention that falls within the prior art may be explicitly excluded from any one or more of the claims. Because such embodiments are deemed to be known to one of ordinary skill in the art, they may be excluded even if the exclusion is not set forth explicitly herein. Any particular embodiment of the invention can be excluded from any claim, for any reason, whether or not related to the existence of prior art.

Those skilled in the art will recognize or be able to ascertain using no more than routine experimentation many equivalents to the specific embodiments described herein. The scope of the present embodiments described herein is not intended to be limited to the above Description, but rather is as set forth in the appended claims. Those of ordinary skill in the art will appreciate that various changes and modifications to this description may be made without departing from the spirit or scope of the present invention, as defined in the following claims.

What is claimed is:

1. A compound of Formula (I): 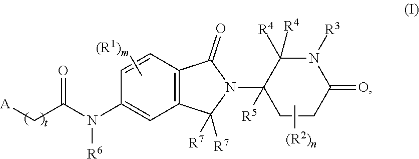
or a pharmaceutically acceptable salt thereof, wherein:
each R¹ is independently halogen, —OR^{A}, or substituted or unsubstituted C₁-C₆ alkyl;
each R² is independently halogen or C₁-C₃ alkyl;
R³ is hydrogen or C₁-C₃ alkyl;
each R⁴ is independently hydrogen or C₁-C₃ alkyl; or two R⁴, together with the carbon atom to which they are attached, form a C═O, C₃-C₆ carbocycle, or a 4-6-membered heterocycle;
R⁵ is hydrogen, halogen, or C₁-C₃ alkyl;
R⁶ is hydrogen, substituted or unsubstituted acyl, substituted or unsubstituted alkyl, or a nitrogen protecting group;
each R⁷ is independently hydrogen or C₁-C₃ alkyl; or each R⁷, together with the carbon atom to which they are attached, form a C═O;
A is 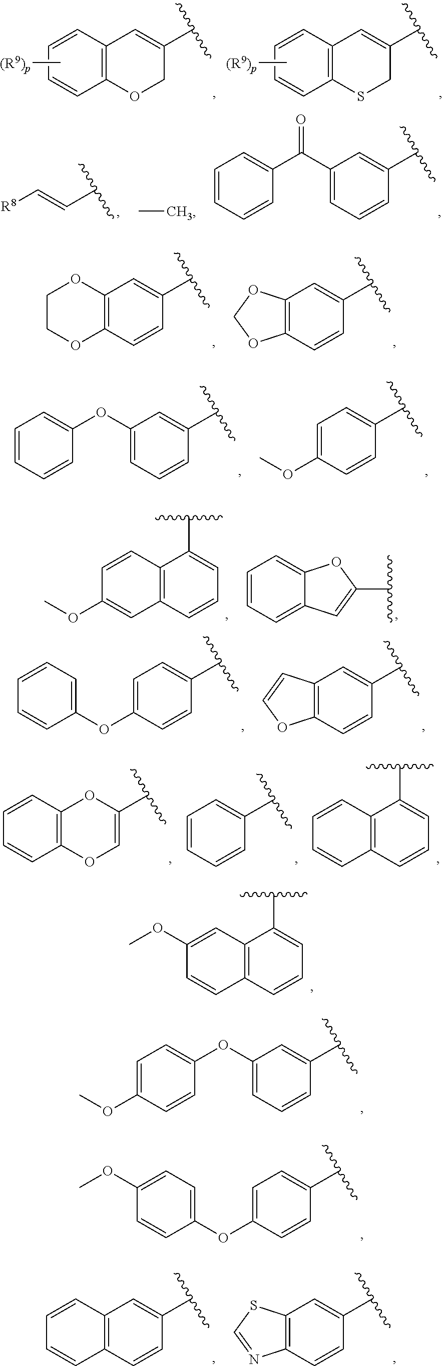 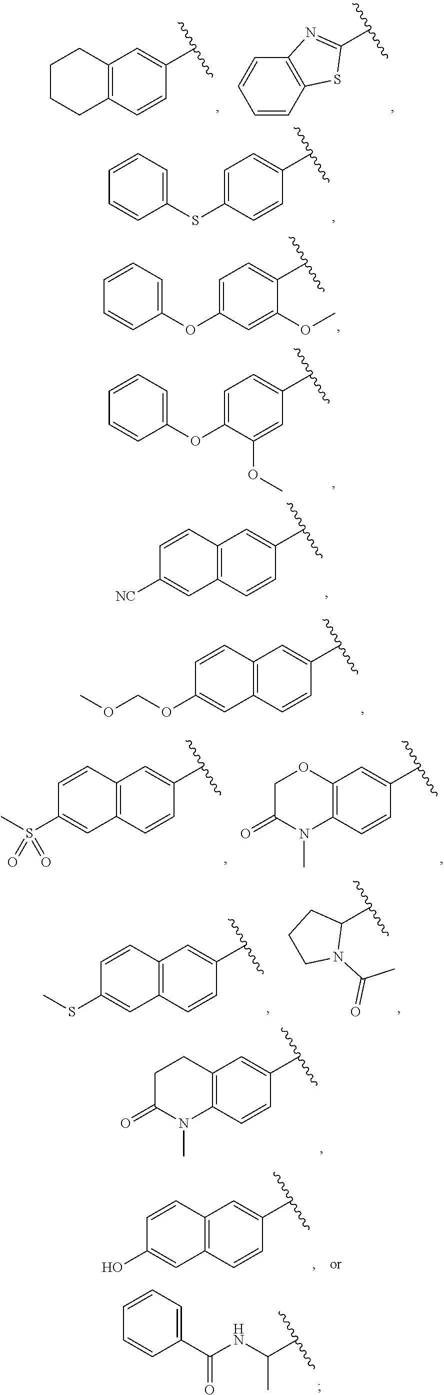
R⁸ is hydrogen, substituted or unsubstituted alkyl, substituted or unsubstituted heteroalkyl, substituted or unsubstituted carbocyclyl, substituted or unsubstituted heterocyclyl, substituted or unsubstituted aryl, or substituted or unsubstituted heteroaryl;
each R⁹ is independently halogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, substituted or unsubstituted heteroaliphatic, —OR^{A}, —N(R^{A})₂, —SR^{A}, —CN, —SCN, —C(═NR^{A})R^{A}, —C(═NR^{A})OR^{A}, —C(═NR^{A})N(R^{A})₂, —C(═O)R^{A}, —C(═O)OR^{A}, —C(═O)N(R^{A})₂, —NO₂, —NR—C(═O)R^{A}, —NR^{A}C(═O)OR^{A}, —NR^{A}C(═O)N(R^{A})₂, —NR^{A}C(═NR^{A})N(R^{A})₂, —OC(═O)R^{A}, —OC(═O)OR^{A}, —OC(═O)N(R^{A})₂, —NR^{A}S(O)₂R^{A}, —OS(O)₂R^{A}, or —S(O)₂R^{A}; or two R⁹ groups are joined to form a substituted or unsubstituted heterocyclyl ring, or a substituted or unsubstituted carbocyclyl ring;
each R^{A} is independently hydrogen, substituted or unsubstituted acyl, substituted or unsubstituted alkyl, substituted or unsubstituted alkenyl, substituted or unsubstituted alkynyl, substituted or unsubstituted heteroaliphatic, substituted or unsubstituted carbocyclyl, substituted or unsubstituted carbocyclylalkyl, substituted or unsubstituted heterocyclyl, substituted or unsubstituted aryl, substituted or unsubstituted aralkyl, substituted or unsubstituted heteroaryl, substituted or unsubstituted hetaralkyl, a nitrogen protecting group when attached to a nitrogen atom, an oxygen protecting group when attached to an oxygen atom, or a sulfur protecting group when attached to a sulfur atom, or two R^{A} groups are joined to form a substituted or unsubstituted heterocyclyl ring, or a substituted or unsubstituted heteroaryl ring;
m is 0, 1, 2 or 3;
n is 0, 1, or 2;
t is 0, 1, or 2; and
p is 0, 1, 2, 3, or 4;
provided that the compound is not of formula:

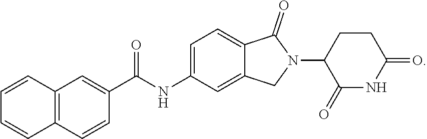 [SMILES: O=C1CCC(N2Cc3cc(NC(=O)c4ccc5ccccc5c4)ccc3C2=O)C(=O)N1]

2. The compound of claim 1, or a pharmaceutically acceptable salt thereof, wherein:
m is 0.

3. The compound of claim 1 or 2, or a pharmaceutically acceptable salt thereof, wherein:
n is 0.

4. The compound of any of claims 1-3, or a pharmaceutically acceptable salt thereof, wherein:
R³ is hydrogen.

5. The compound of any of claims 1-4, or a pharmaceutically acceptable salt thereof, wherein:
each R⁷ is hydrogen.

6. The compound of any of claims 1-4, or a pharmaceutically acceptable salt thereof, wherein:
each R⁷, together with the carbon atom to which they are attached, form a C═O.

7. The compound of any of claims 1-6, or a pharmaceutically acceptable salt thereof, wherein:
each R⁴ is, independently, hydrogen or C₁-C₃ alkyl; or two R⁴, together with the carbon atom to which they are attached, form a C═O.

8. The compound of any of claims 1-7, or a pharmaceutically acceptable salt thereof, wherein:
each R⁴, together with the carbon atom to which they are attached, form a C═O.

9. The compound of any of claims 1-8, or a pharmaceutically acceptable salt thereof, wherein:
R⁵ is hydrogen or halogen.

10. The compound of any of claims 1-8, or a pharmaceutically acceptable salt thereof, wherein:
R⁵ is hydrogen or C₁-C₃ alkyl.

11. The compound of any of claims 1-10, or a pharmaceutically acceptable salt thereof, wherein:
R⁵ is hydrogen.

12. The compound of any of claims 1-11, or a pharmaceutically acceptable salt thereof, wherein:
R⁵ is deuterium.

13. The compound of any of claims 1-12, or a pharmaceutically acceptable salt thereof, wherein:
R⁶ is hydrogen.

14. The compound of any of claims 1-13, or a pharmaceutically acceptable salt thereof, wherein:
t is 0.

15. The compound of any of claims 1-14, or a pharmaceutically acceptable salt thereof, wherein:
t is 1.

16. The compound of any of claims 1-14, or a pharmaceutically acceptable salt thereof, wherein:
t is 2.

17. The compound of any of claims 1-16, or a pharmaceutically acceptable salt thereof, wherein:
A is —CH₃.

18. The compound of any of claims 1-16, or a pharmaceutically acceptable salt thereof, wherein:
A is

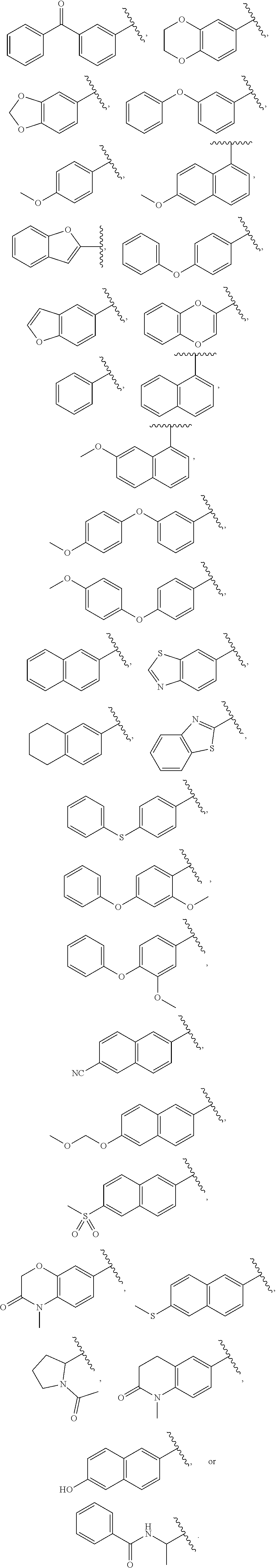

19. The compound of any of claims 1-16, or a pharmaceutically acceptable salt thereof, wherein:
A is

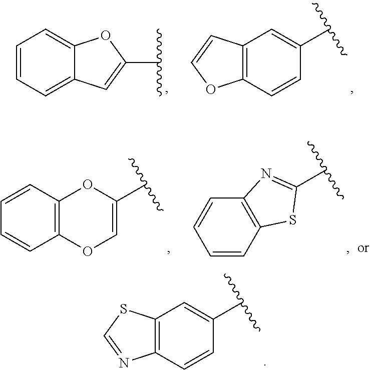

20. The compound of any of claims 1-16, or a pharmaceutically acceptable salt thereof, wherein:
A is

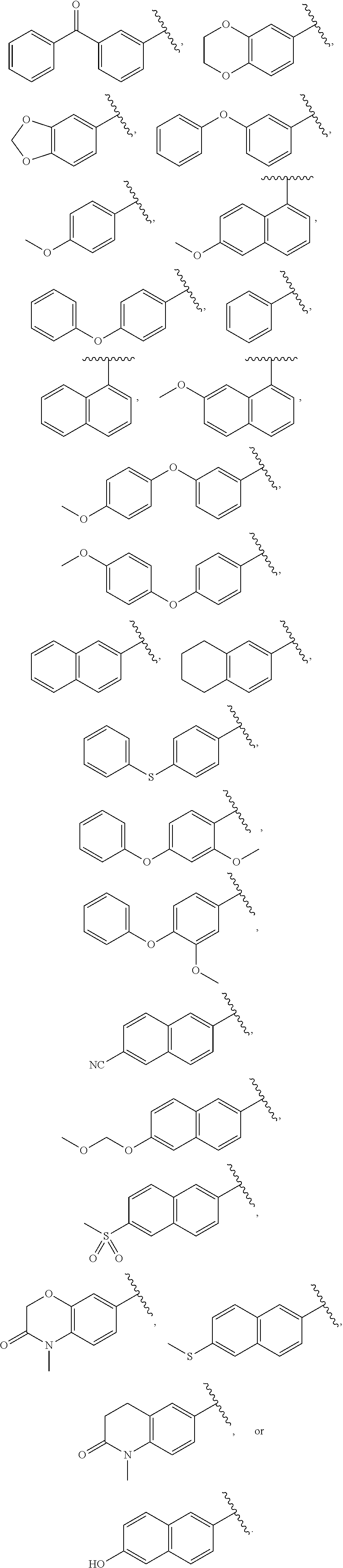

21. The compound of any of claims 1-16, or a pharmaceutically acceptable salt thereof, wherein:
A is

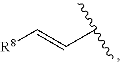

22. The compound of any of claims 1-16, or a pharmaceutically acceptable salt thereof, wherein:
A is 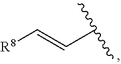
and R⁸ is substituted or unsubstituted carbocyclyl, substituted or unsubstituted heterocyclyl, substituted or unsubstituted aryl, or substituted or unsubstituted heteroaryl.

23. The compound of any of claims 1-16, or a pharmaceutically acceptable salt thereof, wherein:
A is 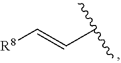
and R⁸ is substituted or unsubstituted aryl.

24. The compound of any of claims 1-16, or a pharmaceutically acceptable salt thereof, wherein:
A is 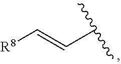
and R⁸ is substituted or unsubstituted phenyl.

25. The compound of any of claims 1-16, or a pharmaceutically acceptable salt thereof, wherein:
A is 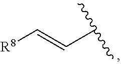
and R⁸ is unsubstituted phenyl or phenyl substituted with alkoxy.

26. The compound of any of claims 1-16, or a pharmaceutically acceptable salt thereof, wherein:
A is 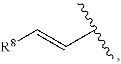
and R⁸ is unsubstituted phenyl or phenyl substituted with C₁₋₄ alkoxy.

27. The compound of any of claims 1-16, or a pharmaceutically acceptable salt thereof, wherein:
A is

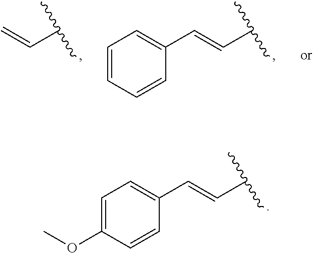

28. The compound of any of claims 1-16, or a pharmaceutically acceptable salt thereof, wherein:
A is

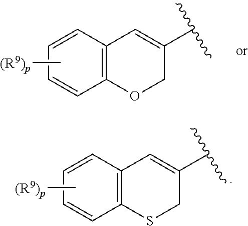

29. The compound of any of claims 1-16, or a pharmaceutically acceptable salt thereof, wherein:
A is 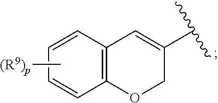
and each R⁹ is independently halogen, substituted or unsubstituted alkyl, substituted or unsubstituted heteroaliphatic, —OR^{A}, —N(R^{A})₂, —SR^{A}, or —CN.

30. The compound of any of claims 1-16, or a pharmaceutically acceptable salt thereof, wherein:
A is 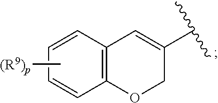
and each R⁹ is independently halogen, substituted or unsubstituted alkyl, or —OR^{A}.

31. The compound of any of claims 1-16, or a pharmaceutically acceptable salt thereof, wherein:
A is 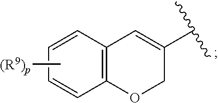
wherein each R⁹ is independently halogen, unsubstituted alkyl, haloalkyl, or —OR^{A}; and R^{A} is haloalkyl or unsubstituted alkyl.

32. The compound of any of claims 1-16, or a pharmaceutically acceptable salt thereof, wherein:
A is 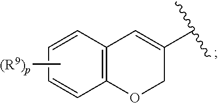
and each R⁹ is independently halogen, C₁₋₄ alkyl, C₁₋₄haloalkyl, —OC₁₋₄ haloalkyl, or —OC₁₋₄ alkyl.

33. The compound of any of claims 1-16, or a pharmaceutically acceptable salt thereof, wherein:
A is 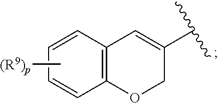
and each R⁹ is independently —Br, —CH₃, —CF₃, —OCF₃, or —OCH₃.

34. The compound of any of claim 1-16 or 28-33, or a pharmaceutically acceptable salt thereof, wherein:
p is 0, 1, or 2.

35. The compound of any of claim 1-16 or 28-33, or a pharmaceutically acceptable salt thereof, wherein:
p is 1.

36. The compound of any of claim 1-16 or 28-35, or a pharmaceutically acceptable salt thereof, wherein:
A is

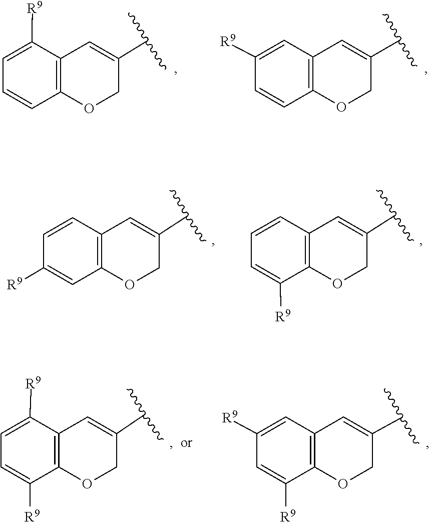

37. The compound of any of claim 1-16 or 28-36, or a pharmaceutically acceptable salt thereof, wherein:
A is

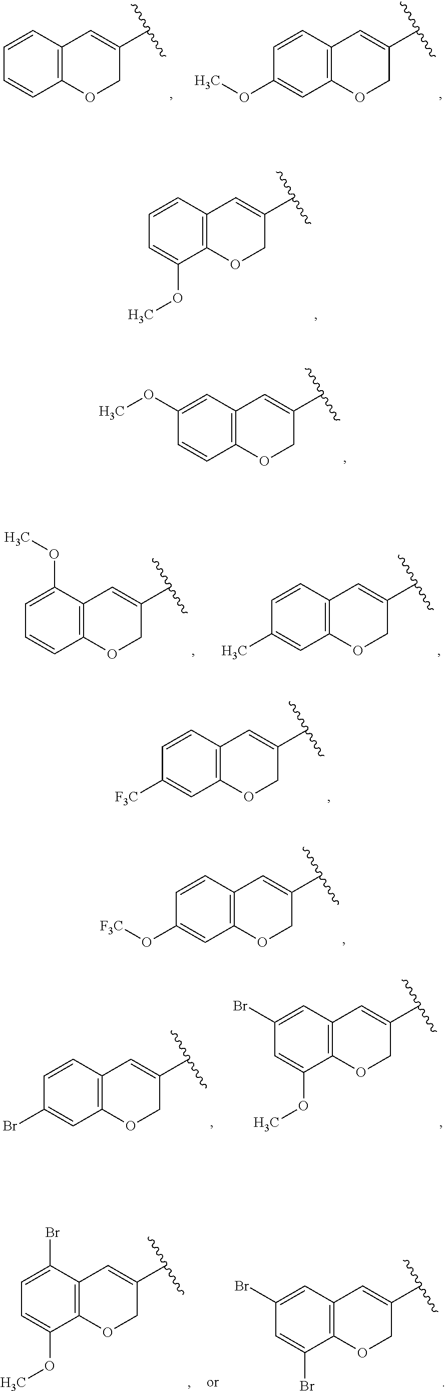

38. The compound of claim 1, wherein the compound is of Formula (I-a): 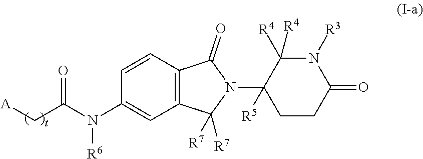
or a pharmaceutically acceptable salt thereof.

39. The compound of claim 1, wherein the compound is of Formula (I-b): 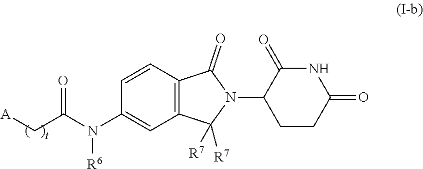
or a pharmaceutically acceptable salt thereof.

40. The compound of claim 1, wherein the compound is of Formula (I-c): 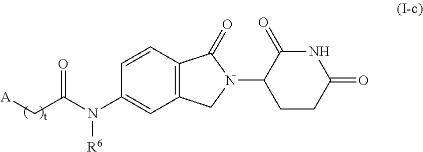
or a pharmaceutically acceptable salt thereof.

41. The compound of claim 1, wherein the compound is of Formula (I-d): 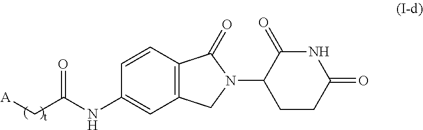
or a pharmaceutically acceptable salt thereof.

42. The compound of claim 1, wherein the compound is of Formula (I-e): 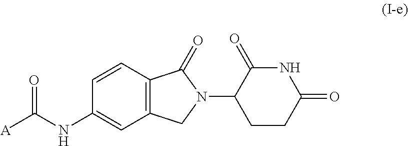
or a pharmaceutically acceptable salt thereof.

43. The compound of claim 1, wherein the compound is of Formula (I-f): 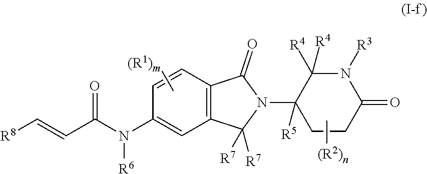
or a pharmaceutically acceptable salt thereof.

44. The compound of claim 1, wherein the compound is of Formula (I-g): 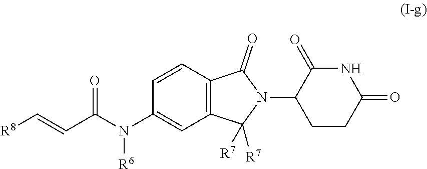
or a pharmaceutically acceptable salt thereof.

45. The compound of claim 1, wherein the compound is of Formula (I-h): 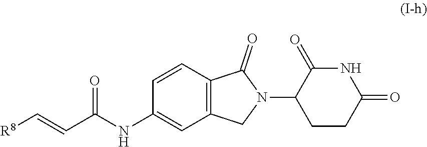
or a pharmaceutically acceptable salt thereof.

46. The compound of claim 1, wherein the compound is of Formula (I-i): 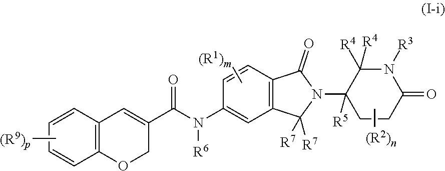
or a pharmaceutically acceptable salt thereof.

47. The compound of claim 1, wherein the compound is of Formula (I-j): 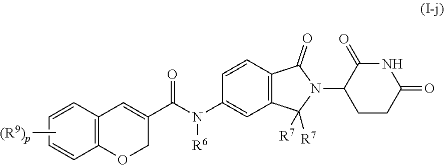
or a pharmaceutically acceptable salt thereof.

48. The compound of claim 1, wherein the compound is of Formula (I-k): 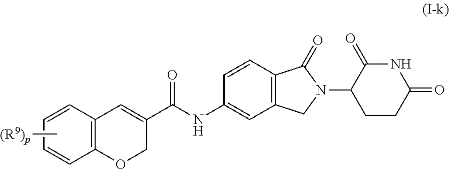
or a pharmaceutically acceptable salt thereof.

49. The compound of claim 1, wherein the compound is of Formula (I-l): 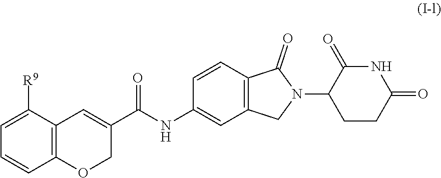
or a pharmaceutically acceptable salt thereof.

50. The compound of claim 1, wherein the compound is of Formula (I-m): 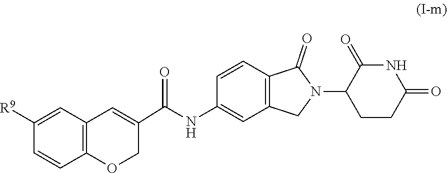
or a pharmaceutically acceptable salt thereof.

51. The compound of claim 1, wherein the compound is of Formula (I-n): 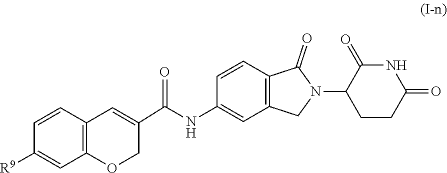
or a pharmaceutically acceptable salt thereof.

52. The compound of claim 1, wherein the compound is of Formula (I-o): 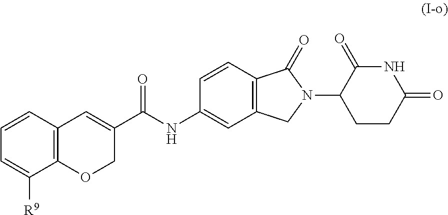
or a pharmaceutically acceptable salt thereof.

53. The compound of claim 1, wherein the compound is of Formula (I-p): 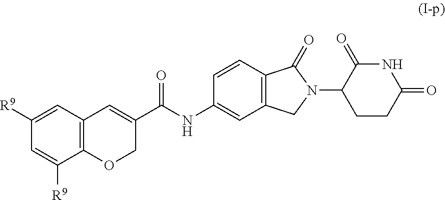
or a pharmaceutically acceptable salt thereof.

54. The compound of claim 1, wherein the compound is of Formula (I-q): 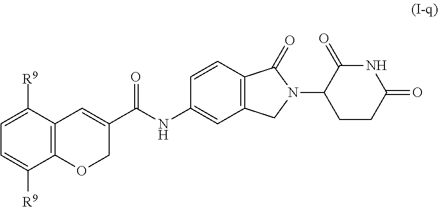
or a pharmaceutically acceptable salt thereof.

55. The compound of claim 1, wherein the compound is 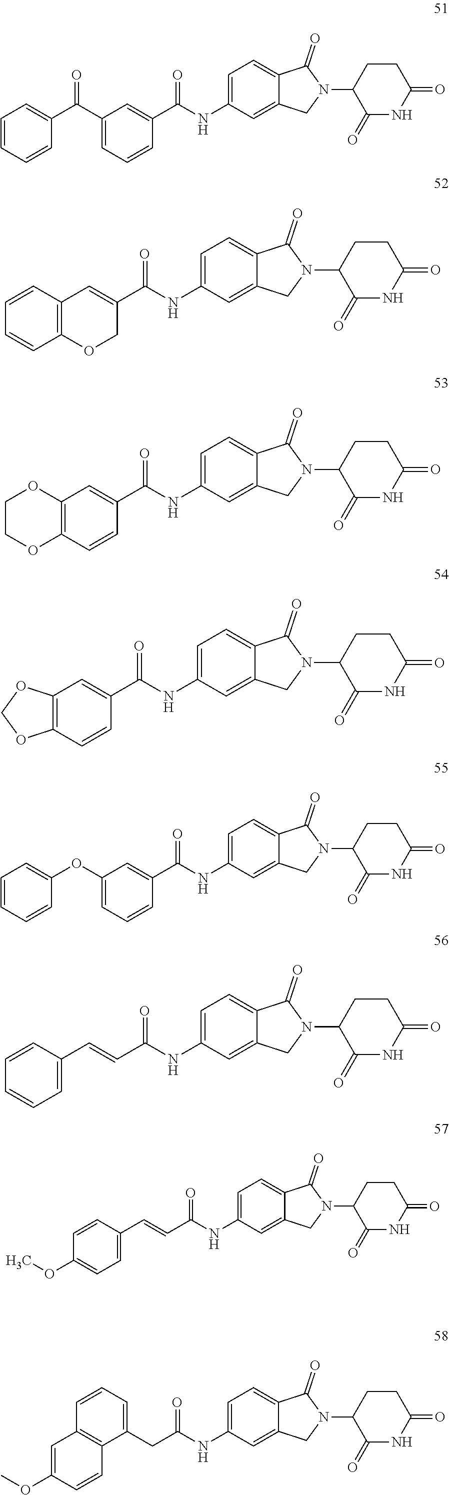 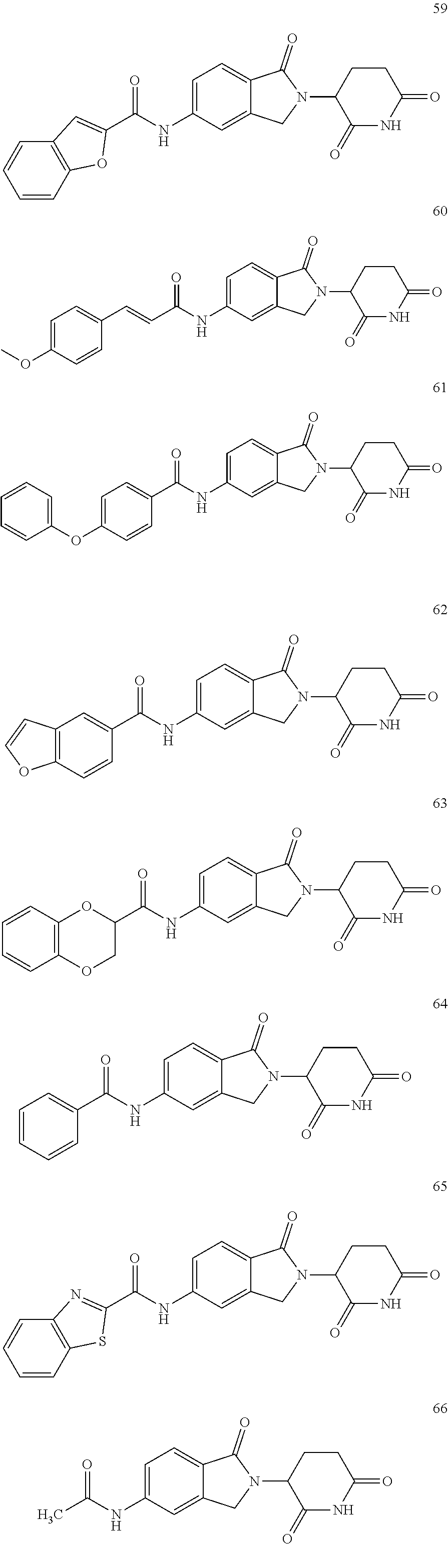 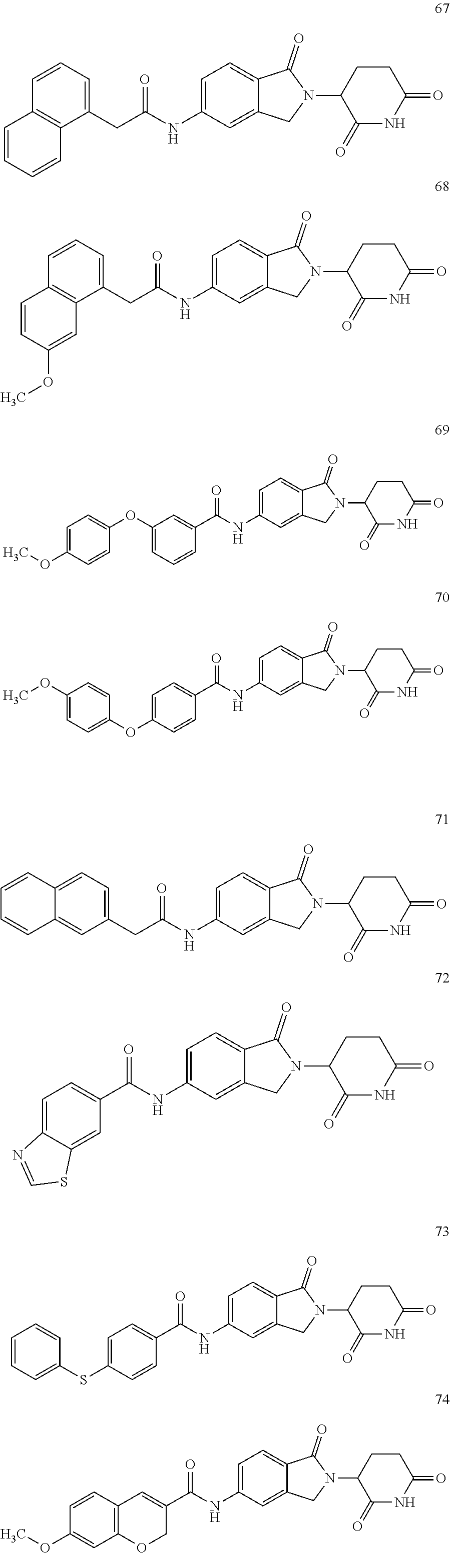 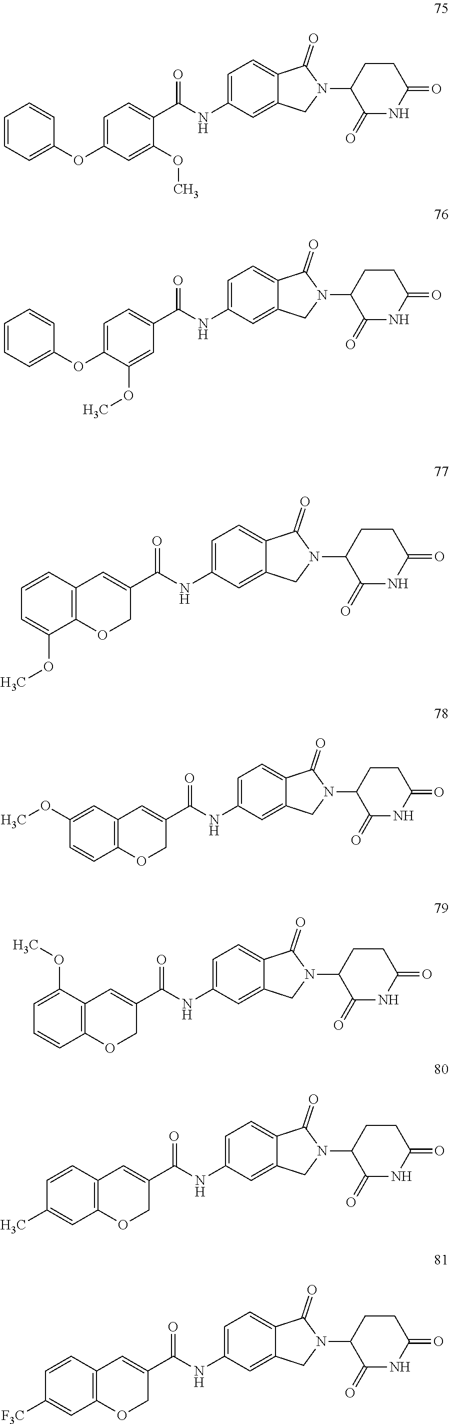 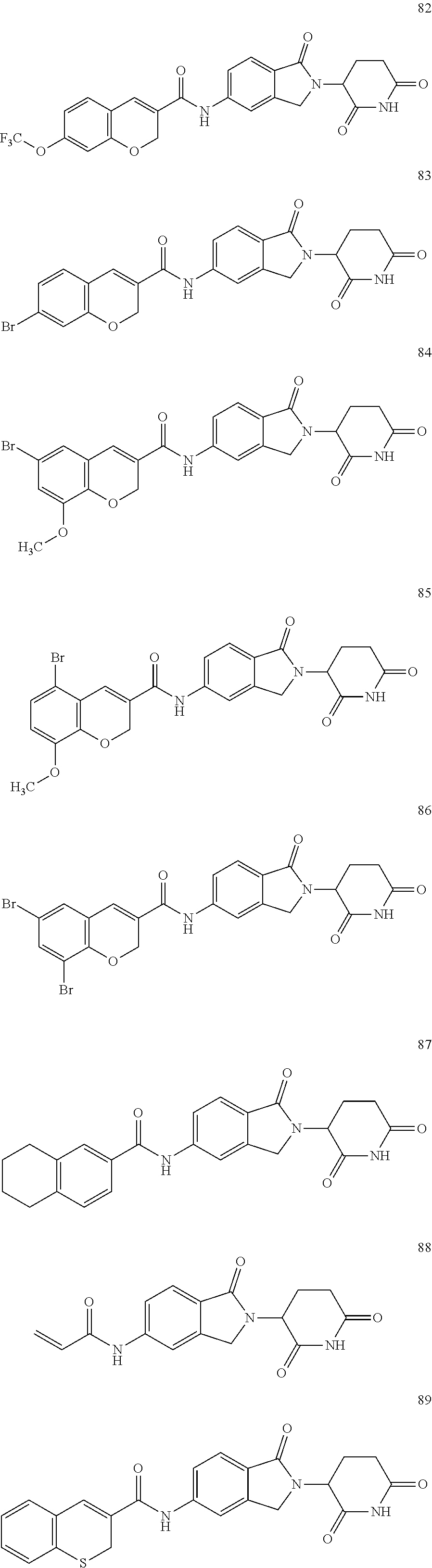 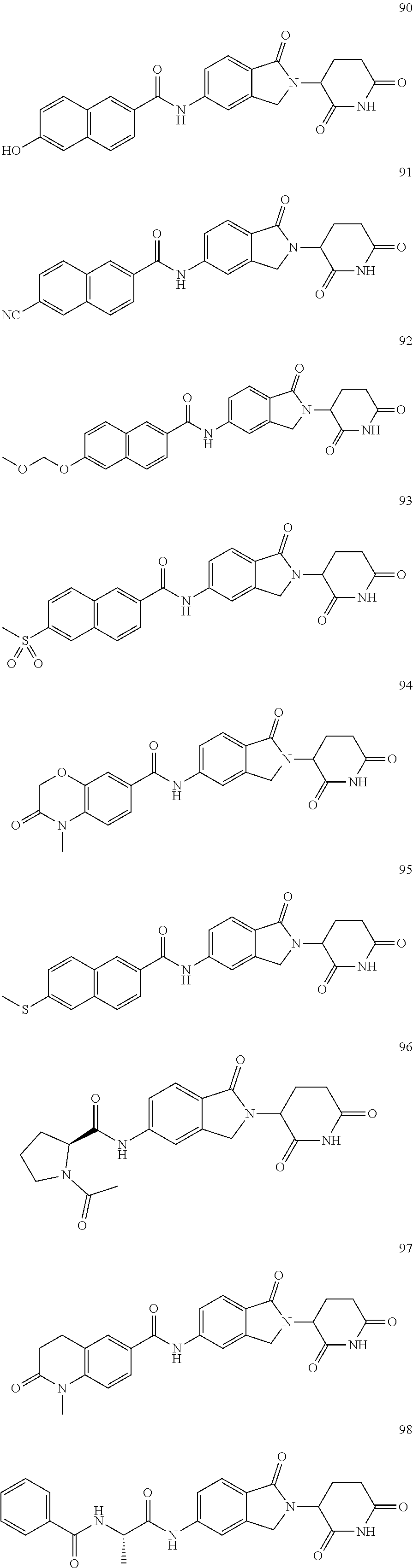
or a pharmaceutically acceptable salt thereof.

56. A pharmaceutical composition comprising a compound of any one of claims 1-55, or a pharmaceutically acceptable salt thereof, and a pharmaceutically acceptable excipient.

57. A method of treating cancer in a subject in need thereof, the method comprising administering a compound of any one of claims 1-55, or a pharmaceutically acceptable salt thereof, or a pharmaceutical composition of claim 56 to the subject.

58. The method of claim 57, wherein the cancer is a pediatric cancer (e.g., childhood acute leukemia (AL) or medulloblastoma (MB)), lung cancer, breast cancer, colon cancer, colorectal cancer, ovarian cancer, gastric cancer, or a hematological cancer.

59. The method of claim 57 or 58, wherein the cancer is a hematological cancer.

60. The method of any of claims 57-59, wherein the cancer is a leukemia or a lymphoma.

61. The method of any one of claims 57-60, wherein the cancer is primary effusion lymphoma (PEL), del(5q) myelodysplastic syndrome (MDS), chronic myeloid leukemia (CML), chronic lymphoid leukemia (CLL), acute myeloid leukemia (AML), hairy cell leukemia, chronic myelomonocytic leukemia (CMML), juvenile myelomonocyte leukemia (JMML), large granular lymphocytic leukemia (LGL), acute lymphoblastic leukemia (ALL), B-cell acute lymphoblastic leukemia (B-ALL), or T-cell acute lymphoblastic leukemia (T-ALL), B-cell lymphoma, diffuse large B-cell lymphoma (DLBCL), T-cell-lymphoma, Hodgkin's lymphoma, non-Hodgkin's lymphoma, hairy cell lymphoma, or Burkett's lymphoma.

62. The method of any one of claims 57-61, wherein the cancer is acute myeloid leukemia (AML).

63. The method of claim 57 or 58, wherein the cancer is ovarian cancer.

64. A method of promoting the degradation of IKAROS family zinc finger 1 (IKZF1), the method comprising contacting IKZF1 with a compound of any one of claims 1-56, or a pharmaceutically acceptable salt thereof, or a pharmaceutical composition of claim 57.

65. A method of promoting the degradation of IKAROS family zinc finger 2 (IKZF2), the method comprising contacting IKZF2 with a compound of any one of claims 1-56, or a pharmaceutically acceptable salt thereof, or a pharmaceutical composition of claim 57.

66. A method of promoting the degradation of casein kinase 1α (CK1α), the method comprising contacting CK1α with a compound of any one of claims 1-56, or a pharmaceutically acceptable salt thereof, or a pharmaceutical composition of claim 57.

67. A method of promoting the degradation of G1 to S phase transition 1 protein (GSPT1), the method comprising contacting GSPT1 with a compound of any one of claims 1-56, or a pharmaceutically acceptable salt thereof, or a pharmaceutical composition of claim 57.

68. The method of any of claims 64-67, wherein the degradation is in a cell.

69. The method of any of claims 64-68, wherein the degradation is in a subject.

70. The method of any of claims 64-69, wherein the degradation is in a biological sample.

71. A compound of any of claims 1-56, or a pharmaceutically acceptable salt thereof, or a pharmaceutical composition of claim 57, for use in treating cancer in a subject in need thereof.

72. The compound or pharmaceutical composition of claim 71, wherein the cancer is a pediatric cancer (e.g., childhood acute leukemia (AL) or medulloblastoma (MB)), lung cancer, breast cancer, colon cancer, colorectal cancer, ovarian cancer, gastric cancer, or a hematological cancer.

73. The compound or pharmaceutical composition of claim 71 or 72, wherein the cancer is a hematological cancer.

74. The compound or pharmaceutical composition of any of claims 71-73, wherein the cancer is a leukemia or a lymphoma.

75. The compound or pharmaceutical composition of any of claims 71-74, wherein the cancer is primary effusion lymphoma (PEL), del(5q) myelodysplastic syndrome (MDS), chronic myeloid leukemia (CML), chronic lymphoid leukemia (CLL), acute myeloid leukemia (AML), hairy cell leukemia, chronic myelomonocytic leukemia (CMML), juvenile myelomonocyte leukemia (JMML), large granular lymphocytic leukemia (LGL), acute lymphoblastic leukemia (ALL), B-cell acute lymphoblastic leukemia (B-ALL), or T-cell acute lymphoblastic leukemia (T-ALL), B-cell lymphoma, diffuse large B-cell lymphoma (DLBCL), T-cell-lymphoma, Hodgkin's lymphoma, non-Hodgkin's lymphoma, hairy cell lymphoma, or Burkett's lymphoma.

76. The compound or pharmaceutical composition of any of claims 70-74, wherein the cancer is acute myeloid leukemia (AML).

77. The compound or pharmaceutical composition of claim 71, wherein the cancer is ovarian cancer.

78. A kit comprising a compound of any one of claims 1-56, or a pharmaceutically acceptable salt thereof, or a pharmaceutical composition of claim 57; and instructions for administering the compound, the pharmaceutically acceptable salt thereof, or the pharmaceutical composition to a subject.
